(19) United States
(12) Patent Application Publication
(10) Pub. No.: US 2024/0206414 A1
(43) Pub. Date: Jun. 27, 2024

(54) INTROGRESSION OF TOLCNDV-ES RESISTANCE CONFERRING QTLS IN CUCUMIS SATIVUS PLANTS

(71) Applicant: NUNHEMS B.V., Nunhem (NL)

(72) Inventors: Daniele LIBERTI, El Ejido Almeria (ES); Hans Peter KOELEWIJN, Nunhem (NL); Nicky DRIEDONKS, Nunhem (NL); Gulay CANGAL, Nunhem (NL); Robert CHYNOWETH, La Palma-Cartagena (ES)

(21) Appl. No.: 18/287,713

(22) PCT Filed: Apr. 19, 2022
(86) PCT No.: PCT/EP2022/060297

§ 371 Date: Oct. 20, 2023

(30) Foreign Application Priority Data
Apr. 22, 2021 (EP) 21169794.1

(51) Int. Cl. A01H 1/00; A01H 1/04; A01H 5/08; A01H 6/34; C12Q 1/6895

(52) U.S. Cl. CPC A01H 1/126; A01H 1/045; A01H 5/08; A01H 6/346; C12Q 1/6895; C12Q 2600/156

(57) ABSTRACT

The present invention relates to cultivated cucumber plants comprising one or more QTLs on chromosome 1, 2 and/or 3 of their genome conferring enhanced ToLCNDV-ES resistance, and to methods for generating such plants, and their use.

### FIELD

The present invention relates to the field of cucumber breeding. Provided are four Quantitative Trait Loci (QTLs) located on chromosome 1 (QTL1.1 and QTL1.2), on chromosome 2 (QTL2.1) and on chromosome 3 (QTL3.1) of the cucumber genome, which can be used alone or in combinations to increase ToLCNDV-ES resistance in cultivated cucumbers (Cucumis sativus var. sativus), such as pickling cucumbers (e.g. American pickling, European pickling types), slicing cucumbers (e.g. American slicing), long cucumbers, short cucumbers, European greenhouse cucumbers or Beit-Alpha type cucumbers.

Tomato Leaf Curl New Delhi Virus (ToLCNDV) is a bi-partite begonovirus which is transmitted by white fly vectors, Bemisia tabaci Genn. (Hemiptera: Aleyrodidae) and causes severe symptoms of leaf yellowing and curling and plant stunting in susceptible hosts.

Until 2016 ToLCNDV was limited to India and other Asian countries, where it was mainly prevalent on Solanaceae host plants, especially tomato, but since then it has been reported in Spain and other countries of the Mediterranean basin, were it is mostly prevalent on Cucurbitaceae hosts (Fortes et al., Viruses 2016, 8, 307; doi:10.3390/v8110307).

The Mediterranean ToLCNDV isolates (also referred to as the European ToLCNDV isolates) are referred to as ToLCNDV-ES and it is now known that all ToLCNDV-ES isolates are highly genetically uniform (>99% identity, Juarez et al, 2019, Natural hosts and genetic diversity of the emerging tomato leaf curl New Delhi virus in Spain. Frontiers Microbiology, 10, 140. https://doi.org/10.3389/fnicb.2019.00140) and are distinct from all other ToLCNDV isolates found outside Europe. Although ToLCNDV-ES is infective to tomato (Ruiz et al., 2017, Biological characterization of Tomato leaf curl New Delhi virus from Spain. Plant Pathology, 66, 376-382), TolCNDV-ES appears poorly adapted to this host; in contrast, it very efficiently infects Cucurbitaceae like squash, melon, cucumber and pumpkin.

This genetic uniformity of the European isolates is also thought to explain their preference and adaptation to Cucurbitaceae hosts.

As the ToLCNDV-ES isolates are prevalent in all cucumber growing areas where Bemisia tabaci is found, especially in Spain, Italy, Greece and other Mediterranean countries, but also in greenhouse cultivation in northern European countries, there is a need to provide cucumber plants which are resistant to ToLCNDV-ES.

WO2021019069 (and the priority document WO2021019272) describes cucumber plants that are tolerant to Tomato Leaf Curl New Delhi Virus comprising in its genome a first QTL, QTL1, on chromosome 1 and a second QTL, QTL2 on chromosome 2, whereby at least one of the QTLs is in homozygous form. Twelve SNP markers are said to be linked to QTL1 (Table 3) and 15 to QTL2 (Table 4), with the nucleotide indicative of ‘Tolerance’ being shown under the heading T-allele in Tables 3 and 4. Which ToLCNDV isolates the resistance is effective against is not mentioned. QTL1 and QTL2 are from a tolerant donor called CUC29 (landrace). Both QTL1 and QTL2 are said to be necessary to confer the tolerance (p7, line 24-25) and the reached level of tolerance seems to be a level of around 7 (more resistant than IR but not resistant), on a scale of 1 (very susceptible) to 9 (resistant), see FIG. 4. A seed deposit was made of a BC1F3 line containing QTL1 and QTL2 in homozygous form from CUC29, which was given accession number NCIMB43427.

Saez et al. (Microorganisms 9, 913) published on 24 Apr. 2021 a study entitled “Resistant Sources and Genetic Control of Resistance to ToLCNDV in cucumber”. In the study they find that resistance of accession CGN23089 fits a recessive monogenic inheritance model (abstract). They conclude “Our inheritance analyses indicate that the resistance to ToLCNDV in the CGN23089 accession is mainly controlled by one recessive gene, and this was supported by the detection of one QTL on chromosome 2 of the C. sativus genome. The closest marker mapped was SNPC2_3 located at physical position 12,760,375 bp of the cucumber Gy_14v2 genome (corresponding to position 12,910,596 of the Chinese long V3 genome). In Table S3 they identify more than 490 genes that lie in the region around SNPC2_3 which should contain candidate genes for the ToLCNDV resistance.

There is, therefore, a need to provide resistance to ToLCNDV-ES isolates in cucumber, to protect cucumber plants from these isolates.

Four QTLs have been mapped herein, referred to as QTL1.1, QTL1.2, QTL2.1 and QTL3.1, see FIG. 1. The three QTLs on chromosome 1 and chromosome 2 are major QTLs, the QTL on chromosome 3 is a more minor, but still significant QTL. The wild QTL donor and seeds of an F3 line into which all four QTLs have been introgressed (and of which a representative sample of seeds has been deposited by the applicant under accession number NCIMB43745) have a high resistance to ToLCNDV-ES, with no symptoms developing (average disease score of 9.0 on a scale of 2.0 to 9.0, with 2.0 being ‘leaves fully covered with yellowing mosaic’ and 9.0 being free of symptoms).

For each of the four QTLs SNP markers (Single Nucleotide Polymorphism) are provided which are linked to the QTLs and which can be used to detect plants or plant parts comprising one or more of the QTLs, to select one or more of the QTLs in breeding programs or to identify other wild donors which comprise one or more of the QTLs.

For example, other donors have been identified herein which contain the same, or almost the same, SNP haplotype for these four QTLs and which are resistant against ToLCNDV-ES.

Thus, in one aspect cultivated cucumber plants and plant parts are provided herein comprising one or more of the following introgression fragments: an introgression fragment on chromosome 1 comprising QTL1.1, an introgression fragment on chromosome 1 comprising QTL1.2, an introgression fragment on chromosome 2 comprising QTL2.1 and an introgression fragment on chromosome 3 comprising QTL3.1, whereby the (one or more) introgression fragments significantly increase resistance against ToLCNDV-ES of the cultivated cucumber comprising the (one or more) introgression fragments compared to the same (or control) cultivated cucumber lacking all of the introgression fragments.

The introgression fragments, comprising QTL1.1, QTL1.2, QTL2.1 and/or QTL3.1, are from a wild or primitive cucumber donor, which was highly resistant to ToLCNDV-ES. The QTLs were identified in different mapping populations, obtained by crossing the donor plant with different cultivated cucumber lines, such as an French slicer elite line, a European long cucumber elite line and a Spanish black slicer elite line. The seeds that were deposited and comprise all four QTLs in homozygous form are from one of the slicer populations. From this type each of the QTLs can easily be transferred into any other cultivated cucumber type, such as short cucumber types, or into long cucumber breeding lines or varieties or into cucumber slicer type lines or varieties. Seeds comprising the introgression fragments comprising QTL1.1, QTL1.2, QTL2.1 and QTL3.1 were deposited under accession number NCIMB43745.

Also one or more molecular markers (especially Single Nucleotide Polymorphisms or SNPs) which are present on each of the introgression fragments and which are indicative of the presence of the introgression fragment and methods of using such markers are provided herein. Likewise seeds, plant parts, cells and/or tissues comprising QTL1.1 and/or QTL1.2 and/or QTL2.1 and/or QTL3.1 in their genome and comprising otherwise a genome of cultivated cucumber in their genome are provided. It is noted that the term “genome of cultivated cucumber” does not exclude that there are other introgression fragments in the entire genome, e.g. on other chromosomes and/or for other traits.

Likewise seeds, plant parts, cells and/or tissues of cultivated cucumber comprising QTL1.1 and/or QTL1.2 and/or QTL2.1 and/or QTL3.1 are provided. In a preferred aspect seeds, plant parts and/or tissues of cultivated cucumber comprising QTL1.1 and QTL1.2 are provided, or comprising QTL1.1 and QTL1.2 and QTL2.1 are provided, or comprising QTL1.1 and QTL2.1 are provided, or comprising QTL1.2 and QTL2.1 are provided. As QTL1.1, QTL1.2 and QTL3.1 each have an additive effect, plants and plant parts comprising one or more of these three QTLs are also an embodiment herein, e.g. in heterozygous or homozygous form. Also plants and plant parts comprising all four QTLs are encompassed herein, preferably in homozygous form or at least QTL2.1 being in homozygous form.

A QTL having an additive effect means that when the QTL is in heterozygous form the effect on ToLCNDV-ES resistance is less than when the QTL is in homozygous form, but still significantly above the control line lacking the QTL. QTL1.1, QTL1.2 and QTL3.1 each have such an additive effect, meaning they these QTLs can be used in heterozygous form or in homozygous form. QTL2.1, on the other hand, has a recessive or partially recessive effect, meaning the effect on ToLCNDV-ES resistance is mainly seen when the QTL2.1 is in homozygous form. In certain backgrounds also a slight effect may be seen when QTL2.1 is in heterozygous form (i.e. QTL2.1 is partially recessive in certain backgrounds).

As shown in the Examples, the presence of all four QTLs results in the highest resistance level. However, also the combinations of three QTLs resulted in very high resistance, with an average score of above 8.0. Individual QTLs slightly increased resistance, with a higher increase for two QTLs. Therefore, plants and plant parts comprising at least three QTLs selected from QTL1.1, QTL1.2, QTL2.1 and QTL3.1 is one aspect herein. Also, plants and plant parts comprising at least QTL1.1 alone or in combination with at least one further QTL selected from QTL1.2, QTL2.1 and QTL3.1, or with at least two further QTLs selected from QTL1.2, QTL2.1 and QTL3.1 is one embodiment herein. In another aspect plants and plant parts comprising at least QTL3.1 alone or in combination with at least one further QTL selected from QTL1.1, QTL1.2 and QTL2.1, or with at least two further QTLs selected from selected from QTL1.1, QTL1.2 and QTL2.1 is one embodiment herein.

In one aspect the cultivated cucumber plants, seeds, plant parts, cells and/or tissues comprise the introgression fragment from a wild or primitive cucumber, whereby the introgression fragment comprises QTL1.1, which is located physically in the region starting at nucleotide 8235142 (corresponding to SNP_01 at nucleotide 51 of SEQ ID NO: 01) and ending at nucleotide 16209127 (corresponding to SNP_16 at nucleotide 51 of SEQ ID NO: 16) of chromosome 1, with reference to the chromosome 1 of the Chinese Long V3 reference genome (cucurbitgenomics.org). In one aspect the introgression fragment comprising the QTL1.1 comprises the donor nucleotide for at least 5, 6, 7, 8, 9, 10, 11, 12 or more SNP markers selected from SNP_01 to SNP_16, especially for at least 5, 6, 7, 8, 9, 10, 11, 12 or more SNP markers selected from SNP_02 to SNP_15. In one aspect said at least 5 markers include one or more of the following SNP markers: SNP_10 at nucleotide 51 of SEQ ID NO: 10 or at the equivalent position in a sequence comprising at least 95% sequence identity to SEQ ID NO: 10, SNP_11 at nucleotide 51 of SEQ ID NO: 11 or at the equivalent position in a sequence comprising at least 95% sequence identity to SEQ ID NO: 11 and/or SNP_15 at nucleotide 51 of SEQ ID NO: 15 or at the equivalent position in a sequence comprising at least 95% sequence identity to SEQ ID NO: 15.

In one aspect the cultivated cucumber plants, seeds, plant parts, cells and/or tissues comprise the introgression fragment from a wild or primitive cucumber, whereby the introgression fragment comprises QTL1.2, which is located physically in the region starting at nucleotide 22942981 (corresponding to SNP_17 at nucleotide 51 of SEQ ID NO: 17) and ending at nucleotide 25543032 (corresponding to SNP_31 at nucleotide 51 of SEQ ID NO: 31) of chromosome 1, with reference to the chromosome 1 of the Chinese Long V3 reference genome (cucurbitgenomics.org). In one aspect the introgression fragment comprising the QTL1.2 comprises the donor nucleotide for at least 5, 6, 7, 8, 9 10, 11, 12 or more SNP markers selected from SNP_17 to SNP_31, especially for at least 5, 6, 7, 8, 9, 10, 11, 12 or more SNP markers selected from SNP_18 to SNP_30. In one aspect said at least 5 markers include one or more of the following SNP markers: SNP_22 at nucleotide 51 of SEQ ID NO: 22 or at the equivalent position in a sequence comprising at least 95% sequence identity to SEQ ID NO: 22 and/or SNP_23 at nucleotide 51 of SEQ ID NO: 23 or at the equivalent position in a sequence comprising at least 95% sequence identity to SEQ ID NO: 23 and/or SNP_25 at nucleotide 51 of SEQ ID NO: 25 or at the equivalent position in a sequence comprising at least 95% sequence identity to SEQ ID NO: 25 and/or SNP_26 at nucleotide 51 of SEQ ID NO: 26 or at the equivalent position in a sequence comprising at least 95% sequence identity to SEQ ID NO: 26 and/or SNP_28 at nucleotide 51 of SEQ ID NO: 28 or at the equivalent position in a sequence comprising at least 95% sequence identity to SEQ ID NO: 28 and/or SNP_29 at nucleotide 51 of SEQ ID NO: 29 or at the equivalent position in a sequence comprising at least 95% sequence identity to SEQ ID NO: 29.

In one aspect the cultivated cucumber plants, seeds, plant parts, cells and/or tissues comprise the introgression fragment from a wild or primitive cucumber, whereby the introgression fragment comprises QTL2.1, which is located physically in the region starting at nucleotide 15218569 (corresponding to SNP_32 at nucleotide 51 of SEQ ID NO: 32) and ending at nucleotide 19535432 (corresponding to SNP 47 at nucleotide 51 of SEQ ID NO: 47) of chromosome 2, with reference to the chromosome 2 of the Chinese Long V3 reference genome (cucurbitgenomics.org). In one aspect the introgression fragment comprising the QTL2.1 comprises the donor nucleotide for at least 5, 6, 7, 8, 9, 10, 11, 12 or more SNP markers selected from SNP_32 to SNP_47, especially for at least 5, 6, 7, 8, 9, 10, 11, 12 or more SNP markers selected from SNP_33 to SNP_46. In one aspect said at least 5 markers include one or more of the following SNP markers: SNP_41 at nucleotide 51 of SEQ ID NO: 41 or at the equivalent position in a sequence comprising at least 95% sequence identity to SEQ ID NO: 41 and/or SNP_45 at nucleotide 51 of SEQ ID NO: 45 or at the equivalent position in a sequence comprising at least 95% sequence identity to SEQ ID NO: 45.

In one aspect the cultivated plants, seeds, plant parts, cells and/or tissues comprise the introgression fragment from a wild or primitive cucumber, whereby the introgression fragment comprises QTL3.1, which is located physically in the region starting at nucleotide 3637 (corresponding to SNP_48 at nucleotide 51 of SEQ ID NO: 48) and ending at nucleotide 3885803 (corresponding to SNP_62 at nucleotide 51 of SEQ ID NO: 62) of chromosome 3, with reference to the chromosome 3 of the Chinese Long V3 reference genome (cucurbitgenomics.org). In one aspect the introgression fragment comprising the QTL3.2 comprises the donor nucleotide for at least 5, 6, 7, 8, 9, 10, 11, 12 or more SNP markers selected from SNP_48 to SNP_62, especially for at least 5, 6, 7, 8, 9, 10, 11, 12 or more SNP markers selected from SNP_49 to SNP_61. In one aspect said at least 5 markers include one or more of the following SNP markers: SNP_49 at nucleotide 51 of SEQ ID NO: 49 or at the equivalent position in a sequence comprising at least 95% sequence identity to SEQ ID NO: 49 and/or SNP_50 at nucleotide 51 of SEQ ID NO: 50 or at the equivalent position in a sequence comprising at least 95% sequence identity to SEQ ID NO: 50 and/or SNP_51 at nucleotide 51 of SEQ ID NO: 51 or at the equivalent position in a sequence comprising at least 95% sequence identity to SEQ ID NO: 51 and/or SNP_52 at nucleotide 51 of SEQ ID NO: 52 or at the equivalent position in a sequence comprising at least 95% sequence identity to SEQ ID NO: 52 and/or SNP_55 at nucleotide 51 of SEQ ID NO: 55 or at the equivalent position in a sequence comprising at least 95% sequence identity to SEQ ID NO: 55 and/or SNP_57 at nucleotide 51 of SEQ ID NO: 57 or at the equivalent position in a sequence comprising at least 95% sequence identity to SEQ ID NO: 57 and/or SNP_58 at nucleotide 51 of SEQ ID NO: 58 or at the equivalent position in a sequence comprising at least 95% sequence identity to SEQ ID NO: 58 and/or SNP_60 at nucleotide 51 of SEQ ID NO: 60 or at the equivalent position in a sequence comprising at least 95% sequence identity to SEQ ID NO: 60.

In one aspect a cultivated Cucumis sativus var. sativus plant comprising one or more introgression fragments from a wild donor cucumber, selected from QTL1.1, QTL1.2, QTL2.1 and QTL3.1, in homozygous or heterozygous form is provided, wherein said introgression fragment comprises
a Quantitative Trait Locus (QTL1.1) located between the Single Nucleotide Polymorphism marker SNP_01 at nucleotide 51 of SEQ ID NO: 1 (or of a variant of SEQ ID NO: 1) and the Single Nucleotide Polymorphism marker SNP_16 at nucleotide 51 of SEQ ID NO: 16 (or of a variant of SEQ ID NO: 16), which QTL confers an increase in ToLCNDV-ES resistance when in homozygous or heterozygous form,
a Quantitative Trait Locus (QTL1.2) located between the Single Nucleotide Polymorphism marker SNP_17 at nucleotide 51 of SEQ ID NO: 17 (or of a variant of SEQ ID NO: 17) and the Single Nucleotide Polymorphism marker SNP_31 at nucleotide 51 of SEQ ID NO: 31 (or of a variant of SEQ ID NO: 31), which QTL confers an increase in ToLCNDV-ES resistance when in homozygous or heterozygous form,
a Quantitative Trait Locus (QTL2.1) located between the Single Nucleotide Polymorphism marker SNP_32 at nucleotide 51 of SEQ ID NO: 32 (or of a variant of SEQ ID NO: 32) and the Single Nucleotide Polymorphism marker SNP_47 at nucleotide 51 of SEQ ID NO: 47 (or of a variant of SEQ ID NO: 47), which QTL confers an increase in ToLCNDV-ES resistance when in homozygous form,
a Quantitative Trait Locus (QTL3.1) located between the Single Nucleotide Polymorphism marker SNP_48 at nucleotide 51 of SEQ ID NO: 48 (or of a variant of SEQ ID NO: 48) and the Single Nucleotide Polymorphism marker SNP-62 at nucleotide 51 of SEQ ID NO: 62 (or of a variant of SEQ ID NO: 62), which QTL confers an increase in ToLCNDV-ES resistance when in homozygous or heterozygous form.

In one aspect QTL1.1, QTL1.2, QTL2.1 and/or QTL3.1 (i.e. the introgression fragment comprising the QTL) is present in heterozygous form in a cultivated cucumber plant, cell or tissue, especially in long cucumber or slicer cucumber. In one aspect at least QTL2.1 is present in homozygous form in a cultivated cucumber plant, cell or tissue.

In another aspect QTL1.1, QTL1.2, QTL2.1 and/or QTL3.1 (i.e. the introgression fragment comprising the QTL) is present in homozygous form in a cultivated cucumber plant, cell or tissue, especially in long cucumber or slicer cucumber.

In a specific aspect the cultivated cucumber plant is an F1 hybrid, especially an F1 hybrid generated by crossing two inbred parent lines, whereby at least one of the parent lines comprises the QTL1.1, QTL1.2, QTL2.1 and/or QTL3.1 (i.e. the introgression fragment comprising the QTL), preferably in homozygous form. In one aspect at least one, preferably both parent lines comprise QTL2.1 in homozygous form, so that the F1 hybrid comprises QTL2.1 in homozygous form.

In one aspect the donor of one or more of the QTLs is selected from a donor comprising the same SNP haplotype for at least 5, 6, 7, 8, 9, 10, 11, 12, 13, 14, 15 or 16 SNP markers for each of the four QTLs as present in seeds deposited under NCIMB 43745, deposited under the Budapest Treaty on Mar. 5, 2021 by Nunhems B.V. Therefore, in one aspect seeds, or progeny of seeds, deposited under NCIMB43745 (comprising all four QTLs) can be used as donor for one or more of the four QTLs, or other donors, such as PI605996, PI197087, CGN22263 or CGN22932, or a wild donor which has the same SNP haplotype for at least 5, 6, 7, 8, 9, 10, 11, 12, 13, 14, 15 or 16 SNP markers described herein for the QTLs present in NCIMB43745 can be used as donors for one or more of the four QTLs.

### BACKGROUND

Cultivated cucumber (Cucumis sativus var. sativus L.) is an important vegetable crop worldwide. It belongs to the family Cucurbitaceae. It is thought to originate from South East Asia from wild ancestors with small, bitter fruits, such as Cucumis sativus var. hardwickii.

The cultivated cucumber genome has seven pairs of chromosomes (n=7) and a haploid genome size of about 367 Mb (Megabases) with an estimated total of about 26,682 genes. The cucumber genome was the first vegetable genome to be sequenced (Huang et al. 2009, Nature Genetics, Volume 41, Number 12, p 1275-1283).

In the recent years isolates of ToLCNDV developed in Europe which is more adapted to Cucurbitaceae host species, such as cucumber. These isolates, referred to as ToLCNDV-ES, causes yield losses in the field, but also in protected environments, during cucumber cultivation in areas where whiteflies carry and transmit the virus. No resistant cucumber varieties are available to date. There is, therefore, a need to provide cultivated cucumbers which are resistant to ToLCNDV-ES. There is also a need to provide e.g. wild cucumber donors comprising QTLs which confer or increase resistance to ToLCNDV-ES and methods of identifying such donors.

### FIGURES

FIG. 1 shows a schematic diagram of chromosomes 1, 2 and 3, comprising QTL1.1 linked to SNP_01 to SNP_16, QTL1.2 linked to SNP_17 to SNP_31, QTL2.1 linked to SNP_32 to SNP_47 and QTL3.1 linked to SNP_48 to SNP_62.

FIG. 2 shows photographs of cucumber leaves having a certain disease score used in the ToLCNDV-ES disease assay. The phenotyping is done on a scale of Score 2.0 to Score 9.0 (free of symptoms), as e.g. described in the Examples. It is noted that symptoms are difficult to reproduce in black and white.

FIG. 3 shows schematically the positions of QTL1 and QTL2 from donor CUC29 of Vilmorin WO2021/019069 using black bars. The SNP markers of the CUC29 donor are listed on the left hand of the black bar. The SNP markers of the CUC29 donor which have a different SNP nucleotide in the genome compared to the donor of the instant invention are highlighted in bold. Markers SNP_29, SNP_30 and SNP_31 of the instant invention (of QTL1.2) are shown to lie within the region of QTL1 of the CUC29 donor. Markers SNP_41 to SNP_45 of the instant invention (of QTL2.1) are shown to lie within the region of QTL2 of the CUC29 donor.

FIG. 4 shows a boxplot of ToLCNDV-ES virus levels in the upper leaves of different genotypes at two time points after inoculation. The resistant genotypes comprise very low levels of virus compared to the susceptible plants.

### GENERAL DEFINITIONS

The indefinite article “a” or “an” does not exclude the possibility that more than one of the element is present, unless the context clearly requires that there be one and only one of the elements. The indefinite article “a” or “an” thus usually means “at least one”.

As used herein, the term “plant” includes the whole plant or any parts or derivatives thereof, such as plant organs (e.g., harvested or non-harvested storage organs, tubers, fruits, leaves, seeds, etc.), plant cells, plant protoplasts, plant cell or tissue cultures from which whole plants can be regenerated, plant calli, plant cell clumps, and plant cells that are intact in plants, or parts of plants, such as embryos, pollen, ovules, ovaries, fruits (e.g., harvested tissues or organs, such as harvested cucumber fruits or parts thereof), flowers, leaves, seeds, tubers, bulbs, clonally propagated plants, roots, root-stocks, stems, root tips and the like. Also any developmental stage is included, such as seedlings, immature and mature, etc. When “seeds of a plant” are referred to, these either refer to seeds from which the plant can be grown or to seeds produced on the plant, after self-fertilization or cross-fertilization.

“Plant variety” is a group of plants within the same botanical taxon of the lowest grade known, which (irrespective of whether the conditions for the recognition of plant breeder's rights are fulfilled or not) can be defined on the basis of the expression of characteristics that result from a certain genotype or a combination of genotypes, can be distinguished from any other group of plants by the expression of at least one of those characteristics, and can be regarded as an entity, because it can be multiplied without any change. Therefore, the term “plant variety” cannot be used to denote a group of plants, even if they are of the same kind, if they are all characterized by the presence of a few loci or genes (or phenotypic characteristics due to these specific loci or genes), but which can otherwise differ from one another enormously as regards the other loci or genes. Thus, e.g. a plant defined only by the presence of one or more of QTL1.1, QTL1.2, QTL2.1 and QTL3.1 not a plant variety, as thousands of other genes which define a plant variety are undefined and a plant defined only by the presence of QTL1.1, QTL1.2, QTL2.1 and QTL3.1 is not uniform and stable for these thousands of genes and the characteristics conferred by these genes. QTL1.1, QTL1.2, QTL2.1 and QTL3.1 can be used to develop many different plant varieties, e.g. a long cucumber variety which is uniform and stable for all its physiological and morphological characteristics such as leaf size or shape, leaf margins and color, fruit size and color, warts, bitterness, plant height, etc. and which also comprises one or more of QTL1.1, QTL1.2, QTL2.1 and QTL3.1.

“F1, F2, F3, etc.” refers to the consecutive related generations following a cross between two parent plants or parent lines. The plants grown from the seeds produced by crossing two plants or lines is called the F1 generation. Selfing the F1 plants results in the F2 generation, etc.

“F1 hybrid” plant (or F1 hybrid seed) is the generation obtained from crossing two inbred parent lines. Thus, F1 hybrid seeds are seeds from which F1 hybrid plants grow. F1 hybrids are more vigorous and higher yielding, due to heterosis. Inbred lines are essentially homozygous at most loci in the genome.

A “plant line” or “breeding line” refers to a plant and its progeny. As used herein, the term “inbred line” refers to a plant line which has been repeatedly selfed and is nearly homozygous. Thus, an “inbred line” or “parent line” refers to a plant which has undergone several generations (e.g. at least 5, 6, 7 or more) of inbreeding, resulting in a plant line with a high uniformity.

The term “allele(s)” means any of one or more alternative forms of a gene at a particular locus, all of which alleles relate to one trait or characteristic at a specific locus. In a diploid cell of an organism, alleles of a given gene are located at a specific location, or locus (loci plural) on a chromosome. One allele is present on each chromosome of the pair of homologous chromosomes. A diploid plant species may comprise a large number of different alleles at a particular locus. These may be identical alleles of the gene (homozygous) or two different alleles (heterozygous). Thus, for example reference may herein be made to a “ToLCNDV-ES resistance allele” of QTL1.1, QTL1.2, QTL2.1 or QTL3.1.

The term “gene” means a (genomic) DNA sequence comprising a region (transcribed region), which is transcribed into a messenger RNA molecule (mRNA) in a cell, and an operably linked regulatory region (e.g. a promoter). Different alleles of a gene are thus different alternatives form of the gene, which may be in the form of e.g. differences in one or more nucleotides of the genomic DNA sequence (e.g. in the promoter sequence, the exon sequences, intron sequences, etc.), mRNA and/or amino acid sequence of the encoded protein.

The term “locus” (loci plural) means a specific place or places or a site on a chromosome where for example a QTL, a gene or genetic marker is found. The ToLCNDV-ES locus (or ToLCNDV-ES resistance locus) is, thus, the location in the genome of cucumber, where QTL1.1, QTL1.2, QTL2.1 or QTL3.1 is found. In cultivated cucumber the QTLs are found on chromosome 1, 2 and 3 (using the chromosome assignment of Huang et al. 2009, Nature Genetics, Volume 41, Number 12, p 1275-1283) and world wide web at //cucurbitgenomics.org/, the genome of Cucumber (Chinese Long) v3) i.e. they are introgressed into the cultivated cucumber genome (i.e. onto chromosome 1, 2 and/or 3) from a wild or primitive cucumber donor.

A “quantitative trait locus”, or “QTL” is a chromosomal locus that encodes for one or more alleles that affect the expressivity of a continuously distributed (quantitative) phenotype. The ToLCNDV-ES resistance conferring quantitative trait loci are named QTL1.1, QTL1.2, QTL2.1 and QTL3.1 herein.

“Cucumber genome” and “physical position on the cucumber genome” and “chromosome 1, 2 or 3” refers to the physical genome of cultivated cucumber, world wide web at //cucurbitgenomics.org/, the genome of Cucumber (Chinese Long) v3), and the physical chromosomes and the physical position on the chromosomes. So, for example SNP_01 is located at the nucleotide (or ‘base’) positioned physically at nucleotide 8235142 of chromosome 1.

“Physical distance” between loci (e.g. between molecular markers and/or between phenotypic markers) on the same chromosome is the actually physical distance expressed in bases or base pairs (bp), kilo bases or kilo base pairs (kb) or megabases or mega base pairs (Mb).

“Genetic distance” between loci (e.g. between molecular markers and/or between phenotypic markers) on the same chromosome is measured by frequency of crossing-over, or recombination frequency (RF) and is indicated in centimorgans (cM). One cM corresponds to a recombination frequency of 1%. If no recombinants can be found, the RF is zero and the loci are either extremely close together physically or they are identical. The further apart two loci are, the higher the RF.

“Introgression fragment” or “introgression segment” or “introgression region” refers to a chromosome fragment (or chromosome part or region) which has been introduced into another plant of the same or related species by crossing or traditional breeding techniques, such as backcrossing, i.e. the introgressed fragment is the result of breeding methods referred to by the verb “to introgress” (such as backcrossing). In cucumber, wild or primitive cucumber accessions (e.g. landraces) or wild relatives of cultivated cucumber can be used to introgress fragments of the wild genome into the genome of cultivated cucumber, Cucumis sativus var. sativus L. Such a cultivated cucumber plant thus has a “genome of cultivated Cucumis sativus var. sativus”, but comprises in the genome a fragment of a wild or primitive cucumber or of a wild relative of cucumber, e.g. an introgression fragment of a related wild Cucumis sativus genome, such as Cucumis sativus var. hardwickii, C. sativus var. sikkimensis Cucumis sativus var. xishuangbannesis, or another wild cucumber or wild relative of cucumber. So, for example, a cultivated cucumber is provided herein comprising a genome of cultivated cucumber, and in that genome one, two, three or four introgression fragments on chromosome 1, 2 and/or 3 of cultivated cucumber which confer enhanced ToLCNDV-ES resistance compared to the cultivated cucumber genome lacking the introgression fragments (and having a chromosomes 1, 2 and 3 of cultivated cucumber, without the introgression fragments). It is understood that the term “introgression fragment” never includes a whole chromosome, but only a part of a chromosome. The introgression fragment can be large, e.g. even three quarter or half of a chromosome, but is preferably smaller, such as about 15 Mb or less, such as about 10 Mb or less, about 9 Mb or less, about 8 Mb or less, about 7 Mb or less, about 6 Mb or less, about 5 Mb or less, about 4 Mb or less, about 3 Mb or less, about 2.5 Mb or 2 Mb or less, about 1 Mb (equals 1,000,000 base pairs) or less, or about 0.5 Mb (equals 500,000 base pairs) or less, such as about 200,000 bp (equals 200 kilo base pairs) or less, about 100,000 bp (100 kb) or less, about 50,000 bp (50 kb) or less, about 25,000 bp (25 kb) or less.

“Cultivated cucumber” or “domesticated cucumber” refers to plants of Cucumis sativus var. sativus i.e. varieties, breeding lines or cultivars, cultivated by humans and having good agronomic characteristics, especially producing edible and marketable fruits of good size and quality and uniformity; such plants are not “wild cucumber” or “primitive cucumber” plants, i.e. plants which generally have much poorer yields and poorer agronomic characteristics than cultivated plants and are less uniform genetically and in their physiological and/or morphological characteristics. “Wild plants” of “wild cucumber” include for example ecotypes, landraces or wild accessions or wild relatives of a species. Cultivated cucumber plants (lines or varieties) can also be distinguished from wild or primitive cucumber accessions by the significantly lower amount of SNPs (less than 2,000,000 SNPs) and INDELs (insertions/deletions of shorter than 5 bp; less than 150,000 INDELs) in the genome and their significantly lower nucleotide diversity (equal to or less than 2.3×10⁻³ π), as described in Table 1 of Qi et al, Nature Genetics December 2013, Vol 45, No. 12, pages 1510-1518. SNP numbers, INDEL numbers and nucleotide diversity can be determined as described herein, especially in the section ‘Online Methods’.

“Indian cucumber group” refers to wild or wild relatives of cucumbers from India, having a high amount of SNPs (more than 3,000,000 SNPs) and INDELs (insertions/deletions of shorter than 5 bp; more than 200,000 INDELs) in the genome and high nucleotide diversity (more than 3.0×10⁻³ π or even more than 4.0×10⁻³ π).

“Eurasian cucumber group” refers to cultivated cucumbers from central or western Asia, Europe and the United States, having a low amount of SNPs (less than 2,000,000 SNPs, or less than 1,500,000 SNPs) and INDELs (insertions/deletions of shorter than 5 bp; less than 150,000 INDELs) in the genome and a low nucleotide diversity (equal to or less than 2.3×10⁻³ π, preferably less than 2.0×10⁻³ π).

“East Asian cucumber group” refers to cultivated cucumbers from East Asia, such as China, Korea and Japan, having a low amount of SNPs (less than 2,000,000 SNPs, or less than 1,500,000 SNPs) and INDELs (insertions/deletions of shorter than 5 bp; less than 150,000 INDELs, preferably less than 100,000) in the genome and a low nucleotide diversity (equal to or less than 2.3×10⁻³ π, preferably less than 2.0×10⁻³ π or even less than 1.5×10⁻³ π).

“Xishuangbanna cucumber group” refers to cucumbers from the Xishuangbanna region of China, having a low amount of SNPs (less than 2,000,000 SNPs, or less than 1,500,000 SNPs or even less than 100,000 SNPs) and INDELs (insertions/deletions of shorter than 5 bp; less than 150,000 INDELs, preferably less than 100,000) in the genome and a low nucleotide diversity (equal to or less than 2.3×10⁻³ π, preferably less than 2.0×10⁻³ π or even less than 1.5×10⁻³ π).

“Wild cucumber” or “primitive cucumber” refers to C. sativus var. sativus which generally have much poorer yields and poorer agronomic characteristics than cultivated plants and are less uniform genetically and in their physiological and/or morphological characteristics. Wild plants include for example ecotypes, landraces or wild accessions or wild relatives of a species.

“Wild relatives of cucumber” refer to Cucumis sativus var. hardwickii, C. sativus var. sikkimensis, Cucumis sativus var. xishuangbannesis.

“Landrace(s)” refers to primitive cultivars of Cucumis sativus var. sativus developed in local geographic regions, which often show a high degree of genetic variation in their genome and exhibit a high degree of morphological and/or physiological variation within the landrace (e.g. large variation in fruit size, etc.), i.e. are significantly less uniform than cultivated cucumber. Landraces are, therefore, herein included in the group “wild cucumber”, which is distinct from “cultivated cucumber”.

“Uniformity” or “uniform” relates to the genetic and phenotypic characteristics of a plant line or variety. Inbred lines are genetically highly uniform as they are produced by several generations of inbreeding. Likewise, and the F1 hybrids which are produced from such inbred lines are highly uniform in their genotypic and phenotypic characteristics and performance.

The term “ToLCNDV-ES resistance allele” refers to an allele found at the locus QTL1.1, QTL1.2, QTL2.1 and/or QTL3.1 introgressed into cultivated cucumber (onto cultivated C. sativus var. sativus chromosome 1, 2 and/or 3) from a wild or primitive cucumber. The term “ToLCNDV-ES resistance allele”, thus, also encompasses alleles obtainable from other Cucumis accessions. When one or two ToLCNDV-ES resistance alleles are present at the locus QTL1.1, QTL1.2 and/or QTL3.1 in the genome (i.e. in heterozygous or homozygous form) or when two ToLCNDV-ES resistance alleles are present at the locus of QTL2.1 (i.e. in homozygous form), the plant line or variety comprises a significantly higher resistance to ToLCNDV-ES than the control or genetic control lacking the QTLs. In cultivated cucumber plant lacking the introgression fragments, the C. sativus var. sativus allele found at the same locus on chromosome 1, 2 and 3 is herein referred to as “wild type” allele (wt). As QTL2.1 is recessive or partially recessive, this QTL is preferably in homozygous form, while the other QTLs may be in heterozygous or homozygous form. The genotype of the SNP markers provided herein is also indicative of the wild type or of either of the QTLs in homozygous or heterozygous form. E.g. the genotype of SNP_01 indicative of QTL1.1 is ‘TG’ (QTL1.1/wt) or ‘TT’ (QTL1.1 QTL1.1) while the genotype indicative of the wild type, i.e. of the cultivated cucumber, is ‘GG’ (wt/wt). The genotype of SNP_02 indicative of QTL1.1 is ‘TC’ (QTL1.1/wt) or ‘TT’ (QTL1.1 QTL1.1) while the genotype indicative of the wild type, i.e. of the cultivated cucumber, is ‘CC’ (wt/wt). Likewise the SNP haplotype for SNP_01 and SNP_02 is ‘T-T’ and the SNP genotype for SNP_01 and SNP_02 is ‘TT-TT’).

A genetic element, an introgression fragment, or a gene or allele conferring a trait (such as ToLCNDV-ES resistance) is said to be “obtainable from” or can be “obtained from” or “derivable from” or can be “derived from” or “as present in” or “as found in” a plant or seed or tissue or cell if it can be transferred from the plant or seed in which it is present into another plant or seed in which it is not present (such as a line or variety) using traditional breeding techniques without resulting in a phenotypic change of the recipient plant apart from the addition of the trait conferred by the genetic element, locus, introgression fragment, gene or allele. The terms are used interchangeably and the genetic element, locus, introgression fragment, gene or allele can thus be transferred into any other genetic background lacking the trait. Not only seeds deposited and comprising the genetic element, locus, introgression fragment, gene or allele can be used, but also progeny/descendants from such seeds which have been selected to retain the genetic element, locus, introgression fragment, gene or allele, can be used and are encompassed herein, such as commercial varieties developed from the deposited seeds or from descendants thereof. Whether a plant (or genomic DNA, cell or tissue of a plant) comprises the same genetic element, locus, introgression fragment, gene or allele as obtainable from the deposited seeds can be determined by the skilled person using one or more techniques known in the art, such as phenotypic assays, whole genome sequencing, molecular marker analysis, trait mapping, chromosome painting, allelism tests and the like, or combinations of techniques.

“SNP marker” refer herein to single nucleotide polymorphisms of a genomic sequence linked to QTL1.1, QTL1.2, QTL2.1 or to QTL3.1, whereby a specific nucleotide, which is also referred to as the donor SNP nucleotide, (e.g. for SNP_01 a Thymine at nucleotide 51 of SEQ ID NO:1, or an Thymine at nucleotide 51 of a sequence comprising at least 95%, 96%, 97%, 98%, 99% sequence identity to SEQ ID NO: 1), or sequence comprising the specific nucleotide, is linked to the QTL. This nucleotide, or sequence comprising the nucleotide, is also referred to as the ‘SNP genotype’ or ‘SNP nucleotide’ of the plant or plant part, and SNP_01 may be ‘T’ (haploid, on one chromosome) or ‘TT’ (diploid, on both chromosomes). Markers SNP_01 to SNP_16 are linked to QTL1.1 and are present on the introgression fragment which comprises QTL1.1. Markers SNP_17 to SNP_31 are linked to QTL1.2 and the markers SNP_17 to SNP_31 are present on the introgression fragment which comprises QTL1.2. Markers SNP_32 to SNP_47 are linked to QTL2.1 and the markers SNP_32 to SNP_47 are present on the introgression fragment which comprises QTL2.1. Markers SNP_48 to SNP_62 are linked to QTL3.1 and the markers SNP_48 to SNP_62 are present on the introgression fragment which comprises QTL3.1.

The ‘haplotype’ or “haploid genotype” refers to the haploid genotype of several genetic loci in a plant, especially of several SNP markers or several sequences comprising the SNP markers. For QTL1.1 the SNP haplotype may thus be the haploid genotype of at least 5, 6, 7, 8, 9, 10, 11, 12 or more (e.g. all 16) SNP markers of SNP_01 to SNP_16 (or of the sequences comprising the SNP markers). For example, the plant comprising QTL1.1 may comprise a ‘T’ for SNP_01 at nucleotide 51 in SEQ ID NO: 1 (or at nucleotide 51 of a sequence which is at least 95%, 96%, 97%, 98% or 99% identical to SEQ ID NO: 1), a ‘T’ for SNP_02 at nucleotide 51 in SEQ ID NO: 2 (or at nucleotide 51 of a sequence which is at least 95%, 96%, 97%, 98% or 99% identical to SEQ ID NO: 2), a ‘T’ for SNP_03 in SEQ ID NO: 3, it thus has the SNP haplotype T-T-T for SNP_01 to SNP_03, which is the SNP haplotype of SNP_01 to SNP_03 of the wild cucumber donor (also referred to as donor SNP haplotype). A diploid plant homozygous for the QTL would have the SNP genotype TT-TT-TT for SNP_01 to SNP_03.

A “Variant” or “orthologous” sequence or a “variant QTL1.1, QTL1.2, QTL2.1 or QTL3.1” or a “variant of QTL1.1, QTL1.2, QTL2.1 or QTL3.1” refers to a ToLCNDV-ES resistance conferring QTL (QTL1.1, QTL1.2, QTL2.1 or QTL3.1), or an introgression fragment comprising the QTL, which is derived from a different wild or primitive cucumber donor plant than the QTL1.1, QTL1.2, QTL2.1 or QTL3.1 present in NCIMB43745. Such a variant QTL can e.g. be identified as having the same SNP haplotype as the QTLs present in NCIMB43745 for at least 5, 6, 7, 8, 9, 10, 11, 12, 13, 14, 15 or 16 markers (preferably consecutive markers) selected from SNP_01 to SNP_16 for a variant of QTL1.1, selected from SNP_17 to SNP_31 for a variant of QTL1.2, selected from SNP_32 to SNP_47 for a variant of QTL2.1 and selected from SNP_48 to SNP_62 for a variant of QTL3.1. So for example a plant comprising a variant QTL1.1 may comprise a SNP haplotype T-T-T-T-A for SNP_01 to SNP_05, i.e. a ‘T’ for SNP_01 at nucleotide 51 in SEQ ID NO: 1 (or at nucleotide 51 of a sequence which is at least 95%, 96%, 97%, 98% or 99% identical to SEQ ID NO: 1), a ‘T’ for SNP_02 at nucleotide 51 in SEQ ID NO: 2 (or at nucleotide 51 of a sequence which is at least 95%, 96%, 97%, 98% or 99% identical to SEQ ID NO: 2), a ‘T’ for SNP_03 at nucleotide 51 in SEQ ID NO: 3 (or at nucleotide 51 of a sequence which is at least 95%, 96%, 97%, 98% or 99% identical to SEQ ID NO: 3), a ‘T’ for SNP_04 at nucleotide 51 in SEQ ID NO: 4 (or at nucleotide 51 of a sequence which is at least 95%, 96%, 97%, 98% or 99% identical to SEQ ID NO: 4), and an ‘A’ for SNP_05 at nucleotide 51 in SEQ ID NO: 5 (or at nucleotide 51 of a sequence which is at least 95%, 96%, 97%, 98% or 99% identical to SEQ ID NO: 5). In addition the variant QTL confers (at least in homozygous form) reduced susceptibility/increased resistance to ToLCNDV-ES infection, as described herein.

“Resistant” or “being resistant to” shall be understood in context of the present invention to mean a plant which is a host species of a particular pathogen and can therefore be infected by a given pathogen, but wherein the plant comprises one or more genetic element (e.g. one or more introgression fragments) resulting in reduction of pathogen growth and/or spreading in the plant after infection compared to the susceptible plant lacking the genetic elements. In context of the present invention “resistant” or “being resistant to” or in particular refers to plant cells or plants being resistant to ToLCNDV-ES. Resistance is a relative term which can span a range of (different) reactions in the plant cell or plant, triggered by pathogen infection. The effect of those reactions by the plant cell or plant can be measured by various means. Typically the effect is measured by defining a symptom level appearing in the plant part or plant. Typically average symptoms (average disease score) of several plants of a line (e.g. 5, 6, 7, 8, 9, 10 or more) are compared to average symptoms (average disease score) of several plants of a control line or variety, preferably a susceptible control line or variety. Thus at least 5 6, 7, 8, 9, 10 or more individual plants of a line or variety are scored at one or more time points (e.g. 25 dpi, 32 dpi and 46 dpi, days post infection/inoculation) and the average disease score is calculated. Concerning the present invention, the following symptom levels (or disease score) are applied according to phenotypic observations taken after ToLCNDV-ES infection:
Score 2.0: cucumber leaves with fully covered yellowing mosaics (about 90 to 100% of leaf area)
Score 3.0: cucumber leaves with strong yellowing mosaics (about 70 to 80% of leaf area)
Score 4.0: cucumber leaves with clear yellowing mosaics on fully expanded leaves (about 40 to 60% of leaf area)
Score 5.0: cucumber leaves with yellowing mosaics (about 30 to 40% of leaf area) evenly distributed in interveinal spaces
Score 6.0: cucumber leaves with yellowing mosaics (about 20-30% of leaf area) evenly distributed in interveinal spaces
Score 7.0: cucumber leaves with mild yellowing mosaics (about 10% of leaf area)
Score 8.0: cucumber leaves with presence of very faint yellowing mosaic symptom (vein bending)
Score 9.0: healthy leaves with no symptoms

For determining the symptom level (or disease score) preferably young plants are infected with ToLCNDV-ES. Young plants are preferably plants having the age of the first true leaf being expanded, preferably approximately 10 to 14 days after sowing. Infection is preferably carried out via feeding of the vector (Bemisia) carrying the virus. For this purpose plants are germinated and grown under optimal or close to optimal conditions. The symptom level is preferably determined at least once at e.g. 25 days after infection (dpi) (or e.g. 21, 22, 23 or 24 dpi). Optionally symptom level is determined twice or even three times at different time-points following infection to confirm the result, e.g. a first scoring at approximately 25 days after infection (or e.g. 21, 22, 23 or 24 dpi) and a second scoring at approximately 32 days after infection (or 33, 34, 35 or 36 dpi) and optionally the third scoring approximately 46 days after infection (or e.g. 47, 48, 49 dpi) with ToLCNDV-ES. See also the Examples. The average disease score is calculated for each line or variety at each time point. In one aspect a plant line or variety is said to be “resistant” against ToLCNDV-ES infection if it has an average disease score (at least at one but preferably at two or even three time points after inoculation) of at least 7.5, while the susceptible control line or variety has the expected disease score, such as variety Renoir has an average disease score of about 3.0 or less, when grown under the same conditions and infected in the same way. Preferably several susceptible control varieties are included, which need to show the expected symptoms to know that the infection has worked. In another aspect a plant line or variety is said to be “highly resistant” against ToLCNDV-ES infection if it has an average disease score (at least at one but preferably at two or even three time points after inoculation) of 8.0 or higher, preferably if it has an average disease score of at least 8.4 or 8.5, more preferably of at least 9.0, while the susceptible control line or variety shows the expected disease score, such as variety Renoir has an average disease score of about 3.0 or less, when grown under the same conditions and infected in the same way.

An “increased (or enhanced) resistance against ToLCNDV-ES infection” or a “significantly increased (or enhanced) ToLCNDV-ES resistance” or a “reduced susceptibility to ToLCNDV-ES infection” refers to a cultivated cucumber plant, plant line, hybrid or variety comprising QTL1.1, QTL1.2, QTL2.1 and/or QTL3.1 (or a variant of any of these), having (due to the one or more QTLs, especially when in homozygous form) a higher average disease score (on the scale of 2.0 to 9.0 described above) at one or more of the measured time-points after infection (e.g. at 25 dpi) compared to the control plant lacking the QTLs, preferably the genetic control plant or recurrent parent. Preferably the average disease score of the line or variety is increased by at least 1.0 point, 1.5 points, 2.0 points, 2.5 points, 3.0 points, 3.5 points, 4.0 points or more on the scale of 2.0 to 9.0. For example if the recurrent parent plant line lacking the QTLs has an average disease score of 5.0, the introduction of one or more of QTL1.1, QTL1.2, QTL2.1 and/or QTL3.1 into that line, especially in homozygous form, increases the average disease score of the line to at least 6.0, preferably at least 6.5, 7.0, 7.5, 8.0, 8.5 or 9.0.

“Control plant” is a cultivated cucumber genotype, breeding line, hybrid or variety lacking the introgression fragments. The control plant is preferably of the same type as the plant comprising the introgression fragment(s), e.g. long cucumber type, pickling type, short cucumber type, slicer type, etc. For example, the original (e.g. susceptible) parent line into which the QTLs are/were introgressed (also referred to as the recurrent parent) is a suitable control. Other controls are e.g. known susceptible varieties such as Renoir F1 (Nunhems variety, slicer type), Mastil F1 (Nunhems variety, long cucumber type), Squisito F1 (Nunhems variety, long cucumber type) and Taray F1 (Nunhems variety, long cucumber type). Also seeds deposited under NCIMB 43744 are suitable as a control.

“Genetic control” is a cultivated cucumber genotype, breeding line, variety or hybrid which has the same or very similar cultivated genome as the cucumber plant comprising the one or more introgression fragments except that it lacks the introgressions, i.e. chromosome 1, 2 and 3 are “wild type”, i.e. cultivated cucumber genome. This is for example a backcross line in the backcrossing program which does not contain the introgression fragments. For example seeds deposited under NCIMB 43744 are suitable as a genetic control.

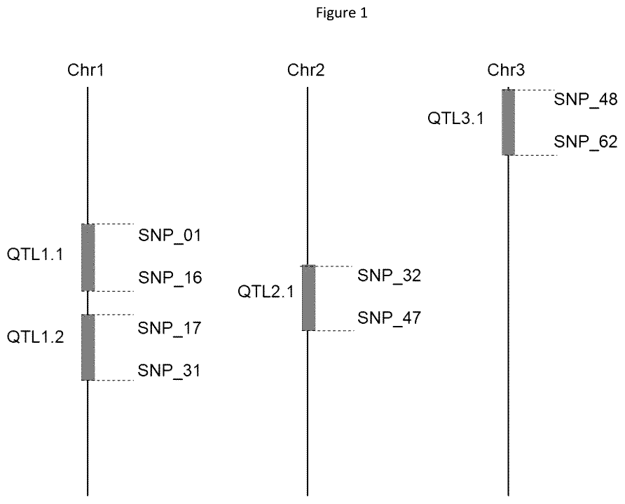

The term “marker assay” refers to a molecular marker assay which can be used to test whether on cultivated C. sativus var. sativus chromosome 1, 2 and/or 3 an introgression from a wild or primitive cucumber is present which introgression fragment comprises the ToLCNDV-ES resistance QTL1.1, QTL1.2, QTL2.1 and/or QTL3.1 (or a variant of any of these), by determining the genotype or haplotype of any one or more markers linked to the QTL1.1, QTL1.2, QTL2.1 and/or QTL3.1, e.g. the genotype or haplotype of one or more SNP markers selected from SNP_01 to SNP_16 for QTL1.1, or the genotype or haplotype of one or more SNP markers selected from SNP_17 to SNP_31 for QTL1.2, or the genotype or haplotype of one or more SNP markers selected from SNP_32 to SNP_47 for QTL2.1, or the genotype or haplotype of one or more SNP markers selected from SNP_48 to SNP_62 for QTL3.1 (see also FIG. 1).

“Flanking markers” are markers which are on either side of the QTL, i.e. the QTL is located on the chromosomal region in-between the flanking markers, e.g. the QTL1.1 (or a variant QTL1.1) is in one aspect in between SNP_01 at nucleotide 51 of SEQ ID NO: 1 and SNP_16 at nucleotide 51 of SEQ ID NO: 16, QTL1.2 (or a variant QTL1.2) is in one aspect in between SNP_17 at nucleotide 51 of SEQ ID NO: 17 and SNP_31 at nucleotide 51 of SEQ ID NO: 31, QTL2.1 (or a variant QTL2.1) is in one aspect in between SNP_32 at nucleotide 51 of SEQ ID NO: 32 and SNP_47 at nucleotide 51 of SEQ ID NO: 47, QTL3.1 (or a variant QTL3.1) is in one aspect in between SNP_48 at nucleotide 51 of SEQ ID NO: 48 and SNP_62 at nucleotide 51 of SEQ ID NO: 62.

The SNP markers provided herein are located in the given order on the introgression fragment (see FIG. 1). “Consecutive” markers refers to markers in the same consecutive order, so e.g. two consecutive markers may be SNP_01 and SNP 02; SNP_02 and SNP 03; SNP_03 and SNP_04, etc. and three consecutive markers may be SNP_01 and SNP_02 and SNP_03; SNP_02 and SNP_03 and SNP_04; etc.

“Average” or “mean” refers herein to the arithmetic mean and both terms are used interchangeably. The term “average” or “mean” thus refers to the arithmetic mean of several measurements. The skilled person understands that the phenotype of a plant line or variety depends to some extent on growing conditions and that, therefore, arithmetic means of at least 5, 6, 7, 8, 9, 10, 15, 20, 30, 40, 50 or more plants (or plant parts) are measured, preferably in randomized experimental designs with several replicates and suitable control plants grown under the same conditions in the same experiment. “Statistically significant” or “statistically significantly” different or “significantly” different refers to a characteristic of a plant line or variety that, when compared to a suitable control (e.g. the genetic control) show a statistically significant difference in that characteristic (e.g. the p-value is less than 0.05, p<0.05, using ANOVA) from the (mean of the) control.

A “recombinant chromosome” refers to a chromosome having a new genetic makeup arising through crossing-over between homologous chromosomes, e.g. a “recombinant chromosome 1”, i.e. a chromosome 1 which is not present in either of the parent plants and arose through a rare double crossing-over event between homologous chromosomes of a chromosome 1 pair. Herein, for example, recombinant cucumber chromosome 1 is provided comprising an introgression fragment from a wild or primitive cucumber donor. The same applies for chromosome 2 or 3.

The term “traditional breeding techniques” encompasses herein crossing, backcrossing, selfing, selection, double haploid production, embryo rescue, protoplast fusion, marker assisted selection, mutation breeding etc., all as known to the breeder (i.e. methods other than genetic modification/transformation/transgenic methods), by which, for example, a recombinant chromosome 1, 2 or 3 can be obtained, identified and/or transferred.

“Backcrossing” refers to a breeding method by which a (single) trait, such as a ToLCNDV-ES resistance QTL, can be transferred from a (generally inferior) genetic background (e.g. a wild or primitive cucumber; also referred to as “donor”) into a (generally superior) genetic background (also referred to as “recurrent parent”), e.g. cultivated cucumber. An offspring of a cross (e.g. an F1 plant obtained by crossing a wild or primitive cucumber with a cultivated cucumber; or an F2 plant or F3 plant, etc., obtained from selfing the F1) is “backcrossed” to the parent with the superior genetic background, e.g. to the cultivated parent. After repeated backcrossing, the trait of the (generally inferior) genetic background will have been incorporated into the (generally superior) genetic background.

“Marker assisted selection” or “MAS” is a process of using the presence of molecular markers, which are genetically linked to a particular locus or to a particular chromosome region (e.g. introgression fragment), to select plants for the presence of the specific locus or region (introgression fragment). For example, a molecular marker physically linked to a ToLCNDV-ES resistance QTL, can be used to detect and/or select cucumber plants comprising the ToLCNDV-ES resistance QTLs on chromosome 1, 2 and/or 3. The closer the linkage of the molecular marker to the locus, the less likely it is that the marker is dissociated from the locus through meiotic recombination. Likewise, the closer two markers are linked to each other the less likely it is that the two markers will be separated from one another (and the more likely they will co-segregate as a unit).

A marker “within 7 cM or within 5 cM, 3 cM, 2 cM, or 1 cM” of another marker refers to a marker which genetically maps to within the 7 cM or 5 cM, 3 cM, 2 cM, or 1 cM region flanking the marker (i.e. either side of the marker). Similarly, a marker within 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less of another marker refers to a marker which is physically located within the 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less, of the genomic DNA region flanking the marker (i.e. either side of the marker).

“LOD-score” (logarithm (base 10) of odds) refers to a statistical test often used for linkage analysis in animal and plant populations. The LOD score compares the likelihood of obtaining the test data if the two loci (molecular marker loci and/or a phenotypic trait locus) are indeed linked, to the likelihood of observing the same data purely by chance. Positive LOD scores favor the presence of linkage and a LOD score greater than 3.0 is considered evidence for linkage. A LOD score of +3 indicates 1000 to 1 odds that the linkage being observed did not occur by chance.

“Vegetative propagation”, “vegetative reproduction” or “clonal propagation” are used interchangeably herein and mean the method of taking part of a plant and allowing that plant part to form at least roots where plant part is, e.g., defined as or derived from (e.g. by cutting of) leaf, pollen, embryo, cotyledon, hypocotyl, cells, protoplasts, meristematic cell, root, root tip, pistil, anther, flower, shoot tip, shoot, stem, fruit, petiole, etc. When a whole plant is regenerated by vegetative propagation, it is also referred to as a vegetative propagation. In one aspect propagation by grafting, e.g. a scion onto a rootstock, is included herein.

“Cell culture” or “tissue culture” refers to the in vitro culture of cells or tissues of a plant.

“Regeneration” refers to the development of a plant from cell culture or tissue culture or vegetative propagation.

“Non-propagating cell” refers to a cell which cannot be regenerated into a whole plant.

“Transgene” or “chimeric gene” refers to a genetic locus comprising a DNA sequence, such as a recombinant gene, which has been introduced into the genome of a plant by transformation, such as Agrobacterium mediated transformation. A plant comprising a transgene stably integrated into its genome is referred to as “transgenic plant”.

An “isolated nucleic acid sequence” or “isolated DNA” refers to a nucleic acid sequence which is no longer in the natural environment from which it was isolated, e.g. the nucleic acid sequence in a bacterial host cell or in the plant nuclear or plastid genome. When referring to a “sequence” herein, it is understood that the molecule having such a sequence is referred to, e.g. the nucleic acid molecule.

A “host cell” or a “recombinant host cell” or “transformed cell” are terms referring to a new individual cell (or organism) arising as a result of at least one nucleic acid molecule, having been introduced into said cell. The host cell is preferably a plant cell or a bacterial cell. The host cell may contain the nucleic acid as an extra-chromosomally (episomal) replicating molecule, or comprises the nucleic acid integrated in the nuclear or plastid genome of the host cell, or as introduced chromosome, e.g. minichromosome.

“Sequence identity” and “sequence similarity” can be determined by alignment of two peptide or two nucleotide sequences using global or local alignment algorithms. Sequences may then be referred to as “substantially identical” or “essentially similar” when they are optimally aligned by for example the programs GAP or BESTFIT or the Emboss program “Needle” (using default parameters, see below) share at least a certain minimal percentage of sequence identity (as defined further below). These programs use the Needleman and Wunsch global alignment algorithm to align two sequences over their entire length, maximizing the number of matches and minimises the number of gaps. Generally, the default parameters are used, with a gap creation penalty=10 and gap extension penalty=0.5 (both for nucleotide and protein alignments). For nucleotides the default scoring matrix used is DNAFULL and for proteins the default scoring matrix is Blosum62 (Henikoff & Henikoff, 1992, PNAS 89, 10915-10919). Sequence alignments and scores for percentage sequence identity may for example be determined using computer programs, such as EMBOSS as available on the world wide web under ebi.ac.uk/Tools/psa/emboss_needle/). Alternatively sequence similarity or identity may be determined by searching against databases such as FASTA, BLAST, etc., but hits should preferably be retrieved and aligned pairwise to compare sequence identity. Two proteins or two protein domains, or two nucleic acid sequences have “substantial sequence identity” if the percentage sequence identity is at least 95%, 96%, 97%, 98% or 99% or more (as e.g. determined by Emboss “needle” using default parameters, i.e. gap creation penalty=10, gap extension penalty=0.5, using scoring matrix DNAFULL for nucleic acids and Blosum62 for proteins). For marker sequences comprising a SNP nucleotide in between flanking sequence regions, a ‘variant sequence’ (or a ‘sequence comprising substantial sequence identity’) is for example a sequence comprising the same SNP nucleotide at the equivalent position in a sequence comprising at least 95%, 96%, 97%, 98% or 99% sequence identity to the other sequence. The % identity is measured over sequences of the same length, e.g. two sequences of 101 nucleotides in length with a SNP at nucleotide 51. This can be done e.g. by pairwise alignment using e.g. Needle or by BLAST analysis of the sequence against e.g. the genomic sequence or chromosome sequence. For example a ‘SNP nucleotide at nucleotide 51 of SEQ ID NO: 1 or at nucleotide 51 in a variant sequence/or at the equivalent nucleotide in a variant sequence’, refers to the SNP nucleotide at the same position (e.g. nucleotide 51) but the flanking nucleotides to the right and to the left of the SNP may not be 100% identical to the flanking nucleotides to the left and to the right of nucleotide 51 in SEQ ID NO: 1. Both sequences of the same length, when aligned, may therefore only have 95%, 96%, 97%, 98% or 99% sequence identity. In another embodiment ad nucleotide sequence is considered to have substantially identical to the given nucleotide sequence if it can be identified using stringent hybridisation conditions.

“Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions.

“Fine-mapping” refers to methods by which the position of a QTL can be determined more accurately (narrowed down) and by which the size of the introgression fragment comprising the QTL is reduced. For example Near Isogenic Lines for the QTL (QTL-NILs) can be made, which contain different, overlapping fragments of the introgression fragment within an otherwise uniform genetic background of the recurrent parent. Such lines can then be used to map on which fragment the QTL is located and to identify a line having a shorter introgression fragment comprising the QTL.

### DETAILED DESCRIPTION

The present invention relates to a cultivated Cucumis sativus var. sativus plant comprising one or more ToLCNDV-ES resistance conferring QTLs, selected from QTL1.1 (on chromosome 1), QTL1.2 (on chromosome 1), QTL2.1 (on chromosome 2) and QTL3.1 (on chromosome 3) introgressed from a wild or primitive cucumber. Thus, the increased ToLCNDV-ES resistance is conferred by an introgression fragment on cultivated cucumber chromosome 1, 2 or 3 (comprising QTL1.1, QTL1.2, QTL2.1 and/or QTL3.1 or a variant of any of these), wherein said introgression fragment is from a wild or primitive cucumber, referred to as the ‘donor’ of the QTL.

When reference is made herein to an introgression fragment on chromosome 1, 2 or 3 having a ToLCNDV-ES resistance conferring QTL this encompasses various sizes of introgression fragments, e.g. the fragment as found in NCIMB 43745 comprising the donor SNP nucleotide or all SNP markers linked to the QTL (for QTL1.1: SNP_01 to SNP_16, or any marker in between these; for QTL1.2: SNP_17 to SNP_31 or any marker in between these; for QTL2.1: SNP_32 to SNP_47 or any marker in between these; for QTL3.1: SNP_48 to SNP_62 or any marker in between these), but also smaller introgression fragments (comprising less than these 15 or 16 SNP markers such as only e.g. 5, 6, 7, 8, 9, 10, 11, 12, 13, 14 or 15 of the SNP markers), where however the fragment remains large enough to confer significantly enhanced ToLCNDV-ES resistance (compared to the control or genetic control) when the introgression fragment is in heterozygous or preferably in homozygous form in the cultivated cucumber genome. In other words, the fragment retains QTL1.1, QTL1.2, QTL2.1 or QTL3.1, or a variant of any of these, i.e. it still confers significantly enhanced ToLCNDV-ES resistance (compared to the control, e.g. the genetic control) when the introgression fragment is in heterozygous or preferably in homozygous form in the cultivated cucumber genome.

Further, when reference is made herein to an introgression fragment on chromosome 1, 2 or 3 having a ToLCNDV-ES resistance conferring QTL this encompasses introgression fragments from various donors which comprise the same or variant QTL of the QTLs present in e.g. NCIMB 43745 (as e.g. described in Tables 1 to 4). Such variant QTLs have the same SNP haplotype or SNP genotype for at least 5, 6, 7, 8, 9, 10 or more of the SNP makers described e.g. in Table 1 for QTL1.1, in Table 2 for QTL1.2, in Table 3 for QTL2.1 and in Table 4 for QTL3.1. For example, donors PT605996, PT197087 (both available at the ARS-GRIN collection in the US (see world wide web at npgsweb.ars-grin.gov/gringlobal/search) or CGN22263 or CGN22932 (both available at the Center for Genetic Resources, Wageningen University, //cgngenis.wur.nl/), or other accessions, which comprise the same SNP haplotype or genotype for at least 5, 6, 7, 8, 9, 10, 11, 12 or more SNP markers of one or more of the QTLs as described in Tables 1 to 4, may be used. Preferably the donor which comprises the same SNP haplotype or genotype for at least 5, 6, 7, 8, 9, 10, 11, 12 or more SNP markers of one or more of the QTLs as described in Tables 1 to 4 further also has an average ToLCNDV-ES disease score of at least 7.5, 8.0, 8.5 or preferably 9.0.

Herein below aspects are described for each of QTL1.1, QTL1.2, QTL2.1 and QTL3.1 (or variants of any of these) individually, but it is understood that not only plants and plant parts comprising individual QTLs are encompassed herein, but that plants and plant parts comprising various combinations of QTLs are encompassed. Preferred combinations are plants and plant parts comprising one or more of the major QTLs, selected from QTL1.1, QTL1.2 and QTL2.1 (or variants of any of these), plants and plant parts comprising two or three additive QTLs selected from QT1.1, QTL1.2 and QTL3.1 (or variants of any of these), plants and plant parts comprising QTL1.1 and QTL1.2 (or variants of any of these), plants and plant parts comprising QTL1.1 and/or QTL1.2 and QTL3.1 (or variants of any of these), plants and plant parts comprising QTL1.1 and/or QTL1.2 and QTL2.1 (or variants of any of these), and plants and plant parts comprising all four QTLs. As the three additive QTLs also enhance ToLCNDV-ES resistance when they are in heterozygous form, one or more of these QTLs may be present in homozygous or heterozygous form. For example a plant may comprise QTL1.1 and/or QTL1.2 and/or QTL3.1 (or variants of any of these) in heterozygous form. Or a plant may comprise QTL1.1 and/or QTL1.2 and/or QTL3.1 (or variants of any of these) in homozygous form. QTL2.1, which is recessive or partially recessive, depending on the background, is preferably present in homozygous form in the plant. Certainly, for measuring the effect on ToLCNDV-ES resistance of QTL2.1 (or a variant thereof) the QTL should be in homozygous form.

In one aspect a cultivated Cucumis sativus var. sativus plant is provided comprising at least one or two introgression fragments on chromosome 1, 2 and/or 3 from a wild cucumber donor wherein said at least one fragment comprises QTL1.1 and the other fragment comprises a QTL selected from QTL1.2, QTL2.1 and QTL3.1. Thus, in one aspect QTL1.1 is present in the plant (or plant part or seed), preferably in homozygous form, and optionally combined with any one or more of the other QTLs, selected from QTL1.2, QTL2.1 and QTL3.1. The one or more other QTLs are preferably also in homozygous form.

In another aspect a cultivated Cucumis sativus var. sativus plant is provided comprising at least three introgression fragments on chromosome 1, 2 and/or 3 from a wild cucumber donor wherein each of said introgression fragments comprises a Quantitative Trait Locus (QTL) selected from the QTLs designated QTL1.1, QTL1.2, QTL2.1 and QTL3.1. As was shown in the Examples, three QTLs (especially in homozygous form) provide very high resistance levels, only slightly below the level of all four QTLs.

In yet another aspect a cultivated Cucumis sativus var. sativus plant is provided comprising at least one or two introgression fragments on chromosome 1, 2 and/or 3 from a wild cucumber donor wherein said at least one fragment comprises QTL3.1 and the other fragment comprises a QTL selected from QTL1.1, QTL1.2 and QTL2.1. Thus, in one aspect QTL3.1 is present in the plant (or plant part or seed), preferably in homozygous form, and optionally combined with any one or more of the other QTLs, selected from QTL1.1, QTL1.2, and QTL2.1. The one or more other QTLs are preferably also in homozygous form.

### QTL1.1 and Variants of QTL1.1 on Chromosome 1

Thus, in one aspect a cultivated cucumber plant is provided comprising an introgression fragment from a wild or primitive cucumber, wherein the introgression fragment comprises QTL1.1, or a variant thereof, and wherein the introgression fragment comprises all or part of the region starting at nucleotide (or base) 8235142 of chromosome 1 (corresponding to SNP_01) and ending at nucleotide (or base) 16209127 of chromosome 1 (corresponding to SNP_16). In other words, all or part of the region starting at nucleotide 8235142 of chromosome 1 (SNP_01) and ending at nucleotide 16209127 of chromosome 1 (SNP_16) is, in one aspect, from a wild donor cucumber and comprises QTL1.1 or a variant thereof. Which sub-region contains QTL1.1 can be identified by e.g. fine-mapping. So, for example if QTL1.1 is found to be in between SNP_01 and SNP_10, then the plant of the invention only needs to comprise the introgression region starting at nucleotide 8235142 of chromosome 1 (SNP_01) and ending at nucleotide 10780728 (SNP_10) of chromosome 1.

In one aspect QTL1.1 (or a variant thereof) is located in-between marker SNP_01 at nucleotide 51 of SEQ ID NO: 1 (or at nucleotide 51 in a variant sequence of SEQ ID NO: 1) and marker SNP_16 at nucleotide 51 of SEQ ID NO: 16 (or at nucleotide 51 in a variant sequence of SEQ ID NO: 16). In another aspect QTL1.1 (or a variant thereof) is located in-between marker SNP_01 at nucleotide 51 of SEQ ID NO: 1 (or at nucleotide 51 in a variant sequence of SEQ ID NO: 1) and marker SNP_05 at nucleotide 51 of SEQ ID NO: 05 (or at nucleotide 51 in a variant sequence of SEQ ID NO: 5). In a further aspect QTL1.1 (or a variant thereof) is located in-between marker SNP_05 at nucleotide 51 of SEQ ID NO: 5 (or at nucleotide 51 in a variant sequence of SEQ ID NO: 5) and marker SNP_10 at nucleotide 51 of SEQ ID NO: 10 (or at nucleotide 51 in a variant sequence of SEQ ID NO: 10). In a further aspect QTL1.1 (or a variant thereof) is located in-between marker SNP_10 at nucleotide 51 of SEQ ID NO: 10 (or at nucleotide 51 in a variant sequence of SEQ ID NO: 10) and marker SNP_16 at nucleotide 51 of SEQ ID NO: 16 (or nucleotide 51 in a variant sequence of SEQ ID NO: 16). In a further aspect QTL1.1 (or a variant thereof) is located in-between marker SNP_04 at nucleotide 51 of SEQ ID NO: 4 (or at nucleotide 51 in a variant sequence of SEQ ID NO: 4) and marker SNP_12 at nucleotide 51 of SEQ ID NO: 12 (or nucleotide 51 in a variant sequence of SEQ ID NO: 12).

In another aspect the introgression fragment of the invention (comprising QTL1.1 or a variant thereof) is a fragment comprising a smaller fragment (part) of the region starting at nucleotide (or base) 8235142 of chromosome 1 and ending at nucleotide (or base) 16209127 of chromosome 1, e.g. having a size of e.g. 8.5 Mb, 8.0 Mb, 7.9 Mb, 7.0 Mb, 6.0 Mb, 5.0 Mb, 4.0 Mb, 3.0 Mb, 2.5 Mb, 2 Mb, 1 Mb, 0.5 Mb, 100 kb, 50 kb, 35 kb, 30 kb, 20 kb, or less and comprising the QTL or a variant thereof. In one aspect the part is at least 5 kb, 10 kb, 20 kb in size, or more.

In one aspect the cultivated cucumber plant of the invention comprises an introgression fragment from a wild or primitive cucumber, which introgression fragment comprises QTL1.1 or a variant thereof, wherein the introgression fragment comprises all of part of the region starting at 8.2 Mb and ending at 16.3 Mb of the physical chromosome 1.

In one aspect the introgression fragment on chromosome 1 comprising QTL1.1, or a variant thereof, is obtainable by crossing a plant grown from NCIMB43745 with another cucumber plant, especially a cultivated cucumber plant, in one aspect a long cucumber type or a pickling or slicer type.

In one aspect the cultivated cucumber plant of the invention comprising QTL1.1, or a variant thereof, is a plant wherein said introgression fragment on chromosome 1 is obtainable by crossing a plant grown from seeds deposited under accession number NCIMB43745 with another cucumber plant. Thus, in one aspect the QTL is the QTL present in seeds deposited under accession number NCIMB43745.

In a further aspect the cultivated cucumber plant of the invention comprising QTL1.1, or a variant thereof, is a plant wherein said introgression fragment on chromosome 1 is obtainable by crossing a plant comprising the same SNP haplotype or SNP genotype for at least 5, 6, 7, 8, 9, 10 or more SNP markers linked to the QTL (i.e. SNP_01 to SNP_16 for QTL1.1 as shown in Table 1) with another cucumber plant, especially with a cultivated cucumber elite breeding line. Thus, in one aspect the QTL is the variant QTL present in wild donor accessions which comprise the same SNP haplotype or genotype for at least 5, 6, 7, 8, 9, 10, 11, 12 or more of the SNP markers e.g. as present in NCIMB43745. Preferably the donor also comprises a resistance phenotype having an average ToLCNDV-ES disease score of at least 7.5, preferably at least 8.0, 8.1, 8.2, 8.3, 8.4, 8.5, 8.6, 8.7, 8.8, 8.9 or 9.0.

When referring to the SNP markers herein, which are indicative of the presence of the introgression fragment (and the ToLCNDV-ES resistance QTL present on the introgression fragment), it is understood that the SNP genotype or haplotype which is indicative of the introgression fragment is referred to, i.e. the SNP genotype or haplotype as provided e.g. in Tables 1 to 4. It is noted that the SNP marker genotype can distinguish between the introgression fragment being in homozygous or heterozygous form. In homozygous form the nucleotide is identical, while in heterozygous form the nucleotide is not identical. The SNP genotype of the ‘wild type’ chromosome lacking the introgression fragment is the other haplotype, e.g. the haplotype of the recurrent parent). So, e.g. the genotype of SNP_01 indicative of the introgression fragment comprising QTL1.1 is ‘TG’ (QTL1.1/wt) or ‘T’ (QTL1.1 QTL1.1) while the SNP genotype indicative of the wild type/genetic control (lacking the introgression fragment) is e.g. ‘GG’ (wt/wt). This can also be written as genotype TX′ (QTL1.1/wt) or ‘T’ (QTL1.1 QTL1.1) while the SNP genotype indicative of the wild type/genetic control (lacking the introgression fragment) is e.g. ‘XX’ (wt/wt). X may be any nucleotide (A, T, C or G). Thus, when referring to a plant or plant part (e.g. cell) comprising the introgression fragment in homozygous or heterozygous form, it is understood that the SNP markers linked to the introgression fragment have the corresponding SNP genotype or haplotype.

So, in one aspect, a cultivated Cucumis sativus var. sativus plant is provided comprising an introgression fragment on chromosome 1 in homozygous or heterozygous form, wherein said introgression fragment confers an increase in ToLCNDV-ES resistance compared to the cucumber plant lacking the introgression fragment on chromosome 1, e.g. the genetic control or control variety, when grown under the same conditions.

The increase in ToLCNDV-ES resistance is phenotypically expressed as a higher average disease score (less yellowing, measured e.g. in a disease assay as described herein) of the cultivated cucumber plant line or variety comprising the introgression fragment on chromosome 1 in homozygous or heterozygous form compared to the genetic control line or variety lacking the introgression fragment on chromosome 1 when grown under the same environment. The average disease score is preferably increased by at least 1.0, 1.5, 2.0, 2.5, 3.0 or more points on the disease scale of 2.0 (90-100% of leaf area is covered with yellowing mosaic symptoms) to 9.0 (no symptoms). So, for example if QTL1.1 is introduced into a susceptible cucumber line or variety having an average disease score of about 4.0, the introduction of QTL1.1 preferably increases the average disease score to an average score of at least 5.0, 5.5, 6.0, 6.5, 7.0 or more. As QTL1.1 was found to be additive, the effect of QTL1.1 in heterozygous form is less than the effect in homozygous form. Therefore the effect on ToLCNDV-ES resistance is preferably measured when it is in homozygous form.

It is known that all four QTLs together in homozygous form (QTL1.1, QTL1.2, QTL2.1 and QTL3.1) lead to an average disease score of 9.0 when introduced into a susceptible plant having an average disease score of about 4.0 to 5.0. The effect of the individual QTLs, even when in homozygous form, will be smaller than the combined effect, but will still be effective in reducing ToLCNDV-ES symptoms. The effect of QTL1.1 alone can be determined by introducing the QTL alone into a susceptible cucumber plant, in heterozygous or preferably in homozygous form. For example NCIMB43745 can be crossed with a susceptible plant and QTL1.1 alone can be transferred into the susceptible background.

The plants of the invention therefore comprise a genome of cultivated cucumber, with at least one or two recombinant chromosomes, namely one or two recombinant chromosomes 1 (i.e. heterozygous or homozygous). The recombinant chromosomes comprise a fragment of a wild donor cucumber, which is easily distinguishable from the cultivated cucumber genome by molecular marker analysis, whole genome sequencing, chromosome painting and similar techniques.

In one aspect the introgression fragment on chromosome 1 is from a wild or primitive cucumber, comprises the ToLCNDV-ES QTL1.1, or a variant thereof, and comprises all or part of the region starting at nucleotide SNP_01 and ending at SNP_16. Thus, the introgression fragment comprises the QTL1.1 or a variant thereof and one or more or all (e.g. 5, 6, 7, 8, 9, 10, 11, 12, 13, 14, 15) SNP markers of the wild donor selected from SNP_01 to SNP_16 as shown in Table 1.

In one aspect the introgression fragment comprises QTL1.1 and one or more or all of SEQ ID NO: 1 to SEQ ID NO: 16.

In one aspect the presence of the introgression fragment on chromosomes 1 comprising QTL1.1 in the genome of the plant or plant cell or plant tissue (or in the DNA extracted therefrom) is detectable by a molecular marker assay which detects one or more molecular markers of the introgression fragment, especially the donor SNP haplotype or genotype for at least 5, 6, 7, 8, 9, 10 or more of SNP_01 to SNP_16, at nucleotide 51 of SEQ ID NO: 1 to 16, respectively. However, as mentioned, other techniques may be used, e.g. the SNP genotype of the markers may also be determined by sequencing or by using alternative markers located in between the SNP markers provided herein or within 7 cM, or within 5 cM, of a marker provided herein; or within 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less of a marker provided herein.

In one aspect the presence of the introgression fragment on chromosomes 1 comprising QTL1.1 in the genome of the plant or plant cell or plant tissue (or in the DNA extracted therefrom) is detectable by detecting the presence of one or more or all of SEQ ID NO: 1 to SEQ ID NO: 16.

When reference is made herein to one or more molecular markers or sequences being “detectable” by e.g. a molecular marker assay, this means of course that the plant or plant part comprises the one or more markers or sequences in its genome, as the marker or sequence would otherwise not be detectable.

### Cucumber Plants Comprising an Introgression Fragment on Chromosome 1 (QTL 1.1)

In one aspect a cultivated Cucumis sativus var. sativus plant comprising an introgression fragment from a wild or primitive cucumber on chromosome 1 in homozygous or heterozygous form is provided, wherein said introgression fragment comprises a Quantitative Trait Locus (QTL) located between the Single Nucleotide Polymorphism marker SNP_01 at nucleotide 51 of SEQ ID NO: 1 (or at nucleotide 51 of a variant of SEQ ID NO: 1) and the Single Nucleotide Polymorphism marker SNP_16 at nucleotide 51 of SEQ ID NO: 16 (or at nucleotide 51 of a variant of SEQ ID NO: 1), which QTL confers an increase in ToLCNDV-ES resistance. In one aspect the QTL is located between base 8235142 (SNP_01) and base 16209127 (SNP_16) of chromosome 1.

Thus, in one aspect QTL1.1 (or a variant thereof) is located in the region between SNP_01 in SEQ ID NO: 1 (or in a variant thereof) and SNP_16 in SEQ ID NO: 16 (or in a variant thereof).

Therefore, in one aspect a cultivated Cucumis sativus var. sativus plant is provided comprising an introgression fragment on chromosome 1 in homozygous or heterozygous form, wherein said introgression fragment confers an increase in ToLCNDV-ES resistance (compared to the plant lacking the introgression fragment, e.g. the genetic control) and wherein said introgression fragment comprises the SNP marker haplotype or genotype of at least 5, 6, 7, 8, 9, 10, 11, 12, 13, 14, 15 or 16 of the SNP markers selected from the group consisting of:
a) the TX or TT genotype for the Single Nucleotide Polymorphism marker SNP_01 at nucleotide 51 of SEQ ID NO: 1 (or at nucleotide 51 in a variant thereof);
b) the TX or TT genotype for the Single Nucleotide Polymorphism marker SNP_02 at nucleotide 51 of SEQ ID NO: 2 (or at nucleotide 51 in a variant thereof);
c) the TX or TT genotype for the Single Nucleotide Polymorphism marker SNP_03 at nucleotide 51 of SEQ ID NO: 3 (or at nucleotide 51 in a variant thereof);
d) the TX or TT genotype for the Single Nucleotide Polymorphism marker SNP_04 at nucleotide 51 of SEQ ID NO: 4 (or at nucleotide 51 in a variant thereof);
e) the AX or AA genotype for the Single Nucleotide Polymorphism marker SNP_05 at nucleotide 51 of SEQ ID NO: 5 (or at nucleotide 51 in a variant thereof);
f) the CX or CC genotype for the Single Nucleotide Polymorphism marker SNP_06 at nucleotide 51 of SEQ ID NO: 6 (or at nucleotide 51 in a variant thereof);
g) the TX or TT genotype for the Single Nucleotide Polymorphism marker SNP_07 at nucleotide 51 of SEQ ID NO: 7 (or at nucleotide 51 in a variant thereof);
h) the AX or AA genotype for the Single Nucleotide Polymorphism marker SNP_08 at nucleotide 51 of SEQ ID NO: 8 (or at nucleotide 51 in a variant thereof);
i) the GX or GG genotype for the Single Nucleotide Polymorphism marker SNP_09 at nucleotide 51 of SEQ ID NO: 9 (or at nucleotide 51 in a variant thereof); or the AX or AA genotype for the Single Nucleotide Polymorphism marker SNP_09 at nucleotide 51 of SEQ ID NO: 9 (or at nucleotide 51 in a variant thereof);
j) the CX or CC genotype for the Single Nucleotide Polymorphism marker SNP_10 at nucleotide 51 of SEQ ID NO: 10 (or at nucleotide 51 in a variant thereof);
k) the CX or CC genotype for the Single Nucleotide Polymorphism marker SNP_11 at nucleotide 51 of SEQ ID NO: 11 (or at nucleotide 51 in a variant thereof);
l) the CX or CC genotype for the Single Nucleotide Polymorphism marker SNP_12 at nucleotide 51 of SEQ ID NO: 12 (or at nucleotide 51 in a variant thereof);
m) the TX or TT genotype for the Single Nucleotide Polymorphism marker SNP_13 at nucleotide 51 of SEQ ID NO: 13 (or at nucleotide 51 in a variant thereof);
n) the AX or AA genotype for the Single Nucleotide Polymorphism marker SNP_14 at nucleotide 51 of SEQ ID NO: 14 (or at nucleotide 51 in a variant thereof);
o) the CX or CC genotype for the Single Nucleotide Polymorphism marker SNP_15 at nucleotide 51 of SEQ ID NO: 15 (or at nucleotide 51 in a variant thereof); or the TX or TT genotype for the Single Nucleotide Polymorphism marker SNP_15 at nucleotide 51 of SEQ ID NO: 15 (or at nucleotide 51 in a variant thereof);
p) the GX or GG genotype for the Single Nucleotide Polymorphism marker SNP_16 at nucleotide 51 of SEQ ID NO: 16 (or at nucleotide 51 in a variant thereof).

When referring to a SNP in a variant sequence, that variant sequence comprises at least 95%, 96%, 97%, 98% or 99% sequence identity with the mentioned sequence. X refers to any nucleotide for the sequence on the other chromosome 1 of the pair of chromosomes. In one aspect X may be the nucleotide of the recurrent parent as described in Table 1.

In one aspect said at least 5, 6, 7, 8, 9, 10, 11, 12, 13, 14 or 15 markers are consecutive markers.

The fragment comprising the QTL1.1 may, thus, be large (comprising SNP_01 to SNP_16), or may be smaller and lack markers having the genotype or haplotype of the wild cucumber (i.e. the markers have the cultivated cucumber genotype or haplotype instead, see also Table 1 (SNP haplotype of recurrent parent), but it may still confer enhanced ToLCNDV-ES resistance on the cultivated cucumber plant, i.e. it can still comprise the ToLCNDV-ES allele (QTL1.1 or a variant). Such smaller introgression fragments are an embodiment of the invention. Plants having smaller introgression fragments which still confer the enhanced ToLCNDV-ES resistance (i.e. contain the resistance allele) can be generated using known techniques, such as fine-mapping or similar techniques. For example by starting with a plant comprising the introgression fragment as found in seeds deposited under accession number NCIMB 43745 and crossing such a plant with another cultivated cucumber plant and selfing the progeny of said cross, and/or backcrossing the progeny, to generate a population of plants which may contain recombinants having a smaller introgression fragment on chromosome 1, which fragments still confer enhanced ToLCNDV-ES resistance in relation to a plant lacking the introgression fragment (such as the genetic control, e.g. plants grown from seeds deposited under NCIMB42344), e.g. a fragment comprising markers SNP_01 to SNP_05, or SNP_05 to SNP_10 or SNP_10 to SNP_16 or SNP_04 to SNP_12. Marker assays can be used to determine the size of the smaller introgression fragment. One or more of the SNP markers with the genotype or haplotype of the wild donor cucumber may be missing. The cultivated cucumber genotype or haplotype is then detected for these SNP markers. The ToLCNDV-ES resistance of plants comprising such a smaller introgression fragment can then be compared in a disease assay as described herein, i.e. growing a plurality of plants comprising the smaller introgression fragment in experiments together with suitable control plants, lacking the introgression fragments. The control plants are preferably a genetic control, such as NCIMB43744. If the average ToLCNDV-ES disease score remains significantly higher than in the control, then the smaller introgression fragment has retained the QTL1.1.

Alternatively, the same or variant QTL (QTL1.1 or variant QTL1.1) may be introgressed from a different wild donor accessions, whereby optionally not all SNP markers disclosed herein may be present, i.e. the SNP haplotype of the donor accession may only be identical to the SNP haplotype of the QTL1.1 present in seeds of NCIMB43745 for at least 5, 6, 7, 8, 9, 10 or more SNPs. Such alternative wild cucumber sources can be identified using the SNP markers provided herein, by screening germplasm (i.e. accessions of) wild or primitive cucumber using a marker assay to detect the genotype or haplotype of one or more of markers SNP_01 to SNP 16, or of markers SNP_01 to SNP 05, SNP_05 to SNP 10, SNP_10 to SNP 16, or SNP_04 to SNP_12, or even only a smaller subgroup of these markers (e.g. 2, 3 or 4). For example, Table 1 shows various donors (P1605996, CGN22263, CGN22932, also known as P1197087) which have the same or similar SNP haplotype for SNP_01 to SNP_16. In the same way other donors, having QTL1.1 or a variant thereof, may be identified. Plants comprising the same or variant QTL1.1 from these donors or from other sources are also an embodiment of the invention. Thus, as long as at least 5, 6, 7, 8, 9, 10 or more (or all) of the SNPs of SNP_01 to SNP 16, or of the SNPs of SNP_01 to SNP_05, or of the SNPs of SNP_05 to SNP 10, or of the SNPs of SNP_10 to SNP_16, or of the SNPs of SNP_04 to SNP_12 are present, the donor may contain QTL1.1 (or a variant thereof) and is encompassed herein. The skilled person can then introgress the QTL1.1 (or a variant thereof) into cultivated cucumber in order to enhance ToLCNDV-ES resistance as described herein and in order to confirm that the QTL enhances ToLCNDV-ES resistance when present in cultivated cucumber. Prior to introgression the wild donor may also be tested for ToLCNDV-ES resistance in an assay as described and e.g. a donor may be selected that comprises an average ToLCNDV-ES score of e.g. at least 7.5, 8.0, 8.5, or 9.0.

As described above, in one embodiment the cultivated cucumber plant of the invention comprises an introgression fragment comprising at least a subset of SNP markers with the genotype (or haplotype) of the wild donor cucumber, i.e. at least 5, 6, 7, 8, 9, 10, 11, 12 or more markers of SNP_01 to SNP 16, or at least 3 markers of SNP_01 to SNP 05, or of SNP_05 to SNP 10, or of SNP_10 to SNP 16, or of SNP_04 to SNP_12. In one aspect the cultivated cucumber plant comprises all, or all except 1 or 2 markers of SNP_01 to SNP 16, or of SNP_01 to SNP 5, or of SNP_05 to SNP 10, or of SNP_10 to SNP 16, or of SNP_04 to SNP_12.

Thus, the introgression fragment (and a cultivated cucumber plant or plant part, e.g., a cell, comprising the introgression fragment) can be detected in a marker assay by detecting the SNP genotype or haplotype of the introgression fragment (i.e. of the wild donor cucumber germplasm) of one or more or all of the markers above, preferably at least 5, 6, 7, 8, 9, 10 or more.

Thus, in one aspect, a Quantitative Trait Locus (QTL1.1) was found to be present on chromosome 1 of a wild cucumber donor which, when transferred (introgressed) into a cultivated cucumber variety or breeding line, and when present in heterozygous or homozygous form, confers significantly enhanced ToLCNDV-ES resistance onto the cultivated cucumber plant. The QTL, or the introgression fragment comprising the QTL (comprising the ToLCNDV resistance allele), is thus additive, i.e. it is sufficient to have the introgression fragment on one of the chromosomes 1 (one recombinant chromosome 1), while the homologous chromosome 1 of the pair may be a (non-recombinant) chromosome 1 of cultivated C. sativus var. sativus lacking the introgression fragment.

Although the present source of the QTL1.1 which was used to map and introgress the QTL is a single, specific wild source, there are other wild accessions which comprise QTL1.1 (or a variant) at the same locus on chromosome 1. For example the wild accessions PI605996, CGN22263 and CGN22932 (also known as P1197087), were found to comprise the same or a very similar SNP haplotype for markers SNP_01 to SNP_16 (as shown in Table 1) and were found to be resistant to ToLCNDV-ES. The (variant) QTL1.1 from these or other donors can, thus, be introgressed into cultivated cucumber, optionally in combination with one or more of QTL1.2, QTL2.1 and QTL3.1. Similarly, other wild or primitive cucumber accessions can be screened for the SNP haplotype or genotype of one or more or all of SNP_01 to SNP_16.

Such other donors may comprise a ToLCNDV-ES resistance allele which has a slightly different nucleotide sequences, i.e. variants of the allele (QTL1.1) found herein. Such variant QTLs can also be identified and introgressed into cultivated cucumber as described herein, to generate a cultivated cucumber plant comprising a genome of cultivated C. sativus var. sativus and a recombinant chromosome 1, whereby the recombinant chromosome 1 comprises an introgression fragment, which confers an enhanced ToLCNDV-ES resistance onto the cultivated cucumber plant when present in homozygous or heterozygous form. To identify such wild donor accessions comprising QTL1.1, wild accessions can be screened, e.g. in a marker assay or by sequence comparison or other methods, for the presence of one or more of the SNP markers provided herein. The putative QTL (or variant QTL) can then be introgressed into cultivated cucumber, e.g. using MAS, i.e. using one or more (or all) of the SNP markers provided herein to detect and/or select progeny plants (e.g. backcross plants) comprising a recombinant chromosome 1. The selected plants, i.e. the cultivated cucumber plants comprising an introgression fragment on chromosome 1, wherein the introgression fragment on chromosome 1 is detectable by 5, 6, 7, 8, 9, 10 or more of the SNP markers SNP_01 to SNP_16 can then be phenotyped in a ToLCNDV-ES disease assay together with the suitable control plants, preferably at least the genetic control, in order to determine whether the introgression fragment indeed causes a significant increase in ToLCNDV-ES resistance.

Accessions of wild or primitive cucumber, are obtainable from e.g. the USDA National Plant Germplasm System collection or other seed collections, and can thus be screened for the presence of QTL1.1 using e.g. a marker assay as described herein, and accessions comprising 5 or more of the SNP markers (e.g. at least 5, 6, 7, 8, 9, 10 or more SNP markers indicative of QTL1.1, or a variant) can be crossed with a cultivated cucumber plant having normal wild-type, non-recombinant chromosomes 1. The F1 or F2 generation (or further generation, such as the F3 or a backcross generation) can then be screened for recombinant plants having the introgression fragment or a part thereof, using the molecular marker assays described herein.

In one aspect, the introgression fragment is from a donor comprising the SNP haplotype for QTL1.1 as shown in Table 1 for the introgression donor (NCIMB43745), for P1605996, for CGN22263 or for CGN22932, also known as PI197087.

In a specific embodiment, the introgression fragment comprising the ToLCNDV-ES QTL1.1 (or a variant thereof) is derivable from (or derived from) or obtainable from (or obtained from; or as present in) seeds, a representative sample of which has been deposited under accession number NCIMB 43745, or from progeny thereof, or from seeds having accession number PI605996 (USDA ARS-GRIN collection), or from seeds having accession number CGN22263 or CGN22932 (Wageningen CGN collection) or from seeds having accession number PI197087 (USDA ARS-GRIN collection) or from progeny of any of these. The progeny may be any progeny which retain the SNP markers or haplotype indicative of (and linked to) the QTL, as described. Thus, progeny are not limited to F1 or F2 progeny of the deposit or accession, but can be any progeny, whether obtained by selfing and/or crossing with another cucumber plant.

In one embodiment the introgression fragment comprising QTL1.1 (or a variant) is identifiable by one or more of the markers described elsewhere herein, especially markers SNP_01 to SNP_16 for the introgression fragment on chromosome 1, or a subset of markers, such as one or more of the markers selected from SNP markers SNP_01 to SNP_05, or from SNP markers SNP_05 to SNP_10, or from of the SNP markers SNP_10 to SNP_16, or from SNP markers SNP_04 to SNP_12. In one aspect the invention provides a cultivated cucumber plant, having a genome of cultivated (domesticated) cucumber which comprises enhanced ToLCNDV-ES resistance, wherein the enhanced resistance is conferred by an introgression fragment on the cultivated cucumber chromosome 1, wherein said introgression fragment is obtained by (or obtainable by) crossing a cultivated plant grown from seeds deposited under NCIMB 43745 or progeny of this plant (which comprises one or more the markers disclosed herein linked to the QTL) with a cultivated cucumber plant.

Thus in one aspect the cultivated cucumber plant of the invention comprises the same introgression fragment and the same recombinant chromosome 1 as present in NCIMB 43745 (comprising all of the wild donor haplotype for SNP markers SNP_01 to SNP_16 or comprising SEQ ID NO: 1 to 16), or it comprises a shorter fragment of that introgression fragment, whereby the shorter fragment retains the genetic element conferring ToLCNDV-ES resistance (QTL1.1).

Thus in one aspect the invention relates to a plant of the invention i.e. a cultivated Cucumis sativus var. sativus plant comprising an introgression fragment comprising QTL1.1 from a wild cucumber on chromosome 1 in homozygous or heterozygous form and wherein said introgression fragment is the introgression fragment “as in”/is “identical to”/is “the same as in” the seeds deposited under number NCIMB 43745, or is a shorter fragment thereof, but still confers enhanced ToLCNDV-ES resistance due to the presence of QTL1.1.

As SEQ ID NO: 1 to 16 are from the wild donor used to generate NCIMB43745, they can identify the introgression fragment or sub-fragments of the specific donor.

In yet another embodiment the invention relates to a plant of the invention i.e. a cultivated Cucumis sativus var. sativus plant comprising an introgression fragment comprising QTL1.1 (or a variant) from a wild cucumber on chromosome 1 in homozygous or heterozygous form and wherein said introgression fragment is the introgression fragment is a variant of the introgression fragment seeds deposited under number NCIMB 43745, i.e. it comprises the QTL 1.1 (or a variant), but the genomic sequence may be different. As wild accessions will be genetically divergent, the genomic sequence of an introgression fragment comprising QTL1.1 from other wild or primitive cucumbers will most likely not be identical to the genomic sequence as introgressed into NCIMB 43745, and even the ToLCNDV-ES conferring gene (comprising a promoter, introns and exons) may be divergent in nucleotide sequence, but the function will be the same, i.e. conferring enhanced resistance. The divergence can be seen in that certain SNP markers linked to QTL1.1 may be commonly found in various accessions, while other SNP markers may only be found in specific accessions. So for example not all of SNP_01 to SNP_16 may be found in other wild cucumber donors. For example PI605996 has a slightly different SNP haplotype for SNP_01 to SNP_16, with SNP_09 and SNP_15 having a different nucleotide. However, QTL1.1 (comprising e.g. a variant or ortholog of the ToLCNDV-ES resistance allele) may still be present in such wild accessions. The skilled person is capable of identifying and introgressing the QTL1.1 comprising region found in other wild cucumber donors into cultivated cucumber, e.g. detecting wild accessions comprising the SNP markers or a subset thereof and transferring these SNP markers (or subset) into a cultivated cucumber line or variety and assessing the ToLCNDV-ES resistance of the cultivated line or variety compared to the line or variety lacking the SNP markers (or subset), i.e. lacking the introgression fragment. Even in cases where the SNP haplotype for SNP_01 to SNP_16 is identical to the SNP haplotype of QTL1.1 found in seeds of NCIMB 43745, the actual nucleotide sequences flanking the SNP at nucleotide 51 of SEQ ID NO: 1 to 16 may be different in other donors. So other donors may comprise the same SNP nucleotide at nucleotide 51, but in a sequence comprising at least 95%, 96%, 97%, 98% or 99% sequence identity to SEQ ID NO: 01 to 16 when e.g. aligned pairwise. This variation can be seen by sequencing the donors and aligning sequences of SEQ ID NO: 1 to SEQ ID NO: 16 with that sequence.

In one embodiment the presence of the introgression fragment comprising QTL1.1, or the chromosome 1 region (or variant or orthologous chromosome 1 region), comprising QTL1.1, is detectable by a molecular marker assay which detects at least 5, 6, 7, 8, 9, 10, 11, 12, 13, 14, 15 or 16 Single Nucleotide Polymorphism (SNP) markers selected from the group consisting of:
a) the TX or TT genotype for the Single Nucleotide Polymorphism marker SNP_01 at nucleotide 51 of SEQ ID NO: 1 (or at nucleotide 51 in a variant thereof);
b) the TX or TT genotype for the Single Nucleotide Polymorphism marker SNP_02 at nucleotide 51 of SEQ ID NO: 2 (or at nucleotide 51 in a variant thereof);
c) the TX or TT genotype for the Single Nucleotide Polymorphism marker SNP_03 at nucleotide 51 of SEQ ID NO: 3 (or at nucleotide 51 in a variant thereof);
d) the TX or TT genotype for the Single Nucleotide Polymorphism marker SNP_04 at nucleotide 51 of SEQ ID NO: 4 (or at nucleotide 51 in a variant thereof);
e) the AX or AA genotype for the Single Nucleotide Polymorphism marker SNP_05 at nucleotide 51 of SEQ ID NO: 5 (or at nucleotide 51 in a variant thereof);
f) the CX or CC genotype for the Single Nucleotide Polymorphism marker SNP_06 at nucleotide 51 of SEQ ID NO: 6 (or at nucleotide 51 in a variant thereof);
g) the TX or TT genotype for the Single Nucleotide Polymorphism marker SNP_07 at nucleotide 51 of SEQ ID NO: 7 (or at nucleotide 51 in a variant thereof);
h) the AX or AA genotype for the Single Nucleotide Polymorphism marker SNP_08 at nucleotide 51 of SEQ ID NO: 8 (or at nucleotide 51 in a variant thereof);
i) the GX or GG genotype for the Single Nucleotide Polymorphism marker SNP_09 at nucleotide 51 of SEQ ID NO: 9 (or at nucleotide 51 in a variant thereof); or the AX or AA genotype for the Single Nucleotide Polymorphism marker SNP_09 at nucleotide 51 of SEQ ID NO: 9 (or at nucleotide 51 in a variant thereof);
j) the CX or CC genotype for the Single Nucleotide Polymorphism marker SNP_10 at nucleotide 51 of SEQ ID NO: 10 (or at nucleotide 51 in a variant thereof);
k) the CX or CC genotype for the Single Nucleotide Polymorphism marker SNP_11 at nucleotide 51 of SEQ ID NO: 11 (or at nucleotide 51 in a variant thereof);
l) the CX or CC genotype for the Single Nucleotide Polymorphism marker SNP_12 at nucleotide 51 of SEQ ID NO: 12 (or at nucleotide 51 in a variant thereof);
m) the TX or TT genotype for the Single Nucleotide Polymorphism marker SNP_13 at nucleotide 51 of SEQ ID NO: 13 (or at nucleotide 51 in a variant thereof);
n) the AX or AA genotype for the Single Nucleotide Polymorphism marker SNP_14 at nucleotide 51 of SEQ ID NO: 14 (or at nucleotide 51 in a variant thereof);
o) the CX or CC genotype for the Single Nucleotide Polymorphism marker SNP_15 at nucleotide 51 of SEQ ID NO: 15 (or at nucleotide 51 in a variant thereof); or the TX or TT genotype for the Single Nucleotide Polymorphism marker SNP_15 at nucleotide 51 of SEQ ID NO: 15 (or at nucleotide 51 in a variant thereof);
p) the GX or GG genotype for the Single Nucleotide Polymorphism marker SNP_16 at nucleotide 51 of SEQ ID NO: 16 (or at nucleotide 51 in a variant thereof).

In one aspect said at least 5, 6, 7, 8, 9, 10, 11, 12, 13, 14, 15 or 16 markers which are detected are consecutive markers.

Thus, in one embodiment the plants according to the invention comprise at least a Thymine (T) (i.e. the TT or TX genotype) at nucleotide 51 of SEQ ID NO: 1 (referred to as SNP_01) or at the equivalent nucleotide of a genomic sequence comprising substantial sequence identity to SEQ ID NO:1 (in other words there is a Thymine at the physical position of chromosome 1 shown in Table 1);
and/or at least a Thymine (T) (i.e. the TT or TX genotype) at nucleotide 51 of SEQ ID NO: 2 (referred to as SNP_02) or at the equivalent nucleotide of a genomic sequence comprising substantial sequence identity to SEQ ID NO:2 (in other words there is a Thymine at the physical position of chromosome 1 shown in Table 1);
and/or at least a Thymine (T) (i.e. the TT or TX genotype) at nucleotide 51 of SEQ ID NO: 3 (referred to as SNP_03) or at the equivalent nucleotide of a genomic sequence comprising substantial sequence identity to SEQ ID NO:3 (in other words there is a Thymine at the physical position of chromosome 1 shown in Table 1);
and/or at least a Thymine (T) (i.e. the TT or TX genotype) at nucleotide 51 of SEQ ID NO: 4 (referred to as SNP_04) or at the equivalent nucleotide of a genomic sequence comprising substantial sequence identity to SEQ ID NO:4 (in other words there is a Thymine at the physical position of chromosome 1 shown in Table 1);
and/or at least an Adenine (A) (i.e. the AA or AX genotype) at nucleotide 51 of SEQ ID NO: 5 (referred to as SNP_05) or at the equivalent nucleotide of a genomic sequence comprising substantial sequence identity to SEQ ID NO:5 (in other words there is an Adenine at the physical position of chromosome 1 shown in Table 1);
and/or at least a Cytosine (C) (i.e. the CC or CX genotype) at nucleotide 51 of SEQ ID NO: 6 (referred to as SNP_06) or at the equivalent nucleotide of a genomic sequence comprising substantial sequence identity to SEQ ID NO:6 (in other words there is a Cytosine at the physical position of chromosome 1 shown in Table 1);
and/or at least a Thymine (T) (i.e. the TT or TX genotype) at nucleotide 51 of SEQ ID NO: 7 (referred to as SNP_07) or at the equivalent nucleotide of a genomic sequence comprising substantial sequence identity to SEQ ID NO:7 (in other words there is a Thymine at the physical position of chromosome 1 shown in Table 1);
and/or at least an Adenine (A) (i.e. the AA or AX genotype) at nucleotide 51 of SEQ ID NO: 8 (referred to as SNP 08) or at the equivalent nucleotide of a genomic sequence comprising substantial sequence identity to SEQ ID NO:8 (in other words there is an Adenine at the physical position of chromosome 1 shown in Table 1);
and/or at least a Guanine (G) (i.e. the GG or GX genotype) at nucleotide 51 of SEQ ID NO: 9 (referred to as SNP_09) or at the equivalent nucleotide of a genomic sequence comprising substantial sequence identity to SEQ ID NO:9 (in other words there is a Guanine at the physical position of chromosome 1 shown in Table 1); and/or at least an Adenine (A) (i.e. the AA or AX genotype) at nucleotide 51 of SEQ ID NO: 9 (referred to as SNP_09) or at the equivalent nucleotide of a genomic sequence comprising substantial sequence identity to SEQ ID NO:9 (in other words there is an Adenine at the physical position of chromosome 1 shown in Table 1);
and/or at least a Cytosine (C) (i.e. the CC or CX genotype) at nucleotide 51 of SEQ ID NO: 10 (referred to as SNP_10) or at the equivalent nucleotide of a genomic sequence comprising substantial sequence identity to SEQ ID NO: 10 (in other words there is a Cytosine at the physical position of chromosome 1 shown in Table 1);
and/or at least a Cytosine (C) (i.e. the CC or CX genotype) at nucleotide 51 of SEQ ID NO: 11 (referred to as SNP_11) or at the equivalent nucleotide of a genomic sequence comprising substantial sequence identity to SEQ ID NO: 11 (in other words there is a Cytosine at the physical position of chromosome 1 shown in Table 1);
and/or at least a Cytosine (C) (i.e. the CC or CX genotype) at nucleotide 51 of SEQ ID NO: 12 (referred to as SNP_12) or at the equivalent nucleotide of a genomic sequence comprising substantial sequence identity to SEQ ID NO: 12 (in other words there is a Cytosine at the physical position of chromosome 1 shown in Table 1);
and/or at least a Thymine (T) (i.e. the TT or TX genotype) at nucleotide 51 of SEQ ID NO: 13 (referred to as SNP_13) or at the equivalent nucleotide of a genomic sequence comprising substantial sequence identity to SEQ ID NO: 13 (in other words there is a Thymine at the physical position of chromosome 1 shown in Table 1);
and/or at least an Adenine (A) (i.e. the AA or AX genotype) at nucleotide 51 of SEQ ID NO: 14 (referred to as SNP_14) or at the equivalent nucleotide of a genomic sequence comprising substantial sequence identity to SEQ ID NO: 14 (in other words there is an Adenine at the physical position of chromosome 1 shown in Table 1);
and/or at least a Cytosine (C) (i.e. the CC or CX genotype) at nucleotide 51 of SEQ ID NO: 15 (referred to as SNP 15) or at the equivalent nucleotide of a genomic sequence comprising substantial sequence identity to SEQ ID NO: 15 (in other words there is a Cytosine at the physical position of chromosome 1 shown in Table 1); and/or at least a Thymine (T) (i.e. the TT or TX genotype) at nucleotide 51 of SEQ ID NO: 15 (referred to as SNP_15) or at the equivalent nucleotide of a genomic sequence comprising substantial sequence identity to SEQ ID NO: 15 (in other words there is a Thymine at the physical position of chromosome 1 shown in Table 1);
and/or at least a Guanine (G) (i.e. the GG or GX genotype) at nucleotide 51 of SEQ ID NO: 16 (referred to as SNP_16) or at the equivalent nucleotide of a genomic sequence comprising substantial sequence identity to SEQ ID NO: 16 (in other words there is a Guanine at the physical position of chromosome 1 shown in Table 1).

In a further one embodiment the presence of the introgression fragment, or the chromosome 1 region (or variant or orthologous chromosome 1 region), comprising QTL1.1, is detectable by a molecular marker assay which detects at least 3, 4 or 5 Single Nucleotide Polymorphism (SNP) markers of the sub-groups consisting of: SNP_01 to SNP05; SNP_05 to SNP 10; SNP_10 to SNP 15; or SNP_04 to SNP_12.

The SNP genotype refers to two nucleotides, and genomic sequences comprising one of these two nucleotides, one on each chromosome 1. So a plant having a TT genotype for SNP_01 has an identical nucleotide (T) on both chromosomes (i.e. is homozygous), while a plant having an TX genotype for SNP_01 has one chromosome with an T at nucleotide 51 of SEQ ID NO: 1 (or at the equivalent nucleotide of a genomic sequence comprising substantial sequence identity to SEQ ID NO:1) and one chromosome with a X at nucleotide 51 of SEQ ID NO: 1 (or at the equivalent nucleotide of a genomic sequence comprising substantial sequence identity to SEQ ID NO: 1) and is heterozygous, whereby X may be any nucleotide. As the genomic sequences around the SNP markers provided herein may vary slightly in introgression fragments from other wild cucumber donors (i.e. variants or orthologous chromosome 1 regions) it is clear that the nucleotide sequences before and after the SNP may not be 100% identical to the sequences provided herein. Therefore sequences having substantial sequence identity (i.e. at least 95% identity) to the sequences provided herein, but which comprise the same SNP, are encompassed herein.

In one aspect, the introgression fragment comprising QTL1.1, or the chromosome 1 region (or variant or orthologous chromosome 1 region) comprising the QTL (QTL1.1 or variant), which is detectable by the above one or more markers is from a wild or primitive cucumber, and in one aspect the wild or primitive cucumber is a member of the Indian Cucumber Group. In one aspect it is the same introgression fragment as found on chromosome 1 in seeds deposited under accession number NCIMB 43745, or a smaller fragment retaining the QTL. SNP markers SNP_01 to SNP_16 span a region of about 8 Mb. In one aspect the introgression fragment on chromosome 1 is equal to or less than 8 Mb in size, preferably equal to or less than 7.98 Mb in size, more preferably equal to or less than 7, 6, 5, 4, 3 or 2.5 Mb in size, e.g. equal to or less than 2 Mb. In one aspect the introgression fragment is at least 0.2 Mb, 0.5 Mb, 1.0 Mb, 1.5 Mb, 1.9 Mb, 2.0 Mb, 2.5 Mb, 2.7 Mb or 3 Mb in size. Thus, various ranges of introgression sizes are encompassed herein, such as fragments less than 8 Mb but more than 0.2 Mb, less than 6 Mb or 3 Mb but more than 0.2 Mb, 0.5 MB or 1 Mb, etc., which retain the QTL1.1 and one or more of the SNP markers of SNP_01 to SNP_16, or of the subgroups of SNP_01 to SNP 05; SNP 05 to SNP 10; SNP 10 to SNP_16 or SNP_04 to SNP_12. As mentioned before, the location of the QTL1.1 in the region spanning SNP_01 to SNP_16 can be determined by finemapping and recombinants comprising QTL1.1 on a smaller introgression fragment can be generated. The size of an introgression fragment can be easily determined by e.g. whole genome sequencing or Next Generation Sequencing, e.g. as described in Qi et al. 2013 (supra) or in Huang et al. 2009 (supra). Especially introgression regions can be easily distinguished from cultivated genomic regions due to the larger amount of genetic variation (SNPs, INDELs, etc.) in the introgression region.

To obtain the introgression fragment present on chromosome 1 (comprising QTL1.1) from the deposited seeds (NCIMB43745), i.e. to transfer the introgression fragments comprising the QTL to another cultivated cucumber plant, a plant is grown from the seed and the plant is crossed with a cultivated cucumber plant to obtain F1 seeds. As NCIMB43745 contains two recombinant chromosomes 1 (comprising the introgression fragment) all of the F1 seed and plants grown therefrom, contain one recombinant chromosome 1 from the NCIMB43745 parent and one non-recombinant chromosome 1 from the other cultivated parent. Thus, by traditional breeding one can transfer the recombinant chromosome 1 from NCIMB43745 into other cultivated cucumber lines or varieties. Plants which comprise the QTL1.1 can be screened for, and selected for, by the presence of one or more of the above SNP markers in order to identify plants comprising a recombinant chromosome 1.

To generate shorter introgression fragments (comprising QTL1.1) meiosis needs to take place and plants comprising the recombinant chromosomes 1, and especially new meiotic recombination events within the introgression fragment, need to be identified. For example, seeds of NCIMB43745 can be selfed one or more times to produce F1, F2 or F3 plants (or further selfing generations), and/or F1, F2 or F3 plants (etc.) comprising a recombinant chromosome 1 can be backcrossed to a cultivated parent. Plants which comprise the recombinant chromosome 1 can be screened for, and selected for, by the presence of one or more of the above SNP markers in order to identify plants comprising a smaller introgression fragment. Such new recombinants can then be tested for the presence of the QTL1.1 on the smaller introgression fragment by determining the average disease score in a ToLCNDV-ES disease assay compared to the (genetic) control lacking the introgression fragment.

Similarly, cultivated cucumber plants comprising QTL1.1 (or a variant thereof) can be generated and/or identified using different methods. For example, to obtain a cultivated cucumber plant comprising a introgression fragment from a wild donor, a wild donor is identified which comprises one or more of the SNP markers linked to QTL1.1 disclosed herein, e.g. any one, or more, or all of the markers described herein above. This has for example been done for various wild accessions, see Examples. The identified plant is crossed with a cultivated cucumber plant to obtain F1 seeds. The F1 can be selfed to produce F2, F3, etc. plants, and/or F2 plants or F3 plants, etc., can be backcrossed to the cultivated cucumber parent. Plants which are comprising QTL1.1 (or a variant thereof) can be screened for, and/or selected for, by the presence of one or more of the above SNP markers and/or screened for, and/or selected for, an increased ToLCNDV-ES resistance phenotype compared to the initial cultivated parent (lacking the introgressions). Alternatively or in addition, QTL mapping can be carried out in order to identify further molecular markers linked to the QTL1.1 (or a variant thereof) and/or to generate cultivated cucumber plants comprising an introgression fragment on chromosome 1 which confers significantly enhanced ToLCNDV-ES resistance.

In one embodiment the presence of the introgression fragment in a cultivated cucumber plant, or the chromosome 1 region (or orthologous chromosome 1 region), comprising QTL1.1, is detectable by a molecular marker assay which detects at least 5, 6, 7, 8, 9, 10 or more of the markers selected from the group consisting of:
a) the TT or TX genotype for the Single Nucleotide Polymorphism marker SNP_01 in SEQ ID NO: 1 (or in a variant thereof);
b) the GG or GX genotype for the Single Nucleotide Polymorphism marker SNP_16 in SEQ ID NO: 16 (or in a variant thereof);
c) any wild cucumber genome-specific marker in between marker SNP_01 and SNP_16.

In one aspect the markers of c) are one or more of SNP_02 to SNP_15. In one aspect, at least 5, 6, 7, 8, 9, 10 or more markers are detected from the markers of a), b) and/or c) above. In one embodiment at least the marker of a) and/or b) is detected and optionally at least one, two, three or more markers of c) are detected. In one aspect the markers detected are consecutive markers.

Any wild cucumber genome-specific marker in between two markers refers to any molecular marker which maps genetically to the chromosome 1 region in-between the two markers and/or which lies physically in-between the two markers, and which is indicative of the wild cucumber chromosome 1 region. This means that the marker is polymorphic between the cultivated cucumber genome and the wild cucumber genome. In one aspect, the marker is a Single Nucleotide Polymorphism (SNP), but other molecular markers such as RFLP, AFLP, RAPD, DNA sequencing, etc. may equally be used.

The introgression fragment in the plants of the invention is in one aspect a fragment of chromosome 1 (comprising QTL1.1) which is present in seeds deposited under accession number NCIMB43745 or a smaller version of that fragment retaining the QTL (generated by e.g. recombination within the introgression fragment).

The introgression fragment is in one aspect equal to or less than 8 Mb in size, preferably equal to or less than 7 Mb, 5 Mb, 3 Mb, 2.5 Mb, 2 Mb, 1.5 Mb, 1 Mb in size. In a further aspect the introgression fragment is at least 0.5 Mb or at least 1 Mb in size.

Also provided are seeds from which a plant of the invention can be grown, as are cucumber fruits harvested from a plant of the invention and comprising the recombinant chromosome 1 in their genome (comprising QTL1.1 or a variant). Likewise a plant cell, tissue or plant part of a plant or of a seed is provided comprising at least one recombinant chromosome 1 (comprising QTL1.1 or a variant), wherein said recombinant chromosome 1 comprises an introgression fragment from a wild or primitive cucumber and wherein said introgression fragment comprises an allele conferring significantly enhanced ToLCNDV-ES resistance.

The molecular markers described herein may be detected according to standard method. For example SNP markers can easily be detected using a KASP-assay (see www.kpbioscience.co.uk) or other SNP genotyping assays. For developing a KASP-assay, for example 50 or 70 base pairs upstream and 50 or 70 base pairs downstream of the SNP can be selected and two allele-specific forward primers and one allele specific reverse primer can be designed. See e.g. Allen et al. 2011, Plant Biotechnology J. 9, 1086-1099, especially p 097-1098 for KASP assay method.

Thus, in one aspect, the SNP markers and the presence/absence of the marker associated with QTL1.1 is determined using a KASP assay, but equally other SNP genotyping assays can be used. For example, a TaqMan SNP genotyping assay, a High Resolution Melting (HRM) assay, SNP-genotyping arrays (e.g. Fluidigm, Illumina, etc.) or DNA sequencing may equally be used.

The physical size of an introgression fragment can be determined by various methods, such as physical mapping, sequencing or by visualization of the introgression using Fluorescent in situ hybridization (FISH) images (Verlaan et al. 2011, Plant Journal 68: 1093-1103).

Cultivated cucumber plants with smaller introgression fragments on chromosome 1 (comprising QTL1.1 or a variant) can be generated by generating new recombinant plants from a population of plants derived from a cross between a cultivated cucumber plant (lacking the introgressions) and a plant of the invention and selecting recombinant progeny having smaller introgression sizes. Such plants are thus in one aspect derived from (progeny or descendants of) the recombinant chromosome 1 present in plants of which seeds have been deposited under NCIMB43745. Such progeny or descendants which retain the QTL1.1, and thus the higher ToLCNDV-ES resistance compared to plants lacking an introgression as described herein, are encompassed herein.

### QTL1.2 or a Variant QTL1.2 on Chromosome 1

Thus, in one aspect a cultivated cucumber plant is provided comprising an introgression fragment from a wild or primitive cucumber, wherein the introgression fragment comprises QTL1.2, or a variant thereof, and wherein the introgression fragment comprises all or part of the region starting at nucleotide (or base) 22942981 of chromosome 1 (corresponding to SNP_17) and ending at nucleotide (or base) 25543032 of chromosome 1 (corresponding to SNP_31). In other words, all or part of the region starting at nucleotide 22942981 (SNP_17) of chromosome 1 and ending at nucleotide 25543032 (SNP_31) of chromosome 1 is, in one aspect, from a wild donor cucumber and comprises QTL1.2 or a variant thereof. Which sub-region contains QTL1.2 can be identified by e.g. fine-mapping. So, for example if QTL1.2 is found to be in between SNP_17 and SNP_22, then the plant of the invention only needs to comprise the introgression region starting at nucleotide 22942981 of chromosome 1 (SNP_17) and ending at nucleotide 23844859 (SNP 22) of chromosome 1.

In one aspect QTL1.2 (or a variant thereof) is located in-between marker SNP_17 at nucleotide 51 of SEQ ID NO: 17 (or at nucleotide 51 in a variant sequence of SEQ ID NO: 17) and marker SNP_31 at nucleotide 51 of SEQ ID NO: 31 (or at nucleotide 51 in a variant sequence of SEQ ID NO: 31). In another aspect QTL1.2 (or a variant thereof) is located in-between marker SNP_17 at nucleotide 51 of SEQ ID NO: 17 (or at nucleotide 51 in a variant sequence of SEQ ID NO: 17) and marker SNP_22 at nucleotide 51 of SEQ ID NO: 22 (or at nucleotide 51 in a variant sequence of SEQ ID NO: 22). In a further aspect QTL1.2 (or a variant thereof) is located in-between marker SNP_22 at nucleotide 51 of SEQ ID NO: 22 (or at nucleotide 51 in a variant sequence of SEQ ID NO: 22) and marker SNP_27 at nucleotide 51 of SEQ ID NO: 27 (or at nucleotide 51 in a variant sequence of SEQ ID NO: 27). In a further aspect QTL1.2 (or a variant thereof) is located in-between marker SNP_27 at nucleotide 51 of SEQ ID NO: 27 (or at nucleotide 51 in a variant sequence of SEQ ID NO: 27) and marker SNP_31 at nucleotide 51 of SEQ ID NO: 31 (or nucleotide 51 in a variant sequence of SEQ ID NO: 31). In a further aspect QTL1.1 (or a variant thereof) is located in-between marker SNP_20 at nucleotide 51 of SEQ ID NO: 20 (or at nucleotide 51 in a variant sequence of SEQ ID NO: 20) and marker SNP_29 at nucleotide 51 of SEQ ID NO: 29 (or nucleotide 51 in a variant sequence of SEQ ID NO: 29).

In another aspect the introgression fragment of the invention (comprising QTL1.1 or a variant thereof) is a fragment comprising a smaller fragment (part) of the region starting at nucleotide (or base) 22942981 of chromosome 1 and ending at nucleotide (or base) 25543032 of chromosome 1, e.g. having a size of e.g. 2.5 Mb, 2 Mb, 1 Mb, 0.5 Mb, 100 kb, 50 kb, 35 kb, 30 kb, 20 kb, or less and comprising the QTL or a variant thereof. In one aspect the part is at least 5 kb, 10 kb, 20 kb in size, or more.

In one aspect the cultivated cucumber plant of the invention comprises an introgression fragment from a wild or primitive cucumber, which introgression fragment comprises QTL1.2 or a variant thereof, wherein the introgression fragment comprises all of part of the region starting at 22.8 Mb and ending at 25.6 Mb of the physical chromosome 1.

In one aspect the introgression fragment on chromosome 1 comprising QTL1.2, or a variant thereof, is obtainable by crossing a plant grown from NCIMB43745 with another cucumber plant, especially a cultivated cucumber plant, in one aspect a long cucumber type or a pickling or slicer type.

In one aspect the cultivated cucumber plant of the invention comprising QTL1.2, or a variant thereof, is a plant wherein said introgression fragment on chromosome 1 is obtainable by crossing a plant grown from seeds deposited under accession number NCIMB43745 with another cucumber plant. Thus, in one aspect the QTL is the QTL present in seeds deposited under accession number NCIMB43745.

In a further aspect the cultivated cucumber plant of the invention comprising QTL1.2, or a variant thereof, is a plant wherein said introgression fragment on chromosome 1 is obtainable by crossing a plant comprising the same SNP haplotype or SNP genotype for at least 5, 6, 7, 8, 9, 10 or more SNP markers linked to the QTL (i.e. SNP_17 to SNP_31 for QTL1.2 as shown in Table 2) with another cucumber plant, especially with a cultivated cucumber elite breeding line. Thus, in one aspect the QTL is the QTL present in wild donor accessions which comprise the same SNP haplotype or genotype for at least 5, 6, 7, 8, 9, 10 or more of the SNP markers, e.g. as found in NCIMB43745. Preferably the donor also comprises a resistance phenotype having an average ToLCNDV-ES disease score of at least 7.5, preferably at least 8.0, 8.1, 8.2, 8.3, 8.4, 8.5, 8.6, 8.7, 8.8, 8.9 or 9.0.

When referring to the SNP markers herein, which are indicative of the presence of the introgression fragment (and the ToLCNDV-ES resistance QTL present on the introgression fragment), it is understood that the SNP genotype or haplotype which is indicative of the introgression fragment is referred to, i.e. the SNP genotype or haplotype as provided e.g. in Tables 1 to 4. It is noted that the SNP marker genotype can distinguish between the introgression fragment being in homozygous or heterozygous form. In homozygous form the nucleotide is identical, while in heterozygous form the nucleotide is not identical. The SNP genotype of the ‘wild type’ chromosome lacking the introgression fragment is the other haplotype, e.g. the haplotype of the recurrent parent). So, e.g. the genotype of SNP_17 indicative of the introgression fragment comprising QTL1.2 is ‘CT’ (QTL1.2 wt) or ‘CC’ (QTL1.2 QTL1.2) while the SNP genotype indicative of the wild type/genetic control (lacking the introgression fragment) is e.g. ‘TT’ (wt/wt). This can also be written as genotype CX′ (QTL1.2 wt) or ‘CC’ (QTL1.2 QTL1.2) while the SNP genotype indicative of the wild type/genetic control (lacking the introgression fragment) is e.g. ‘XX’ (wt/wt). X may be any nucleotide (A, T, C or G). Thus, when referring to a plant or plant part (e.g. cell) comprising the introgression fragment in homozygous or heterozygous form, it is understood that the SNP markers linked to the introgression fragment have the corresponding SNP genotype or haplotype.

So, in one aspect, a cultivated Cucumis sativus var. sativus plant is provided comprising an introgression fragment on chromosome 1 in homozygous or heterozygous form, wherein said introgression fragment confers an increase in ToLCNDV-ES resistance compared to the cucumber plant lacking the introgression fragment on chromosome 1, e.g. the genetic control or control variety, when grown under the same conditions.

The increase in ToLCNDV-ES resistance is phenotypically expressed as a higher average disease score (less yellowing, measured e.g. in a disease assay as described herein) of the cultivated cucumber plant line or variety comprising the introgression fragment on chromosome 1 in homozygous or heterozygous form compared to the genetic control line or variety lacking the introgression fragment on chromosome 1 when grown under the same environment. The average disease score is preferably increased by at least 1.0, 1.5, 2.0, 2.5, 3.0 or more points on the disease scale of 2.0 (90-100% of leaf area is covered with yellowing mosaic symptoms) to 9.0 (no symptoms). So, for example if QTL1.2 is introduced into a susceptible cucumber line or variety having an average disease score of about 4.0, the introduction of QTL1.2 preferably increases the average disease score to an average score of at least 5.0, 5.5, 6.0, 6.5, 7.0 or more. As QTL1.2 was found to be additive, the effect of QTL1.2 in heterozygous form is less than the effect in homozygous form. Therefore the effect on ToLCNDV-ES resistance is preferably measured when it is in homozygous form.

It is known that all four QTLs together in homozygous form (QTL1.1, QTL1.2, QTL2.1 and QTL3.1) lead to an average disease score of 9.0 when introduced into a susceptible plant having an average disease score of about 4.0 to 5.0. The effect of the individual QTLs, even when in homozygous form, will be smaller than the combined effect, but will still be effective in reducing ToLCNDV-ES symptoms. The effect of QTL1.2 alone can be determined by introducing the QTL alone into a susceptible cucumber plant, in heterozygous or preferably in homozygous form. For example NCIMB43745 can be crossed with a susceptible plant and QTL1.2 alone can be transferred into the susceptible background.

The plants of the invention therefore comprise a genome of cultivated cucumber, with at least one or two recombinant chromosomes, namely one or two recombinant chromosomes 1 (i.e. heterozygous or homozygous). The recombinant chromosomes comprise a fragment of a wild donor cucumber, which is easily distinguishable from the cultivated cucumber genome by molecular marker analysis, whole genome sequencing, chromosome painting and similar techniques.

In one aspect the introgression fragment on chromosome 1 is from a wild or primitive cucumber, comprises the ToLCNDV-ES QTL1.2, or a variant thereof, and comprises all or part of the region starting at nucleotide SNP_17 and ending at SNP_31. Thus, the introgression fragment comprises the QTL1.2 or a variant thereof and one or more or all (e.g. 5, 6, 7, 8, 9, 10, 11, 12, 13, 14, 15) SNP markers of the wild donor selected from SNP_17 to SNP_31 as shown in Table 2.

In one aspect the introgression fragment comprises QTL1.2 and one or more or all of SEQ ID NO: 17 to SEQ ID NO: 31.

In one aspect the presence of the introgression fragment on chromosomes 1 comprising QTL1.2 in the genome of the plant or plant cell or plant tissue (or in the DNA extracted therefrom) is detectable by a molecular marker assay which detects one or more molecular markers of the introgression fragment, especially the donor SNP haplotype or genotype for at least 5, 6, 7, 8, 9, 10 or more of SNP_17 to SNP_31, at nucleotide 51 of SEQ ID NO: 17 to 31, respectively. However, as mentioned, other techniques may be used, e.g. the SNP genotype of the markers may also be determined by sequencing or by using alternative markers located in between the SNP markers provided herein or within 7 cM, or within 5 cM, of a marker provided herein; or within 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less of a marker provided herein.

In one aspect the presence of the introgression fragment on chromosomes 1 comprising QTL1.2 in the genome of the plant or plant cell or plant tissue (or in the DNA extracted therefrom) is detectable by detecting the presence of one or more or all of SEQ ID NO: 17 to SEQ ID NO: 31.

When reference is made herein to one or more molecular markers or sequences being “detectable” by e.g. a molecular marker assay, this means of course that the plant or plant part comprises the one or more markers or sequences in its genome, as the marker or sequence would otherwise not be detectable.

### Cucumber Plants Comprising an Introgression Fragment on Chromosome 1 (QTL 1.2) or a Variant

In one aspect a cultivated Cucumis sativus var. sativus plant comprising an introgression fragment from a wild or primitive cucumber on chromosome 1 in homozygous or heterozygous form is provided, wherein said introgression fragment comprises a Quantitative Trait Locus (QTL) located between the Single Nucleotide Polymorphism marker SNP_17 at nucleotide 51 of SEQ ID NO: 17 (or at nucleotide 51 of a variant of SEQ ID NO: 17) and the Single Nucleotide Polymorphism marker SNP_31 at nucleotide 51 of SEQ ID NO: 31 (or at nucleotide 51 of a variant of SEQ ID NO: 31), which QTL confers an increase in ToLCNDV-ES resistance. In one aspect the QTL is located between base 22942981 (SNP_17) and base 25543032 (SNP_31) of chromosome 1.

Thus, in one aspect QTL1.2 (or a variant thereof) is located in the region between SNP_17 in SEQ ID NO: 1 (or in a variant thereof) and SNP_31 in SEQ ID NO: 31 (or in a variant thereof).

Therefore, in one aspect a cultivated Cucumis sativus var. sativus plant is provided comprising an introgression fragment on chromosome 1 in homozygous or heterozygous form, wherein said introgression fragment confers an increase in ToLCNDV-ES resistance (compared to the plant lacking the introgression fragment, e.g. the genetic control) and wherein said introgression fragment comprises the SNP marker haplotype or genotype of at least 5, 6, 7, 8, 9, 10, 11, 12, 13, 14 or 15 of the SNP markers selected from the group consisting of:
a) the CX or CC genotype for the Single Nucleotide Polymorphism marker SNP_17 at nucleotide 51 of SEQ ID NO: 17 (or at nucleotide 51 in a variant thereof);
b) the GX or GG genotype for the Single Nucleotide Polymorphism marker SNP_18 at nucleotide 51 of SEQ ID NO: 18 (or at nucleotide 51 in a variant thereof);
c) the GX or GG genotype for the Single Nucleotide Polymorphism marker SNP_19 at nucleotide 51 of SEQ ID NO: 19 (or at nucleotide 51 in a variant thereof);
d) the TX or TT genotype for the Single Nucleotide Polymorphism marker SNP_20 at nucleotide 51 of SEQ ID NO: 20 (or at nucleotide 51 in a variant thereof);
e) the GX or GG genotype for the Single Nucleotide Polymorphism marker SNP_21 at nucleotide 51 of SEQ ID NO: 21 (or at nucleotide 51 in a variant thereof);
f) the GX or GG genotype for the Single Nucleotide Polymorphism marker SNP_22 at nucleotide 51 of SEQ ID NO: 22 (or at nucleotide 51 in a variant thereof);
g) the TX or TT genotype for the Single Nucleotide Polymorphism marker SNP_23 at nucleotide 51 of SEQ ID NO: 23 (or at nucleotide 51 in a variant thereof);
h) the CX or CC genotype for the Single Nucleotide Polymorphism marker SNP_24 at nucleotide 51 of SEQ ID NO: 24 (or at nucleotide 51 in a variant thereof);
i) the AX or AA genotype for the Single Nucleotide Polymorphism marker SNP_25 at nucleotide 51 of SEQ ID NO: 25 (or at nucleotide 51 in a variant thereof);
j) the CX or CC genotype for the Single Nucleotide Polymorphism marker SNP_26 at nucleotide 51 of SEQ ID NO: 26 (or at nucleotide 51 in a variant thereof);
k) the CX or CC genotype for the Single Nucleotide Polymorphism marker SNP_27 at nucleotide 51 of SEQ ID NO: 27 (or at nucleotide 51 in a variant thereof);
l) the CX or CC genotype for the Single Nucleotide Polymorphism marker SNP_28 at nucleotide 51 of SEQ ID NO: 28 (or at nucleotide 51 in a variant thereof);
m) the CX or CC genotype for the Single Nucleotide Polymorphism marker SNP_29 at nucleotide 51 of SEQ ID NO: 29 (or at nucleotide 51 in a variant thereof);
n) the AX or AA genotype for the Single Nucleotide Polymorphism marker SNP_30 at nucleotide 51 of SEQ ID NO: 30 (or at nucleotide 51 in a variant thereof);
o) the GX or GG genotype for the Single Nucleotide Polymorphism marker SNP_31 at nucleotide 51 of SEQ ID NO: 31 (or at nucleotide 51 in a variant thereof).

When referring to a SNP in a variant sequence, that variant sequence comprises at least 95%, 96%, 97%, 98% or 99% sequence identity with the mentioned sequence. X refers to any nucleotide for the sequence on the other chromosome 1 of the pair of chromosomes. In one aspect X may be the nucleotide of the recurrent parent as described in Table 2.

In one aspect said at least 5, 6, 7, 8, 9, 10, 11, 12, 13, 14 or 15 markers are consecutive markers.

The fragment comprising the QTL1.2 may, thus, be large (comprising SNP_17 to SNP_31), or may be smaller and lack markers having the genotype or haplotype of the wild cucumber (i.e. the markers have the cultivated cucumber genotype or haplotype instead, see also Table 2, SNP haplotype of recurrent parent), but it may still confer enhanced ToLCNDV-ES resistance on the cultivated cucumber plant, i.e. it can still comprise the ToLCNDV-ES allele (QTL1.2 or a variant). Such smaller introgression fragments are an embodiment of the invention. Plants having smaller introgression fragments which still confer the enhanced ToLCNDV-ES resistance (i.e. contain the resistance allele) can be generated using known techniques, such as fine-mapping or similar techniques. For example by starting with a plant comprising the introgression fragment as found in seeds deposited under accession number NCIMB 43745 and crossing such a plant with another cultivated cucumber plant and selfing the progeny of said cross, and/or backcrossing the progeny, to generate a population of plants which may contain recombinants having a smaller introgression fragment on chromosome 1, which fragments still confer enhanced ToLCNDV-ES resistance in relation to a plant lacking the introgression fragment (such as the genetic control, e.g. plants grown from seeds deposited under NCIMB42344), e.g. a fragment comprising markers SNP_17 to SNP 22, or SNP_22 to SNP_27 or SNP_27 to SNP_31 or SNP_20 to SNP_29. Marker assays can be used to determine the size of the smaller introgression fragment. One or more of the SNP markers with the genotype or haplotype of the wild donor cucumber may be missing. The cultivated cucumber genotype or haplotype is then detected for these SNP markers. The ToLCNDV-ES resistance of plants comprising such a smaller introgression fragment can then be compared in a disease assay as described herein, i.e. growing a plurality of plants comprising the smaller introgression fragment in experiments together with suitable control plants, lacking the introgression fragments. The control plants are preferably a genetic control, such as NCIMB43744. If the average ToLCNDV-ES disease score remains significantly higher than in the control, then the smaller introgression fragment has retained the QTL1.2.

Alternatively, the same or variant QTL (QTL1.2 or variant QTL1.2) may be introgressed from a different wild donor accessions, whereby optionally not all SNP markers disclosed herein may be present, i.e. the SNP haplotype of the donor accession may only be identical to the SNP haplotype of the QTL1.2 present in seeds of NCIMB43745 for at least 5, 6, 7, 8, 9, 10 or more SNPs. Such alternative wild cucumber sources can be identified using the SNP markers provided herein, by screening germplasm (i.e. accessions of) wild or primitive cucumber using a marker assay to detect the genotype or haplotype of one or more markers of markers SNP_17 to SNP_31, or of markers SNP_17 to SNP 22, SNP_22 to SNP 27, SNP_27 to SNP_31, or SNP_20 to SNP 29, or even only a smaller subgroup of these markers (e.g. 2, 3 or 4). For example, Table 2 shows various donors (PI605996, CGN22263 and CGN22932, also known as PI197087) which have the same SNP haplotype for SNP_17 to SNP_31. In the same way other donors, having QTL1.2 or a variant thereof, may be identified. Plants comprising the same or variant QTL1.2 from these donors or from other sources are also an embodiment of the invention. Thus, as long as at least 5, 6, 7, 8, 9, 10 or more (or all) of the SNPs of SNP_17 to SNP 31, or of the SNPs of SNP_17 to SNP_22, or of the SNPs of SNP_22 to SNP 27, or of the SNPs of SNP_27 to SNP_31, or of the SNPs of SNP_20 to SNP_29 are present, the donor may contain QTL1.2 (or a variant thereof) and is encompassed herein. The skilled person can then introgress the QTL1.2 (or a variant thereof) into cultivated cucumber in order to enhance ToLCNDV-ES resistance as described herein and in order to confirm that the QTL enhances ToLCNDV-ES resistance when present in cultivated cucumber. Prior to introgression the wild donor may also be tested for ToLCNDV-ES resistance in an assay as described and e.g. a donor may be selected that comprises an average ToLCNDV-ES score of e.g. at least 7.5, 8.0, 8.5, or 9.0.

As described above, in one embodiment the cultivated cucumber plant of the invention comprises an introgression fragment comprising at least a subset of SNP markers with the genotype (or haplotype) of the wild donor cucumber, i.e. at least 5, 6, 7, 8, 9 or more markers of SNP_17 to SNP 31, or at least 3 markers of SNP_17 to SNP 22, or of SNP_22 to SNP_27, or of SNP_27 to SNP 31, or of SNP_20 to SNP_29. In one aspect the cultivated cucumber plant comprises all, or all except 1 or 2 markers of SNP_17 to SNP_31, or of SNP_17 to SNP 22, or of SNP_22 to SNP 27, or of SNP_27 to SNP_31, or of SNP_20 to SNP_29.

Thus, the introgression fragment (and a cultivated cucumber plant or plant part, e.g., a cell, comprising the introgression fragment) can be detected in a marker assay by detecting the SNP genotype or haplotype of the introgression fragment (i.e. of the wild cucumber germplasm) of one or more or all of the markers above, preferably at least 5, 6, 7, 8 or more.

Thus, in one aspect, a Quantitative Trait Locus (QTL1.2) was found to be present on chromosome 1 of a wild cucumber donor which, when transferred (introgressed) into a cultivated cucumber variety or breeding line, and when present in heterozygous or homozygous form, confers significantly enhanced ToLCNDV-ES resistance onto the cultivated cucumber plant. The QTL, or the introgression fragment comprising the QTL (comprising the ToLCNDV resistance allele), is thus additive, i.e. it is sufficient to have the introgression fragment on one of the chromosomes 1 (one recombinant chromosome 1), while the homologous chromosome 1 of the pair may be a (non-recombinant) chromosome 1 of cultivated C. sativus var. sativus lacking the introgression fragment.

Although the present source of the QTL1.2 which was used to map and introgress the QTL is a single, specific wild source, there are other wild accessions which comprise QTL1.2 at the same locus on chromosome 1. For example the wild accessions PI605996, CGN22263 and CGN22932 (also known as PI197087) were found to comprise the same SNP haplotype for markers SNP_17 to SNP_31 (as shown in Table 2) and were found to be resistant to ToLCNDV-ES. The (variant) QTL1.2 from these or other donors can, thus, be introgressed into cultivated cucumber, optionally in combination with one or more of QTL1.1, QTL2.1 and QTL3.1. Similarly, other wild or primitive cucumber accessions can be screened for the SNP haplotype or genotype of one or more or all of SNP_17 to SNP_31.

Such other donors may comprise a ToLCNDV-ES resistance allele which has a slightly different nucleotide sequences, i.e. variants of the allele (QTL1.2) found herein. Such variant QTLs can also be identified and introgressed into cultivated cucumber as described herein, to generate a cultivated cucumber plant comprising a genome of cultivated C. sativus var. sativus and a recombinant chromosome 1, whereby the recombinant chromosome 1 comprises an introgression fragment, which confers an enhanced ToLCNDV-ES resistance onto the cultivated cucumber plant when present in homozygous or heterozygous form. To identify such wild donor accessions comprising QTL1.2, wild accessions can be screened, e.g. in a marker assay or by sequence comparison or other methods, for the presence of one or more of the SNP markers provided herein. The putative QTL (or variant QTL) can then be introgressed into cultivated cucumber, e.g. using MAS, i.e. using one or more (or all) of the SNP markers provided herein to detect and/or select progeny plants (e.g. backcross plants) comprising a recombinant chromosome 1. The selected plants, i.e. the cultivated cucumber plants comprising an introgression fragment on chromosome 1, wherein the introgression fragment on chromosome 1 is detectable by 5, 6, 7, 8, 9, 10 or more of the SNP markers SNP_17 to SNP_31 can then be phenotyped in a ToLCNDV-ES disease assay together with the suitable control plants, preferably at least the genetic control, in order to determine whether the introgression fragment indeed causes a significant increase in ToLCNDV-ES resistance.

Accessions of wild or primitive cucumber, are obtainable from e.g. the USDA National Plant Germplasm System collection or other seed collections, and can thus be screened for the presence of QTL1.2 using e.g. a marker assay as described herein, and accessions comprising 5 or more of the SNP markers (e.g. at least 5, 6, 7, 8, 9, 10 or more SNP markers indicative of QTL1.2) can be crossed with a cultivated cucumber plant having normal wild-type, non-recombinant chromosomes 1. The F1 or F2 generation (or further generation, such as the F3 or a backcross generation) can then be screened for recombinant plants having the introgression fragment or a part thereof, using the molecular marker assays described herein.

In one aspect, the introgression fragment is from a donor comprising the SNP haplotype for QTL1.2 as shown in Table 2 for the introgression donor (NCIMB43745), for P1605996, for CGN22263, for CGN22932 also known as PI197087.

In a specific embodiment, the introgression fragment comprising the ToLCNDV-ES QTL1.2 (or a variant) is derivable from (or derived from) or obtainable from (or obtained from; or as present in) seeds, a representative sample of which has been deposited under accession number NCIMB 43745, or from progeny thereof, or from seeds having accession number PI605996 (USDA ARS-GRIN collection), or from seeds having accession number CGN22263 or CGN22932 (Wageningen CGN collection) or from seeds having accession number PI197087 (USDA ARS-GRIN collection) or from progeny of any of these. The progeny may be any progeny which retain the SNP markers or haplotype indicative of (and linked to) the QTL, as described. Thus, progeny are not limited to F1 or F2 progeny of the deposit or accession, but can be any progeny, whether obtained by selfing and/or crossing with another cucumber plant.

In one embodiment the introgression fragment comprising QTL1.2 is identifiable by one or more of the markers described elsewhere herein, especially markers SNP_17 to SNP_31 for the introgression fragment on chromosome 1, or a subset of markers, such as one or more of the markers selected from SNP markers SNP_17 to SNP 22, or from SNP markers SNP_22 to SNP 27, or from of the SNP markers SNP_27 to SNP_31, or from SNP markers SNP_20 to SNP_29. In one aspect the invention provides a cultivated cucumber plant, having a genome of cultivated (domesticated) cucumber which comprises enhanced ToLCNDV-ES resistance, wherein the enhanced resistance is conferred by an introgression fragment on the cultivated cucumber chromosome 1, wherein said introgression fragment is obtained by (or obtainable by) crossing a cultivated plant grown from seeds deposited under NCIMB 43745 or progeny of this plant (which comprises one or more the markers disclosed herein linked to the QTL) with a cultivated cucumber plant. Thus in one aspect the cultivated cucumber plant of the invention comprises the same introgression fragment and the same recombinant chromosome 1 as present in NCIMB 43745 (comprising all of the wild donor haplotype for SNP markers SNP_17 to SNP_31 or comprising SEQ ID NO: 17 to 31), or it comprises a shorter fragment of that introgression fragment, whereby the shorter fragment retains the genetic element conferring ToLCNDV-ES resistance (QTL1.2).

Thus in one aspect the invention relates to a plant of the invention i.e. a cultivated Cucumis sativus var. sativus plant comprising an introgression fragment comprising QTL1.2 from a wild cucumber on chromosome 1 in homozygous or heterozygous form and wherein said introgression fragment is the introgression fragment “as in”/is “identical to”/is “the same as in” the seeds deposited under number NCIMB 43745, or is a shorter fragment thereof, but still confers enhanced ToLCNDV-ES resistance due to the presence of QTL1.2.

As SEQ ID NO: 17 to 31 are from the wild donor used to generate NCIMB43745, they can identify the introgression fragment or sub-fragments of the specific donor.

In yet another embodiment the invention relates to a plant of the invention i.e. a cultivated Cucumis sativus var. sativus plant comprising an introgression fragment comprising QTL1.2 from a wild cucumber on chromosome 1 in homozygous or heterozygous form and wherein said introgression fragment is the introgression fragment is a variant of the introgression fragment seeds deposited under number NCIMB 43745, i.e. it comprises the QTL 1.2, but the genomic sequence may be different. As wild accessions will be genetically divergent, the genomic sequence of an introgression fragment comprising QTL1.2 from other wild or primitive cucumbers will most likely not be identical to the genomic sequence as introgressed into NCIMB 43745, and even the ToLCNDV-ES conferring gene (comprising a promoter, introns and exons) may be divergent in nucleotide sequence, but the function will be the same, i.e. conferring enhanced resistance. The divergence can be seen in that certain SNP markers linked to QTL1.2 may be commonly found in various accessions, while other SNP markers may only be found in specific accessions. So for example not all of SNP_17 to SNP_31 may be found in other wild cucumber donors. For example an accession may have a slightly different SNP haplotype for SNP_17 to SNP_31, with one or two SNP markers having a different nucleotide. However, QTL1.2 (comprising e.g. a variant or ortholog of the ToLCNDV-ES resistance allele) may still be present in such wild accessions. The skilled person is capable of identifying and introgressing the QTL1.2 comprising region found in other wild cucumber donors into cultivated cucumber, e.g. detecting wild accessions comprising the SNP markers or a subset thereof and transferring these SNP markers (or subset) into a cultivated cucumber line or variety and assessing the ToLCNDV-ES resistance of the cultivated line or variety compared to the line or variety lacking the SNP markers (or subset), i.e. lacking the introgression fragment. Even in cases where the SNP haplotype for SNP_17 to SNP_31 is identical to the SNP haplotype of QTL1.2 found in seeds of NCIMB 43745, the actual nucleotide sequences flanking the SNP at nucleotide 51 of SEQ ID NO: 17 to 31 may be different in other donors. So other donors may comprise the same SNP nucleotide at nucleotide 51, but in a sequence comprising at least 95%, 96%, 97%. 98% or 99% sequence identity to SEQ ID NO: 17 to 31 when e.g. aligned pairwise. This variation can be seen by sequencing the donors and aligning sequences of SEQ ID NO: 17 to SEQ ID NO: 31 with that sequence.

In one embodiment the presence of the introgression fragment comprising QTL1.2, or the chromosome 1 region (or variant or orthologous chromosome 1 region), comprising QTL1.2, is detectable by a molecular marker assay which detects at least 5, 6, 7, 8, 9, 10, 11, 12, 13, 14 or 15 Single Nucleotide Polymorphism (SNP) markers selected from the group consisting of:
a) the CX or CC genotype for the Single Nucleotide Polymorphism marker SNP_17 at nucleotide 51 of SEQ ID NO: 17 (or at nucleotide 51 in a variant thereof);
b) the GX or GG genotype for the Single Nucleotide Polymorphism marker SNP_18 at nucleotide 51 of SEQ ID NO: 18 (or at nucleotide 51 in a variant thereof);
c) the GX or GG genotype for the Single Nucleotide Polymorphism marker SNP_19 at nucleotide 51 of SEQ ID NO: 19 (or at nucleotide 51 in a variant thereof);
d) the TX or TT genotype for the Single Nucleotide Polymorphism marker SNP_20 at nucleotide 51 of SEQ ID NO: 20 (or at nucleotide 51 in a variant thereof);
e) the GX or GG genotype for the Single Nucleotide Polymorphism marker SNP_21 at nucleotide 51 of SEQ ID NO: 21 (or at nucleotide 51 in a variant thereof);
f) the GX or GG genotype for the Single Nucleotide Polymorphism marker SNP_22 at nucleotide 51 of SEQ ID NO: 22 (or at nucleotide 51 in a variant thereof);
g) the TX or TT genotype for the Single Nucleotide Polymorphism marker SNP_23 at nucleotide 51 of SEQ ID NO: 23 (or at nucleotide 51 in a variant thereof);
h) the CX or CC genotype for the Single Nucleotide Polymorphism marker SNP_24 at nucleotide 51 of SEQ ID NO: 24 (or at nucleotide 51 in a variant thereof);
i) the AX or AA genotype for the Single Nucleotide Polymorphism marker SNP_25 at nucleotide 51 of SEQ ID NO: 25 (or at nucleotide 51 in a variant thereof);
j) the CX or CC genotype for the Single Nucleotide Polymorphism marker SNP_26 at nucleotide 51 of SEQ ID NO: 26 (or at nucleotide 51 in a variant thereof);
k) the CX or CC genotype for the Single Nucleotide Polymorphism marker SNP_27 at nucleotide 51 of SEQ ID NO: 27 (or at nucleotide 51 in a variant thereof);
l) the CX or CC genotype for the Single Nucleotide Polymorphism marker SNP_28 at nucleotide 51 of SEQ ID NO: 28 (or at nucleotide 51 in a variant thereof);
m) the CX or CC genotype for the Single Nucleotide Polymorphism marker SNP_29 at nucleotide 51 of SEQ ID NO: 29 (or at nucleotide 51 in a variant thereof);
n) the AX or AA genotype for the Single Nucleotide Polymorphism marker SNP_30 at nucleotide 51 of SEQ ID NO: 30 (or at nucleotide 51 in a variant thereof);
o) the GX or GG genotype for the Single Nucleotide Polymorphism marker SNP_31 at nucleotide 51 of SEQ ID NO: 31 (or at nucleotide 51 in a variant thereof).

In one aspect said at least 5, 6, 7, 8, 9, 10, 11, 12, 13, 14 or 15 markers which are detected are consecutive markers.

Thus, in one embodiment the plants according to the invention comprise at least a Cytosine (C) (i.e. the CC or CX genotype) at nucleotide 51 of SEQ ID NO: 17 (referred to as SNP_17) or at the equivalent nucleotide of a genomic sequence comprising substantial sequence identity to SEQ ID NO: 17 (in other words there is a Cytosine at the physical position of chromosome 1 shown in Table 2);
and/or at least a Guanine (G) (i.e. the GG or GX genotype) at nucleotide 51 of SEQ ID NO: 18 (referred to as SNP_18) or at the equivalent nucleotide of a genomic sequence comprising substantial sequence identity to SEQ ID NO: 18 (in other words there is a Guanine at the physical position of chromosome 1 shown in Table 2);
and/or at least a Guanine (G) (i.e. the GG or GX genotype) at nucleotide 51 of SEQ ID NO: 19 (referred to as SNP_19) or at the equivalent nucleotide of a genomic sequence comprising substantial sequence identity to SEQ ID NO: 19 (in other words there is a Guanine at the physical position of chromosome 1 shown in Table 2);
and/or at least a Thymine (T) (i.e. the TT or TX genotype) at nucleotide 51 of SEQ ID NO: 20 (referred to as SNP 20) or at the equivalent nucleotide of a genomic sequence comprising substantial sequence identity to SEQ ID NO:20 (in other words there is a Thymine at the physical position of chromosome 1 shown in Table 2);
and/or at least a Guanine (G) (i.e. the GG or GX genotype) at nucleotide 51 of SEQ ID NO: 21 (referred to as SNP 21) or at the equivalent nucleotide of a genomic sequence comprising substantial sequence identity to SEQ ID NO:21 (in other words there is a Guanine at the physical position of chromosome 1 shown in Table 2);
and/or at least a Guanine (G) (i.e. the GG or GX genotype) at nucleotide 51 of SEQ ID NO: 22 (referred to as SNP_22) or at the equivalent nucleotide of a genomic sequence comprising substantial sequence identity to SEQ ID NO:22 (in other words there is a Guanine at the physical position of chromosome 1 shown in Table 2);
and/or at least a Thymine (T) (i.e. the TT or TX genotype) at nucleotide 51 of SEQ ID NO: 23 (referred to as SNP_23) or at the equivalent nucleotide of a genomic sequence comprising substantial sequence identity to SEQ ID NO:23 (in other words there is a Thymine at the physical position of chromosome 1 shown in Table 2);
and/or at least a Cytosine (C) (i.e. the CC or CX genotype) at nucleotide 51 of SEQ ID NO: 24 (referred to as SNP 24) or at the equivalent nucleotide of a genomic sequence comprising substantial sequence identity to SEQ ID NO:24 (in other words there is a Cytosine at the physical position of chromosome 1 shown in Table 2);
and/or at least an Adenine (A) (i.e. the AA or AX genotype) at nucleotide 51 of SEQ ID NO: 25 (referred to as SNP_25) or at the equivalent nucleotide of a genomic sequence comprising substantial sequence identity to SEQ ID NO:25 (in other words there is an Adenine at the physical position of chromosome 1 shown in Table 2);
and/or at least a Cytosine (C) (i.e. the CC or CX genotype) at nucleotide 51 of SEQ ID NO: 26 (referred to as SNP 26) or at the equivalent nucleotide of a genomic sequence comprising substantial sequence identity to SEQ ID NO:26 (in other words there is a Cytosine at the physical position of chromosome 1 shown in Table 2);
and/or at least a Cytosine (C) (i.e. the CC or CX genotype) at nucleotide 51 of SEQ ID NO: 27 (referred to as SNP 27) or at the equivalent nucleotide of a genomic sequence comprising substantial sequence identity to SEQ ID NO:27 (in other words there is a Cytosine at the physical position of chromosome 1 shown in Table 2);
and/or at least a Cytosine (C) (i.e. the CC or CX genotype) at nucleotide 51 of SEQ ID NO: 28 (referred to as SNP 28) or at the equivalent nucleotide of a genomic sequence comprising substantial sequence identity to SEQ ID NO:28 (in other words there is a Cytosine at the physical position of chromosome 1 shown in Table 2);
and/or at least a Cytosine (C) (i.e. the CC or CX genotype) at nucleotide 51 of SEQ ID NO: 29 (referred to as SNP_29) or at the equivalent nucleotide of a genomic sequence comprising substantial sequence identity to SEQ ID NO:29 (in other words there is a Cytosine at the physical position of chromosome 1 shown in Table 2);
and/or at least an Adenine (A) (i.e. the AA or AX genotype) at nucleotide 51 of SEQ ID NO: 30 (referred to as SNP_30) or at the equivalent nucleotide of a genomic sequence comprising substantial sequence identity to SEQ ID NO:30 (in other words there is an Adenine at the physical position of chromosome 1 shown in Table 2);
and/or at least a Guanine (G) (i.e. the GG or GX genotype) at nucleotide 51 of SEQ ID NO: 31 (referred to as SNP_31) or at the equivalent nucleotide of a genomic sequence comprising substantial sequence identity to SEQ ID NO:31 (in other words there is a Guanine at the physical position of chromosome 1 shown in Table 2).

In a further one embodiment the presence of the introgression fragment, or the chromosome 1 region (or variant or orthologous chromosome 1 region), comprising QTL1.2, is detectable by a molecular marker assay which detects at least 3, 4 or 5 Single Nucleotide Polymorphism (SNP) markers of the sub-groups consisting of: SNP_17 to SNP 22; SNP_22 to SNP 27; SNP_27 to SNP_31; or SNP_20 to SNP_29.

The SNP genotype refers to two nucleotides, and genomic sequences comprising one of these two nucleotides, one on each chromosome 1. So a plant having a CC genotype for SNP_17 has an identical nucleotide (C) on both chromosomes (i.e. is homozygous), while a plant having an CX genotype for SNP_17 has one chromosome with an C at nucleotide 51 of SEQ ID NO: 17 (or at the equivalent nucleotide of a genomic sequence comprising substantial sequence identity to SEQ ID NO: 17) and one chromosome with a X at nucleotide 51 of SEQ ID NO: 17 (or at the equivalent nucleotide of a genomic sequence comprising substantial sequence identity to SEQ ID NO: 17) and is heterozygous, whereby X may be any nucleotide. As the genomic sequences around the SNP markers provided herein may vary slightly in introgression fragments from other wild cucumber donors (i.e. variants or orthologous chromosome 1 regions) it is clear that the nucleotide sequences before and after the SNP may not be 100% identical to the sequences provided herein. Therefore sequences having substantial sequence identity (i.e. at least 95% identity) to the sequences provided herein, but which comprise the same SNP, are encompassed herein.

In one aspect, the introgression fragment comprising QTL1.2, or the chromosome 1 region (or variant or orthologous chromosome 1 region) comprising the QTL (QTL1.2 or variant), which is detectable by the above one or more markers is from a wild or primitive cucumber, and in one aspect the wild or primitive cucumber is a member of the Indian Cucumber Group. In one aspect it is the same introgression fragment as found on chromosome 1 in seeds deposited under accession number NCIMB 43745, or a smaller fragment retaining the QTL. SNP markers SNP_17 to SNP_31 span a region of about 2.6 Mb. In one aspect the introgression fragment on chromosome 1 is equal to or less than 2.7 Mb in size, preferably equal to or less than 2.6 Mb in size, more preferably equal to or less than 2.5, 2.4, 2.3, 2.2, 2.0, 1.9, 1.8, 1.7, 1.6, 1.5 Mb in size, e.g. equal to or less than 1.0 Mb. In one aspect the introgression fragment is at least 0.2 Mb, 0.5 Mb, 1.0 Mb, 1.5 Mb, 1.9 Mb, 2.0 Mb, 2.5 Mb, 2.7 Mb or 3 Mb in size. Thus, various ranges of introgression sizes are encompassed herein, such as fragments less than 2.7 Mb but more than 0.2 Mb, less than 2.0 Mb or 1.5 Mb but more than 0.2 Mb, 0.5 MB or 1 Mb, etc., which retain the QTL1.2 and one or more of the SNP markers of SNP_17 to SNP_31, or of the subgroups of SNP_17 to SNP_22; SNP_22 to SNP 27; SNP_27 to SNP_31 or SNP_20 to SNP_29. As mentioned before, the location of the QTL1.2 in the region spanning SNP_17 to SNP_31 can be determined by finemapping and recombinants comprising QTL1.2 on a smaller introgression fragment can be generated. The size of an introgression fragment can be easily determined by e.g. whole genome sequencing or Next Generation Sequencing, e.g. as described in Qi et al. 2013 (supra) or in Huang et al. 2009 (supra). Especially introgression regions can be easily distinguished from cultivated genomic regions due to the larger amount of genetic variation (SNPs, INDELs, etc.) in the introgression region.

To obtain the introgression fragment present on chromosome 1 (comprising QTL1.2) from the deposited seeds (NCIMB43745), i.e. to transfer the introgression fragments comprising the QTL to another cultivated cucumber plant, a plant is grown from the seed and the plant is crossed with a cultivated cucumber plant to obtain F1 seeds. As NCIMB43745 contains two recombinant chromosomes 1 (comprising the introgression fragment) all of the F1 seed and plants grown therefrom, contain one recombinant chromosome 1 from the NCIMB43745 parent and one non-recombinant chromosome 1 from the other cultivated parent. Thus, by traditional breeding one can transfer the recombinant chromosome 1 from NCIMB43745 into other cultivated cucumber lines or varieties. Plants which comprise the QTL1.2 can be screened for, and selected for, by the presence of one or more of the above SNP markers in order to identify plants comprising a recombinant chromosome 1.

To generate shorter introgression fragments (comprising QTL1.2) meiosis needs to take place and plants comprising the recombinant chromosomes 1, and especially new meiotic recombination events within the introgression fragment, need to be identified. For example, seeds of NCIMB43745 can be selfed one or more times to produce F1, F2 or F3 plants (or further selfing generations), and/or F1, F2 or F3 plants (etc.) comprising a recombinant chromosome 1 can be backcrossed to a cultivated parent. Plants which comprise the recombinant chromosome 1 can be screened for, and selected for, by the presence of one or more of the above SNP markers in order to identify plants comprising a smaller introgression fragment. Such new recombinants can then be tested for the presence of the QTL1.2 on the smaller introgression fragment by determining the average disease score in a ToLCNDV-ES disease assay compared to the (genetic) control lacking the introgression fragment.

Similarly, cultivated cucumber plants comprising QTL1.2 (or a variant thereof) can be generated and/or identified using different methods. For example, to obtain a cultivated cucumber plant comprising a introgression fragment from a wild donor, a wild donor is identified which comprises one or more of the SNP markers linked to QTL1.2 disclosed herein, e.g. any one, or more, or all of the markers described herein above. This has for example been done for various wild accessions, see Examples. The identified plant is crossed with a cultivated cucumber plant to obtain F1 seeds. The F1 can be selfed to produce F2, F3, etc. plants, and/or F2 plants or F3 plants, etc., can be backcrossed to the cultivated cucumber parent. Plants which are comprising QTL1.2 (or a variant thereof) can be screened for, and/or selected for, by the presence of one or more of the above SNP markers and/or screened for, and/or selected for, an increased ToLCNDV-ES resistance phenotype compared to the initial cultivated parent (lacking the introgressions). Alternatively or in addition, QTL mapping can be carried out in order to identify further molecular markers linked to the QTL1.2 (or a variant thereof) and/or to generate cultivated cucumber plants comprising an introgression fragment on chromosome 1 which confers significantly enhanced ToLCNDV-ES resistance.

In one embodiment the presence of the introgression fragment in a cultivated cucumber plant, or the chromosome 1 region (or orthologous chromosome 1 region), comprising QTL1.2, is detectable by a molecular marker assay which detects at least 5, 6, 7, 8, 9, 10 or more of the markers selected from the group consisting of:
a) the CC or CX genotype for the Single Nucleotide Polymorphism marker SNP_17 in SEQ ID NO: 17 (or in a variant thereof);
b) the GG or GX genotype for the Single Nucleotide Polymorphism marker SNP_31 in SEQ ID NO: 31 (or in a variant thereof);
c) any wild cucumber genome-specific marker in between marker SNP_17 and SNP_31.

In one aspect the markers of c) are one or more of SNP_18 to SNP_30. In one aspect, at least 5, 6, 7, 8, 9, 10 or more markers are detected from the markers of a), b) and/or c) above. In one embodiment at least the marker of a) and/or b) is detected and optionally at least one, two, three or more markers of c) are detected. In one aspect the markers detected are consecutive markers.

Any wild cucumber genome-specific marker in between two markers refers to any molecular marker which maps genetically to the chromosome 1 region in-between the two markers and/or which lies physically in-between the two markers, and which is indicative of the wild cucumber chromosome 1 region. This means that the marker is polymorphic between the cultivated cucumber genome and the wild cucumber genome. In one aspect, the marker is a Single Nucleotide Polymorphism (SNP), but other molecular markers such as RFLP, AFLP, RAPD, DNA sequencing, etc. may equally be used.

The introgression fragment in the plants of the invention is in one aspect a fragment of chromosome 1 (comprising QTL1.2) which is present in seeds deposited under accession number NCIMB43745 or a smaller version of that fragment retaining the QTL (generated by e.g. recombination within the introgression fragment).

The introgression fragment is in one aspect equal to or less than 3 Mb in size, preferably equal to or less than 2.7 Mb, 2.6 Mb, 2.5 Mb, 2 Mb, 1.5 Mb, 1 Mb in size. In a further aspect the introgression fragment is at least 0.5 Mb or at least 1 Mb in size.

Also provided are seeds from which a plant of the invention can be grown, as are cucumber fruits harvested from a plant of the invention and comprising the recombinant chromosome 1 in their genome (comprising QTL1.2 or a variant). Likewise a plant cell, tissue or plant part of a plant or of a seed is provided comprising at least one recombinant chromosome 1 (comprising QTL1.2 or a variant), wherein said recombinant chromosome 1 comprises an introgression fragment from a wild or primitive cucumber and wherein said introgression fragment comprises an allele conferring significantly enhanced ToLCNDV-ES resistance.

The molecular markers described herein may be detected according to standard method. For example SNP markers can easily be detected using a KASP-assay (see www.kpbioscience.co.uk) or other SNP genotyping assays. For developing a KASP-assay, for example 50 or 70 base pairs upstream and 50 or 70 base pairs downstream of the SNP can be selected and two allele-specific forward primers and one allele specific reverse primer can be designed. See e.g. Allen et al. 2011, Plant Biotechnology J. 9, 1086-1099, especially p 097-1098 for KASP assay method.

Thus, in one aspect, the SNP markers and the presence/absence of the marker associated with QTL1.2 is determined using a KASP assay, but equally other SNP genotyping assays can be used. For example, a TaqMan SNP genotyping assay, a High Resolution Melting (HRM) assay, SNP-genotyping arrays (e.g. Fluidigm, Illumina, etc.) or DNA sequencing may equally be used.

The physical size of an introgression fragment can be determined by various methods, such as physical mapping, sequencing or by visualization of the introgression using Fluorescent in situ hybridization (FISH) images (Verlaan et al. 2011, Plant Journal 68: 1093-1103).

Cultivated cucumber plants with smaller introgression fragments on chromosome 1 (comprising QTL1.2 or a variant) can be generated by generating new recombinant plants from a population of plants derived from a cross between a cultivated cucumber plant (lacking the introgressions) and a plant of the invention and selecting recombinant progeny having smaller introgression sizes. Such plants are thus in one aspect derived from (progeny or descendants of) the recombinant chromosome 1 present in plants of which seeds have been deposited under NCIMB43745. Such progeny or descendants which retain the QTL1.2, and thus the higher ToLCNDV-ES resistance compared to plants lacking an introgression as described herein, are encompassed herein.

### QTL2.1 or a Variant of QTL2.1 on Chromosome 2

Thus, in one aspect a cultivated cucumber plant is provided comprising an introgression fragment from a wild or primitive cucumber, wherein the introgression fragment comprises QTL2.1, or a variant thereof, and wherein the introgression fragment comprises all or part of the region starting at nucleotide (or base) 15218569 (corresponding to SNP_32) of chromosome 2 and ending at nucleotide (or base) 19535432 of chromosome 2 (corresponding to SNP_47). In other words, all or part of the region starting at nucleotide 15218569 of chromosome 2 and ending at nucleotide 19535432 of chromosome 2 is, in one aspect, from a wild donor cucumber and comprises QTL2.1 or a variant thereof. Which sub-region contains QTL2.1 can be identified by e.g. fine-mapping. So, for example if QTL2.1 is found to be in between SNP_32 and SNP_37, then the plant of the invention only needs to comprise the introgression region starting at nucleotide 15218569 of chromosome 2 (SNP_32) and ending at nucleotide 16378312 (SNP_37) of chromosome 2.

In one aspect QTL2.1 (or a variant thereof) is located in-between marker SNP_32 at nucleotide 51 of SEQ ID NO: 32 (or at nucleotide 51 in a variant sequence of SEQ ID NO: 32) and marker SNP_47 at nucleotide 51 of SEQ ID NO: 47 (or at nucleotide 51 in a variant sequence of SEQ ID NO: 47). In another aspect QTL2.1 (or a variant thereof) is located in-between marker SNP_32 at nucleotide 51 of SEQ ID NO: 32 (or at nucleotide 51 in a variant sequence of SEQ ID NO: 32) and marker SNP_37 at nucleotide 51 of SEQ ID NO: 37 (or at nucleotide 51 in a variant sequence of SEQ ID NO: 37). In a further aspect QTL2.1 (or a variant thereof) is located in-between marker SNP_37 at nucleotide 51 of SEQ ID NO: 37 (or at nucleotide 51 in a variant sequence of SEQ ID NO: 37) and marker SNP_42 at nucleotide 51 of SEQ ID NO: 42 (or at nucleotide 51 in a variant sequence of SEQ ID NO: 42). In a further aspect QTL2.1 (or a variant thereof) is located in-between marker SNP_42 at nucleotide 51 of SEQ ID NO: 42 (or at nucleotide 51 in a variant sequence of SEQ ID NO: 42) and marker SNP_47 at nucleotide 51 of SEQ ID NO: 47 (or nucleotide 51 in a variant sequence of SEQ ID NO: 47). In a further aspect QTL2.1 (or a variant thereof) is located in-between marker SNP_34 at nucleotide 51 of SEQ ID NO: 34 (or at nucleotide 51 in a variant sequence of SEQ ID NO: 34) and marker SNP_44 at nucleotide 51 of SEQ ID NO: 44 (or nucleotide 51 in a variant sequence of SEQ ID NO: 44).

In another aspect the introgression fragment of the invention (comprising QTL2.1 or a variant thereof) is a fragment comprising a smaller fragment (part) of the region starting at nucleotide (or base) 15218569 of chromosome 2 and ending at nucleotide (or base) 19535432 of chromosome 2, e.g. having a size of e.g. 4.4 Mb, 4.3 Mb, 4.2 Mb, 4.0 Mb, 3.0 Mb, 2.5 Mb, 2 Mb, 1 Mb, 0.5 Mb, 100 kb, 50 kb, 35 kb, 30 kb, 20 kb, or less and comprising the QTL or a variant thereof. In one aspect the part is at least 5 kb, 10 kb, 20 kb in size, or more.

In one aspect the cultivated cucumber plant of the invention comprises an introgression fragment from a wild or primitive cucumber, which introgression fragment comprises QTL2.1 or a variant thereof, wherein the introgression fragment comprises all of part of the region starting at 15.2 Mb and ending at 19.6 Mb of the physical chromosome 2.

In one aspect the introgression fragment on chromosome 2 comprising QTL2.1, or a variant thereof, is obtainable by crossing a plant grown from NCIMB43745 with another cucumber plant, especially a cultivated cucumber plant, in one aspect a long cucumber type or a pickling or slicer type.

In one aspect the cultivated cucumber plant of the invention comprising QTL2.1, or a variant thereof, is a plant wherein said introgression fragment on chromosome 2 is obtainable by crossing a plant grown from seeds deposited under accession number NCIMB43745 with another cucumber plant. Thus, in one aspect the QTL is the QTL present in seeds deposited under accession number NCIMB43745.

In a further aspect the cultivated cucumber plant of the invention comprising QTL2.1, or a variant thereof, is a plant wherein said introgression fragment on chromosome 2 is obtainable by crossing a plant comprising the same SNP haplotype or SNP genotype for at least 5, 6, 7, 8, 9, 10 or more SNP markers linked to the QTL (i.e. SNP_32 to SNP_47 for QTL2.1 as shown in Table 3) with another cucumber plant, especially with a cultivated cucumber elite breeding line. Thus, in one aspect the QTL is the QTL present in wild donor accessions which comprise the same SNP haplotype or genotype for at least 5, 6, 7, 8, 9, 10 or more of the SNP markers, e.g. as in NCIMB43745. Preferably the donor also comprises a resistance phenotype having an average ToLCNDV-ES disease score of at least 7.5, preferably at least 8.0, 8.1, 8.2, 8.3, 8.4, 8.5, 8.6, 8.7, 8.8, 8.9 or 9.0.

When referring to the SNP markers herein, which are indicative of the presence of the introgression fragment (and the ToLCNDV-ES resistance QTL present on the introgression fragment), it is understood that the SNP genotype or haplotype which is indicative of the introgression fragment is referred to, i.e. the SNP genotype or haplotype as provided e.g. in Tables 1 to 4. It is noted that the SNP marker genotype can distinguish between the introgression fragment being in homozygous or heterozygous form. In homozygous form the nucleotide is identical, while in heterozygous form the nucleotide is not identical. The SNP genotype of the ‘wild type’ chromosome lacking the introgression fragment is the other haplotype, e.g. the haplotype of the recurrent parent). So, e.g. the genotype of SNP_32 indicative of the introgression fragment comprising QTL2.1 is ‘TC’ (QTL2.1/wt) or ‘TT’ (QTL2.1 QTL2.1) while the SNP genotype indicative of the wild type/genetic control (lacking the introgression fragment) is e.g. ‘CC’ (wt/wt). This can also be written as genotype TX′ (QTL2.1/wt) or ‘TT’ (QTL2.1 QTL2.1) while the SNP genotype indicative of the wild type/genetic control (lacking the introgression fragment) is e.g. ‘XX’ (wt/wt). X may be any nucleotide (A, T, C or G). Thus, when referring to a plant or plant part (e.g. cell) comprising the introgression fragment in homozygous or heterozygous form, it is understood that the SNP markers linked to the introgression fragment have the corresponding SNP genotype or haplotype.

So, in one aspect, a cultivated Cucumis sativus var. sativus plant is provided comprising an introgression fragment on chromosome 2 in homozygous or heterozygous form, wherein said introgression fragment confers an increase in ToLCNDV-ES resistance (at least when the fragment is in homozygous form) compared to the cucumber plant lacking the introgression fragment on chromosome 2, e.g. the genetic control or control variety, when grown under the same conditions.

The increase in ToLCNDV-ES resistance is phenotypically expressed as a higher average disease score (less yellowing, measured e.g. in a disease assay as described herein) of the cultivated cucumber plant line or variety comprising the introgression fragment on chromosome 2 in homozygous form compared to the genetic control line or variety lacking the introgression fragment on chromosome 2 when grown under the same environment. The average disease score is preferably increased by at least 1.0, 1.5, 2.0, 2.5, 3.0 or more points on the disease scale of 2.0 (90-100% of leaf area is covered with yellowing mosaic symptoms) to 9.0 (no symptoms). So, for example if QTL2.1 is introduced into a susceptible cucumber line or variety having an average disease score of about 4.0, the introduction of QTL2.1 preferably increases the average disease score to an average score of at least 5.0, 5.5, 6.0, 6.5, 7.0 or more, at least when QTL2.1 is in homozygous form. As QTL2.1 was found to be recessive or partially recessive, the effect of QTL2.1 on ToLCNDV-ES resistance is preferably measured when it is in homozygous form.

It is known that all four QTLs together in homozygous form (QTL1.1, QTL1.2, QTL2.1 and QTL3.1) lead to an average disease score of 9.0 when introduced into a susceptible plant having an average disease score of about 4.0 to 5.0. The effect of the individual QTLs will be smaller than the combined effect, but will still be effective in reducing ToLCNDV-ES symptoms. The effect of QTL2.1 alone can be determined by introducing the QTL alone into a susceptible cucumber plant. As QTL2.1 is recessive or partially recessive, the effect of QTL2.1 on the phenotype is preferably analyzed when QTL2.1 is in homozygous form.

The plants of the invention therefore comprise a genome of cultivated cucumber, with at least one or two recombinant chromosomes, namely one or two recombinant chromosomes 2 (i.e. heterozygous or homozygous). The recombinant chromosomes comprise a fragment of a wild donor cucumber, which is easily distinguishable from the cultivated cucumber genome by molecular marker analysis, whole genome sequencing, chromosome painting and similar techniques.

In one aspect the introgression fragment on chromosome 2 is from a wild or primitive cucumber, comprises the ToLCNDV-ES QTL2.1, or a variant thereof, and comprises all or part of the region starting at nucleotide SNP_32 and ending at SNP 47. Thus, the introgression fragment comprises the QTL2.1 or a variant thereof and one or more or all (e.g. 2, 3, 4, 5, 6, 7, 8, 9, 10, 11, 12, 13, 14, 15, 16) SNP markers of the wild donor selected from SNP_32 to SNP_47 as shown in Table 3.

In one aspect the introgression fragment comprises QTL2.1 and one or more or all of SEQ ID NO: 32 to SEQ ID NO: 47.

In one aspect the presence of the introgression fragment on chromosomes 2 comprising QTL2.1 in the genome of the plant or plant cell or plant tissue (or in the DNA extracted therefrom) is detectable by a molecular marker assay which detects one or more molecular markers of the introgression fragment, especially the donor SNP haplotype or genotype for at least 5, 6, 7, 8, 9, 10 or more of SNP_32 to SNP 47, at nucleotide 51 of SEQ ID NO: 32 to 47, respectively. However, as mentioned, other techniques may be used, e.g. the SNP genotype of the markers may also be determined by sequencing or by using alternative markers located in between the SNP markers provided herein or within 7 cM, or within 5 cM, of a marker provided herein; or within 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less of a marker provided herein.

In one aspect the presence of the introgression fragment on chromosomes 2 comprising QTL2.1 in the genome of the plant or plant cell or plant tissue (or in the DNA extracted therefrom) is detectable by detecting the presence of one or more or all of SEQ ID NO: 32 to SEQ ID NO: 47.

When reference is made herein to one or more molecular markers or sequences being “detectable” by e.g. a molecular marker assay, this means of course that the plant or plant part comprises the one or more markers or sequences in its genome, as the marker or sequence would otherwise not be detectable.

### Cucumber Plants Comprising an Introgression Fragment on Chromosome 2 (QTL 2.1) or a Variant

In one aspect a cultivated Cucumis sativus var. sativus plant comprising an introgression fragment from a wild or primitive cucumber on chromosome 2 in homozygous or heterozygous form is provided, wherein said introgression fragment comprises a Quantitative Trait Locus (QTL) located between the Single Nucleotide Polymorphism marker SNP_32 at nucleotide 51 of SEQ ID NO: 32 (or at nucleotide 51 of a variant of SEQ ID NO: 32) and the Single Nucleotide Polymorphism marker SNP_47 at nucleotide 51 of SEQ ID NO: 47 (or at nucleotide 51 of a variant of SEQ ID NO: 47), which QTL confers an increase in ToLCNDV-ES resistance at least when QTL2.1 (or a variant) is in homozygous form. In one aspect the QTL is located between base 15218569 (SNP_32) and base 19535432 (SNP 47) of chromosome 2.

Thus, in one aspect QTL2.1 (or a variant thereof) is located in the region between SNP_32 in SEQ ID NO: 32 (or in a variant thereof) and SNP_47 in SEQ ID NO: 47 (or in a variant thereof).

Therefore, in one aspect a cultivated Cucumis sativus var. sativus plant is provided comprising an introgression fragment on chromosome 2 in homozygous or heterozygous form, wherein said introgression fragment confers an increase in ToLCNDV-ES resistance at least when the introgression fragment is in homozygous form (compared to the plant lacking the introgression fragment, e.g. the genetic control) and wherein said introgression fragment comprises the SNP marker haplotype or genotype of at least 5, 6, 7, 8, 9, 10, 11, 12, 13, 14, 15 or 16 of the SNP markers selected from the group consisting of:
a) the TX or TT genotype for the Single Nucleotide Polymorphism marker SNP_32 at nucleotide 51 of SEQ ID NO: 32 (or at nucleotide 51 in a variant thereof);
b) the TX or TT genotype for the Single Nucleotide Polymorphism marker SNP_33 at nucleotide 51 of SEQ ID NO: 33 (or at nucleotide 51 in a variant thereof);
c) the CX or CC genotype for the Single Nucleotide Polymorphism marker SNP_34 at nucleotide 51 of SEQ ID NO: 34 (or at nucleotide 51 in a variant thereof);
d) the AX or AA genotype for the Single Nucleotide Polymorphism marker SNP_35 at nucleotide 51 of SEQ ID NO: 35 (or at nucleotide 51 in a variant thereof);
e) the TX or TT genotype for the Single Nucleotide Polymorphism marker SNP_36 at nucleotide 51 of SEQ ID NO: 36 (or at nucleotide 51 in a variant thereof);
f) the CX or CC genotype for the Single Nucleotide Polymorphism marker SNP_37 at nucleotide 51 of SEQ ID NO: 37 (or at nucleotide 51 in a variant thereof);
g) the GX or GG genotype for the Single Nucleotide Polymorphism marker SNP_38 at nucleotide 51 of SEQ ID NO: 38 (or at nucleotide 51 in a variant thereof);
h) the CX or CC genotype for the Single Nucleotide Polymorphism marker SNP_39 at nucleotide 51 of SEQ ID NO: 39 (or at nucleotide 51 in a variant thereof);
i) the GX or GG genotype for the Single Nucleotide Polymorphism marker SNP_40 at nucleotide 51 of SEQ ID NO: 40 (or at nucleotide 51 in a variant thereof);
j) the TX or TT genotype for the Single Nucleotide Polymorphism marker SNP_41 at nucleotide 51 of SEQ ID NO: 41 (or at nucleotide 51 in a variant thereof);
k) the AX or AA genotype for the Single Nucleotide Polymorphism marker SNP_42 at nucleotide 51 of SEQ ID NO: 42 (or at nucleotide 51 in a variant thereof);
l) the TX or TT genotype for the Single Nucleotide Polymorphism marker SNP_43 at nucleotide 51 of SEQ ID NO: 43 (or at nucleotide 51 in a variant thereof);
m) the TX or TT genotype for the Single Nucleotide Polymorphism marker SNP_44 at nucleotide 51 of SEQ ID NO: 44 (or at nucleotide 51 in a variant thereof);
n) the GX or GG genotype for the Single Nucleotide Polymorphism marker SNP_45 at nucleotide 51 of SEQ ID NO: 45 (or at nucleotide 51 in a variant thereof);
o) the AX or AA genotype for the Single Nucleotide Polymorphism marker SNP_46 at nucleotide 51 of SEQ ID NO: 46 (or at nucleotide 51 in a variant thereof);
p) the GX or GG genotype for the Single Nucleotide Polymorphism marker SNP_47 at nucleotide 51 of SEQ ID NO: 47 (or at nucleotide 51 in a variant thereof).

When referring to a SNP in a variant sequence, that variant sequence comprises at least 95%, 96%, 97%, 98% or 99% sequence identity with the mentioned sequence. X refers to any nucleotide for the sequence on the other chromosome 1 of the pair of chromosomes. In one aspect X may be the nucleotide of the recurrent parent as described in Table 3.

In one aspect said at least 5, 6, 7, 8, 9, 10, 11, 12, 13, 14, 15 or 16 markers are consecutive markers.

The fragment comprising the QTL2.1 may, thus, be large (comprising SNP_32 to SNP 47), or may be smaller and lack markers having the genotype or haplotype of the wild cucumber (i.e. the markers have the cultivated cucumber genotype or haplotype instead, see also Table 3, SNP haplotype of recurrent parent), but it may still confer enhanced ToLCNDV-ES resistance on the cultivated cucumber plant, i.e. it can still comprise the ToLCNDV-ES allele (QTL2.1 or a variant). Such smaller introgression fragments are an embodiment of the invention. Plants having smaller introgression fragments which still confer the enhanced ToLCNDV-ES resistance (i.e. contain the resistance allele) can be generated using known techniques, such as fine-mapping or similar techniques. For example by starting with a plant comprising the introgression fragment as found in seeds deposited under accession number NCIMB 43745 and crossing such a plant with another cultivated cucumber plant and selfing the progeny of said cross, and/or backcrossing the progeny, to generate a population of plants which may contain recombinants having a smaller introgression fragment on chromosome 2, which fragments still confer enhanced ToLCNDV-ES resistance in relation to a plant lacking the introgression fragment (such as the genetic control, e.g. plants grown from seeds deposited under NCIMB42344), e.g. a fragment comprising markers SNP_32 to SNP_37, or SNP_37 to SNP_42 or SNP_42 to SNP_47 or SNP_34 to SNP_44. Marker assays can be used to determine the size of the smaller introgression fragment. One or more of the SNP markers with the genotype or haplotype of the wild donor cucumber may be missing. The cultivated cucumber genotype or haplotype is then detected for these SNP markers. The ToLCNDV-ES resistance of plants comprising such a smaller introgression fragment can then be compared in a disease assay as described herein, i.e. growing a plurality of plants comprising the smaller introgression fragment in experiments together with suitable control plants, lacking the introgression fragments. The control plants are preferably a genetic control, such as NCIMB43744. If the average ToLCNDV-ES disease score remains significantly higher than in the control, then the smaller introgression fragment has retained the QTL2.1.

Alternatively, the same or variant QTL (QTL2.1 or variant QTL2.1) may be introgressed from a different wild donor accessions, whereby optionally not all SNP markers disclosed herein may be present, i.e. the SNP haplotype of the donor accession may only be identical to the SNP haplotype of the QTL2.1 present in seeds of NCIMB43745 for at least 5, 6, 7, 8, 9, 10 or more SNPs. Such alternative wild cucumber sources can be identified using the SNP markers provided herein, by screening germplasm (i.e. accessions of) wild or primitive cucumber using a marker assay to detect the genotype or haplotype of one or more markers of markers SNP_32 to SNP_47, or of markers SNP_32 to SNP 37, SNP_37 to SNP 42, SNP_42 to SNP_47, or SNP_34 to SNP 44, or even only a smaller subgroup of these markers (e.g. 2, 3 or 4). For example, Table 3 shows various donors (PI605996, CGN22263 and CGN22932, also known as PI197087) which have the same SNP haplotype for SNP_32 to SNP_47. In the same way other donors, having QTL2.1 or a variant thereof, may be identified. Plants comprising the same or variant QTL2.1 from these donors or from other sources are also an embodiment of the invention. Thus, as long as at least 5, 6, 7, 8, 9, 10 or more (or all) of the SNPs of SNP_32 to SNP 47, or of the SNPs of SNP_32 to SNP_37, or of the SNPs of SNP_37 to SNP 42, or of the SNPs of SNP_42 to SNP 47, or of the SNPs of SNP_34 to SNP_44 are present, the donor may contain QTL2.1 (or a variant thereof) and is encompassed herein. The skilled person can then introgress the QTL2.1 (or a variant thereof) into cultivated cucumber in order to enhance ToLCNDV-ES resistance as described herein and in order to confirm that the QTL enhances ToLCNDV-ES resistance when present in cultivated cucumber. Prior to introgression the wild donor may also be tested for ToLCNDV-ES resistance in an assay as described and e.g. a donor may be selected that comprises an average ToLCNDV-ES score of e.g. at least 7.5, 8.0, 8.5, or 9.0.

As described above, in one embodiment the cultivated cucumber plant of the invention comprises an introgression fragment comprising at least a subset of SNP markers with the genotype (or haplotype) of the wild donor cucumber, i.e. at least 5, 6, 7, 8, 9 or more markers of SNP_32 to SNP 47, or at least 3 markers of SNP_32 to SNP 37, or of SNP_37 to SNP_42, or of SNP_42 to SNP 47, or of SNP_34 to SNP_44. In one aspect the cultivated cucumber plant comprises all, or all except 1 or 2 markers of SNP_32 to SNP_47, or of SNP_32 to SNP_37, or of SNP_37 to SNP_42, or of SNP_42 to SNP_47, or of SNP_34 to SNP_44.

Thus, the introgression fragment (and a cultivated cucumber plant or plant part, e.g., a cell, comprising the introgression fragment) can be detected in a marker assay by detecting the SNP genotype or haplotype of the introgression fragment (i.e. of the wild cucumber germplasm) of one or more or all of the markers above, preferably at least 5, 6, 7, 8 or more.

Thus, in one aspect, a Quantitative Trait Locus (QTL2.1) was found to be present on chromosome 2 of a wild cucumber donor which, when transferred (introgressed) into a cultivated cucumber variety or breeding line, and at least when present in homozygous form, confers significantly enhanced ToLCNDV-ES resistance onto the cultivated cucumber plant. The QTL, or the introgression fragment comprising the QTL (comprising the ToLCNDV resistance allele), is partially recessive, i.e. it is preferred to have the introgression fragment on both of the chromosomes 2 (two recombinant chromosomes 2).

Although the present source of the QTL2.1 which was used to map and introgress the QTL is a single, specific wild source, there are other wild accessions which comprise QTL2.1 (or a variant) at the same locus on chromosome 2. For example the wild accessions PI605996, CGN22263 and CGN22932, also known as PI197087, were found to comprise the same SNP haplotype for markers SNP_32 to SNP_47 (as shown in Table 3) and were found to be resistant to ToLCNDV-ES. The (variant) QTL2.1 from these or other donors can, thus, be introgressed into cultivated cucumber, optionally in combination with one or more of QTL1.2, QTL1.1 and QTL3.1. Similarly, other wild or primitive cucumber accessions can be screened for the SNP haplotype or genotype of one or more or all of SNP_32 to SNP_47.

Such other donors may comprise a ToLCNDV-ES resistance allele which has a slightly different nucleotide sequences, i.e. variants of the allele (QTL2.1) found herein. Such variant QTLs can also be identified and introgressed into cultivated cucumber as described herein, to generate a cultivated cucumber plant comprising a genome of cultivated C. sativus var. sativus and a recombinant chromosome 2, whereby the recombinant chromosome 2 comprises an introgression fragment, which confers an enhanced ToLCNDV-ES resistance onto the cultivated cucumber plant at least when present in homozygous form. To identify such wild donor accessions comprising QTL2.1, wild accessions can be screened, e.g. in a marker assay or by sequence comparison or other methods, for the presence of one or more of the SNP markers provided herein. The putative QTL (or variant QTL) can then be introgressed into cultivated cucumber, e.g. using MAS, i.e. using one or more (or all) of the SNP markers provided herein to detect and/or select progeny plants (e.g. backcross plants) comprising a recombinant chromosome 2. The selected plants, i.e. the cultivated cucumber plants comprising an introgression fragment on chromosome 2, wherein the introgression fragment on chromosome 2 is detectable by 5, 6, 7, 8, 9, 10 or more of the SNP markers SNP_32 to SNP_47 can then be phenotyped in a ToLCNDV-ES disease assay together with the suitable control plants, preferably at least the genetic control, in order to determine whether the introgression fragment indeed causes a significant increase in ToLCNDV-ES resistance.

Accessions of wild or primitive cucumber, are obtainable from e.g. the USDA National Plant Germplasm System collection or other seed collections, and can thus be screened for the presence of QTL2.1 using e.g. a marker assay as described herein, and accessions comprising 5 or more of the SNP markers (e.g. at least 5, 6, 7, 8, 9, 10 or more SNP markers indicative of QTL2.1) can be crossed with a cultivated cucumber plant having normal wild-type, non-recombinant chromosomes 2. The F1 or F2 generation (or further generation, such as the F3 or a backcross generation) can then be screened for recombinant plants having the introgression fragment or a part thereof, using the molecular marker assays described herein.

In one aspect, the introgression fragment is from a donor comprising the SNP haplotype for QTL2.1 as shown in Table 3 for the introgression donor (NCIMB43745), for P1605996, for CGN22263 or for CGN22932, also known as PI197087.

In a specific embodiment, the introgression fragment comprising the ToLCNDV-ES QTL2.1 (or a variant) is derivable from (or derived from) or obtainable from (or obtained from; or as present in) seeds, a representative sample of which has been deposited under accession number NCIMB 43745, or from progeny thereof, or from seeds having accession number PI605996 (USDA ARS-GRIN collection), or from seeds having accession number CGN22263 or CGN22932 (Wageningen CGN collection) or from seeds having accession number PI197087 (USDA ARS-GRIN collection) or from progeny of any of these. The progeny may be any progeny which retain the SNP markers or haplotype indicative of (and linked to) the QTL, as described. Thus, progeny are not limited to F1 or F2 progeny of the deposit or accession, but can be any progeny, whether obtained by selfing and/or crossing with another cucumber plant.

In one embodiment the introgression fragment comprising QTL2.1 is identifiable by one or more of the markers described elsewhere herein, especially markers SNP_32 to SNP_47 for the introgression fragment on chromosome 2, or a subset of markers, such as one or more of the markers selected from SNP markers SNP_32 to SNP_37, or from SNP markers SNP_37 to SNP_42, or from of the SNP markers SNP_42 to SNP_47, or from SNP markers SNP_34 to SNP_44. In one aspect the invention provides a cultivated cucumber plant, having a genome of cultivated (domesticated) cucumber which comprises enhanced ToLCNDV-ES resistance, wherein the enhanced resistance is conferred by an introgression fragment on the cultivated cucumber chromosome 2, wherein said introgression fragment is obtained by (or obtainable by) crossing a cultivated plant grown from seeds deposited under NCIMB 43745 or progeny of this plant (which comprises one or more the markers disclosed herein linked to the QTL) with a cultivated cucumber plant. Thus in one aspect the cultivated cucumber plant of the invention comprises the same introgression fragment and the same recombinant chromosome 2 as present in NCIMB 43745 (comprising all of the wild donor haplotype for SNP markers SNP_32 to SNP_47 or comprising SEQ ID NO: 32 to 47), or it comprises a shorter fragment of that introgression fragment, whereby the shorter fragment retains the genetic element conferring ToLCNDV-ES resistance (QTL2.1).

Thus in one aspect the invention relates to a plant of the invention i.e. a cultivated Cucumis sativus var. sativus plant comprising an introgression fragment comprising QTL2.1 from a wild cucumber on chromosome 2 in homozygous or heterozygous form and wherein said introgression fragment is the introgression fragment “as in”/is “identical to”/is “the same as in” the seeds deposited under number NCIMB 43745, or is a shorter fragment thereof, but still confers enhanced ToLCNDV-ES resistance due to the presence of QTL2.1.

As SEQ ID NO: 32 to 47 are from the wild donor used to generate NCIMB43745, they can identify the introgression fragment or sub-fragments of the specific donor.

In yet another embodiment the invention relates to a plant of the invention i.e. a cultivated Cucumis sativus var. sativus plant comprising an introgression fragment comprising QTL21.1 from a wild cucumber on chromosome 2 in homozygous or heterozygous form and wherein said introgression fragment is the introgression fragment is a variant of the introgression fragment seeds deposited under number NCIMB 43745, i.e. it comprises the QTL 2.1, but the genomic sequence may be different. As wild accessions will be genetically divergent, the genomic sequence of an introgression fragment comprising QTL2.1 from other wild or primitive cucumbers will most likely not be identical to the genomic sequence as introgressed into NCIMB 43745, and even the ToLCNDV-ES conferring gene (comprising a promoter, introns and exons) may be divergent in nucleotide sequence, but the function will be the same, i.e. conferring enhanced resistance. The divergence can be seen in that certain SNP markers linked to QTL2.1 may be commonly found in various accessions, while other SNP markers may only be found in specific accessions. So for example not all of SNP_32 to SNP_47 may be found in other wild cucumber donors. For example a wild donor may have a slightly different SNP haplotype for SNP_32 to SNP_47, with e.g. one or two SNP markers having a different nucleotide. However, QTL2.1 (comprising e.g. a variant or ortholog of the ToLCNDV-ES resistance allele) may still be present in such wild accessions. The skilled person is capable of identifying and introgressing the QTL2.1 comprising region found in other wild cucumber donors into cultivated cucumber, e.g. detecting wild accessions comprising the SNP markers or a subset thereof and transferring these SNP markers (or subset) into a cultivated cucumber line or variety and assessing the ToLCNDV-ES resistance of the cultivated line or variety (preferably comprising QTL2.1 in homozygous form) compared to the line or variety lacking the SNP markers (or subset), i.e. lacking the introgression fragment. Even in cases where the SNP haplotype for SNP_32 to SNP_47 is identical to the SNP haplotype of QTL2.1 found in seeds ofNCIMB 43745, the actual nucleotide sequences flanking the SNP at nucleotide 51 of SEQ ID NO: 32 to 47 may be different in other donors. So other donors may comprise the same SNP nucleotide at nucleotide 51, but in a sequence comprising at least 95%, 96%, 97%, 98% or 99% sequence identity to SEQ ID NO: 32 to 47 when e.g. aligned pairwise. This variation can be seen by sequencing the donors and aligning sequences of SEQ ID NO: 32 to SEQ ID NO: 47 with that sequence.

In one embodiment the presence of the introgression fragment comprising QTL2.1, or the chromosome 2 region (or variant or orthologous chromosome 2 region), comprising QTL2.1, is detectable by a molecular marker assay which detects at least 5, 6, 7, 8, 9, 10, 11, 12, 13, 14, 15 or 16 Single Nucleotide Polymorphism (SNP) markers selected from the group consisting of:
a) the TX or TT genotype for the Single Nucleotide Polymorphism marker SNP_32 at nucleotide 51 of SEQ ID NO: 32 (or at nucleotide 51 in a variant thereof);
b) the TX or TT genotype for the Single Nucleotide Polymorphism marker SNP_33 at nucleotide 51 of SEQ ID NO: 33 (or at nucleotide 51 in a variant thereof);
c) the CX or CC genotype for the Single Nucleotide Polymorphism marker SNP_34 at nucleotide 51 of SEQ ID NO: 34 (or at nucleotide 51 in a variant thereof);
d) the AX or AA genotype for the Single Nucleotide Polymorphism marker SNP_35 at nucleotide 51 of SEQ ID NO: 35 (or at nucleotide 51 in a variant thereof);
e) the TX or TT genotype for the Single Nucleotide Polymorphism marker SNP_36 at nucleotide 51 of SEQ ID NO: 36 (or at nucleotide 51 in a variant thereof);
f) the CX or CC genotype for the Single Nucleotide Polymorphism marker SNP_37 at nucleotide 51 of SEQ ID NO: 37 (or at nucleotide 51 in a variant thereof);
g) the GX or GG genotype for the Single Nucleotide Polymorphism marker SNP_38 at nucleotide 51 of SEQ ID NO: 38 (or at nucleotide 51 in a variant thereof);
h) the CX or CC genotype for the Single Nucleotide Polymorphism marker SNP_39 at nucleotide 51 of SEQ ID NO: 39 (or at nucleotide 51 in a variant thereof);
i) the GX or GG genotype for the Single Nucleotide Polymorphism marker SNP_40 at nucleotide 51 of SEQ ID NO: 40 (or at nucleotide 51 in a variant thereof);
j) the TX or TT genotype for the Single Nucleotide Polymorphism marker SNP_41 at nucleotide 51 of SEQ ID NO: 41 (or at nucleotide 51 in a variant thereof);
k) the AX or AA genotype for the Single Nucleotide Polymorphism marker SNP_42 at nucleotide 51 of SEQ ID NO: 42 (or at nucleotide 51 in a variant thereof);
l) the TX or TT genotype for the Single Nucleotide Polymorphism marker SNP_43 at nucleotide 51 of SEQ ID NO: 43 (or at nucleotide 51 in a variant thereof);
m) the TX or TT genotype for the Single Nucleotide Polymorphism marker SNP_44 at nucleotide 51 of SEQ ID NO: 44 (or at nucleotide 51 in a variant thereof);
n) the GX or GG genotype for the Single Nucleotide Polymorphism marker SNP_45 at nucleotide 51 of SEQ ID NO: 45 (or at nucleotide 51 in a variant thereof);
o) the AX or AA genotype for the Single Nucleotide Polymorphism marker SNP_46 at nucleotide 51 of SEQ ID NO: 46 (or at nucleotide 51 in a variant thereof);
p) the GX or GG genotype for the Single Nucleotide Polymorphism marker SNP_47 at nucleotide 51 of SEQ ID NO: 47 (or at nucleotide 51 in a variant thereof).

In one aspect said at least 5, 6, 7, 8, 9, 10, 11, 12, 13, 14, 15 or 16 markers which are detected are consecutive markers.

Thus, in one embodiment the plants according to the invention comprise at least a Thymine (T) (i.e. the TT or TX genotype) at nucleotide 51 of SEQ ID NO: 32 (referred to as SNP_32) or at the equivalent nucleotide of a genomic sequence comprising substantial sequence identity to SEQ ID NO:32 (in other words there is a Thymine at the physical position of chromosome 2 shown in Table 3);
and/or at least a Thymine (T) (i.e. the TT or TX genotype) at nucleotide 51 of SEQ ID NO: 33 (referred to as SNP_33) or at the equivalent nucleotide of a genomic sequence comprising substantial sequence identity to SEQ ID NO:33 (in other words there is a Thymine at the physical position of chromosome 2 shown in Table 3);
and/or at least a Cytosine (C) (i.e. the CC or CX genotype) at nucleotide 51 of SEQ ID NO: 34 (referred to as SNP_34) or at the equivalent nucleotide of a genomic sequence comprising substantial sequence identity to SEQ ID NO:34 (in other words there is a Cytosine at the physical position of chromosome 2 shown in Table 3);
and/or at least an Adenine (A) (i.e. the AA or AX genotype) at nucleotide 51 of SEQ ID NO: 35 (referred to as SNP_35) or at the equivalent nucleotide of a genomic sequence comprising substantial sequence identity to SEQ ID NO:35 (in other words there is a Adenine at the physical position of chromosome 2 shown in Table 3);
and/or at least a Thymine (T) (i.e. the TT or TX genotype) at nucleotide 51 of SEQ ID NO: 36 (referred to as SNP_36) or at the equivalent nucleotide of a genomic sequence comprising substantial sequence identity to SEQ ID NO:36 (in other words there is a Thymine at the physical position of chromosome 2 shown in Table 3);
and/or at least a Cytosine (C) (i.e. the CC or CX genotype) at nucleotide 51 of SEQ ID NO: 37 (referred to as SNP_37) or at the equivalent nucleotide of a genomic sequence comprising substantial sequence identity to SEQ ID NO:37 (in other words there is a Cytosine at the physical position of chromosome 2 shown in Table 3);
and/or at least a Guanine (G) (i.e. the GG or GX genotype) at nucleotide 51 of SEQ ID NO: 38 (referred to as SNP_38) or at the equivalent nucleotide of a genomic sequence comprising substantial sequence identity to SEQ ID NO:38 (in other words there is a Guanine at the physical position of chromosome 2 shown in Table 3);
and/or at least a Cytosine (C) (i.e. the CC or CX genotype) at nucleotide 51 of SEQ ID NO: 39 (referred to as SNP_39) or at the equivalent nucleotide of a genomic sequence comprising substantial sequence identity to SEQ ID NO:39 (in other words there is a Cytosine at the physical position of chromosome 2 shown in Table 3);
and/or at least a Guanine (G) (i.e. the GG or GX genotype) at nucleotide 51 of SEQ ID NO: 40 (referred to as SNP_40) or at the equivalent nucleotide of a genomic sequence comprising substantial sequence identity to SEQ ID NO:40 (in other words there is a Guanine at the physical position of chromosome 2 shown in Table 3);
and/or at least a Thymine (T) (i.e. the TT or TX genotype) at nucleotide 51 of SEQ ID NO: 41 (referred to as SNP_41) or at the equivalent nucleotide of a genomic sequence comprising substantial sequence identity to SEQ ID NO:41 (in other words there is a Thymine at the physical position of chromosome 2 shown in Table 3);
and/or at least an Adenine (A) (i.e. the AA or AX genotype) at nucleotide 51 of SEQ ID NO: 42 (referred to as SNP_42) or at the equivalent nucleotide of a genomic sequence comprising substantial sequence identity to SEQ ID NO:42 (in other words there is a Adenine at the physical position of chromosome 2 shown in Table 3);
and/or at least a Thymine (T) (i.e. the TT or TX genotype) at nucleotide 51 of SEQ ID NO: 43 (referred to as SNP_43) or at the equivalent nucleotide of a genomic sequence comprising substantial sequence identity to SEQ ID NO:43 (in other words there is a Thymine at the physical position of chromosome 2 shown in Table 3);
and/or at least a Thymine (T) (i.e. the TT or TX genotype) at nucleotide 51 of SEQ ID NO: 44 (referred to as SNP_44) or at the equivalent nucleotide of a genomic sequence comprising substantial sequence identity to SEQ ID NO:44 (in other words there is a Thymine at the physical position of chromosome 2 shown in Table 3);
and/or at least a Guanine (G) (i.e. the GG or GX genotype) at nucleotide 51 of SEQ ID NO: 45 (referred to as SNP_45) or at the equivalent nucleotide of a genomic sequence comprising substantial sequence identity to SEQ ID NO:45 (in other words there is a Guanine at the physical position of chromosome 2 shown in Table 3);
and/or at least an Adenine (A) (i.e. the AA or AX genotype) at nucleotide 51 of SEQ ID NO: 46 (referred to as SNP_46) or at the equivalent nucleotide of a genomic sequence comprising substantial sequence identity to SEQ ID NO:46 (in other words there is a Adenine at the physical position of chromosome 2 shown in Table 3);
and/or at least a Guanine (G) (i.e. the GG or GX genotype) at nucleotide 51 of SEQ ID NO: 47 (referred to as SNP_47) or at the equivalent nucleotide of a genomic sequence comprising substantial sequence identity to SEQ ID NO:47 (in other words there is a Guanine at the physical position of chromosome 2 shown in Table 3).

In a further one embodiment the presence of the introgression fragment, or the chromosome 2 region (or variant or orthologous chromosome 2 region), comprising QTL2.1, is detectable by a molecular marker assay which detects at least 3, 4 or 5 Single Nucleotide Polymorphism (SNP) markers of the sub-groups consisting of: SNP_32 to SNP_37; SNP_37 to SNP_42; SNP_42 to SNP_47; or SNP_34 to SNP_44.

The SNP genotype refers to two nucleotides, and genomic sequences comprising one of these two nucleotides, one on each chromosome 2. So a plant having a TT genotype for SNP_32 has an identical nucleotide (T) on both chromosomes (i.e. is homozygous), while a plant having an TX genotype for SNP_32 has one chromosome with an T at nucleotide 51 of SEQ ID NO: 32 (or at the equivalent nucleotide of a genomic sequence comprising substantial sequence identity to SEQ ID NO:32) and one chromosome with a X at nucleotide 51 of SEQ ID NO: 32 (or at the equivalent nucleotide of a genomic sequence comprising substantial sequence identity to SEQ ID NO:32) and is heterozygous, whereby X may be any nucleotide. As the genomic sequences around the SNP markers provided herein may vary slightly in introgression fragments from other wild cucumber donors (i.e. variants or orthologous chromosome 2 regions) it is clear that the nucleotide sequences before and after the SNP may not be 100% identical to the sequences provided herein. Therefore sequences having substantial sequence identity (i.e. at least 95% identity) to the sequences provided herein, but which comprise the same SNP, are encompassed herein.

In one aspect, the introgression fragment comprising QTL2.1, or the chromosome 2 region (or variant or orthologous chromosome 2 region) comprising the QTL (QTL2.1 or variant), which is detectable by the above one or more markers is from a wild or primitive cucumber, and in one aspect the wild or primitive cucumber is a member of the Indian Cucumber Group. In one aspect it is the same introgression fragment as found on chromosome 2 in seeds deposited under accession number NCIMB 43745, or a smaller fragment retaining the QTL. SNP markers SNP_32 to SNP_47 span a region of about 4.4 Mb. In one aspect the introgression fragment on chromosome 2 is equal to or less than 4.5 Mb in size, preferably equal to or less than 4.4 Mb in size, more preferably equal to or less than 3 or 2.5 Mb in size, e.g. equal to or less than 2 Mb. In one aspect the introgression fragment is at least 0.2 Mb, 0.5 Mb, 1.0 Mb, 1.5 Mb, 1.9 Mb, 2.0 Mb, 2.5 Mb, 2.7 Mb or 3 Mb in size. Thus, various ranges of introgression sizes are encompassed herein, such as fragments less than 4.5 Mb but more than 0.2 Mb, less than 4.4 Mb or 3 Mb but more than 0.2 Mb, 0.5 MB or 1 Mb, etc., which retain the QTL2.1 and one or more of the SNP markers of SNP_32 to SNP_47, or of the subgroups of SNP_32 to SNP_37; SNP_37 to SNP_42; SNP_42 to SNP_47 or SNP_34 to SNP_44. As mentioned before, the location of the QTL2.1 in the region spanning SNP_32 to SNP_47 can be determined by finemapping and recombinants comprising QTL2.1 on a smaller introgression fragment can be generated. The size of an introgression fragment can be easily determined by e.g. whole genome sequencing or Next Generation Sequencing, e.g. as described in Qi et al. 2013 (supra) or in Huang et al. 2009 (supra). Especially introgression regions can be easily distinguished from cultivated genomic regions due to the larger amount of genetic variation (SNPs, INDELs, etc.) in the introgression region.

To obtain the introgression fragment present on chromosome 2 (comprising QTL2.1) from the deposited seeds (NCIMB43745), i.e. to transfer the introgression fragments comprising the QTL to another cultivated cucumber plant, a plant is grown from the seed and the plant is crossed with a cultivated cucumber plant to obtain F1 seeds. As NCIMB43745 contains two recombinant chromosomes 2 (comprising the introgression fragment) all of the F1 seed and plants grown therefrom, contain one recombinant chromosome 2 from the NCIMB43745 parent and one non-recombinant chromosome 2 from the other cultivated parent. Thus, by traditional breeding one can transfer the recombinant chromosome 2 from NCIMB43745 into other cultivated cucumber lines or varieties. Plants which comprise the QTL2.1 can be screened for, and selected for, by the presence of one or more of the above SNP markers in order to identify plants comprising a recombinant chromosome 2.

To generate shorter introgression fragments (comprising QTL2.1) meiosis needs to take place and plants comprising the recombinant chromosomes 2, and especially new meiotic recombination events within the introgression fragment, need to be identified. For example, seeds of NCIMB43745 can be selfed one or more times to produce F1, F2 or F3 plants (or further selfing generations), and/or F1, F2 or F3 plants (etc.) comprising a recombinant chromosome 2 can be backcrossed to a cultivated parent. Plants which comprise the recombinant chromosome 2 can be screened for, and selected for, by the presence of one or more of the above SNP markers in order to identify plants comprising a smaller introgression fragment. Such new recombinants can then be tested for the presence of the QTL2.1 on the smaller introgression fragment (preferably in homozygous form) by determining the average disease score in a ToLCNDV-ES disease assay compared to the (genetic) control lacking the introgression fragment.

Similarly, cultivated cucumber plants comprising QTL2.1 (or a variant thereof) can be generated and/or identified using different methods. For example, to obtain a cultivated cucumber plant comprising a introgression fragment from a wild donor, a wild donor is identified which comprises one or more of the SNP markers linked to QTL2.1 disclosed herein, e.g. any one, or more, or all of the markers described herein above. This has for example been done for various wild accessions, see Examples. The identified plant is crossed with a cultivated cucumber plant to obtain F1 seeds. The F1 can be selfed to produce F2, F3, etc. plants, and/or F2 plants or F3 plants, etc., can be backcrossed to the cultivated cucumber parent. Plants which are comprising QTL2.1 (or a variant thereof) can be screened for, and/or selected for, by the presence of one or more of the above SNP markers and/or screened for, and/or selected for, an increased ToLCNDV-ES resistance phenotype (especially when QTL2.1 is in homozygous form) compared to the initial cultivated parent (lacking the introgressions). Alternatively or in addition, QTL mapping can be carried out in order to identify further molecular markers linked to the QTL2.1 (or a variant thereof) and/or to generate cultivated cucumber plants comprising an introgression fragment on chromosome 2 which confers significantly enhanced ToLCNDV-ES resistance, at least when in homozygous form.

In one embodiment the presence of the introgression fragment in a cultivated cucumber plant, or the chromosome 2 region (or orthologous chromosome 2 region), comprising QTL2.1, is detectable by a molecular marker assay which detects at least 5, 6, 7, 8, 9, 10 or more of the markers selected from the group consisting of:
a) the TT or TX genotype for the Single Nucleotide Polymorphism marker SNP_32 in SEQ ID NO: 32 (or in a variant thereof);
b) the GG or GX genotype for the Single Nucleotide Polymorphism marker SNP_47 in SEQ ID NO: 47 (or in a variant thereof);
c) any wild cucumber genome-specific marker in between marker SNP_32 and SNP_47.

In one aspect the markers of c) are one or more of SNP_33 to SNP_46. In one aspect, at least 5, 6, 7, 8, 9, 10 or more markers are detected from the markers of a), b) and/or c) above. In one embodiment at least the marker of a) and/or b) is detected and optionally at least one, two, three or more markers of c) are detected. In one aspect the markers detected are consecutive markers.

Any wild cucumber genome-specific marker in between two markers refers to any molecular marker which maps genetically to the chromosome 2 region in-between the two markers and/or which lies physically in-between the two markers, and which is indicative of the wild cucumber chromosome 2 region. This means that the marker is polymorphic between the cultivated cucumber genome and the wild cucumber genome. In one aspect, the marker is a Single Nucleotide Polymorphism (SNP), but other molecular markers such as RFLP, AFLP, RAPD, DNA sequencing, etc. may equally be used.

The introgression fragment in the plants of the invention is in one aspect a fragment of chromosome 2 (comprising QTL2.1) which is present in seeds deposited under accession number NCIMB43745 or a smaller version of that fragment retaining the QTL (generated by e.g. recombination within the introgression fragment).

The introgression fragment is in one aspect equal to or less than 4.5 Mb in size, preferably equal to or less than 4.4 Mb, 4.3 Mb, 4.0 Mb, 3.0 Mb, 2.5 Mb, 2 Mb, 1.5 Mb, 1 Mb in size. In a further aspect the introgression fragment is at least 0.5 Mb or at least 1 Mb in size.

Also provided are seeds from which a plant of the invention can be grown, as are cucumber fruits harvested from a plant of the invention and comprising the recombinant chromosome 2 in their genome (comprising QTL2.1 or a variant). Likewise a plant cell, tissue or plant part of a plant or of a seed is provided comprising at least one recombinant chromosome 2 (comprising QTL2.1 or a variant), wherein said recombinant chromosome 2 comprises an introgression fragment from a wild or primitive cucumber and wherein said introgression fragment comprises an allele conferring significantly enhanced ToLCNDV-ES resistance, at least when in homozygous form.

The molecular markers described herein may be detected according to standard method. For example SNP markers can easily be detected using a KASP-assay (see www.kpbioscience.co.uk) or other SNP genotyping assays. For developing a KASP-assay, for example 50 or 70 base pairs upstream and 50 or 70 base pairs downstream of the SNP can be selected and two allele-specific forward primers and one allele specific reverse primer can be designed. See e.g. Allen et al. 2011, Plant Biotechnology J. 9, 1086-1099, especially p 097-1098 for KASP assay method.

Thus, in one aspect, the SNP markers and the presence/absence of the marker associated with QTL2.1 is determined using a KASP assay, but equally other SNP genotyping assays can be used. For example, a TaqMan SNP genotyping assay, a High Resolution Melting (HRM) assay, SNP-genotyping arrays (e.g. Fluidigm, Illumina, etc.) or DNA sequencing may equally be used.

The physical size of an introgression fragment can be determined by various methods, such as physical mapping, sequencing or by visualization of the introgression using Fluorescent in situ hybridization (FISH) images (Verlaan et al. 2011, Plant Journal 68: 1093-1103).

Cultivated cucumber plants with smaller introgression fragments on chromosome 2 (comprising QTL2.1 or a variant) can be generated by generating new recombinant plants from a population of plants derived from a cross between a cultivated cucumber plant (lacking the introgressions) and a plant of the invention and selecting recombinant progeny having smaller introgression sizes. Such plants are thus in one aspect derived from (progeny or descendants of) the recombinant chromosome 2 present in plants of which seeds have been deposited under NCIMB43745. Such progeny or descendants which retain the QTL2.1, and thus the higher ToLCNDV-ES resistance (at least when QTL2.1 is in homozygous form) compared to plants lacking an introgression as described herein, are encompassed herein.

### QTL3.1 or Variants of QTL3.1 on Chromosome 3

Thus, in one aspect a cultivated cucumber plant is provided comprising an introgression fragment from a wild or primitive cucumber, wherein the introgression fragment comprises QTL3.1, or a variant thereof, and wherein the introgression fragment comprises all or part of the region starting at nucleotide (or base) 3637 of chromosome 3 (corresponding to SNP_48) and ending at nucleotide (or base) 3885803 of chromosome 3 (corresponding to SNP_62). In other words, all or part of the region starting at nucleotide 3637 of chromosome 3 and ending at nucleotide 3885803 of chromosome 3 is, in one aspect, from a wild donor cucumber and comprises QTL3.1 or a variant thereof. Which sub-region contains QTL3.1 can be identified by e.g. fine-mapping. So, for example if QTL3.1 is found to be in between SNP_48 and SNP_52, then the plant of the invention only needs to comprise the introgression region starting at nucleotide 3637 of chromosome 3 (SNP_48) and ending at nucleotide 376848 (SNP_52) of chromosome 3.

In one aspect QTL3.1 (or a variant thereof) is located in-between marker SNP_48 at nucleotide 51 of SEQ ID NO: 48 (or at nucleotide 51 in a variant sequence of SEQ ID NO: 48) and marker SNP_62 at nucleotide 51 of SEQ ID NO: 62 (or at nucleotide 51 in a variant sequence of SEQ ID NO: 62). In another aspect QTL3.1 (or a variant thereof) is located in-between marker SNP_48 at nucleotide 51 of SEQ ID NO: 48 (or at nucleotide 51 in a variant sequence of SEQ ID NO: 48) and marker SNP_52 at nucleotide 51 of SEQ ID NO: 52 (or at nucleotide 51 in a variant sequence of SEQ ID NO: 52). In a further aspect QTL3.1 (or a variant thereof) is located in-between marker SNP_52 at nucleotide 51 of SEQ ID NO: 52 (or at nucleotide 51 in a variant sequence of SEQ ID NO: 52) and marker SNP_57 at nucleotide 51 of SEQ ID NO: 57 (or at nucleotide 51 in a variant sequence of SEQ ID NO: 57). In a further aspect QTL3.1 (or a variant thereof) is located in-between marker SNP_57 at nucleotide 51 of SEQ ID NO: 57 (or at nucleotide 51 in a variant sequence of SEQ ID NO: 57) and marker SNP_62 at nucleotide 51 of SEQ ID NO: 62 (or nucleotide 51 in a variant sequence of SEQ ID NO: 62). In a further aspect QTL3.1 (or a variant thereof) is located in-between marker SNP_50 at nucleotide 51 of SEQ ID NO: 50 (or at nucleotide 51 in a variant sequence of SEQ ID NO: 50) and marker SNP_59 at nucleotide 51 of SEQ ID NO: 59 (or nucleotide 51 in a variant sequence of SEQ ID NO: 59).

In another aspect the introgression fragment of the invention (comprising QTL3.1 or a variant thereof) is a fragment comprising a smaller fragment (part) of the region starting at nucleotide (or base) 3634 of chromosome 3 and ending at nucleotide (or base) 3885803 of chromosome 3, e.g. having a size of e.g. 3.9 Mb 3.8 Mb, 3.5 Mb, 3.0 Mb, 2.5 Mb, 2 Mb, 1 Mb, 0.5 Mb, 100 kb, 50 kb, 35 kb, 30 kb, 20 kb, or less and comprising the QTL or a variant thereof. In one aspect the part is at least 5 kb, 10 kb, 20 kb in size, or more.

In one aspect the cultivated cucumber plant of the invention comprises an introgression fragment from a wild or primitive cucumber, which introgression fragment comprises QTL3.1 or a variant thereof, wherein the introgression fragment comprises all of part of the region starting at 0.003 Mb and ending at 3.9 Mb of the physical chromosome 3.

In one aspect the introgression fragment on chromosome 3 comprising QTL3.1, or a variant thereof, is obtainable by crossing a plant grown from NCIMB43745 with another cucumber plant, especially a cultivated cucumber plant, in one aspect a long cucumber type or a pickling or slicer type.

In one aspect the cultivated cucumber plant of the invention comprising QTL3.1, or a variant thereof, is a plant wherein said introgression fragment on chromosome 3 is obtainable by crossing a plant grown from seeds deposited under accession number NCIMB43745 with another cucumber plant. Thus, in one aspect the QTL is the QTL present in seeds deposited under accession number NCIMB43745.

In a further aspect the cultivated cucumber plant of the invention comprising QTL3.1, or a variant thereof, is a plant wherein said introgression fragment on chromosome 3 is obtainable by crossing a plant comprising the same SNP haplotype or SNP genotype for at least 5, 6, 7, 8, 9, 10 or more SNP markers linked to the QTL (i.e. SNP_48 to SNP_62 for QTL3.1 as shown in Table 4) with another cucumber plant, especially with a cultivated cucumber elite breeding line. Thus, in one aspect the QTL is the QTL present in wild donor accessions which comprise the same SNP haplotype or genotype for at least 5, 6, 7, 8, 9, 10 or more of the SNP markers, e.g. as in NCIMB43745. Preferably the donor also comprises a resistance phenotype having an average ToLCNDV-ES disease score of at least 7.5, preferably at least 8.0, 8.1, 8.2, 8.3, 8.4, 8.5, 8.6, 8.7, 8.8, 8.9 or 9.0.

When referring to the SNP markers herein, which are indicative of the presence of the introgression fragment (and the ToLCNDV-ES resistance QTL present on the introgression fragment), it is understood that the SNP genotype or haplotype which is indicative of the introgression fragment is referred to, i.e. the SNP genotype or haplotype as provided e.g. in Tables 1 to 4. It is noted that the SNP marker genotype can distinguish between the introgression fragment being in homozygous or heterozygous form. In homozygous form the nucleotide is identical, while in heterozygous form the nucleotide is not identical. The SNP genotype of the ‘wild type’ chromosome lacking the introgression fragment is the other haplotype, e.g. the haplotype of the recurrent parent). So, e.g. the genotype of SNP_48 indicative of the introgression fragment comprising QTL3.1 is ‘AC’ (QTL3.1/wt) or ‘AA’ (QTL3.1 QTL3.1) while the SNP genotype indicative of the wild type/genetic control (lacking the introgression fragment) is e.g. ‘CC’ (wt/wt). This can also be written as genotype AX′ (QTL3.1/wt) or ‘AA’ (QTL3.1 QTL3.1) while the SNP genotype indicative of the wild type/genetic control (lacking the introgression fragment) is e.g. ‘XX’ (wt/wt). X may be any nucleotide (A, T, C or G). Thus, when referring to a plant or plant part (e.g. cell) comprising the introgression fragment in homozygous or heterozygous form, it is understood that the SNP markers linked to the introgression fragment have the corresponding SNP genotype or haplotype.

So, in one aspect, a cultivated Cucumis sativus var. sativus plant is provided comprising an introgression fragment on chromosome 3 in homozygous or heterozygous form, wherein said introgression fragment confers an increase in ToLCNDV-ES resistance compared to the cucumber plant lacking the introgression fragment on chromosome 3, e.g. the genetic control or control variety, when grown under the same conditions. The increase in ToLCNDV-ES resistance is phenotypically expressed as a higher average disease score (less yellowing, measured e.g. in a disease assay as described herein) of the cultivated cucumber plant line or variety comprising the introgression fragment on chromosome 3 in homozygous or heterozygous form compared to the genetic control line or variety lacking the introgression fragment on chromosome 3 when grown under the same environment. The average disease score is preferably increased by at least 1.0, 1.5, 2.0, 2.5, 3.0 or more points on the disease scale of 2.0 (90-100% of leaf area is covered with yellowing mosaic symptoms) to 9.0 (no symptoms). So, for example if QTL3.1 is introduced into a susceptible cucumber line or variety having an average disease score of about 4.0, the introduction of QTL3.1 preferably increases the average disease score to an average score of at least 5.0, 5.5, 6.0, 6.5, 7.0 or more. As QTL3.1 was found to be additive, the effect of QTL3.1 in heterozygous form is less than the effect in homozygous form. Therefore the effect on ToLCNDV-ES resistance is preferably measured when it is in homozygous form.

It is known that all four QTLs together in homozygous form (QTL1.1, QTL1.2, QTL2.1 and QTL3.1) lead to an average disease score of 9.0 when introduced into a susceptible plant having an average disease score of about 4.0 to 5.0. The effect of the individual QTLs, even when in homozygous form, will be smaller than the combined effect, but will still be effective in reducing ToLCNDV-ES symptoms. The effect of QTL3.1 alone can be determined by introducing the QTL alone into a susceptible cucumber plant, in heterozygous or preferably in homozygous form. For example NCIMB43745 can be crossed with a susceptible plant and QTL3.1 alone can be transferred into the susceptible background.

The plants of the invention therefore comprise a genome of cultivated cucumber, with at least one or two recombinant chromosomes, namely one or two recombinant chromosomes 3 (i.e. heterozygous or homozygous). The recombinant chromosomes comprise a fragment of a wild donor cucumber, which is easily distinguishable from the cultivated cucumber genome by molecular marker analysis, whole genome sequencing, chromosome painting and similar techniques.

In one aspect the introgression fragment on chromosome 3 is from a wild or primitive cucumber, comprises the ToLCNDV-ES QTL3.1, or a variant thereof, and comprises all or part of the region starting at nucleotide SNP_48 and ending at SNP_62. Thus, the introgression fragment comprises the QTL3.1 or a variant thereof and one or more or all (e.g. 2, 3, 4, 5, 6, 7, 8, 9, 10, 11, 12, 13, 14, 15) SNP markers of the wild donor selected from SNP_48 to SNP_62 as shown in Table 4.

In one aspect the introgression fragment comprises QTL3.1 and one or more or all of SEQ ID NO: 48 to SEQ ID NO: 62.

In one aspect the presence of the introgression fragment on chromosomes 3 comprising QTL3.1 in the genome of the plant or plant cell or plant tissue (or in the DNA extracted therefrom) is detectable by a molecular marker assay which detects one or more molecular markers of the introgression fragment, especially the donor SNP haplotype or genotype for at least 5, 6, 7, 8, 9, 10 or more of SNP_48 to SNP_62, at nucleotide 51 of SEQ ID NO: 48 to 62, respectively. However, as mentioned, other techniques may be used, e.g. the SNP genotype of the markers may also be determined by sequencing or by using alternative markers located in between the SNP markers provided herein or within 7 cM, or within 5 cM, of a marker provided herein; or within 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less of a marker provided herein.

In one aspect the presence of the introgression fragment on chromosomes 3 comprising QTL3.1 in the genome of the plant or plant cell or plant tissue (or in the DNA extracted therefrom) is detectable by detecting the presence of one or more or all of SEQ ID NO: 48 to SEQ ID NO: 62.

When reference is made herein to one or more molecular markers or sequences being “detectable” by e.g. a molecular marker assay, this means of course that the plant or plant part comprises the one or more markers or sequences in its genome, as the marker or sequence would otherwise not be detectable.

### Cucumber Plants Comprising an Introgression Fragment on Chromosome 3 (QTL 3.1) or a Variant

In one aspect a cultivated Cucumis sativus var. sativus plant comprising an introgression fragment from a wild or primitive cucumber on chromosome 3 in homozygous or heterozygous form is provided, wherein said introgression fragment comprises a Quantitative Trait Locus (QTL) located between the Single Nucleotide Polymorphism marker SNP_48 at nucleotide 51 of SEQ ID NO: 48 (or at nucleotide 51 of a variant of SEQ ID NO: 48) and the Single Nucleotide Polymorphism marker SNP_62 at nucleotide 51 of SEQ ID NO: 62 (or at nucleotide 51 of a variant of SEQ ID NO: 62), which QTL confers an increase in ToLCNDV-ES resistance. In one aspect the QTL is located between base 3637 (SNP_48) and base 3885803 (SNP_62) of chromosome 3.

Thus, in one aspect QTL3.1 is located in the region between SNP_48 in SEQ ID NO: 48 (or in a variant thereof) and SNP_62 in SEQ ID NO: 62 (or in a variant thereof).

Therefore, in one aspect a cultivated Cucumis sativus var. sativus plant is provided comprising an introgression fragment on chromosome 3 in homozygous or heterozygous form, wherein said introgression fragment confers an increase in ToLCNDV-ES resistance (compared to the plant lacking the introgression fragment, e.g. the genetic control) and wherein said introgression fragment comprises the SNP marker haplotype or genotype of at least 5, 6, 7, 8, 9, 10, 11, 12, 13, 14, 15 or 16 of the SNP markers selected from the group consisting of:
a) the AX or AA genotype for the Single Nucleotide Polymorphism marker SNP_48 at nucleotide 51 of SEQ ID NO: 48 (or at nucleotide 51 in a variant thereof);
b) the CX or CC genotype for the Single Nucleotide Polymorphism marker SNP_49 at nucleotide 51 of SEQ ID NO: 49 (or at nucleotide 51 in a variant thereof);
c) the CX or CC genotype for the Single Nucleotide Polymorphism marker SNP_50 at nucleotide 51 of SEQ ID NO: 50 (or at nucleotide 51 in a variant thereof);
d) the CX or CC genotype for the Single Nucleotide Polymorphism marker SNP_51 at nucleotide 51 of SEQ ID NO: 51 (or at nucleotide 51 in a variant thereof);
e) the TX or TT genotype for the Single Nucleotide Polymorphism marker SNP_52 at nucleotide 51 of SEQ ID NO: 52 (or at nucleotide 51 in a variant thereof);
f) the CX or CC genotype for the Single Nucleotide Polymorphism marker SNP_53 at nucleotide 51 of SEQ ID NO: 53 (or at nucleotide 51 in a variant thereof);
g) the CX or CC genotype for the Single Nucleotide Polymorphism marker SNP_54 at nucleotide 51 of SEQ ID NO: 54 (or at nucleotide 51 in a variant thereof);
h) the CX or CC genotype for the Single Nucleotide Polymorphism marker SNP_55 at nucleotide 51 of SEQ ID NO: 55 (or at nucleotide 51 in a variant thereof);
i) the GX or GG genotype for the Single Nucleotide Polymorphism marker SNP_56 at nucleotide 51 of SEQ ID NO: 56 (or at nucleotide 51 in a variant thereof);
j) the CX or CC genotype for the Single Nucleotide Polymorphism marker SNP_57 at nucleotide 51 of SEQ ID NO: 57 (or at nucleotide 51 in a variant thereof);
k) the TX or TT genotype for the Single Nucleotide Polymorphism marker SNP_58 at nucleotide 51 of SEQ ID NO: 58 (or at nucleotide 51 in a variant thereof);
l) the TX or TT genotype for the Single Nucleotide Polymorphism marker SNP_59 at nucleotide 51 of SEQ ID NO: 59 (or at nucleotide 51 in a variant thereof);
m) the GX or GG genotype for the Single Nucleotide Polymorphism marker SNP_60 at nucleotide 51 of SEQ ID NO: 60 (or at nucleotide 51 in a variant thereof);
n) the AX or AA genotype for the Single Nucleotide Polymorphism marker SNP_61 at nucleotide 51 of SEQ ID NO: 61 (or at nucleotide 51 in a variant thereof);
o) the AX or AA genotype for the Single Nucleotide Polymorphism marker SNP_62 at nucleotide 51 of SEQ ID NO: 62 (or at nucleotide 51 in a variant thereof).

When referring to a SNP in a variant sequence, that variant sequence comprises at least 95%, 96%, 97%, 98% or 99% sequence identity with the mentioned sequence. X refers to any nucleotide for the sequence on the other chromosome 3 of the pair of chromosomes. In one aspect X may be the nucleotide of the recurrent parent as described in Table 4.

In one aspect said at least 5, 6, 7, 8, 9, 10, 11, 12, 13, 14 or 15 markers are consecutive markers.

The fragment comprising the QTL3.1 may, thus, be large (comprising SNP_48 to SNP_62), or may be smaller and lack markers having the genotype or haplotype of the wild cucumber (i.e. the markers have the cultivated cucumber genotype or haplotype instead, see also Table 4, SNP haplotype of recurrent parent), but it may still confer enhanced ToLCNDV-ES resistance on the cultivated cucumber plant, i.e. it can still comprise the ToLCNDV-ES allele (QTL3.1 or a variant). Such smaller introgression fragments are an embodiment of the invention. Plants having smaller introgression fragments which still confer the enhanced ToLCNDV-ES resistance (i.e. contain the resistance allele) can be generated using known techniques, such as fine-mapping or similar techniques. For example by starting with a plant comprising the introgression fragment as found in seeds deposited under accession number NCIMB 43745 and crossing such a plant with another cultivated cucumber plant and selfing the progeny of said cross, and/or backcrossing the progeny, to generate a population of plants which may contain recombinants having a smaller introgression fragment on chromosome 3, which fragments still confer enhanced ToLCNDV-ES resistance in relation to a plant lacking the introgression fragment (such as the genetic control, e.g. plants grown from seeds deposited under NCIMB42344), e.g. a fragment comprising markers SNP_48 to SNP_52, or SNP_52 to SNP_57 or SNP_57 to SNP_62 or SNP_50 to SNP_59. Marker assays can be used to determine the size of the smaller introgression fragment. One or more of the SNP markers with the genotype or haplotype of the wild donor cucumber may be missing. The cultivated cucumber genotype or haplotype is then detected for these SNP markers. The ToLCNDV-ES resistance of plants comprising such a smaller introgression fragment can then be compared in a disease assay as described herein, i.e. growing a plurality of plants comprising the smaller introgression fragment in experiments together with suitable control plants, lacking the introgression fragments. The control plants are preferably a genetic control, such as NCIMB43744. If the average ToLCNDV-ES disease score remains significantly higher than in the control, then the smaller introgression fragment has retained the QTL3.1.

Alternatively, the same or variant QTL (QTL3.1 or variant QTL3.1) may be introgressed from a different wild donor accessions, whereby optionally not all SNP markers disclosed herein may be present, i.e. the SNP haplotype of the donor accession may only be identical to the SNP haplotype of the QTL3.1 present in seeds of NCIMB43745 for at least 5, 6, 7, 8, 9, 10 or more SNPs. Such alternative wild cucumber sources can be identified using the SNP markers provided herein, by screening germplasm (i.e. accessions of) wild or primitive cucumber using a marker assay to detect the genotype or haplotype of one or more markers of markers SNP_48 to SNP_62, or of markers SNP_48 to SNP_52, SNP_52 to SNP_57, SNP_57 to SNP_62, or SNP_50 to SNP_59, or even only a smaller subgroup of these markers (e.g. 2, 3 or 4). For example, Table 4 shows various donors (PI605996, CGN22263 and CGN22932, also known as PI197087) which have the same SNP haplotype for SNP_48 to SNP_62. In the same way other donors, having QTL3.1 or a variant thereof, may be identified. Plants comprising the same or variant QTL3.1 from these donors or from other sources are also an embodiment of the invention. Thus, as long as at least 5, 6, 7, 8, 9, 10 or more (or all) of the SNPs of SNP_48 to SNP_62, or of the SNPs of SNP_48 to SNP_52, or of the SNPs of SNP_52 to SNP_57, or of the SNPs of SNP_57 to SNP_62, or of the SNPs of SNP_50 to SNP_59 are present, the donor may contain QTL3.1 (or a variant thereof) and is encompassed herein. The skilled person can then introgress the QTL3.1 (or a variant thereof) into cultivated cucumber in order to enhance ToLCNDV-ES resistance as described herein and in order to confirm that the QTL enhances ToLCNDV-ES resistance when present in cultivated cucumber. Prior to introgression the wild donor may also be tested for ToLCNDV-ES resistance in an assay as described and e.g. a donor may be selected that comprises an average ToLCNDV-ES score of e.g. at least 7.5, 8.0, 8.5, or 9.0.

As described above, in one embodiment the cultivated cucumber plant of the invention comprises an introgression fragment comprising at least a subset of SNP markers with the genotype (or haplotype) of the wild donor cucumber, i.e. at least 5, 6, 7, 8, 9 or more markers of SNP_48 to SNP_62, or at least 3 markers of SNP_48 to SNP_52, or of SNP_52 to SNP_57, or of SNP_57 to SNP_62, or of SNP_50 to SNP_59. In one aspect the cultivated cucumber plant comprises all, or all except 1 or 2 markers of SNP_48 to SNP_62, or of SNP_48 to SNP_52, or of SNP_52 to SNP_57, or of SNP_57 to SNP_62, or of SNP_50 to SNP_59.

Thus, the introgression fragment (and a cultivated cucumber plant or plant part, e.g., a cell, comprising the introgression fragment) can be detected in a marker assay by detecting the SNP genotype or haplotype of the introgression fragment (i.e. of the wild cucumber germplasm) of one or more or all of the markers above, preferably at least 5, 6, 7, 8 or more.

Thus, in one aspect, a Quantitative Trait Locus (QTL3.1) was found to be present on chromosome 3 of a wild cucumber donor which, when transferred (introgressed) into a cultivated cucumber variety or breeding line, and when present in heterozygous or homozygous form, confers significantly enhanced ToLCNDV-ES resistance onto the cultivated cucumber plant. The QTL, or the introgression fragment comprising the QTL (comprising the ToLCNDV resistance allele), is thus additive, i.e. it is sufficient to have the introgression fragment on one of the chromosomes 3 (one recombinant chromosome 1), while the homologous chromosome 3 of the pair may be a (non-recombinant) chromosome 3 of cultivated C. sativus var. sativus lacking the introgression fragment.

Although the present source of the QTL3.1 which was used to map and introgress the QTL is a single, specific wild source, there are other wild accessions which comprise QTL3.1 at the same locus on chromosome 3. For example the wild accessions PI605996, CGN22263 and CGN22932, also known as PI197087, were found to comprise the same SNP haplotype for markers SNP_48 to SNP_62 (as shown in Table 3) and were found to be resistant to ToLCNDV-ES. The (variant) QTL3.1 from these or other donors can, thus, be introgressed into cultivated cucumber, optionally in combination with one or more of QTL1.1, QTL1.2 and QTL2.1. Similarly, other wild or primitive cucumber accessions can be screened for the SNP haplotype or genotype of one or more or all of SNP_48 to SNP_62.

Such other donors may comprise a ToLCNDV-ES resistance allele which has a slightly different nucleotide sequences, i.e. variants of the allele (QTL3.1) found herein. Such variant QTLs can also be identified and introgressed into cultivated cucumber as described herein, to generate a cultivated cucumber plant comprising a genome of cultivated C. sativus var. sativus and a recombinant chromosome 3, whereby the recombinant chromosome 3 comprises an introgression fragment, which confers an enhanced ToLCNDV-ES resistance onto the cultivated cucumber plant when present in homozygous or heterozygous form. To identify such wild donor accessions comprising QTL3.1, wild accessions can be screened, e.g. in a marker assay or by sequence comparison or other methods, for the presence of one or more of the SNP markers provided herein. The putative QTL (or variant QTL) can then be introgressed into cultivated cucumber, e.g. using MAS, i.e. using one or more (or all) of the SNP markers provided herein to detect and/or select progeny plants (e.g. backcross plants) comprising a recombinant chromosome 3. The selected plants, i.e. the cultivated cucumber plants comprising an introgression fragment on chromosome 3, wherein the introgression fragment on chromosome 3 is detectable by 5, 6, 7, 8, 9, 10 or more of the SNP markers SNP_48 to SNP_62 can then be phenotyped in a ToLCNDV-ES disease assay together with the suitable control plants, preferably at least the genetic control, in order to determine whether the introgression fragment indeed causes a significant increase in ToLCNDV-ES resistance.

Accessions of wild or primitive cucumber, are obtainable from e.g. the USDA National Plant Germplasm System collection or other seed collections, and can thus be screened for the presence of QTL3.1 using e.g. a marker assay as described herein, and accessions comprising 5 or more of the SNP markers (e.g. at least 5, 6, 7, 8, 9, 10 or more SNP markers indicative of QTL3.1 or a variant) can be crossed with a cultivated cucumber plant having normal wild-type, non-recombinant chromosomes 3. The F1 or F2 generation (or further generation, such as the F3 or a backcross generation) can then be screened for recombinant plants having the introgression fragment or a part thereof, using the molecular marker assays described herein.

In one aspect, the introgression fragment is from a donor comprising the SNP haplotype for QTL3.1 as shown in Table 4 for the introgression donor (NCIMB43745), for P1605996, for CGN22263 or for CGN22932, also known as PI197087.

In a specific embodiment, the introgression fragment comprising the ToLCNDV-ES QTL3.1 (or a variant) is derivable from (or derived from) or obtainable from (or obtained from; or as present in) seeds, a representative sample of which has been deposited under accession number NCIMB 43745, or from progeny thereof, or from seeds having accession number PI605996 (USDA ARS-GRIN collection), or from seeds having accession number CGN22263 or CGN22932 (Wageningen CGN collection) or from seeds having accession number PI197087 (USDA ARS-GRIN collection) or from progeny of any of these. The progeny may be any progeny which retain the SNP markers or haplotype indicative of (and linked to) the QTL, as described. Thus, progeny are not limited to F1 or F2 progeny of the deposit or accession, but can be any progeny, whether obtained by selfing and/or crossing with another cucumber plant.

In one embodiment the introgression fragment comprising QTL3.1 is identifiable by one or more of the markers described elsewhere herein, especially markers SNP_48 to SNP_62 for the introgression fragment on chromosome 3, or a subset of markers, such as one or more of the markers selected from SNP markers SNP_48 to SNP_52, or from SNP markers SNP_52 to SNP_57, or from of the SNP markers SNP_57 to SNP_62, or from SNP markers SNP_50 to SNP_59. In one aspect the invention provides a cultivated cucumber plant, having a genome of cultivated (domesticated) cucumber which comprises enhanced ToLCNDV-ES resistance, wherein the enhanced resistance is conferred by an introgression fragment on the cultivated cucumber chromosome 3, wherein said introgression fragment is obtained by (or obtainable by) crossing a cultivated plant grown from seeds deposited under NCIMB 43745 or progeny of this plant (which comprises one or more the markers disclosed herein linked to the QTL) with a cultivated cucumber plant. Thus in one aspect the cultivated cucumber plant of the invention comprises the same introgression fragment and the same recombinant chromosome 3 as present in NCIMB 43745 (comprising all of the wild donor haplotype for SNP markers SNP_48 to SNP_62 or comprising SEQ ID NO: 48 to 62), or it comprises a shorter fragment of that introgression fragment, whereby the shorter fragment retains the genetic element conferring ToLCNDV-ES resistance (QTL3.1).

Thus in one aspect the invention relates to a plant of the invention i.e. a cultivated Cucumis sativus var. sativus plant comprising an introgression fragment comprising QTL3.1 from a wild cucumber on chromosome 3 in homozygous or heterozygous form and wherein said introgression fragment is the introgression fragment “as in”/is “identical to”/is “the same as in” the seeds deposited under number NCIMB 43745, or is a shorter fragment thereof, but still confers enhanced ToLCNDV resistance due to the presence of QTL3.1.

As SEQ ID NO: 48 to 62 are from the wild donor used to generate NCIMB43745, they can identify the introgression fragment or sub-fragments of the specific donor.

In yet another embodiment the invention relates to a plant of the invention i.e. a cultivated Cucumis sativus var. sativus plant comprising an introgression fragment comprising QTL3.1 from a wild cucumber on chromosome 3 in homozygous or heterozygous form and wherein said introgression fragment is the introgression fragment is a variant of the introgression fragment seeds deposited under number NCIMB 43745, i.e. it comprises the QTL 3.1, but the genomic sequence may be different. As wild accessions will be genetically divergent, the genomic sequence of an introgression fragment comprising QTL3.1 from other wild or primitive cucumbers will most likely not be identical to the genomic sequence as introgressed into NCIMB 43745, and even the ToLCNDV-ES conferring gene (comprising a promoter, introns and exons) may be divergent in nucleotide sequence, but the function will be the same, i.e. conferring enhanced resistance. The divergence can be seen in that certain SNP markers linked to QTL3.1 may be commonly found in various accessions, while other SNP markers may only be found in specific accessions. So for example not all of SNP_48 to SNP_62 may be found in other wild cucumber donors. For example another donor may have a slightly different SNP haplotype for SNP_48 to SNP_62, with e.g. one or two SNP markers having a different nucleotide. However, QTL3.1 (comprising e.g. a variant or ortholog of the ToLCNDV-ES resistance allele) may still be present in such wild accessions. The skilled person is capable of identifying and introgressing the QTL3.1 comprising region found in other wild cucumber donors into cultivated cucumber, e.g. detecting wild accessions comprising the SNP markers or a subset thereof and transferring these SNP markers (or subset) into a cultivated cucumber line or variety and assessing the ToLCNDV-ES resistance of the cultivated line or variety compared to the line or variety lacking the SNP markers (or subset), i.e. lacking the introgression fragment. Even in cases where the SNP haplotype for SNP_48 to SNP_62 is identical to the SNP haplotype of QTL3.1 found in seeds of NCIMB 43745, the actual nucleotide sequences flanking the SNP at nucleotide 51 of SEQ ID NO: 48 to 62 may be different in other donors. So other donors may comprise the same SNP nucleotide at nucleotide 51, but in a sequence comprising at least 95%, 96%, 97%, 98% or 99% sequence identity to SEQ ID NO: 48 to 62 when e.g. aligned pairwise. This variation can be seen by sequencing the donors and aligning sequences of SEQ ID NO: 48 to SEQ ID NO: 62 with that sequence.

In one embodiment the presence of the introgression fragment comprising QTL3.1, or the chromosome 3 region (or variant or orthologous chromosome 3 region), comprising QTL3.1, is detectable by a molecular marker assay which detects at least 5, 6, 7, 8, 9, 10, 11, 12, 13, 14, 15 or 16 Single Nucleotide Polymorphism (SNP) markers selected from the group consisting of:
a) the AX or AA genotype for the Single Nucleotide Polymorphism marker SNP_48 at nucleotide 51 of SEQ ID NO: 48 (or at nucleotide 51 in a variant thereof);
b) the CX or CC genotype for the Single Nucleotide Polymorphism marker SNP_49 at nucleotide 51 of SEQ ID NO: 49 (or at nucleotide 51 in a variant thereof);
c) the CX or CC genotype for the Single Nucleotide Polymorphism marker SNP_50 at nucleotide 51 of SEQ ID NO: 50 (or at nucleotide 51 in a variant thereof);
d) the CX or CC genotype for the Single Nucleotide Polymorphism marker SNP_51 at nucleotide 51 of SEQ ID NO: 51 (or at nucleotide 51 in a variant thereof);
e) the TX or TT genotype for the Single Nucleotide Polymorphism marker SNP_52 at nucleotide 51 of SEQ ID NO: 52 (or at nucleotide 51 in a variant thereof);
f) the CX or CC genotype for the Single Nucleotide Polymorphism marker SNP_53 at nucleotide 51 of SEQ ID NO: 53 (or at nucleotide 51 in a variant thereof);
g) the CX or CC genotype for the Single Nucleotide Polymorphism marker SNP_54 at nucleotide 51 of SEQ ID NO: 54 (or at nucleotide 51 in a variant thereof);
h) the CX or CC genotype for the Single Nucleotide Polymorphism marker SNP_55 at nucleotide 51 of SEQ ID NO: 55 (or at nucleotide 51 in a variant thereof);
i) the GX or GG genotype for the Single Nucleotide Polymorphism marker SNP_56 at nucleotide 51 of SEQ ID NO: 56 (or at nucleotide 51 in a variant thereof);
j) the CX or CC genotype for the Single Nucleotide Polymorphism marker SNP_57 at nucleotide 51 of SEQ ID NO: 57 (or at nucleotide 51 in a variant thereof);
k) the TX or TT genotype for the Single Nucleotide Polymorphism marker SNP_58 at nucleotide 51 of SEQ ID NO: 58 (or at nucleotide 51 in a variant thereof);
l) the TX or TT genotype for the Single Nucleotide Polymorphism marker SNP_59 at nucleotide 51 of SEQ ID NO: 59 (or at nucleotide 51 in a variant thereof);
m) the GX or GG genotype for the Single Nucleotide Polymorphism marker SNP_60 at nucleotide 51 of SEQ ID NO: 60 (or at nucleotide 51 in a variant thereof);
n) the AX or AA genotype for the Single Nucleotide Polymorphism marker SNP_61 at nucleotide 51 of SEQ ID NO: 61 (or at nucleotide 51 in a variant thereof);
o) the AX or AA genotype for the Single Nucleotide Polymorphism marker SNP_62 at nucleotide 51 of SEQ ID NO: 62 (or at nucleotide 51 in a variant thereof).

In one aspect said at least 5, 6, 7, 8, 9, 10, 11, 12, 13, 14, 15 or 16 markers which are detected are consecutive markers.

Thus, in one embodiment the plants according to the invention comprise at least an Adenine (A) (i.e. the AA or AX genotype) at nucleotide 51 of SEQ ID NO: 48 (referred to as SNP_48) or at the equivalent nucleotide of a genomic sequence comprising substantial sequence identity to SEQ ID NO:48 (in other words there is a Adenine at the physical position of chromosome 3 shown in Table 4);
and/or at least a Cytosine (C) (i.e. the CC or CX genotype) at nucleotide 51 of SEQ ID NO: 49 (referred to as SNP_49) or at the equivalent nucleotide of a genomic sequence comprising substantial sequence identity to SEQ ID NO:49 (in other words there is a Cytosine at the physical position of chromosome 3 shown in Table 4);
and/or at least a Cytosine (C) (i.e. the CC or CX genotype) at nucleotide 51 of SEQ ID NO: 50 (referred to as SNP_50) or at the equivalent nucleotide of a genomic sequence comprising substantial sequence identity to SEQ ID NO:50 (in other words there is a Cytosine at the physical position of chromosome 3 shown in Table 4);
and/or at least a Cytosine (C) (i.e. the CC or CX genotype) at nucleotide 51 of SEQ ID NO: 51 (referred to as SNP_51) or at the equivalent nucleotide of a genomic sequence comprising substantial sequence identity to SEQ ID NO:51 (in other words there is a Cytosine at the physical position of chromosome 3 shown in Table 4);
and/or at least a Thymine (T) (i.e. the TT or TX genotype) at nucleotide 51 of SEQ ID NO: 52 (referred to as SNP_52) or at the equivalent nucleotide of a genomic sequence comprising substantial sequence identity to SEQ ID NO:52 (in other words there is a Thymine at the physical position of chromosome 3 shown in Table 4);
and/or at least a Cytosine (C) (i.e. the CC or CX genotype) at nucleotide 51 of SEQ ID NO: 53 (referred to as SNP_53) or at the equivalent nucleotide of a genomic sequence comprising substantial sequence identity to SEQ ID NO:53 (in other words there is a Cytosine at the physical position of chromosome 3 shown in Table 4);
and/or at least a Cytosine (C) (i.e. the CC or CX genotype) at nucleotide 51 of SEQ ID NO: 54 (referred to as SNP_54) or at the equivalent nucleotide of a genomic sequence comprising substantial sequence identity to SEQ ID NO:54 (in other words there is a Cytosine at the physical position of chromosome 3 shown in Table 4);
and/or at least a Cytosine (C) (i.e. the CC or CX genotype) at nucleotide 51 of SEQ ID NO: 55 (referred to as SNP_55) or at the equivalent nucleotide of a genomic sequence comprising substantial sequence identity to SEQ ID NO:55 (in other words there is a Cytosine at the physical position of chromosome 3 shown in Table 4);
and/or at least a Guanine (G) (i.e. the GG or GX genotype) at nucleotide 51 of SEQ ID NO: 56 (referred to as SNP_56) or at the equivalent nucleotide of a genomic sequence comprising substantial sequence identity to SEQ ID NO:56 (in other words there is a Guanine at the physical position of chromosome 3 shown in Table 4);
and/or at least a Cytosine (C) (i.e. the CC or CX genotype) at nucleotide 51 of SEQ ID NO: 57 (referred to as SNP_57) or at the equivalent nucleotide of a genomic sequence comprising substantial sequence identity to SEQ ID NO:57 (in other words there is a Cytosine at the physical position of chromosome 3 shown in Table 4);
and/or at least a Thymine (T) (i.e. the TT or TX genotype) at nucleotide 51 of SEQ ID NO: 58 (referred to as SNP_58) or at the equivalent nucleotide of a genomic sequence comprising substantial sequence identity to SEQ ID NO:58 (in other words there is a Thymine at the physical position of chromosome 3 shown in Table 4);
and/or at least a Thymine (T) (i.e. the TT or TX genotype) at nucleotide 51 of SEQ ID NO: 59 (referred to as SNP_59) or at the equivalent nucleotide of a genomic sequence comprising substantial sequence identity to SEQ ID NO:59 (in other words there is a Thymine at the physical position of chromosome 3 shown in Table 4);
and/or at least a Guanine (G) (i.e. the GG or GX genotype) at nucleotide 51 of SEQ ID NO: 60 (referred to as SNP_60) or at the equivalent nucleotide of a genomic sequence comprising substantial sequence identity to SEQ ID NO:60 (in other words there is a Guanine at the physical position of chromosome 3 shown in Table 4);
and/or at least an Adenine (A) (i.e. the AA or AX genotype) at nucleotide 51 of SEQ ID NO: 61 (referred to as SNP_61) or at the equivalent nucleotide of a genomic sequence comprising substantial sequence identity to SEQ ID NO:61 (in other words there is an Adenine at the physical position of chromosome 3 shown in Table 4);
and/or at least an Adenine (A) (i.e. the AA or AX genotype) at nucleotide 51 of SEQ ID NO: 62 (referred to as SNP_62) or at the equivalent nucleotide of a genomic sequence comprising substantial sequence identity to SEQ ID NO:62 (in other words there is an Adenine at the physical position of chromosome 3 shown in Table 4).

In a further one embodiment the presence of the introgression fragment, or the chromosome 3 region (or variant or orthologous chromosome 3 region), comprising QTL3.1, is detectable by a molecular marker assay which detects at least 3, 4 or 5 Single Nucleotide Polymorphism (SNP) markers of the sub-groups consisting of: SNP_48 to SNP_52: SNP_52 to SNP_57: SNP_57 to SNP_62: or SNP_50 to SNP_59.

The SNP genotype refers to two nucleotides, and genomic sequences comprising one of these two nucleotides, one on each chromosome 3. So a plant having a AA genotype for SNP_48 has an identical nucleotide (A) on both chromosomes (i.e. is homozygous), while a plant having an AX genotype for SNP_48 has one chromosome with an A at nucleotide 51 of SEQ ID NO: 48 (or at the equivalent nucleotide of a genomic sequence comprising substantial sequence identity to SEQ ID NO:48) and one chromosome with a X at nucleotide 51 of SEQ ID NO: 48 (or at the equivalent nucleotide of a genomic sequence comprising substantial sequence identity to SEQ ID NO:48) and is heterozygous, whereby X may be any nucleotide. As the genomic sequences around the SNP markers provided herein may vary slightly in introgression fragments from other wild cucumber donors (i.e. variants or orthologous chromosome 3 regions) it is clear that the nucleotide sequences before and after the SNP may not be 100% identical to the sequences provided herein. Therefore sequences having substantial sequence identity (i.e. at least 95% identity) to the sequences provided herein, but which comprise the same SNP, are encompassed herein.

In one aspect, the introgression fragment comprising QTL3.1, or the chromosome 3 region (or variant or orthologous chromosome 3 region) comprising the QTL (QTL3.1 or variant), which is detectable by the above one or more markers is from a wild or primitive cucumber, and in one aspect the wild or primitive cucumber is a member of the Indian Cucumber Group. In one aspect it is the same introgression fragment as found on chromosome 3 in seeds deposited under accession number NCIMB 43745, or a smaller fragment retaining the QTL. SNP markers SNP_48 to SNP_62 span a region of about 4 Mb. In one aspect the introgression fragment on chromosome 3 is equal to or less than 4 Mb in size, preferably equal to or less than 3.9 Mb in size, more preferably equal to or less than 3.8, 3.0 or 2.5 Mb in size, e.g. equal to or less than 2 Mb. In one aspect the introgression fragment is at least 0.2 Mb, 0.5 Mb, 1.0 Mb, 1.5 Mb, 1.9 Mb, 2.0 Mb, 2.5 Mb, 2.7 Mb or 3 Mb in size. Thus, various ranges of introgression sizes are encompassed herein, such as fragments less than 4 Mb but more than 0.2 Mb, less than 3.9 Mb or 3 Mb but more than 0.2 Mb, 0.5 MB or 1 Mb, etc., which retain the QTL3.1 and one or more of the SNP markers of SNP_48 to SNP_62, or of the subgroups of SNP_48 to SNP_52; SNP_52 to SNP_57; SNP_57 to SNP_62 or SNP_50 to SNP_59. As mentioned before, the location of the QTL3.1 in the region spanning SNP_48 to SNP_62 can be determined by finemapping and recombinants comprising QTL3.1 on a smaller introgression fragment can be generated. The size of an introgression fragment can be easily determined by e.g. whole genome sequencing or Next Generation Sequencing, e.g. as described in Qi et al. 2013 (supra) or in Huang et al. 2009 (supra). Especially introgression regions can be easily distinguished from cultivated genomic regions due to the larger amount of genetic variation (SNPs, INDELs, etc.) in the introgression region.

To obtain the introgression fragment present on chromosome 3 (comprising QTL3.1) from the deposited seeds (NCIMB43745), i.e. to transfer the introgression fragments comprising the QTL to another cultivated cucumber plant, a plant is grown from the seed and the plant is crossed with a cultivated cucumber plant to obtain F1 seeds. As NCIMB43745 contains two recombinant chromosomes 3 (comprising the introgression fragment) all of the F1 seed and plants grown therefrom, contain one recombinant chromosome 3 from the NCIMB43745 parent and one non-recombinant chromosome 3 from the other cultivated parent. Thus, by traditional breeding one can transfer the recombinant chromosome 3 from NCIMB43745 into other cultivated cucumber lines or varieties. Plants which comprise the QTL3.1 can be screened for, and selected for, by the presence of one or more of the above SNP markers in order to identify plants comprising a recombinant chromosome 3.

To generate shorter introgression fragments (comprising QTL3.1) meiosis needs to take place and plants comprising the recombinant chromosomes 3, and especially new meiotic recombination events within the introgression fragment, need to be identified. For example, seeds of NCIMB43745 can be selfed one or more times to produce F1, F2 or F3 plants (or further selfing generations), and/or F1, F2 or F3 plants (etc.) comprising a recombinant chromosome 3 can be backcrossed to a cultivated parent. Plants which comprise the recombinant chromosome 3 can be screened for, and selected for, by the presence of one or more of the above SNP markers in order to identify plants comprising a smaller introgression fragment. Such new recombinants can then be tested for the presence of the QTL3.1 on the smaller introgression fragment by determining the average disease score in a ToLCNDV-ES disease assay compared to the (genetic) control lacking the introgression fragment.

Similarly, cultivated cucumber plants comprising QTL3.1 (or a variant thereof) can be generated and/or identified using different methods. For example, to obtain a cultivated cucumber plant comprising an introgression fragment from a wild donor, a wild donor is identified which comprises one or more of the SNP markers linked to QTL3.1 disclosed herein, e.g. any one, or more, or all of the markers described herein above. This has for example been done for various wild accessions, see Examples. The identified plant is crossed with a cultivated cucumber plant to obtain F1 seeds. The F1 can be selfed to produce F2, F3, etc. plants, and/or F2 plants or F3 plants, etc., can be backcrossed to the cultivated cucumber parent. Plants which are comprising QTL3.1 (or a variant thereof) can be screened for, and/or selected for, by the presence of one or more of the above SNP markers and/or screened for, and/or selected for, an increased ToLCNDV-ES resistance phenotype compared to the initial cultivated parent (lacking the introgressions). Alternatively or in addition, QTL mapping can be carried out in order to identify further molecular markers linked to the QTL3.1 (or a variant thereof) and/or to generate cultivated cucumber plants comprising an introgression fragment on chromosome 3 which confers significantly enhanced ToLCNDV-ES resistance.

In one embodiment the presence of the introgression fragment in a cultivated cucumber plant, or the chromosome 3 region (or orthologous chromosome 3 region), comprising QTL3.1, is detectable by a molecular marker assay which detects at least 5, 6, 7, 8, 9, 10 or more of the markers selected from the group consisting of:
a) the AA or AX genotype for the Single Nucleotide Polymorphism marker SNP_48 in SEQ ID NO: 48 (or in a variant thereof);
b) the AA or AX genotype for the Single Nucleotide Polymorphism marker SNP_62 in SEQ ID NO: 62 (or in a variant thereof);
c) any wild cucumber genome-specific marker in between marker SNP_48 and SNP_62.

In one aspect the markers of c) are one or more of SNP_49 to SNP_61. In one aspect, at least 5, 6, 7, 8, 9, 10 or more markers are detected from the markers of a), b) and/or c) above. In one embodiment at least the marker of a) and/or b) is detected and optionally at least one, two, three or more markers of c) are detected. In one aspect the markers detected are consecutive markers.

Any wild cucumber genome-specific marker in between two markers refers to any molecular marker which maps genetically to the chromosome 1 region in-between the two markers and/or which lies physically in-between the two markers, and which is indicative of the wild cucumber chromosome 3 region. This means that the marker is polymorphic between the cultivated cucumber genome and the wild cucumber genome. In one aspect, the marker is a Single Nucleotide Polymorphism (SNP), but other molecular markers such as RFLP, AFLP, RAPD, DNA sequencing, etc. may equally be used.

The introgression fragment in the plants of the invention is in one aspect a fragment of chromosome 3 (comprising QTL3.1) which is present in seeds deposited under accession number NCIMB43745 or a smaller version of that fragment retaining the QTL (generated by e.g. recombination within the introgression fragment).

The introgression fragment is in one aspect equal to or less than 4 Mb in size, preferably equal to or less than 3.9 Mb, 3 Mb, 2.5 Mb, 2 Mb, 1.5 Mb, 1 Mb in size. In a further aspect the introgression fragment is at least 0.5 Mb or at least 1 Mb in size.

Also provided are seeds from which a plant of the invention can be grown, as are cucumber fruits harvested from a plant of the invention and comprising the recombinant chromosome 3 in their genome (comprising QTL3.1 or a variant). Likewise a plant cell, tissue or plant part of a plant or of a seed is provided comprising at least one recombinant chromosome 3 (comprising QTL3.1 or a variant), wherein said recombinant chromosome 3 comprises an introgression fragment from a wild or primitive cucumber and wherein said introgression fragment comprises an allele conferring significantly enhanced ToLCNDV-ES resistance.

The molecular markers described herein may be detected according to standard method. For example SNP markers can easily be detected using a KASP-assay (see www.kpbioscience.co.uk) or other SNP genotyping assays. For developing a KASP-assay, for example 50 or 70 base pairs upstream and 50 or 70 base pairs downstream of the SNP can be selected and two allele-specific forward primers and one allele specific reverse primer can be designed. See e.g. Allen et al. 2011, Plant Biotechnology J. 9, 1086-1099, especially p 097-1098 for KASP assay method.

Thus, in one aspect, the SNP markers and the presence/absence of the marker associated with QTL3.1 is determined using a KASP assay, but equally other SNP genotyping assays can be used. For example, a TaqMan SNP genotyping assay, a High Resolution Melting (HRM) assay, SNP-genotyping arrays (e.g. Fluidigm, Illumina, etc.) or DNA sequencing may equally be used.

The physical size of an introgression fragment can be determined by various methods, such as physical mapping, sequencing or by visualization of the introgression using Fluorescent in situ hybridization (FISH) images (Verlaan et al. 2011, Plant Journal 68: 1093-1103).

Cultivated cucumber plants with smaller introgression fragments on chromosome 3 (comprising QTL3.1 or a variant) can be generated by generating new recombinant plants from a population of plants derived from a cross between a cultivated cucumber plant (lacking the introgressions) and a plant of the invention and selecting recombinant progeny having smaller introgression sizes. Such plants are thus in one aspect derived from (progeny or descendants of) the recombinant chromosome 3 present in plants of which seeds have been deposited under NCIMB43745. Such progeny or descendants which retain the QTL3.1, and thus the higher ToLCNDV-ES resistance compared to plants lacking an introgression as described herein, are encompassed herein.

As mentioned, plants and plant parts may either comprise individual QTLs in homozygous or heterozygous form, selected from QTL1.1, QTL1.2, QTL2.1 and QTL3.1) or combinations of two or more QTLs, in homozygous or heterozygous form. In one aspect the combination of all four QTLs in homozygous form results in a ToLCNDV-ES resistance of 9.0, i.e. without any symptoms. Also, no ToLCNDV-ES virus could be detected in these plants, i.e. they appeared free of virus or the virus levels were below detection level of the DAS-ELISA test. To test the effect of individual QTLs or combinations of two or more QTLs on ToLCNDV-ES resistance, lines have to be generated which contain these QTLs (or combinations). This can for example be done by transferring the QTLs or combinations from e.g. the deposited seeds into separate backgrounds, e.g. using the linked SNP markers in selection of progeny. Thereafter the resistance can be tested in a ToLCNDV-ES disease assay as described herein. In one aspect the cultivated cucumber plant comprising one or more of the four QTLs comprises an average disease score (at least at one time point, preferably at two time points or three time point after inoculation) of at least 7.5, 7.6, 7.7, 7.8, 7.9, 8.0, 8.1, 8.2, 8.3, 8.4, 8.5, 8.6, 8.9, or 9.0 when tested in a ToLCNDV-ES assay as described herein. Obviously a ToLCNDV-ES assay always needs to include appropriate control plants, especially susceptible controls and/or genetic controls, and these control plants need to show the symptoms and average disease score as expected for them, in order to know that the inoculation worked.

In one aspect the plants and plant parts which comprise one or more QTLs selected from QTL1.1, QTL1.2, QTL2.1 and QTL3.1 (or variants thereof) comprise an introgression fragment comprising the QTL that is derivable from or obtainable from (or is derived from or obtained from) NCIMB 43745, PI605996, CGN22263, CGN22932 or PI197087 or from another wild donor which comprises the same SNP haplotype or SNP genotype for at least 5, 6, 7, 8, 9, 10, 11, 12, 13, 14, 15 or 16 of the SNP markers linked to each of the QTLs. Optionally the wild donor comprises an average ToLCNDV-ES disease score (at at least one of the time points, preferable at two or three of the time points measured after inoculation) of at least 7.5, 7.6, 7.7, 7.8, 7.9, 8.0, 8.1, 8.2, 8.3, 8.4, 8.5, 8.6, 8.7, 8.8, 8.9 or 9.0.

The cultivated cucumber plant according to the invention may be an inbred line, an OP (open pollinated variety) or an F1 hybrid. In one aspect the F1 hybrid comprises only one recombinant chromosome (comprising the introgression fragment with the QTL selected from QTL1.1, QTL1.2, QTL2.1 and/or QTL3.1), i.e. the F1 hybrid is heterozygous for the introgression fragment and the SNP marker described herein. Such an F1 hybrid is produced by crossing two inbred parent lines, one of which possesses the introgression fragments of the one or more QTLs (preferably in homozygous form, although not necessarily) and collecting the F1 hybrid seeds from said cross. In another aspect the F1 hybrid may comprise the introgression fragments selected from QTL1.1, QTL1.2, QTL2.1 and/or QTL3.1 in homozygous form, i.e. produced by crossing two inbred parent lines, each comprising the introgression fragments selected from one or more of the QTLs in homozygous form.

The cultivated cucumber plant may be of any type. Preferably it has good agronomic and good fruit quality characteristics. The cultivated cucumber plant is in one aspect uniform, both genetically and phenotypically. Especially fruit characteristics are uniform, e.g. regarding shape, skin color, skin thickness, skin ribs, skin toughness, spines (spine color, spine density, etc.), presence/absence of warts, length and diameter at edible and marketable maturity, flavour, etc. Likewise seed characteristics (i.e. characteristics of the seeds from which the plant is grown) are uniform, e.g. seed size, seed color, etc. Thus, plants of the line or variety comprising the one or more QTLs in homozygous or heterozygous form produce uniform fruits, meaning that there is little variation between fruits of plants grown under the same environmental conditions and when fruits are at the same developmental stage (e.g. for qualitative characteristics at least 98%, 99% or preferably 100% of all plants or plant parts, fruits or seed are identical for the characteristics; for quantitative characteristics at least 90%, 95%, 98% of all plants or plant parts, fruits or seed are identical for the characteristics).

The cultivated cucumber plant comprising the one or more QTLs (or variants thereof) according to the invention may be of any type, e.g. it may be of one of the following cucumber types: pickling cucumbers (e.g. American pickling, European pickling type), slicing cucumbers (e.g. American slicing), long cucumbers, short cucumbers, European greenhouse cucumbers, Beit-Alpha type cucumbers, oriental trellis type cucumbers, Asian cucumbers (e.g. selected from Indian Mottled cucumber, Chinese Long cucumber, Korean cucumber and Japanese cucumber type). In one aspect the cultivated cucumber according to the invention is an inbred line or a F1 hybrid of a pickling cucumber type, slicing cucumber type, long cucumber type, short cucumber type, European greenhouse cucumbers, Beit-Alpha type cucumbers, oriental trellis type cucumbers, Chinese long cucumber type, Korean cucumber type or Japanese cucumber type. In a specific embodiment the cucumber is an inbred line or an F1 hybrid of a European greenhouse cucumber or a slicer type cucumber.

The plant may be a single cross F1 hybrid or an inbred line, comprising the one or more QTLs in homozygous or heterozygous form. In one aspect it is an F1 hybrid produced by crossing an (inbred) parent plant comprising one or more of the QTLs (or variant) in homozygous form with an (inbred) parent plant lacking the QTLs (i.e. lacking introgression fragments comprising the QTLs). Thus in one aspect the F1 hybrid is heterozygous for QTL1.1, QTL1.2, QTL2.1 and/or QTL3.1.

In another aspect it is an F1 hybrid produced by crossing an (inbred) parent plant comprising one or more of the QTLs (or variants thereof) in homozygous form with an (inbred) parent plant that also comprises one or more of the QTLs (or variants thereof) in homozygous form. Thus, in one aspect the F1 hybrid is homozygous for QTL1.1, QTL1.2, QTL2.1 and/or QTL3.1.

In one aspect the F1 hybrid is a European greenhouse cucumber type, suitable for the traditional glasshouse cultivation or for high-wire cultivation. In the traditional glasshouse cultivation method the main stem of the plant is led up to a horizontal iron wire that is suspended at a height of about two meters above the ground.

When the plant reaches this height and attaches to the wire, it is “topped” by removing its growth point in order to terminate further proliferation, whereupon lateral shoots start to develop. These lateral shoots are allowed to grow downward to a height of about 1 meter above the ground, and the growth points are then removed from them. This is followed by flowering and the development of the fruits both on the stem and on the lateral shoots or tendrils, but the fruits on the tendrils develop later than those on the stem. The fruits are harvested about 6 weeks after sowing. In the high-wire cultivation no lateral tendrils are allowed to grow and all the harvest comes from the stem. Specific varieties have been developed by Nunhems which are highly suitable for high-wire cultivation, as they provide a gene called “compact”, see. WO2009/059777, for example varieties High-Jack, Hi-Power, Hi-Lisa.

Thus, in one aspect of the invention the cultivated cucumber plant comprises additionally the compact gene described in WO2009/059777. The compact gene is preferably present in heterozygous form.

In another aspect the one or more introgression fragments are present in a long cucumber type, such as variety Kasja (Nunhems), which is a long cucumber variety producing fruits of 27-38 cm. A “long cucumber type” or “long cucumber plants” are greenhouse cucumbers characterized by fruits of at least about 26 cm or 27 cm to 37 or 38 cm in length or longer (for example 40 cm, 42 cm or more), preferably with parthenocarpic fruit formation. Examples of long cucumber types are the Sabrina and Korinda varieties, or cucumber plants that are awarded a score of 7-9 for the length of the fruit according to the CPVO Protocol (see Point 19 in Annex 1 to this protocol). Other long cucumber varieties are, for example, Bodega, Bologna, Kamaro, Flamingo, Discover, Kalunga, Kasja, Logica, Millagon. Nicola, Milika, Manuela, Frida, Activa, Alaya, Savanna, Sienna, Bella, Sheila, Bornand.

In one aspect the cucumber is the plant of which seeds were deposited under accession numberNCIMB 43745, or progeny thereof, whereby the progeny retain one or more of QTL1.1, QTL1.2, QTL2.1 or QTL3.1 (as detectable by the presence of one or more markers as described elsewhere).

In another aspect the plant according to the invention is not a wild cucumber plant or a wild relative of cucumber or a landrace.

In yet another aspect the plant according to the invention is a cultivated cucumber of the Eurasian cucumber group, the East Asian cucumber group or the Xishuangbanna cucumber group. In another aspect the plant according to the invention is not a cucumber of the Indian cucumber group.

In one embodiment of the invention the cultivated cucumber plant comprising one or more of QTL1.1, QTL1.2, QTL2.1 and/or QTL3.1 (or a variant) produces seedless fruits without pollination, i.e. is parthenocarpic. Most European greenhouse cucumbers are parthenocarpic, i.e. the female flowers produce fruits without pollination, whereby the fruits remain seedless. Parthenocarpy is genetically controlled and it is known to breeders how to introduce the parthenocarpy trait into a cucumber line or variety (see e.g. Chapter 13 entitled “Cucumber” by T. tatlioglu, page 207-209 in the book Genetic Improvement of Vegetable Crops, Editors G. Kalloo and BO Bergh, Pergamon Press, 2012, ISBN0080408265).

In a further embodiment of the invention the cultivated cucumber plant comprising one or more of QTL1.1, QTL1.2, QTL2.1 and/or QTL3.1 (or a variant) is primarily gynoecious or entirely gynoecious (producing 100% female flowers). This means that mostly or only female flowers are produced. This trait is also genetically controlled and it is known to breeders how to introduce the gynoecious trait into a cucumber line or variety (see e.g. Chapter 13 entitled “Cucumber” by T. tatlioglu, page 207-209 in the book Genetic Improvement of Vegetable Crops, Editors G. Kalloo and BO Bergh, Pergamon Press, 2012, ISBN0080408265).

In one aspect the cucumber of the invention is both parthenocarpic and gynoecious. Thus, the plant produces primarily or only female flowers, which produce seedless fruits without pollination. In gynoecious cucumbers male flowers can be induced by treatment with silver nitrate. This method is used to produce pollen and to self-pollinate an inbred gynoecious cucumber line.

In a different aspect the cucumber plant of the invention is monoecious (produces both male and female flowers), optionally parthenocarpic and monoecious.

In a further embodiment of the invention the cultivated cucumber plant comprising one or more of QTL1.1, QTL1.2, QTL2.1 and/or QTL3.1 (or a variant) is uniform and genetically stable regarding the morphological characteristics of the fruits produced by said plant, e.g. regarding fruit shape, fruit color, skin thickness, warts, etc.

Fruit characteristics, such as average fruit length, average fruit diameter, skin thickness, presence/absence of warts, spininess, skin toughness, skin color, fruit neck shape, fruit tapering, shape of medial cross section, presence or absence of seeds (parthenocarpy), etc. depend on the cucumber type, i.e. the cultivated genetic background (gene pool) into which the QTL(s) is/are introgressed. Thus, depending on the cucumber type, various fruit shapes, sizes and fruit types are included herein. In one aspect the fruits are seedless.

The two main types of cucumber fruit grown commercially today in the United States are fresh market (slicing) type and the processing (pickling) type. Varieties and production methods are typically adapted to the end use. Slicing cucumbers are often longer, larger and have darker and thicker skin, whereas pickling/processing cucumbers have a shorter fruit, thinner skin with interior flesh that make them more amenable to pickling. Seedless varieties are generally preferable for both fresh market and for pickling as developing and large seeds are not palatable.

In one aspect the plant of the invention is a pickling type (processing type) and produces fruits which at edible maturity and/or marketable size have an average fruit length of at least 10 cm, or at least 11 cm, or at least 12 cm, or at least 13 cm and/or a fruit length to diameter ratio of at least 2, at least 2.5, at least 3, or more.

In a different aspect the plant of the invention is a fresh market type, e.g. a long cucumber type or slicing type, and produces fruits have an average fruit length at edible maturity and/or marketable size which is longer than the pickling type, e.g. at least 15 cm, 16 cm, 17 cm, 18 cm, 19 cm, 20 cm, 25 cm, 26 cm, 27 cm, 28 cm, 29 cm, 30 cm, 32 cm, 40 cm, or more.

In a preferred aspect the plant of the invention is a long cucumber type producing fruits of marketable size, especially seedless fruits. The fruits of marketable size, and parts thereof, and food or feed products containing these, are also encompassed herein. In one embodiment the SNP markers linked to one or more of QTL1.1, QTL1.2, QTL2.1 and/or QTL3.1 are detectable in the fruits, fruit parts or food or feed products comprising these.

In one aspect the plant is an indeterminate cucumber. In another aspect the cucumber is determinate.

Also seeds from which a plant according to the invention can be grown is provided herein, as are cucumber fruits harvested from a plant according to the invention. These comprise the one or more of QTL1.1, QTL1.2, QTL2.1 and/or QTL3.1 in their genome and can therefore be distinguished from other fruits by the presence of one or more of the SNP markers provided herein.

In one aspect the fruits are bitter free (selected from the groups bitter and bitterfree) at edible maturity and/or at marketable size of the fruits.

In a further aspect the fruit has a thin skin (selected from the groups thick and thin) at edible maturity and/or at marketable size of the fruits.

In a different embodiment the one or more of QTL1.1, QTL1.2, QTL2.1 and/or QTL3.1 are introgressed into a cucumber type called ‘Compact’, as described in U.S. Pat. No. 8,710,303B2. Thus, the cucumber plants according to the invention comprise the compact gene as described in U.S. Pat. No. 8,710,303B2 in homozygous or heterozygous form, e.g. as present in varieties Hi-Jack and Hi-Lisa (both Nunhems).

A further embodiment of the invention is a plant cell, tissue or plant part of a plant or of a seed according to the invention comprising at least one recombinant chromosome 1, 2 and/or 3, wherein said recombinant chromosome 1, 2, and/or 3 comprises an introgression fragment from a wild cucumber and wherein said introgression fragment comprises a QTL conferring enhanced ToLCNDV-ES resistance, wherein the QTLs are one or more of QTL1.1, QTL1.2, QTL2.1 and/or QTL3.1 (or variants thereof).

Also the use of a recombinant chromosome 1, 2 and/or 3 comprising an introgression fragment from a wild donor cucumber (said introgression fragment comprising an allele conferring enhanced ToLCNDV-ES resistance) for breeding cucumber varieties having enhanced ToLCNDV-ES resistance is encompassed herein. In one aspect said recombinant chromosomes 1, 2 and/or 3 is the recombinant chromosome 1, 2 and/or 3 as found in seeds deposited under accession number NCIMB 43745, or is derived from said recombinant chromosome 1, 2 and/or 3 (e.g. is a smaller fragment of the introgression fragment found in said seeds). In another embodiment the recombinant chromosomes comprise one or more of QTL1.1, QTL1.2, QTL2.1 and/or QTL3.1 from NCIMB 43745, PI605996, CGN22263, CGN22932 or PI197087 or from another wild donor which comprises the same SNP haplotype or SNP genotype for at least 5, 6, 7, 8, 9, 10, 11, 12, 13, 14, 15 or 16 of the SNP markers linked to each of the QTLs. Optionally the wild donor comprises an average ToLCNDV-ES disease score (at least one of the time points, preferable at two or three of the time points measured after inoculation) of at least 7.5, 7.6, 7.7, 7.8, 7.9, 8.0, 8.1, 8.2, 8.3, 8.4, 8.5, 8.6, 8.7, 8.8, 8.9 or 9.0.

Likewise, the use of a chromosome 1, 2 and/or 3, or the use of QTL1.1, QTL1.2, QTL2.1 and/or QTL3.1, as found in seeds deposited under accession number NCIMB 43745, or in progeny thereof, or as found in PI605996, CGN22263, CGN22932 or PI197087 or in another wild donor which comprises the same SNP haplotype or SNP genotype for at least 5, 6, 7, 8, 9, 10, 11, 12, 13, 14, 15 or 16 of the SNP markers linked to each of the QTLs, for generating a cultivated cucumber plant comprising an introgression fragment on chromosome 1, 2 and/or 3 is encompassed herein, wherein said introgression fragment confers enhanced ToLCNDV-ES resistance compared to the genetic control cucumber plant lacking said introgression fragment (e.g. such as plants grown from seeds deposited under NCIMB43744).

Similarly the use of plants grown from seeds deposited under accession number NCIMB 43745 or progeny thereof, for generating a cultivated cucumber plant comprising enhanced ToLCNDV-ES resistance is encompassed herein, wherein said enhanced ToLCNDV-ES resistance is conferred by an introgression fragment obtained from chromosome 1, 2 and/or 3 of said plants or progeny thereof.

Also provided is the use of plants grown from seeds deposited under accession number NCIMB43745 or progeny thereof, or the use of plants grown from seeds of PI605996, CGN22263, CGN22932 or PI197087 or from another wild donor which comprises the same SNP haplotype or SNP genotype for at least 5, 6, 7, 8, 9, 10, 11, 12, 13, 14, 15 or 16 of the SNP markers linked to each of the QTLs, for transferring one or more of QTLs QTL1.1, QTL1.2, QTL2.1 and/or QTL3.1 or the introgression fragment or a sub-fragment thereof comprising said QTL to another cucumber plant is provided.

Also a method for identifying (detecting or selecting) a cultivated C. sativus var. sativus plant or plant part comprising an introgression fragment on chromosome 1, 2 and/or 3 comprising QTL1.1, QTL1.2, QTL2.1 and/or QTL3.1 (or a variant of any of these) is provided, wherein said introgression fragment or chromosome region, or said QTL1.1, QTL1.2, QTL2.1 and/or QTL3.1 (or a variant of any of these), is e.g. as found in NCIMB 43745, or in PI605996, CGN22263, CGN22932 or PI197087 or in another wild donor, comprising:
a) providing a cultivated C. sativus var. sativus plant or plant part or DNA of such plant or plant part,
b) screening said plant, plant part or DNA using a molecular marker assay which detects at least 1, 2, 3, 4, 5, 6, 7, 8, 9, 10 or more SNP marker of:
   i) SNP_01 to SNP_16 for detecting the introgression fragment on chromosome 1 comprising QTL1.1 (or a variant); and/or of
   ii) SNP_17 to SNP_31 for detecting the introgression fragment on chromosome 1 comprising QTL1.2 (or a variant); and/or of
   iii) SNP_32 to SNP_47 for detecting the introgression fragment on chromosome 2 comprising QTL2.1 (or a variant); and/or of
   iv) SNP_48 to SNP_62 for detecting the introgression fragment on chromosome 3 comprising QTL3.1 (or a variant);
   and
c) identifying and/or selecting a plant comprising the wild donor SNP nucleotide (the donor SNP haplotype or donor SNP genotype) for at least 1, 2, 3, 4, 5, 6, 7, 8, 9, 10 or more of
   i) SNP_01 to SNP_16 for detecting the introgression fragment on chromosome 1 comprising QTL1.1 (or a variant); and/or of
   ii) SNP_17 to SNP_31 for detecting the introgression fragment on chromosome 1 comprising QTL1.2 (or a variant); and/or of
   iii) SNP_32 to SNP_47 for detecting the introgression fragment on chromosome 2 comprising QTL2.1 (or a variant); and/or of
   iv) SNP_48 to SNP_62 for detecting the introgression fragment on chromosome 3 comprising QTL3.1 (or a variant).

Further a method of producing C. sativus F1 hybrid plants comprising an introgression fragment on chromosome 1, 2 and/or 3 comprising QTL1.1, QTL1.2, QTL2.1 and/or QTL3.1 (or a variant of any of these) comprising:
a) providing a first inbred cucumber plant comprising a recombinant chromosome 1, 2 and/or 3 in homozygous form having an introgression fragment comprising QTL1.1, QTL1.2, QTL2.1 and/or QTL3.1 (or a variant of any of these) and comprising the wild donor SNP nucleotide (the donor SNP haplotype or donor SNP genotype) for at least 1, 2, 3, 4, 5, 6, 7, 8, 9, 10 or more of the SNP markers linked to the QTL, optionally wherein said introgression fragment is derivable from (or derived from) NCIMB 43745, P1605996, CGN22263, CGN22932 or P1197087,
b) providing a second inbred cucumber plant,
c) crossing said cucumber plant of a) with said cucumber plant of b),
d) collecting F1 hybrid seeds from said cross.

The F1 hybrid seeds collected are also an embodiment of the invention.

In another aspect a method for generating progeny of NCIMB 43745 is provided, said method comprising:
a) growing a plant from seeds deposited under accession number NCIMB 43745;
b) selfing said plant one or more times and/or crossing said plant one or more times with another cucumber plant to generate progeny seeds;
c) screening said progeny seeds or plants grown from said seeds or parts of the seeds or plants using a molecular marker assay which detects at least 1, 2, 3, 4, 5, 6, 7, 8, 9, 10 or more SNP markers of:
   i) SNP_01 to SNP_16 for detecting the introgression fragment on chromosome 1 comprising QTL1.1 (or a variant); and/or of
   ii) SNP_17 to SNP_31 for detecting the introgression fragment on chromosome 1 comprising QTL1.2 (or a variant); and/or of
   iii) SNP_32 to SNP_47 for detecting the introgression fragment on chromosome 2 comprising QTL2.1 (or a variant); and/or of
   iv) SNP_48 to SNP_62 for detecting the introgression fragment on chromosome 3 comprising QTL3.1 (or a variant).
d) identifying and/or selecting a progeny plant comprising the wild donor SNP nucleotide (the donor SNP haplotype or donor SNP genotype) for at least 1, 2, 3, 4, 5, 6, 7, 8, 9, 10 or more of
   i) SNP_01 to SNP_16 for detecting the introgression fragment on chromosome 1 comprising QTL1.1 (or a variant); and/or of
   ii) SNP_17 to SNP_31 for detecting the introgression fragment on chromosome 1 comprising QTL1.2 (or a variant); and/or of
   iii) SNP_32 to SNP_47 for detecting the introgression fragment on chromosome 2 comprising QTL2.1 (or a variant); and/or of
   iv) SNP_48 to SNP_62 for detecting the introgression fragment on chromosome 3 comprising QTL3.1 (or a variant).

The donor SNP nucleotide (or haplotype or genotype) is described above and in Tables 1-4.

Further provided is a method for identifying and/or selecting a wild cucumber donor plant comprising a QTL selected from QTL1.1, QTL1.2, QTL2.1 and QTL3.1, comprising:
a) screening seeds or parts of the seeds or plants or parts of plants or DNA of such plants or plant parts of one or more wild cucumber accessions using a molecular marker assay which detects at least 5, 6, 7, 8, 9, 10, 11, 12, 13, 14, 15 or 16 SNP markers of:
   i) SNP_01 to SNP_16 for detecting the introgression fragment on chromosome 1 comprising QTL1.1 (or a variant); and/or of
   ii) SNP_17 to SNP_31 for detecting the introgression fragment on chromosome 1 comprising QTL1.2 (or a variant); and/or of
   iii) SNP_32 to SNP_47 for detecting the introgression fragment on chromosome 2 comprising QTL2.1 (or a variant); and/or of
   iv) SNP_48 to SNP_62 for detecting the introgression fragment on chromosome 3 comprising QTL3.1 (or a variant).
b) identifying and/or selecting a wild cucumber accession comprising the donor SNP nucleotide (the donor SNP haplotype or donor SNP genotype) for at least 5, 6, 7, 8, 9, 10, 11, 12, 13, 14, 15 or 16 of
   i) SNP_01 to SNP_16 for detecting the introgression fragment on chromosome 1 comprising QTL1.1 (or a variant); and/or of
   ii) SNP_17 to SNP_31 for detecting the introgression fragment on chromosome 1 comprising QTL1.2 (or a variant); and/or of
   iii) SNP_32 to SNP_47 for detecting the introgression fragment on chromosome 2 comprising QTL2.1 (or a variant); and/or of
   iv) SNP_48 to SNP_62 for detecting the introgression fragment on chromosome 3 comprising QTL3.1 (or a variant).

The above method may optionally also comprise selfing the wild accessions one or more times, e.g. prior to step a).

Wild accessions may be accessions of wild or primitive cucumber or relatives of cucumber obtained e.g. from seed depositories, such as USDA ARS-GRIN collections, CGN collections, and others. Examples are the CGN and PI accessions mentioned herein.

The method above may also include a step of testing the one or more wild accessions in a ToLCNDV-ES resistance assay. This may e.g. be done prior to the molecular marker assay of step a), prior to selfing the accession(s) or after selfing the accession(s) and/or after step b), i.e. after selection or identification of one or more accessions comprising a SNP haplotype or SNP genotype identical or similar to the one described in Tables 1 to 4, e.g. the SNP haplotype for a QTL having the donor nucleotide for at least 5, 6, 7, 8, 9, 10, 11, 12, 13, 14, 15 or 16 of the SNP markers linked to the QTL. The one or more wild accessions may be selected for accessions (or selfing progenies thereof) having an average ToLCNDV-ES disease score of at least 7.5, 7.6, 7.7, 7.8, 7.9, 8.0, 81, 8.2, 8.3, 8.4, 8.5, 8.6, 8.7, 8.8, 8.9 or 9.0.

Also a method for transferring a QTL selected from QTL1.1, QTL1.2, QTL2.1 and QTL3.1 from a wild donor into a cultivated cucumber plant is provided comprising:
a) providing a wild cucumber accession (or a progeny thereof obtained by selfing one or more times) comprising the donor SNP nucleotide (the donor SNP haplotype or donor SNP genotype) for at least 5, 6, 7, 8, 9, 10, 11, 12, 13, 14, 15 or 16 of
   i) SNP_01 to SNP_16 for detecting the introgression fragment on chromosome 1 comprising QTL1.1 (or a variant); and/or of
   ii) SNP_17 to SNP_31 for detecting the introgression fragment on chromosome 1 comprising QTL1.2 (or a variant); and/or of
   iii) SNP_32 to SNP_47 for detecting the introgression fragment on chromosome 2 comprising QTL2.1 (or a variant); and/or of
   iv) SNP_48 to SNP_62 for detecting the introgression fragment on chromosome 3 comprising QTL3.1 (or a variant),
   and optionally comprising an average ToLCNDV-ES disease score of at least 7.5, preferably at least 8.0, 8.5, 8.6, 8.7, 8.8, 8.9 or 9.0,
b) crossing said accession with a cultivated cucumber plant to obtain progenies of the F1, F2, F3 or further selfing generations or BC1, BC2, BC3 or further backcross generations, and optionally
c) selecting a progeny plant comprising the one or more of the QTLs.

The cucumber plant in step b is preferably a cultivated cucumber, such as a European greenhouse cucumber or long cucumber type or a slicer.

The wild accession in step a) is in one aspect selected from PI605996 (or progeny thereof), CGN22263 (or progeny thereof) or CGN22932 (or progeny thereof), or P1197087 (or progeny thereof).

A progeny plant generated by the above method is also an aspect of the invention.

Also containers and packages containing or comprising seeds from which plants of the invention can be grown are provided herein. These may be labelled as containing cultivated cucumber seeds producing plants having ToLCNDV-ES resistance.

Also progeny seeds and progeny plants of plants of the invention are provided, which retain the introgression on chromosome 1, 2 and/or 3 comprising one or more QTLs selected from QTL1.1, QTL1.2, QTL2.1 and QTL3.1 (or a variant of any of these), or which comprise a smaller introgression of any of the QTLs (e.g. derivable from the fragment as is present in NCIMB 43745) which still confers enhanced ToLCNDV-ES resistance, i.e. which still contains one or more of the QTLs. Progeny may be any generation obtained by selfing a cucumber plant according to the invention and/or crossing a cucumber plant according to the invention with another cucumber plant one or more times. Progeny are, therefore, either the generation (seeds) produced from the first cross (F1) or selfing (S1), or any further generation produced by crossing and/or selfing (F2, F3, etc.) and/or backcrossing (BC1, BC2, etc.) one or more selected plants of the F1 and/or S1 and/or BC1 generation (or plants of any further generation, e.g. the F2) with another cucumber plant (and/or with a wild cucumber). Progeny are preferably selected to retain introgression fragments from a wild cucumber comprising a QTL selected from QTL1.1, QTL1.2, QTL2.1 and/or QTL3.1. Thus progeny also have the increased ToLCNDV-ES resistance phenotype, preferably at least the same average disease score as the plant used in the initial cross or selfing. The presence of (or retention of) the (one or more) introgression fragments comprising the QTLs can be determined phenotypically and/or using the molecular marker assay(s) described herein. Regarding phenotypic assessment, of course consideration needs to be given to the dominance nature of the QTL. QTL2.1 is recessive or partially recessive, while the other three QTLs are additive.

In a further aspect parts of the cucumber plants according to the invention are provided. Parts include for example cells and cell-cultures, tissue cultures, vegetative plant tissues (leaves, roots, etc.), flowers, pollen, embryos, fruits, parts of fruits, etc. The plant parts comprise the one or more introgression fragments on chromosome 1, 2 and/or 3, as described, and as can be detected using one or more of the markers described. Also, when whole plants are regenerated from such cucumber parts, such as cells, cell- or tissue cultures, the regenerated plants comprise the recombinant chromosome 1, 2 and/or 3.

Thus, also provided is a plant cell, tissue or plant part of a plant or of a seed according the invention comprising at least one recombinant chromosome 1, 2 and/or 3, wherein said recombinant chromosome 1, 2 and/or 3 comprises an introgression fragment from a wild donor cucumber plant and wherein said introgression fragment comprises a QTL conferring enhanced ToLCNDV-ES resistance, selected from QTL1.1, QTL1.2, QTL2.1 and QTL3.1 (or variants of any of these).

Also in vitro cell cultures and in vitro tissue cultures are encompassed herein, of cells or tissues comprising a recombinant chromosome 1, 2 and/or 3 described. Preferably the cells or tissues can be regenerated into a whole cucumber plant, i.e. the cells are regenerable cells and the tissues comprise regenerable cells. Thus, also vegetative propagations of the plants according to the invention are an embodiment herein. Thus, a vegetatively propagated cultivated cucumber plant is provided which comprises a recombinant chromosome 1, 2 and/or 3 as described herein. In a different aspect non-propagating cells comprising a QTL conferring enhanced ToLCNDV-ES resistance, selected from QTL1.1, QTL1.2, QTL2.1 and QTL3.1 (or variants of any of these), are encompassed herein, as are tissues comprising such cells.

In a specific aspect a cucumber fruit harvested from a plant according to the invention is provided. Marketable cucumber fruits, especially for the fresh market (slicing), are generally graded according to fruit size and quality characteristics after harvest. See e.g. the United States Standards for Grades of Cucumbers, US Department of Agriculture, Effective Mar. 1, 1985 and reprinted January 1997. Herein different grades of cucumbers are distinguished. Thus, in one aspect harvested fruits are provided of U.S. Fancy grade, U.S. Extra No. 1 grade, U.S. No. 1 grade, U.S. No. 1 Small grade, U.S. No. 1 Large grade, U.S. No. 2 grade. Also containers or packages comprising or consisting of harvested cucumber fruits are provided. Again, the cells of the fruits are distinguishable from other cucumber fruits by the presence of the QTL1.1, QTL1.2, QTL2.1 and/or QTL3.1 (or variants of any of these) (as determinable in one or more of the molecular marker assays).

In another aspect the cucumber is a long cucumber type or a slicer cucumber type and fruits harvested and optionally processed (e.g. sliced or diced) are provided.

In another aspect the cucumber is a pickling type and fruits harvested and optionally pickled are provided.

The invention also provides for a food or feed product comprising or consisting of a plant part described herein preferably a cucumber fruit or part thereof and/or an extract from a plant part described herein. The food or feed product may be fresh or processed, e.g., pickled, canned, steamed, boiled, fried, blanched and/or frozen, etc. For example, containers such as cans, boxes, crates, bags, cartons, Modified Atmosphere Packaging, films (e.g. biodegradable films), etc. comprising plant parts such as fruits or fruit parts (fresh and/or processed) described herein are also provided herein.

### Methods and Uses According to the Invention

In a further embodiment, the invention provides for a method of producing a new cultivated cucumber plant which comprises one or more introgression fragments on chromosome 1, 2 and/or 3, which comprises QTL1.1, QTL1.2, QTL2.1 and/or QTL3.1 or variants of any of these and wherein said one or more QTLs confer enhanced ToLCNDV-ES resistance compared to a control plant, in homozygous or heterozygous form, as described. The method comprises crossing a plant of the invention, or a progeny plant thereof, either as male or as female parent, with a second cucumber plant (or a wild donor cucumber) one or more times, and/or selfing a cucumber plant according to the invention, or a progeny plant thereof, one or more times, and selecting progeny from said crossing and/or selfing.

Thus, a method for transferring the recombinant chromosome 1, 2 and/or 3, comprising QTL1.1, QTL1.2, QTL2.1 and/or QTL3.1 or variants of any of these, from one (cultivated) cucumber plant into another (cultivated) cucumber plant is provided, especially into cucumber varieties or breeding lines for which the ToLCNDV-ES resistance should be increased.

The method comprises the steps of:
a) providing a first cultivate cucumber plant comprising an introgression fragment comprising QTL1.1, QTL1.2, QTL2.1 and/or QTL3.1 (or a variant of any of these), preferably in homozygous form,
b) providing a second cultivated cucumber plant, especially a plant having a wild type (non-recombinant) chromosome 1, 2 and/or 3, or a plant lacking QTL1.1, QTL1.2, QTL2.1 and/or QTL3.1,
c) crossing said cucumber plant of a) with said cucumber plant of b),
d) collecting F1 hybrid seeds from said cross, and
e) optionally selfing the plant grown from said F1 hybrid seeds to produce F2 seeds or further selfing generations and/or backcrossing the seeds to produce backcross generations, and optionally selecting the F2 seeds or further selfing generation seeds or backcross generation seeds having the QTL1.1, QTL1.2, QTL2.1 and/or QTL3.1 (or a variant of any of these), and
f) optionally breeding further with plants grown from said F1 or F2 or further generation selfing seeds or backcross generation seeds to produce a cucumber plant having good agronomic characteristics and QTL1.1, QTL1.2, QTL2.1 and/or QTL3.1 (or a variant of any of these) in homozygous or heterozygous form.

The presence or absence of the introgression fragment comprising QTL1.1, QTL1.2, QTL2.1 and/or QTL3.1 (or a variant of any of these) may be determined by one or more of the molecular marker assays described herein and/or by determining whether the ToLCNDV-ES resistance is significantly increased compared to the plant of e.g. step b). Further breeding in step f) may comprise selfing, crossing, double haploid production, backcrossing, and combinations thereof (e.g. backcrossing and selfing), etc. Plants, plant parts and seeds obtainable by the above method are encompassed herein.

In one aspect the plant of step a) may be a plant grown from seeds deposited under NCIMB 43745, or progeny thereof, or a plant comprising QTL1.1, QTL1.2, QTL2.1 and/or QTL3.1 (or a variant of any of these) as present in seeds deposited under NCIMB 43745, PI605996, CGN22263, CGN22932, PI197087 or another wild donor accession comprising the same SNP haplotype or genotype for at least 5, 6, 7, 8, 9, 10, 11, 12, 13, 14, 15 or 16 markers linked to the QTL (e.g. as described in Tables 1 to 4).

Also provided is a method of producing cultivated cucumber F1 hybrid plants comprising a ToLCNDV-ES resistance QTL (selected from QTL1.1, QTL1.2, QTL2.1 and/or QTL3.1 or a variant of any of these) comprising:
a) providing a first inbred cucumber plant comprising at least one recombinant chromosome comprising an introgression fragment comprising a ToLCNDV-ES resistance QTL selected from selected from QTL1.1, QTL1.2, QTL2.1 and/or QTL3.1 or a variant of any of these,
b) providing a second inbred cucumber plant comprising at least one recombinant chromosome comprising an introgression fragment comprising a ToLCNDV-ES resistance QTL selected from selected from QTL1.1, QTL1.2, QTL2.1 and/or QTL3.1 or a variant of any of these,
c) crossing said cucumber plant of a) with said cucumber plant of b),
d) collecting F1 hybrid seeds from said cross.

The inbred cucumber plant of a) and b) is preferably homozygous for the introgression fragment(s), and they may contain introgression fragments of different sizes and/or of different origin, i.e. from different wild donors and/or for different QTLs (e.g. the plant in a) may be homozygous for QTL1.1 and QTL1.2 and the plant in b) may be homozygous for QTL3.1). So, for example the introgression fragment in a) may be the same or a different introgression fragment than in b). In one aspect the inbred cucumber plant of a) comprises at least two of the QTLs in homozygous form (e.g. QTL1.1 and QTL1.2, or QTL1.1 and/or QTL1.2 and QTL3.1) and/or the inbred cucumber plant of b) comprises the same QTLs in homozygous form. In one aspect the QTL selected from QTL1.1, QTL1.2, QTL2.1 and/or QTL3.1 is the QTL as found in NCIMB43745, PI605996, CGN22263, CGN22932, PI197087, or another wild donor comprising at least 5, 6, 7, 8, 9, 10 or more of the SNP markers linked to the QTL (see e.g. Tables 1-4) and preferably comprising an average disease score of at least 7.5, 7.6, 7.7, 7.8, 7.9, 8.0, 8.1, 8.2, 8.3, 8.4, 8.5, 8.6, 8.7, 8.8, 8.9 or 9.0.

The F1 hybrid seeds preferably comprise at least one recombinant chromosome comprising QTL1.1, QTL 1.2, QTL2.1 and/or QTL3.1 (or a variant of any of these) and the F1 plants grown from the seeds do therefore produce enhanced ToLCNDV-ES resistance compared to the control. Regarding QTL2.1 or a variant, this QTL is preferably in homozygous form, while the others may be in heterozygous or homozygous form.

Plants and seeds obtainable by the above method are encompassed herein.

In a different aspect a method for producing a cultivated cucumber plant comprising an introgression fragment on chromosome 1, 2 and/or 3, wherein said introgression fragment comprises a QTL selected from QTL1.1, QTL1.2, QTL2.1 and/or QTL3.1 or a variant of any of these, is provided, said method comprising the steps:
a) providing a first cultivated cucumber plant,
b) providing a second, wild cucumber, wherein said plant comprises QTL1.1, QTL1.2, QTL2.1 and/or QTL3.1, or a variant of any of these, as determinable by the presence of one or more SNP markers (especially as determinable by the presence of the donor SNP haplotype or genotype for at least 5, 6, 7, 8, 9, 10, 11, 12, 13, 14, 15 or 16 SNP markers linked to the QTL) as described herein,
c) crossing said cucumber plant of a) with said cucumber plant of b),
d) collecting F1 seeds from said cross and backcrossing an F1 plant to the cucumber plant of a) to produce a backcross (BC1) population, or selfing said F1 plants one or more times to produce an F2 or F3 or higher generation selfing population,
e) optionally backcrossing a plant of d) one or more times to the cucumber plant of a) to produce a higher generation backcross population, and
f) identifying a F2, F3, or higher generation selfing, or BC1 or higher generation backcross plant which comprises an introgression on chromosome 1, 2 and/or 3, wherein said introgression fragment comprises QTL1.1, QTL1.2, QTL2.1 and/or QTL3.1, or a variant of any of these.

When referring to backcross populations in the method, the backcross populations may also be selfed, i.e. BC1S1, BC1S2, BC2S1, BC2S2, or others.

In one or more of steps b) to f) the presence of the QTL (or the introgression fragment comprising the QTL) may be tested (and plants may be selected) by carrying out a molecular marker assay as described elsewhere herein, e.g. by determining whether the plant comprises the one or more of the SNP markers (e.g. one or more of SNP_01 to SNP_16 linked to QTL1.1, one or more of SNP_17 to SNP_31 linked to QTL1.2, one or more of SNP_32 to 47 linked to QTL2.1 or one or more of SNP_48 to SNP_62 linked to QTL3.1).

Using this method, one can generate and/or select new cultivated cucumber plants comprising an introgression with QTL1.1, QTL1.2, QTL2.1 and/or QTL3.1, or a variant of any of these, from a wild source, such as a wild or primitive cucumber. In one aspect the wild cucumber in step b) is selected from P1605996, CGN22263, CGN22932, PI197087, or another wild donor comprising at least 5, 6, 7, 8, 9, 10 or more of the SNP markers (SNP haplotype or SNP genotype) linked to the QTL (see e.g. Tables 1-4) and preferably comprising an average ToLCNDV-ES disease score of at least 7.5, 7.6, 7.7, 7.8, 7.9. 8.0, 8.1, 8.2, 8.3, 8.4, 8.5, 8.6, 8.7, 8.8, 8.9 or 9.0.

In one aspect the method for producing a cultivated cucumber plant comprising an introgression fragment on chromosome 1, 2 and/or 3, wherein said introgression fragment comprises QTL1.1, QTL1.2, QTL2.1 and/or QTL3.1, or a variant of any of these, comprises the steps:
a) providing a first cultivated cucumber plant,
b) providing a second wild cucumber comprising at least 5, 6, 7, 8, 9, 10, 11, 12, 13, 14, 15 or more of the SNP markers (SNP haplotype or SNP genotype) linked to at least one QTL selected from QTL1.1, QTL1.2, QTL2.1 and/or QTL3.1, and optionally comprising an average ToLCNDV-ES disease score of at least 7.5, 7.6, 7.7, 7.8, 7.9. 8.0, 8.1, 8.2, 8.3, 8.4, 8.5, 8.6, 8.7, 8.8, 8.9 or 9.0,
c) crossing said plant of a) with said plant of b),
d) collecting F1 seeds from said cross and backcrossing an F1 plant to the cucumber plant of a) to produce a backcross (BC1) population, or selfing said F1 plants one or more times to produce an F2 or F3 population,
e) optionally selfing the backcross population to produce e.g. a BC1S1 or BC1S2 population,
f) identifying a F2, F3, BC1, BC1S1, or BC1S2 plant which comprises the (one or more) SNP markers and/or any wild-relative of cucumber genome-specific marker in between the SNP markers.

Also provided is a method for identifying a wild cucumber comprising a ToLCNDV-ES resistance QTL on chromosome 1, 2 and/or 3, said method comprising:
A) providing a wild or primitive cucumber accession or several accessions;
B) screening said accession(s) using a molecular marker assay which detects at least 1, 2, 3, 4, 5, 6, 7, 8, 9, 10, 11, 12, 13, 14, 15 or more of the SNP markers linked to at least one QTL selected from QTL1.1, QTL1.2, QTL2.1 and/or QTL3.1;
C) identifying and/or selecting an accession from b) comprising the SNP haplotype or SNP genotype of at least 5, 6, 7, 8, 9, 10, 11, 12, 13, 14, 15 or 16 of the SNP markers linked to a QTL, selected from:

For QTL1.1:
a) the TX or TT genotype for the Single Nucleotide Polymorphism marker SNP_01 at nucleotide 51 of SEQ ID NO: 1 (or at nucleotide 51 in a variant thereof);
b) the TX or TT genotype for the Single Nucleotide Polymorphism marker SNP_02 at nucleotide 51 of SEQ ID NO: 2 (or at nucleotide 51 in a variant thereof);
c) the TX or TT genotype for the Single Nucleotide Polymorphism marker SNP_03 at nucleotide 51 of SEQ ID NO: 3 (or at nucleotide 51 in a variant thereof);
d) the TX or TT genotype for the Single Nucleotide Polymorphism marker SNP_04 at nucleotide 51 of SEQ ID NO: 4 (or at nucleotide 51 in a variant thereof);
e) the AX or AA genotype for the Single Nucleotide Polymorphism marker SNP_05 at nucleotide 51 of SEQ ID NO: 5 (or at nucleotide 51 in a variant thereof);
f) the CX or CC genotype for the Single Nucleotide Polymorphism marker SNP_06 at nucleotide 51 of SEQ ID NO: 6 (or at nucleotide 51 in a variant thereof);
g) the TX or TT genotype for the Single Nucleotide Polymorphism marker SNP_07 at nucleotide 51 of SEQ ID NO: 7 (or at nucleotide 51 in a variant thereof);
h) the AX or AA genotype for the Single Nucleotide Polymorphism marker SNP_08 at nucleotide 51 of SEQ ID NO: 8 (or at nucleotide 51 in a variant thereof);
i) the GX or GG genotype for the Single Nucleotide Polymorphism marker SNP_09 at nucleotide 51 of SEQ ID NO: 9 (or at nucleotide 51 in a variant thereof); or the AX or AA genotype for the Single Nucleotide Polymorphism marker SNP_09 at nucleotide 51 of SEQ ID NO: 9 (or at nucleotide 51 in a variant thereof);
j) the CX or CC genotype for the Single Nucleotide Polymorphism marker SNP_10 at nucleotide 51 of SEQ ID NO: 10 (or at nucleotide 51 in a variant thereof);
k) the CX or CC genotype for the Single Nucleotide Polymorphism marker SNP_11 at nucleotide 51 of SEQ ID NO: 11 (or at nucleotide 51 in a variant thereof);
l) the CX or CC genotype for the Single Nucleotide Polymorphism marker SNP_12 at nucleotide 51 of SEQ ID NO: 12 (or at nucleotide 51 in a variant thereof);
m) the TX or TT genotype for the Single Nucleotide Polymorphism marker SNP_13 at nucleotide 51 of SEQ ID NO: 13 (or at nucleotide 51 in a variant thereof);
n) the AX or AA genotype for the Single Nucleotide Polymorphism marker SNP_14 at nucleotide 51 of SEQ ID NO: 14 (or at nucleotide 51 in a variant thereof);
o) the CX or CC genotype for the Single Nucleotide Polymorphism marker SNP_15 at nucleotide 51 of SEQ ID NO: 15 (or at nucleotide 51 in a variant thereof); or the TX or TT genotype for the Single Nucleotide Polymorphism marker SNP_15 at nucleotide 51 of SEQ ID NO: 15 (or at nucleotide 51 in a variant thereof);
p) the GX or GG genotype for the Single Nucleotide Polymorphism marker SNP_16 at nucleotide 51 of SEQ ID NO: 16 (or at nucleotide 51 in a variant thereof).

For QTL1.2:
a) the CX or CC genotype for the Single Nucleotide Polymorphism marker SNP_17 at nucleotide 51 of SEQ ID NO: 17 (or at nucleotide 51 in a variant thereof);
b) the GX or GG genotype for the Single Nucleotide Polymorphism marker SNP_18 at nucleotide 51 of SEQ ID NO: 18 (or at nucleotide 51 in a variant thereof);
c) the GX or GG genotype for the Single Nucleotide Polymorphism marker SNP_19 at nucleotide 51 of SEQ ID NO: 19 (or at nucleotide 51 in a variant thereof);
d) the TX or TT genotype for the Single Nucleotide Polymorphism marker SNP_20 at nucleotide 51 of SEQ ID NO: 20 (or at nucleotide 51 in a variant thereof);
e) the GX or GG genotype for the Single Nucleotide Polymorphism marker SNP_21 at nucleotide 51 of SEQ ID NO: 21 (or at nucleotide 51 in a variant thereof);
f) the GX or GG genotype for the Single Nucleotide Polymorphism marker SNP_22 at nucleotide 51 of SEQ ID NO: 22 (or at nucleotide 51 in a variant thereof);
g) the TX or TT genotype for the Single Nucleotide Polymorphism marker SNP_23 at nucleotide 51 of SEQ ID NO: 23 (or at nucleotide 51 in a variant thereof);
h) the CX or CC genotype for the Single Nucleotide Polymorphism marker SNP_24 at nucleotide 51 of SEQ ID NO: 24 (or at nucleotide 51 in a variant thereof);
i) the AX or AA genotype for the Single Nucleotide Polymorphism marker SNP_25 at nucleotide 51 of SEQ ID NO: 25 (or at nucleotide 51 in a variant thereof);
j) the CX or CC genotype for the Single Nucleotide Polymorphism marker SNP_26 at nucleotide 51 of SEQ ID NO: 26 (or at nucleotide 51 in a variant thereof);
k) the CX or CC genotype for the Single Nucleotide Polymorphism marker SNP_27 at nucleotide 51 of SEQ ID NO: 27 (or at nucleotide 51 in a variant thereof);
l) the CX or CC genotype for the Single Nucleotide Polymorphism marker SNP_28 at nucleotide 51 of SEQ ID NO: 28 (or at nucleotide 51 in a variant thereof);
m) the CX or CC genotype for the Single Nucleotide Polymorphism marker SNP_29 at nucleotide 51 of SEQ ID NO: 29 (or at nucleotide 51 in a variant thereof);
n) the AX or AA genotype for the Single Nucleotide Polymorphism marker SNP_30 at nucleotide 51 of SEQ ID NO: 30 (or at nucleotide 51 in a variant thereof);
o) the GX or GG genotype for the Single Nucleotide Polymorphism marker SNP_31 at nucleotide 51 of SEQ ID NO: 31 (or at nucleotide 51 in a variant thereof).

For QTL2.1:
a) the TX or TT genotype for the Single Nucleotide Polymorphism marker SNP_32 at nucleotide 51 of SEQ ID NO: 32 (or at nucleotide 51 in a variant thereof);
b) the TX or TT genotype for the Single Nucleotide Polymorphism marker SNP_33 at nucleotide 51 of SEQ ID NO: 33 (or at nucleotide 51 in a variant thereof);
c) the CX or CC genotype for the Single Nucleotide Polymorphism marker SNP_34 at nucleotide 51 of SEQ ID NO: 34 (or at nucleotide 51 in a variant thereof);
d) the AX or AA genotype for the Single Nucleotide Polymorphism marker SNP_35 at nucleotide 51 of SEQ ID NO: 35 (or at nucleotide 51 in a variant thereof);
e) the TX or TT genotype for the Single Nucleotide Polymorphism marker SNP_36 at nucleotide 51 of SEQ ID NO: 36 (or at nucleotide 51 in a variant thereof);
f) the CX or CC genotype for the Single Nucleotide Polymorphism marker SNP_37 at nucleotide 51 of SEQ ID NO: 37 (or at nucleotide 51 in a variant thereof);
g) the GX or GG genotype for the Single Nucleotide Polymorphism marker SNP_38 at nucleotide 51 of SEQ ID NO: 38 (or at nucleotide 51 in a variant thereof);
h) the CX or CC genotype for the Single Nucleotide Polymorphism marker SNP_39 at nucleotide 51 of SEQ ID NO: 39 (or at nucleotide 51 in a variant thereof);
i) the GX or GG genotype for the Single Nucleotide Polymorphism marker SNP_40 at nucleotide 51 of SEQ ID NO: 40 (or at nucleotide 51 in a variant thereof);
j) the TX or TT genotype for the Single Nucleotide Polymorphism marker SNP_41 at nucleotide 51 of SEQ ID NO: 41 (or at nucleotide 51 in a variant thereof);
k) the AX or AA genotype for the Single Nucleotide Polymorphism marker SNP_42 at nucleotide 51 of SEQ ID NO: 42 (or at nucleotide 51 in a variant thereof);
l) the TX or TT genotype for the Single Nucleotide Polymorphism marker SNP_43 at nucleotide 51 of SEQ ID NO: 43 (or at nucleotide 51 in a variant thereof);
m) the TX or TT genotype for the Single Nucleotide Polymorphism marker SNP_44 at nucleotide 51 of SEQ ID NO: 44 (or at nucleotide 51 in a variant thereof);
n) the GX or GG genotype for the Single Nucleotide Polymorphism marker SNP_45 at nucleotide 51 of SEQ ID NO: 45 (or at nucleotide 51 in a variant thereof);
o) the AX or AA genotype for the Single Nucleotide Polymorphism marker SNP_46 at nucleotide 51 of SEQ ID NO: 46 (or at nucleotide 51 in a variant thereof);
p) the GX or GG genotype for the Single Nucleotide Polymorphism marker SNP_47 at nucleotide 51 of SEQ ID NO: 47 (or at nucleotide 51 in a variant thereof).

For QTL3.1:
a) the AX or AA genotype for the Single Nucleotide Polymorphism marker SNP_48 at nucleotide 51 of SEQ ID NO: 48 (or at nucleotide 51 in a variant thereof);
b) the CX or CC genotype for the Single Nucleotide Polymorphism marker SNP_49 at nucleotide 51 of SEQ ID NO: 49 (or at nucleotide 51 in a variant thereof);
c) the CX or CC genotype for the Single Nucleotide Polymorphism marker SNP_50 at nucleotide 51 of SEQ ID NO: 50 (or at nucleotide 51 in a variant thereof);
d) the CX or CC genotype for the Single Nucleotide Polymorphism marker SNP_51 at nucleotide 51 of SEQ ID NO: 51 (or at nucleotide 51 in a variant thereof);
e) the TX or TT genotype for the Single Nucleotide Polymorphism marker SNP_52 at nucleotide 51 of SEQ ID NO: 52 (or at nucleotide 51 in a variant thereof);
f) the CX or CC genotype for the Single Nucleotide Polymorphism marker SNP_53 at nucleotide 51 of SEQ ID NO: 53 (or at nucleotide 51 in a variant thereof);
g) the CX or CC genotype for the Single Nucleotide Polymorphism marker SNP_54 at nucleotide 51 of SEQ ID NO: 54 (or at nucleotide 51 in a variant thereof);
h) the CX or CC genotype for the Single Nucleotide Polymorphism marker SNP_55 at nucleotide 51 of SEQ ID NO: 55 (or at nucleotide 51 in a variant thereof);
i) the GX or GG genotype for the Single Nucleotide Polymorphism marker SNP_56 at nucleotide 51 of SEQ ID NO: 56 (or at nucleotide 51 in a variant thereof);
j) the CX or CC genotype for the Single Nucleotide Polymorphism marker SNP_57 at nucleotide 51 of SEQ ID NO: 57 (or at nucleotide 51 in a variant thereof);
k) the TX or TT genotype for the Single Nucleotide Polymorphism marker SNP_58 at nucleotide 51 of SEQ ID NO: 58 (or at nucleotide 51 in a variant thereof);
l) the TX or TT genotype for the Single Nucleotide Polymorphism marker SNP_59 at nucleotide 51 of SEQ ID NO: 59 (or at nucleotide 51 in a variant thereof);
m) the GX or GG genotype for the Single Nucleotide Polymorphism marker SNP_60 at nucleotide 51 of SEQ ID NO: 60 (or at nucleotide 51 in a variant thereof);
n) the AX or AA genotype for the Single Nucleotide Polymorphism marker SNP_61 at nucleotide 51 of SEQ ID NO: 61 (or at nucleotide 51 in a variant thereof);
o) the AX or AA genotype for the Single Nucleotide Polymorphism marker SNP_62 at nucleotide 51 of SEQ ID NO: 62 (or at nucleotide 51 in a variant thereof).
D) optionally introgressing said QTL from said wild accession into cultivated cucumber (e.g. by backcrossing).

In step B), C) and D) a SNP genotyping assay can for example be used (such as a KASP assay), but also other molecular marker assays can be used. This applies also to other methods described herein. With this method one can, thus, screen wild cucumber accessions for the presence of one or more of the markers linked to one or more of the QTLs and introgress the QTL into cultivated cucumber plants. Plants and seeds obtained by this method are also an embodiment of the invention.

In still another aspect a method for identifying a cultivated cucumber plant comprising an introgression fragment on chromosome 1, 2 and/or 3, wherein said introgression fragment comprises a ToLCNDV-ES resistance QTL, is provided, said method comprising: screening a cultivated cucumber plant or a population of cultivated cucumber plants or parts of such cucumber plants (e.g. fruits, cells, DNA) using a molecular marker assay which detects at least one SNP marker (preferably 2, 3, 4, 5 or more; preferably consecutive SNP markers) indicative of (linked to) QTL1.1, QTL1.2, QTL2.1, QTL3.1 (or variants thereof) as described elsewhere herein.

In this method any of the molecular marker tests described elsewhere herein can be used. Thus, using this method one can detect the presence of an introgression fragment comprising QTL1.1, QTL1.2, QTL2.1, QTL3.1 (or variants thereof) in cultivated cucumber plants or plant parts.

In yet another aspect a method for detecting whether a cultivated cucumber plant comprises an introgression fragment on chromosome 1, 2 and/or 3, wherein said introgression fragment comprises QTL1.1, QTL1.2, QTL2.1, QTL3.1 (or variants thereof), is provided, said method comprising:
a) providing cultivated cucumber plant or a plant part,
b) screening said plant or said plant part (or DNA obtained from said plant or plant part) using a molecular marker assay which detects at least one (preferably at least 2, 3, 4, 5, 6, 7, 8, 9, 10 or more) SNP marker selected from the group consisting of:
   SNP_01 to SNP_16 linked to QTL1.1;
   SNP_17 to SNP_31 linked to QTL1.2;
   SNP_32 to SNP_47 linked to QTL2.1; and/or
   SNP_48 to SNP_62 linked to QTL3.1.

Molecular marker screening obviously involves obtaining plant material and analyzing the genomic DNA of the material for the marker haplotype or genotype.

In this method also other molecular marker tests described elsewhere herein can be used.

Also encompassed herein is a method for producing a cultivated cucumber plant comprising one or more introgression fragments on chromosome 1, 2 and/or 3, wherein said introgression fragments comprise QTL1.1, QTL1.2, QTL2.1 and/or QTL3.1 (or variants thereof), comprising:
a) providing a first cultivated cucumber plant, preferably lacking QTL1.1, QTL1.2, QTL2.1 and/or QTL3.1,
b) providing a second cultivated cucumber plant selected from plants grown from seeds deposited under accession number NCIMB43745 or progeny thereof, or providing a second cultivated or wild cucumber plant comprising QTL1.1, QTL1.2, QTL2.1 and/or QTL3.1, or a variant of any of these, and comprising a resistant donor SNP haplotype or genotype for at least 5, 6, 7, 8, 9, 10, 11, 12, 13, 14, 15 or 16 SNP markers linked to the QTL,
c) crossing said plant of a) with said plant of b),
d) collecting F1 seeds from said cross and optionally selfing said F1 plants one or more times to produce an F2 or F3 or further selfing population,
e) optionally backcrossing the F1 plant or an F2 or F3 or further selfing plant to the plant of a) to produce a backcross population,
f) optionally selfing the backcross population one or more times,
g) identifying a F1, F2, F3, further selfing or backcross plant which comprises the resistant donor SNP haplotype or genotype for at least 5, 6, 7, 8, 9, 10, 11, 12, 13, 14, 15 or 16 SNP markers linked to the QTL.

In a further aspect a method of producing F1 hybrid plants is provided comprising:
a) providing a first inbred cucumber plant comprising an introgression fragment comprising QTL1.1, QTL1.2, QTL2.1 and/or QTL3.1, wherein said introgression fragment is the fragment as found in NCIMB43745, or a shorter fragment of that introgression fragment and/or wherein the QTL is the QTL as found in NCIMB43745, or wherein the QTL comprising a resistant donor SNP haplotype or genotype for at least 5, 6, 7, 8, 9, 10, 11, 12, 13, 14, 15 or 16 SNP markers linked to the QTL,
b) providing a second inbred cucumber plant (optionally comprising an introgression fragment comprising QTL1.1, QTL1.2, QTL2.1 and/or QTL3.1, wherein said introgression fragment is the fragment as found in NCIMB43745, or a shorter fragment of that introgression fragment and/or wherein the QTL is the QTL as found in NCIMB43745, or wherein the QTL comprising a resistant donor SNP haplotype or genotype for at least 5, 6, 7, 8, 9, 10, 11, 12, 13, 14, 15 or 16 SNP markers linked to the QTL),
c) crossing said plant of a) with said plant of b),
d) collecting F1 hybrid seeds from said cross.

In another aspect a method for generating progeny of NCIMB43745 retaining QTL1.1, QTL1.2, QTL2.1 and/or QTL3.1 is provided, said method comprising:
a) growing a plant from seeds deposited under accession number NCIMB 43745;
b) selfing said plant one or more times or crossing said plant one or more times with another cultivated cucumber plant to generate progeny seeds;
c) screening said progeny seeds or plants grown from said seeds or parts of the seeds or plants using a molecular marker assay which detects at least 1, 2, 3, 4, 5, 6, 7, 8, 9, 10 or more SNP marker disclosed herein as being linked to one or more QTLs selected from QTL1.1, QTL1.2, QTL2.1 and/or QTL3.1;
d) identifying and/or selecting a progeny plant comprising a resistant donor SNP haplotype or genotype for at least 5, 6, 7, 8, 9, 10, 11, 12, 13, 14, 15 or 16 SNP markers linked to the QTL (as described elsewhere herein); and
e) optionally confirming the enhanced ToLCNDV-ES resistance of said progeny plants.

A method for generating progeny of NCIMB 43745 is provided, said method comprising:
a) growing a plant from seeds deposited under accession number NCIMB 43745;
b) selfing said plant one or more times or crossing said plant one or more times with another cucumber plant to generate progeny seeds;
c) screening said progeny seeds or plants grown from said seeds or parts of the seeds or plants using a molecular marker assay which detects at least 1, 2, 3, 4, 5, 6, 7, 8, 9, 10 or more SNP marker linked to a QTL selected from QTL1.1, QTL1.2, QTL2.1 and/or QTL3.1, wherein the SNP markers are:
   SNP_01 to SNP_16 for detecting the introgression fragment comprising QTL1.1;
   SNP_17 to SNP_31 for detecting the introgression fragment comprising QTL1.2;
   SNP_32 to SNP_47 for detecting the introgression fragment comprising QTL2.1;
   SNP_48 to SNP_62 for detecting the introgression fragment comprising QTL3.1;
d) identifying and/or selecting a progeny plant comprising:
   i) at least 5, 6, 7, 8, 9, 10 or more markers of SNP_01 to SNP_16 which have the resistant donor SNP haplotype; and/or
   ii) at least 5, 6, 7, 8, 9, 10 or more markers of SNP_17 to SNP_31 which have the resistant donor SNP haplotype; and/or
   iii) at least 5, 6, 7, 8, 9, 10 or more markers of SNP_32 to SNP_47 which have the resistant donor SNP haplotype; and/or
   iv) at least 5, 6, 7, 8, 9, 10 or more markers of SNP_48 to SNP_62 which have the resistant donor SNP haplotype;
e) optionally confirming the enhanced ToLCNDV-ES resistance of said progeny plants.

A progeny plant generated by any of the above methods is also an aspect of the invention.

One can also use the methods and the markers described herein to reduce the size of the introgression fragment comprising the QTL, i.e. to generate and select recombinants having a smaller introgression fragment, but which retain the QTL.

In one aspect the invention encompasses the use of a recombinant chromosome 1, 2 and/or 3 comprising an introgression fragment from a wild cucumber, said introgression fragment comprising a QTL selected from QTL1.1, QTL1.2, QTL2.1 and QTL3.1 (or a variant of any of these), for breeding cucumber varieties having enhanced ToLCNDV-ES resistance.

Also provided is the use of a chromosome 1, 2 and/or 3 as found in seeds deposited under accession number NCIMB 43745 or progeny thereof for generating cultivated cucumber plant comprising an introgression fragment of said chromosome 1, 2 or 3.

Also provided is the use of plants grown from seeds deposited under accession number NCIMB 43745, or progeny thereof, for generating a cultivated cucumber plant comprising enhanced ToLCNDV-ES resistance, wherein said enhanced ToLCNDV-ES resistance is conferred by an introgression fragment obtained from chromosome 1, 2 or 3 of said plants or progeny.

When referring to QTL1.1, QTL1.2, QTL2.1 and QTL3.1 as present in the deposited seeds (NCIMB 43745) or progeny thereof, it is noted that sequence flanking the SNP at nucleotide 51 provided in Tables 1 to 4 is the flanking sequence of the specific donor used in the mapping and introgression. The SNP marker at nucleotide 51 may, however, also be present in a sequence comprising less than 100% sequence identity to the sequence provided under SEQ ID NO: 1 to 62, e.g. at nucleotide 51 of a sequence comprising at least 95%, 96%, 97%, 98%, 99% sequence identity to the provided sequence. Sequence identity of the region flanking the SNP can e.g. be analyzed by BLAST analysis or by pairwise alignment of sequences of the same length comprising the SNP at nucleotide 51 (using e.g. Needle, with default parameters).

Also the molecular marker sequences (and isolated nucleic acid molecules comprising the sequence) disclosed herein and their use in detecting and/or generating cucumber plants comprising said QTLs described herein are encompassed herein.

Further a method of growing a plant comprising one or more QTLs selected from QTL1.1, QTL1.2, QTL2.1 and/or QTL3.1 (or variants of any of these) in an area where ToLCNDV-ES is present in the Bemisia populations, e.g. in Mediterranean countries or in Northern European countries, either in the field, or in glasshouses or tunnels, whereby less insecticide treatment is needed compared to plants lacking the QTLs. Optionally no insecticide treatment is needed, e.g. if the plants comprise an average disease score of 9.0 (no symptoms). In the plants comprising all four QTLs in homozygous form no virus was detected by DAS-ELISA. Thus, in one aspect a method for growing cultivated cucumber plants comprising a ToLCNDV-ES disease score of 9.0 (comprising all four QTLs in homozygous form) is provided, said method comprising no chemical treatment to control whiteflies being applied during the cultivation.

### Seed Deposits

A representative sample of seeds of a Cucumis sativus var. sativus of the slicer cucumber type, designated ToLCNDV-R, comprising introgression fragments comprising QTL1.1, QTL1.2, QTL2.1 and QTL3.1 in homozygous form, and a genetic control (GC) lacking the introgression fragments and the QTLs, designated ToLCNDV-GC, were deposited by Nunhems B.V. on 5 Mar. 2021 at the NCIMB Ltd. (Ferguson Building, Craibstone Estate, Bucksburn Aberdeen, Scotland AB21 9YA, UK) according to the Budapest Treaty, under the Expert Solution (EPC 2000, Rule 32(1)). Seeds were given the following deposit numbers NCIMB 43745 (ToLCNDV-R) and NCIMB 43744 (ToLCNDV-GC).

The Applicant requests that samples of the biological material and any material derived therefrom be only released to a designated Expert in accordance with Rule 32(1) EPC or related legislation of countries or treaties having similar rules and regulation, until the mention of the grant of the patent, or for 20 years from the date of filing if the application is refused, withdrawn or deemed to be withdrawn.

Access to the deposit will be available during the pendency of this application to persons determined by the Director of the U.S. Patent Office to be entitled thereto upon request. Subject to 37 C.F.R. § 1.808(b), all restrictions imposed by the depositor on the availability to the public of the deposited material will be irrevocably removed upon the granting of the patent. The deposit will be maintained for a period of 30 years, or 5 years after the most recent request, or for the enforceable life of the patent whichever is longer, and will be replaced if it ever becomes nonviable during that period. Applicant does not waive any rights granted under this patent on this application or under the Plant Variety Protection Act (7 USC 2321 et seq.).

The following non-limiting Examples describe how one can obtain plants comprising QTL1.1, QTL1.2, QTL2.1 and/or QTL3.1 or variants of any of these. Examples provided herein are non-limiting examples. Any patent and non-patents documents mentioned herein are incorporated by reference.

### EXAMPLES

### Example 1—ToLCNDV-ES Disease Assay

For testing plants for resistance to ToLCNDV-ES a protocol was established, using whitefly to transmit the ToLCNDV-ES virus.

A highly virulent Spanish isolate of ToLCNDV-ES (MU_18_006) was used.

Per genotype to be tested 10 plants were infected in two replicates (5 inoculated plants per replicate).

Tests were carried out in the greenhouse, in a randomized block design. Control varieties were included, which have a known response to ToLCNDV-ES infection. These are for example Renoir F1 (average disease score of about 3.0 or less), Mastil F1 (average disease score of about 2.0 or less), Sqisito F1 (average disease score of about 5.0 or less), Taray F1 (average disease score of about 6.0 to 7.0 or less).

Plants are sown in trays and are grown in a nursery. Inoculation of the test plants is done about 10 to 14 days after sowing, when the first leaf expanded.

Inoculation of test plants with ToLCNDV-ES is carried out in three phases. First (phase 1) ‘inoculum plants’ (carrying the ToLCNDV-ES) are placed in the whitefly production area containing high numbers of whiteflies and the whiteflies are left to feed on the inoculum plants for 2 to 3 days to acquire the virus. Secondly, after 2 to 3 days, the trays with test plants are placed into the room (phase 2), the whiteflies are shaken off the inoculum plants (which are removed) so that the whiteflies move to the test plants. About 10 to 15 whiteflies should be present on each test plant. The whiteflies are left for 2 to 3 days on the test plants to allow for virus transmission to the test plants. Thereafter (phase 3) the whiteflies are removed from the plants by applying high pressure water with soap solution to the test plants.

The inoculated test plants are transplanted into bigger pots and are then grown in the greenhouse under 25° C./16 hrs light, 16° C./8 hrs dark, approximately 60% relative humidity in autumn and winter or at 32° C./16 hrs light, 18° C./8 hrs dark, approximately 60% relative humidity in spring or summer.

At e.g. 25 (or 21, 22, 23 or 24) days post inoculation (dpi) and at e.g. 32 (or 33, 34 or 35) dpi a first and second visual disease scoring is carried out, optionally a further scoring at e.g. 46 (or 47, 48, or 49) dpi.

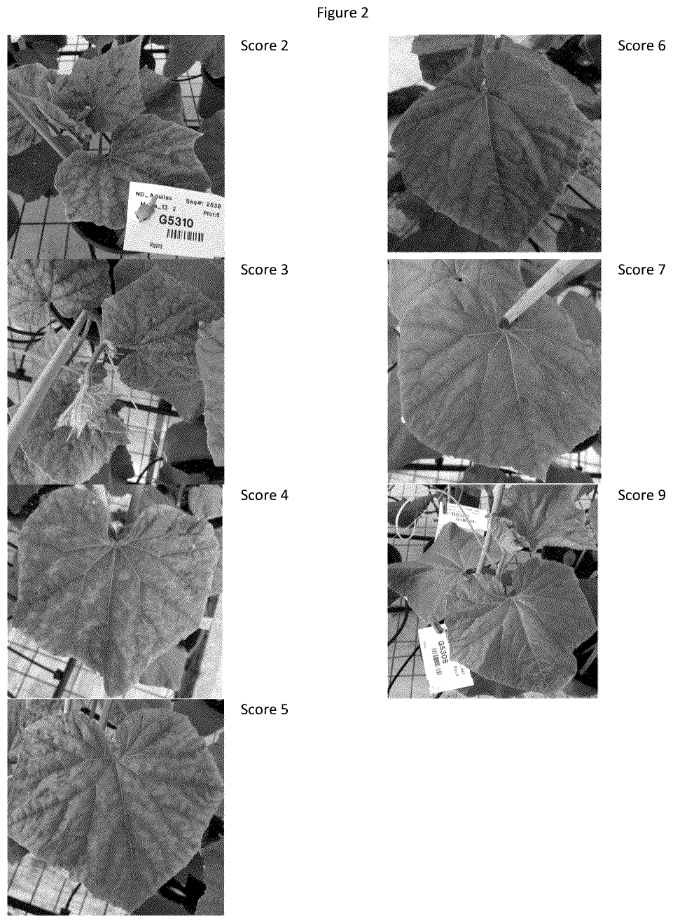

The scoring is carried out visually for ‘yellowing’, using the following scale (see also FIG. 2):
Score 2.0: cucumber leaves with fully covered yellowing mosaics (about 90 to100% of leaf area)
Score 3.0: cucumber leaves with strong yellowing mosaics (about 70 to 80% of leaf area)
Score 4.0: cucumber leaves with clear yellowing mosaics on fully expanded leaves (about 40 to 60% of leaf area)
Score 5.0: cucumber leaves with yellowing mosaics (about 30 to 40% of leaf area) evenly distributed in interveinal spaces
Score 6.0: cucumber leaves with yellowing mosaics (about 20-30% of leaf area) evenly distributed in interveinal spaces
Score 7.0: cucumber leaves with mild yellowing mosaics (about 10% of leaf area)
Score 8.0: cucumber leaves with presence of very faint yellowing mosaic symptom (vein bending)
Score 9.0: healthy leaves with no symptoms

The average disease score per test genotype and per control genotype is calculated.

The overall test schedule is summarized below:

| | Day | |
|---|---|---|
| Week | number | Events |
| 1 | 1 | Sow plants to refresh inoculum |
| 2 | 14 | Refresh inoculum |
| 6 | 40 | Sow trial/test plants |
| 7 | 48 | Germination count |
| 8 | 52 | Phase1 inoculation preparation (inoculum plants placed |
| | | in whitefly area to acquire virus) |
| 8 | 54 | Phase2 inoculation day (test plants are placed in whitefly |
| | | area) |
| 8 | 56 | Phase3 inoculation (remove whiteflies) |
| 9 | 57 | Transplant |
| 11 | 75 | 1^{st} evaluation (e.g. 21 dpi) |
| 13 | 89 | 2^{nd} evaluation (e.g. 35 dpi) |
| 15 | 103 | 3^{rd} evaluation (e.g. 49 dpi, optional) |
| 15 | 105 | Remove trial and disinfect facilities |

### Example 2—DAS-ELISA Assays

DAS-ELISA was carried out with a ToLCNDV antiserum (Agdia EMEA, Grigny, France) using standard methods, see e.g. Romay et al., 2019 Plant disease 11:2913-2919. A total of 10 plants of a genotype (5 plants per replicate) have been sampled individually by collecting 5 leaf pouches from 5 different young leaves collected 30 dpi from adult plants. OD reading is at 60 minutes after DAS-ELISA incubation. The average OD of 10 plants is determined.

### Example 3—Identification of ToLCNDV-ES Resistance Conferring QLs

Three F2 mapping populations were generated in two different genetic backgrounds by crossing a wild donor cucumber with an elite cucumber (slicers and long cucumber types). The population sizes were above 700 plants.

The wild donor had a ToLCNDV-ES disease score of 9.0 at all three time points after inoculation (dpi), i.e. was entirely symptom free.

The F2 populations were analyzed genetically for SNP markers and phenotypically for ToLCNDV-ES symptoms, as described below, in a ToLCNDV-ES disease assay.

In all mapping populations four QTLs were identified, three major QTLs (QTL1.1, QTL1.2 and QTL2.1) with a LOD score above 20 and one minor QTL (QTL3.1) with a lower LOD score (above 4). 2500 seeds of a F3 line containing all four QTLs in homozygous form were deposited under accession number NCIMB 43745. Further 2500 seeds of a BC1F4 line lacking all four QTLs was deposited under accession number NCIMB 43744.

Single Nucleotide Polymorphism markers (SNPs) linked to these QTLs and spanning the introgression fragments are provided below. It is noted that the sequence flanking the SNP is the sequence of the wild donor. Also the SNP position on the physical C. sativus (Chinese Long V3) map is shown. Further the SNP haplotype for each QTL of three ToLCNDV-ES resistant donors (CGN22263, PI605996 and CGN22932, alternative name PI197087) is provided. It is understood that the SNP ‘haplotype’ refers to the SNP nucleotides on a single chromosome. In general, when referring to the SNP haplotype for SNP markers linked to a ToLCNDV resistance conferring QTL, the SNP nucleotides of the resistant donor is referred to.

TABLE 1
SNP markers for QTL1.1 on chromosome 1

| | | | SNP | | | | |
|---|---|---|---|---|---|---|---|
| | | | haplotype | | | | |
| | | SNP | of | | | | |
| | Physical | haplotype | intro- | | | | |
| | position | of | gression | | | | |
| | of the | recurrent | fragment | | | | |
| | SNP | parent | from donor | | | | Sequence |
| SNP | (base | line C13_ | (as in | CG3N | PI | CGN | with SNP (at nucleotide 51 of |
| marker | number) | 4231-7 | NCIMB43745) | 2226 | 605996 | 22932 | the SEQ ID No) highlighted |
| SNP_01 | 8235142 | G | T | T | T | T | AACACAAAAATGAAGATATGAAGTTGAT |
| | | | | | | | GAATTCCATCTGCTGAAATAAA[G/T]G |
| | | | | | | | AAGCTTTACCCTGTAAAACACAATCAAT |
| | | | | | | | CATCATCACCACATTATTATG |
| | | | | | | | (SEQ ID NO: 1) |
| SNP_02 | 8770395 | C | T | T | T | T | TTTAATCAAATCCCTAGCTAGGGATTCA |
| | | | | | | | TTTACAACCAAGTGACATGTAA[T/C]G |
| | | | | | | | TAATGTTCTATTTTGATCCATACATGTA |
| | | | | | | | GCAAAGGGTTAGGGTGATTTA |
| | | | | | | | (SEQ ID NO: 2) |
| SNP_03 | 9058515 | C | T | T | T | T | TGAGATGGTAAATTAAAACTAATATAAA |
| | | | | | | | ATCCTAAATATAAGTAACAAAA[T/C]G |
| | | | | | | | GTATTACAACTTTATAACCATAAAATTA |
| | | | | | | | TCATAGGAACCAAATTTTTAG |
| | | | | | | | (SEQ ID NO: 3) |
| SNP_04 | 9192324 | C | T | T | T | T | AAACAGCAGCTCAAAACTCAGCTTCTTC |
| | | | | | | | ATCATCTTCTAATTAAAGACTT[T/C]G |
| | | | | | | | ATTTCTTCTTTGTATAACCAATCAAACC |
| | | | | | | | CAATCTTCTTTTGGTTTGTGG |
| | | | | | | | (SEQ ID NO: 4) |
| SNP_05 | 9624895 | G | A | A | A | A | ACTATTTCTTGCAGTGTTTAGTATAATT |
| | | | | | | | TATTGGAAAATTGCAATGGACG[A/G]C |
| | | | | | | | TATTTTAGCAATAATAATTAATGATATA |
| | | | | | | | GCAACATTTTAAAAAATTGCA |
| | | | | | | | (SEQ ID NO: 5) |
| SNP_06 | 9792649 | A | C | C | C | C | TATTACTATTTTTGCTCAACTTCATTTC |
| | | | | | | | AAACAAACTCAAACGGATCTTA[C/A]T |
| | | | | | | | AAAATAAGATGATTCTCTCTCTCAAATT |
| | | | | | | | CAACTAGTTATCAAGCAAAAT |
| | | | | | | | (SEQ ID NO: 6) |
| SNP_07 | 10086907 | A | T | T | T | T | TTGTTTATTTAGAAGACTTTAACAAAAA |
| | | | | | | | TCAATTTATTTAGAAGACCTAA[T/A]A |
| | | | | | | | TTGAATCATATATACAAACTAATTATAT |
| | | | | | | | TAAGTATGGAATAATGAGGCC |
| | | | | | | | (SEQ ID NO: 7) |
| SNP_08 | 10091801 | G | A | A | A | A | ATAATCAAATCTAAACAATCGTGTACTA |
| | | | | | | | ATTCAAACAATTAAATCTAAAT[A/G]A |
| | | | | | | | TCGTGTACCAAGTCTGATCGTTTACCAA |
| | | | | | | | AAAAAATCTTGAAAAACATCT |
| | | | | | | | (SEQ ID NO: 8) |
| SNP_09 | 10260090 | A | G | G | A | G | ACAAACAAGAAACAACAACTACAAATTC |
| | | | | | | | GGGTCCTCAAATCAATAATGCT[G/A]A |
| | | | | | | | GTATTGAAGAAAAAATACAACAATTTGC |
| | | | | | | | TCCACTCAAGAAAAATATTGC |
| | | | | | | | (SEQ ID NO: 9) |
| SNP_10 | 10780728 | G | C | C | C | C | AATATTGTAAGTAAAATGGACATTCAGC |
| | | | | | | | ACTATAATTAAGAAACAATTAA[G/C]T |
| | | | | | | | AATCAAATTTAATAAATGCATAGTTGCA |
| | | | | | | | TCCTCTTTTTAACCTTTTTCT |
| | | | | | | | (SEQ ID NO: 10) |
| SNP_11 | 11783787 | G | C | C | C | C | TATCAAATACAAAGTAAGTTGTATCCAC |
| | | | | | | | AATGTTACCAGGATAAGGTACC[G/C]A |
| | | | | | | | GCCTTATCCTTATATTATAGACCCTTTA |
| | | | | | | | AGCTAATCTTGAACATAGATC |
| | | | | | | | (SEQ ID NO: 11) |
| SNP_12 | 13350546 | G | C | C | C | C | TCTTTCCCCTGTCCAAACAGGAAATGAC |
| | | | | | | | ATTTTTTTAGCTATCAATACAA[C/G]C |
| | | | | | | | AAAGATTAATGCACTGTACCATAATCCT |
| | | | | | | | TGAGAGTGATCCTAATGAAAT |
| | | | | | | | (SEQ ID NO: 12) |
| SNP_13 | 13881857 | G | T | T | T | T | ATCAGTAACGTTGCCTATGGATCCCAAT |
| | | | | | | | CTAAAACTCTCAAAAGATGAAG[T/G]A |
| | | | | | | | AAATTCATTGTTGACGAGGATATCACTT |
| | | | | | | | GCTACACAAGATTGATTGGAA |
| | | | | | | | (SEQ ID NO: 13) |
| SNP_14 | 14492460 | G | A | A | A | A | AGGAACGTAAGTGAAAGATCAAAGGACA |
| | | | | | | | TTTTTTTTTTCTTTTATCCGAT[A/G]A |
| | | | | | | | GCATACCAAAACAAATCTGAGAGGTACG |
| | | | | | | | GTAAGAGTAGGTAGTTCAAAT |
| | | | | | | | (SEQ ID NO: 14) |
| SNP_15 | 15379562 | T | C | C | T | C | CAACTATTTCCCTTTATATTGTTCTGTT |
| | | | | | | | TTAAAAAAAATATTTCCCTTTC[T/C]T |
| | | | | | | | CAATTTCCTTTCCTTTACATATTTTAAA |
| | | | | | | | TGTTAGTCTAAAATAAAATGA |
| | | | | | | | (SEQ ID NO: 15) |
| SNP_16 | 16209127 | A | G | G | G | G | TGAACTAATTTAAAATTTTTTAAAAATA |
| | | | | | | | TTTAAAATTATCAGTTTGGTTT[G/A]C |
| | | | | | | | ACCCCTAATGTATGGTAAATTATATGAA |
| | | | | | | | AGAAAAAATAATTTGGTTAAG |
| | | | | | | | (SEQ ID NO: 16) |

TABLE 2
SNP markers for QTL1.2 on chromosome 1

| | | | SNP | | | | |
|---|---|---|---|---|---|---|---|
| | | | haplotype | | | | |
| | | | of | | | | |
| | Physical | | intro- | | | | |
| | position | SNP | gression | | | | |
| | of the | haplotype | fragment | | | | |
| | SNP | of | from donor | | | | Sequence |
| SNP | (base | recurrent | (as in | CGN | PI | CGN | with SNP (at nucleotide 51 of |
| marker | number) | parent | NCIMB43745) | 22263 | 605996 | 22932 | the SEQ ID No) highlighted |
| SNP_17 | 22942981 | T | C | C | C | C | CAGTGAATAGAAATAGAGAACTGACCTT |
| | | | | | | | GAATAGAGTCAACCATCGTGAT[T/C]G |
| | | | | | | | AGTCCTCTTTCCACAAAATAAGGATACC |
| | | | | | | | ACCCGAAGAACCATAAGCTTC |
| | | | | | | | (SEQ ID NO: 17) |
| SNP_18 | 23015144 | T | G | G | G | G | TGAGGAACCAGCATCTTTCTTGATTTTG |
| | | | | | | | TCACCATATGGAGGAAAGATGA[G/T]G |
| | | | | | | | CAAATTTCTGATTTGGAAACTCCATTTC |
| | | | | | | | CACATAGAAAAGGGAATACTT |
| | | | | | | | (SEQ ID NO: 18) |
| SNP_19 | 23039745 | A | G | G | G | G | AACTATTTAAATTAATAATTTAGTTGTT |
| | | | | | | | TTTACTTCCAAACTAAGTTATA[G/A]G |
| | | | | | | | TTTTATTACTAATATTCATATTTATATT |
| | | | | | | | GAGATCAATAGATGATAATTT |
| | | | | | | | (SEQ ID NO: 19) |
| SNP_20 | 23421850 | G | T | T | T | T | ACATATTTAAATGTTGGTTGGAGAGAAT |
| | | | | | | | CTATTTGTTCAATTCACTTAAA[T/G]A |
| | | | | | | | CCTAATTAAAAATTTTCACATACAAACA |
| | | | | | | | ATATTTATTTTTTACAAAAAT |
| | | | | | | | (SEQ ID NO: 20) |
| SNP_21 | 23651359 | A | G | G | G | G | TGGGGAACACTACTCGGGGCCTGCCACA |
| | | | | | | | TTCATGGAAATGTTGAGCTTGC[G/A]G |
| | | | | | | | AAGTGGCATCAAAACATCTATTTGATTT |
| | | | | | | | AGACCCTTTGAACTCTGGGTA |
| | | | | | | | (SEQ ID NO: 21) |
| SNP_22 | 23844859 | C | G | G | G | G | GGAATCGTATTTCTAAATGGGGTTTTGT |
| | | | | | | | GTATGTAGAGAACTTTGTTTTC[C/G]A |
| | | | | | | | TATGGGGTTATCTTCCAAAAAGCAATCC |
| | | | | | | | ATTGGTTTAGGAAGCATTTTC |
| | | | | | | | (SEQ ID NO: 22) |
| SNP_23 | 23980973 | C | T | T | T | T | TTAAATGAATTAGTCACCCTTAAGCTCT |
| | | | | | | | CCTAAACTTGTCAGCTTCTACT[C/T]G |
| | | | | | | | GTGATTTACATGTATGAGCTTAATGTCC |
| | | | | | | | TTTCTAATTATGCTTAGCTTA |
| | | | | | | | (SEQ ID NO: 23) |
| SNP_24 | 24059623 | G | C | C | C | C | TTATAAAACATAACACTTTAAGATATGG |
| | | | | | | | TAATTGAATAGACACATAAAAC[C/G]A |
| | | | | | | | AAAAGGTTTAACCAAAATATGTATTACT |
| | | | | | | | TTATCTGCCACTAAATTAAGT |
| | | | | | | | (SEQ ID NO: 24) |
| SNP_25 | 24180637 | G | A | A | A | A | GCTAATAAACTCGTATAATTTATCGTTG |
| | | | | | | | ATAGACCAATTTTTACAACATA[G/A]T |
| | | | | | | | TTATTAGTGATAGACTTTATCATTGATA |
| | | | | | | | GAATTTGACAAATTTTGCTAT |
| | | | | | | | (SEQ ID NO: 25) |
| SNP_26 | 24200736 | T | C | C | C | C | AAAGGGAGTTCATGGTTTTCAAAGTCAC |
| | | | | | | | ACATACCAAGTGAATCTTGGAG[T/C]A |
| | | | | | | | GTTGTGTGAGTTTACTGTTCCTATAAGG |
| | | | | | | | GACGTGTGCATTCCTTAGAGC |
| | | | | | | | (SEQ ID NO: 26) |
| SNP_27 | 24205819 | A | C | C | C | C | TTCCTTCTCTCACTTGTAAGATTTCTTT |
| | | | | | | | TATTATTATTACAAACAAAAAT[C/A]T |
| | | | | | | | AAGTTTTTCTTCTTTCTTTTTTCCCTTC |
| | | | | | | | TTCCCACCATGGTCCATTGCT |
| | | | | | | | (SEQ ID NO: 27) |
| SNP_28 | 24342052 | T | C | C | C | C | AAAATGTTACAATTTTACCATTGACATC |
| | | | | | | | TAGATTTTATTTCAATTTGGTC[T/C]C |
| | | | | | | | TAGATTACATGTCAATCTCAAATTTTCA |
| | | | | | | | CGAAATACTTATTAATGTTAA |
| | | | | | | | (SEQ ID NO: 28) |
| SNP_29 | 24816687 | G | C | C | C | C | TATACTAAAATATTTACCGTATATAATT |
| | | | | | | | AAATTTTAAATGTTATCATAAC[G/C]T |
| | | | | | | | TTGTTATATTTTATAAATATTTTGTTAA |
| | | | | | | | ATTTGTGATTTTTGATAATTT |
| | | | | | | | (SEQ ID NO: 29) |
| SNP_30 | 25023580 | G | A | A | A | A | GGGCTCTAAGTTTAGTTATGAAGTTTGG |
| | | | | | | | ATTGGAAATTTGTGTTAGGACA[A/G]T |
| | | | | | | | GGAATAAGTTAGACAATTGTGTTATGAC |
| | | | | | | | AATGCAATGAGTTAGATATTT |
| | | | | | | | (SEQ ID NO: 30) |
| SNP_31 | 25543032 | T | G | G | G | G | CGTTGGTTTGAATTATATAGTCTATTTT |
| | | | | | | | TTAAAAAAATAGTTGGATCATA[G/T]A |
| | | | | | | | ATAAATTAATATTTTGGAATTACATCTA |
| | | | | | | | AAAAGTAAAAATGAAGAGAAT |
| | | | | | | | (SEQ ID NO: 31) |

TABLE 3
SNP markers for QTL2.1 on chromosome 2

| | | | SNP | | | | |
|---|---|---|---|---|---|---|---|
| | | | haplotype | | | | |
| | | | of | | | | |
| | Physical | | intro- | | | | |
| | position | SNP | gression | | | | |
| | of the | haplotype | fragment | | | | |
| | SNP | of | from donor | | | | Sequence |
| SNP | (base | recurrent | (as in | CGN | PI | CGN | with SNP (at nucleotide 51 of |
| marker | number) | parent | NCIMB43745) | 22263 | 605996 | 22932 | the SEQ ID No) highlighted |
| SNP_32 | 15218569 | C | T | T | T | T | CATTGGATTTGTAGCCAAAACGTAGTAT |
| | | | | | | | CTGAAGTTATTGGTATGCCGCA[T/C]A |
| | | | | | | | TATAAATTCATTAGTTTGCACCATGCAC |
| | | | | | | | AAGGGGGACAAATATAATTTT |
| | | | | | | | (SEQ ID NO: 32) |
| SNP_33 | 15495712 | C | T | T | T | T | GATTCATTCTCTCTTTTTTTATTTTTAA |
| | | | | | | | ACATATTTCCCTCCAGAAAATG[T/C]A |
| | | | | | | | CAAGGTGGGAGAAATTTGGATGTGACAA |
| | | | | | | | TTTCCACAATCTATCACTCAG |
| | | | | | | | (SEQ ID NO: 33) |
| SNP_34 | 15759464 | T | C | C | C | C | AGATCTAGCTCATGACATTTTGTAACTT |
| | | | | | | | CCAAAATTTGAACGTCAAATCC[C/T]A |
| | | | | | | | AAGTAATGATATAGTTGTTTGATGTGAT |
| | | | | | | | TTAGTGTGATCCTACATCTGT |
| | | | | | | | (SEQ ID NO: 34) |
| SNP_35 | 16014805 | T | A | A | A | A | GGAAACCCTACTTTTAGCTTTGGCTTGA |
| | | | | | | | AGTCCTTATAAAAATAAATAAA[A/T]A |
| | | | | | | | AATAAAATAAAATAGCTTTGGCTTGAAA |
| | | | | | | | TCCAAGGTTGGGATGCCAGAG |
| | | | | | | | (SEQ ID NO: 35) |
| SNP_36 | 16349730 | C | T | T | T | T | AATGCAAAGAGTATAATTCTTAGTCAAG |
| | | | | | | | TTGTGAGAGAAAAACATGTTTT[T/C]A |
| | | | | | | | AAGGGTTAATTTTCATAAATATAATAAA |
| | | | | | | | TCGGAAAATATTTATGACTCG |
| | | | | | | | (SEQ ID NO: 36) |
| SNP_37 | 16378312 | T | C | C | C | C | CATTCAGACATGATTGGAGGCATAATCC |
| | | | | | | | ATAGTTGACAGAGTGGTATGTT[C/T]A |
| | | | | | | | GAGTTACATCATCTGGCATCATCAAGGT |
| | | | | | | | AGTGGCTAAATTCGCCAATGC |
| | | | | | | | (SEQ ID NO: 37) |
| SNP_38 | 16817201 | T | G | G | G | G | GATTTTAAATCCATAAATCTATTAAAGA |
| | | | | | | | AAACTGGGGGTAAATTTTTTTT[G/T]A |
| | | | | | | | CTCCATAATGGTAACAAATAGAGTCAAT |
| | | | | | | | TAAAAAAATTAAAATATTTAT |
| | | | | | | | (SEQ ID NO: 38) |
| SNP_39 | 17009321 | T | C | C | C | C | AGAAATTTAGAAAGAAGGAAAAGAAAAA |
| | | | | | | | ACCCTTCCTTTCTCATTCCATC[C/T]A |
| | | | | | | | TCGCTTCTGCTATACAAATTGAAAACTA |
| | | | | | | | TGGCTTTTCTCTTCCATAGAA |
| | | | | | | | (SEQ ID NO: 39) |
| SNP_40 | 17332159 | A | G | G | G | G | ATGAAAATGAAATGGTTCATCCTCTTTA |
| | | | | | | | CATCAAATTCACAATCTTGTAA[G/A]A |
| | | | | | | | CTAAAATTATGGTGGAGGGACTACGGTG |
| | | | | | | | GTTTTTCTTCTCCATAATTTT |
| | | | | | | | (SEQ ID NO: 40) |
| SNP_41 | 17569912 | C | T | T | T | T | TTTATCTACGGTCTTATATGTTCAACTC |
| | | | | | | | CTCAAATGGAAAAATGCAAGGC[T/C]T |
| | | | | | | | TGTTGTTCTTGTTTTGTTCATATATAT |
| | | | | | | | CTTCATGGAAGATAAGGTTTT |
| | | | | | | | (SEQ ID NO: 41) |
| SNP_42 | 17907091 | T | A | A | A | A | TTTCAAGAACATTCGAAAAAGAAACTTG |
| | | | | | | | CTCCACGGTGCTTATAGATAAG[A/T]A |
| | | | | | | | CAAAAATGGAATGTTAAAAGAAACAATA |
| | | | | | | | AAATATTAGTTAGTAATTATT |
| | | | | | | | (SEQ ID NO: 42) |
| SNP_43 | 18379675 | G | T | T | T | T | ATATTTGTTATAATGTGTCATTTCCAAT |
| | | | | | | | AATTTCCTTTTTTCATAGTATT[T/G]T |
| | | | | | | | TTTTTTCTACTAGGATGAGTCATAGTTT |
| | | | | | | | TTTCTAGTTTTCTTACCTAAA |
| | | | | | | | (SEQ ID NO: 43) |
| SNP_44 | 18479446 | C | T | T | T | T | AACCAAATAGATTTATTGGAAAAACAAT |
| | | | | | | | TAAGATATATTAGTGCACCTCC[T/C]G |
| | | | | | | | ATCTTGTTCTCCTGCTTGTTACTTAACC |
| | | | | | | | TATCAGTGCACCAAGACTAAT |
| | | | | | | | (SEQ ID NO: 44) |
| SNP_45 | 18719413 | A | G | G | G | G | ACTTTCAAATTTTGTTTCTTTTATAATC |
| | | | | | | | TATACAATGGGAATGAGTTGGT[G/A]C |
| | | | | | | | ACCCAATTATCCTAATTAGATTAACATA |
| | | | | | | | ATCTCATCACCCTTCTCATCC |
| | | | | | | | (SEQ ID NO: 45) |
| SNP_46 | 18897159 | G | A | A | A | A | AAAATTAAAGAAAATAATCGAAAGGTCC |
| | | | | | | | ATTTTCGGTGAACATTTTTACT[G/A]T |
| | | | | | | | AAGAACTAAAACAAACCGAGTAAAATGA |
| | | | | | | | ACCAAAATCAAGAGTATAAAG |
| | | | | | | | (SEQ ID NO: 46) |
| SNP_47 | 19535432 | A | G | G | G | G | AAGGGGAAATGGTGTTACCTGTATTTCA |
| | | | | | | | ATCAAATTGCAAAGAATGCAAA[G/A]A |
| | | | | | | | ATGCAAGAATGGGAGGACCAACAAATGC |
| | | | | | | | AGTGTTTTTGGGGGCAAAACT |
| | | | | | | | (SEQ ID NO: 47) |

TABLE 4
SNP markers for QTL3.1 on chromosome 3

| | | | SNP | | | | |
|---|---|---|---|---|---|---|---|
| | | | haplotype | | | | |
| | | | of | | | | |
| | Physical | | intro- | | | | |
| | position | SNP | gression | | | | |
| | of the | haplotype | fragment | | | | |
| | SNP | of | from donor | | | | Sequence |
| SNP | (base | recurrent | (as in | CGN | PI | CGN | with SNP (at nucleotide 51 of |
| marker | number) | parent | NCIMB43745) | 22263 | 605996 | 22932 | the SEQ ID No) highlighted |
| SNP_48 | 3637 | C | A | A | A | A | TTTGGCGCTACCGTCGTACACCACGAAC |
| | | | | | | | AAGCCCCACTCGACCCCTAGGC[C/A]T |
| | | | | | | | GGGCTATTTTTATGTGTCATACAATGTG |
| | | | | | | | TAATATATATGGGTTGCCACA |
| | | | | | | | (SEQ ID NO: 48) |
| SNP_49 | 9568 | A | C | C | C | C | TTAACTCGGTTTGGAATACAGTTTTGGG |
| | | | | | | | CTTTTAGATTCACAAAATTCCA[A/C]G |
| | | | | | | | CCCATTACGCCCTTTATGGATGAAAAAA |
| | | | | | | | TTCGAATTGGAATTAAGTGAC |
| | | | | | | | (SEQ ID NO: 49) |
| SNP_50 | 10011 | T | C | C | C | C | CTGCCAAAGTGCGGTTGTAGAAGGAAGA |
| | | | | | | | TAAATATTTGACCCTCTCTTGA[T/C]T |
| | | | | | | | GGGAAATGAAGTTGTGGAAGACCATAAA |
| | | | | | | | GTTGAAAAGAAGAAATGGTGG |
| | | | | | | | (SEQ ID NO: 50) |
| SNP_51 | 364308 | T | C | C | C | C | AACTACAATCACTTTGATTATTTTAATC |
| | | | | | | | TGTATTTTTTGAGAAATAATCC[T/C]T |
| | | | | | | | TGTTCGTCTTGTTGGACAATTTCTTGTA |
| | | | | | | | TTTAAAAAATTATTGAAAATA |
| | | | | | | | (SEQ ID NO: 51) |
| SNP_52 | 376848 | C | T | T | T | T | ATTGTTATTCTGAAGCCTGGTCCTGATA |
| | | | | | | | AGATGTGCGTCCATGAAGAGCA[C/T]T |
| | | | | | | | GGAAAAATTCCTCAGGGAATTTAGGAGA |
| | | | | | | | AAGAGTTAGTATTGAAGATTT |
| | | | | | | | (SEQ ID NO: 52) |
| SNP_53 | 438098 | A | C | C | C | C | CGCCATGTGTCCAAAAGGTCTCTGCATC |
| | | | | | | | GCCGGGTCGGGGTTATGTGGAT[C/A]T |
| | | | | | | | GGATTTCTGAGGCGATGCGGTTCGGATC |
| | | | | | | | TAACATTTGGGAACCCTAACG |
| | | | | | | | (SEQ ID NO: 53) |
| SNP_54 | 515886 | T | C | C | C | C | ATCTTCATTTTTTCTTTTTCTTTTATGA |
| | | | | | | | TTTACATCTATCAAACAATTTT[C/T]C |
| | | | | | | | GTTTAGTTCATTCACAAAAGAAAAAGGG |
| | | | | | | | TCTTCTGTTCTTGGTTTTTTT |
| | | | | | | | (SEQ ID NO: 54) |
| SNP_55 | 582793 | T | C | C | C | C | CAGAGTAATTTTTTCAATTGTTTGATAA |
| | | | | | | | GCAACACTAGTGATAAAAAAGA[T/C]C |
| | | | | | | | ACTTACTAGTTGTGAATTTGATAGAATT |
| | | | | | | | GCTAATCAAATTTGTGAATAT |
| | | | | | | | (SEQ ID NO: 55) |
| SNP_56 | 741882 | C | G | G | G | G | ACCAAATTTCATTGGAATACAAATAAAC |
| | | | | | | | ATTTGGGGAAGATATATTGAAG[G/C]T |
| | | | | | | | TGTAGTGAAGGTGGACAAAAACAAAAAG |
| | | | | | | | TTTAATATAAATTTATTTGGA |
| | | | | | | | (SEQ ID NO: 56) |
| SNP_57 | 1233995 | T | C | C | C | C | CTCCTAAATTATAATAAAGAGGGTTTTG |
| | | | | | | | GAAGACATGGGAGGGAGAGGTT[T/C]A |
| | | | | | | | GTATTTGCTTCCTCTTACACCCAAACAG |
| | | | | | | | CTTTTTCTTGGTCCCTGTTTC |
| | | | | | | | (SEQ ID NO: 57) |
| SNP_58 | 1424020 | C | T | T | T | T | CTCTTATGATTTATCAGTgAtAGATAA |
| | | | | | | | TATTTCCAACACGATCTATTAG[T/C]G |
| | | | | | | | ATAAACTTCTATCATTGTTAGATTTTGA |
| | | | | | | | CAGAATTTGTTATATTTGTAA |
| | | | | | | | (SEQ ID NO: 58) |
| SNP_59 | 2080392 | C | T | T | T | T | GTTCGATACCATGTTAAATTAAGTTTTC |
| | | | | | | | ATTGAATGAATGAAAGTAAATT[T/C]G |
| | | | | | | | ATAACATGAATCAATACTCAAACAAAAG |
| | | | | | | | AGATATATTTCTAGAGAGGTA |
| | | | | | | | (SEQ ID NO: 59) |
| SNP_60 | 2731901 | A | G | G | G | G | AACTTCTACCAACTTCTATCAGTGTCAC |
| | | | | | | | TTAAGGGTGAGTTAGCCGATCG[A/G]C |
| | | | | | | | ATTAACAAAGtGGTCAGACGAAGATAAG |
| | | | | | | | GAAACGTTCAACTTATTCAAA |
| | | | | | | | (SEQ ID NO: 60) |
| SNP_61 | 3209067 | G | A | A | A | A | TACACTAACATCTTCGTGAAATGAATTC |
| | | | | | | | TCTTTACTACCAAGCTCAAGGC[G/A]C |
| | | | | | | | TTGATTTTTTCCTGAAACTCAGCACCCA |
| | | | | | | | TTCTAACATGAGAATCCATGT |
| | | | | | | | (SEQ ID NO: 61) |
| SNP_62 | 3885803 | C | A | A | A | A | ACCAATTTTTAAAAGTAATCTAAAATTG |
| | | | | | | | CCAATAGCTTCAATTGCAACTG[A/C]T |
| | | | | | | | TTGCCATTGCCCACATAGATGAATCTTT |
| | | | | | | | CAGCATCATTTGGCGGTCGAC |
| | | | | | | | (SEQ ID NO: 62) |

The average ToLCNDV-ES resistance score of the original donor, of the recurrent parent and of the F3 introgression line comprising all four QTLs is provided below in Table 5.

TABLE 5

| | Average | Average | Average | | |
|---|---|---|---|---|---|
| | score | score | score | | |
| | 1^{st} eval- | 2^{nd} eval- | 3^{rd} eval- | | ELISA |
| | uation - | uation - | uation - | ELISA | conclu- |
| Genotype | 25 dpi | 32 dpi | 46 dpi | OD | sion |
| Wild | 9.0 | 9.0 | 9.0 | 0.07 | negative |
| introgression | | | | | |
| donor | | | | | |
| Recurrent | 5.1 | 4.2 | 5.3 | 1.57 | positive |
| parent elite | | | | | |
| line | | | | | |
| C13_4231-7 | | | | | |
| Susceptible | 5.0 | 6.0 | 4.0 | 1.80 | positive |
| control | | | | | |
| NCIMB | 9.0 | 9.0 | 9.0 | 0.07 | negative |
| 43745 | | | | | |
| with QTL1.1, | | | | | |
| QTL1.2, | | | | | |
| QTL2.1 and | | | | | |
| QTL3.1 | | | | | |
| Positive | | | | 0.80 | positive |
| control | | | | | |
| ELISA | | | | | |
| Negative | | | | 0.07 | negative |
| control | | | | | |
| ELISA | | | | | |

In another experiment also a number of wild donors were tested for ToLCNDV-ES resistance. The same disease protocol as described above was used, except that the visual evaluation was done later after inoculation, with only two time points. The results are given in Table 6 below.

TABLE 6

| | Average score | Average score |
|---|---|---|
| Genotype | Evaluation 1 - 39 dpi | Evaluation 2 - 55 dpi |
| PI605996 | 9.0 | 9.0 |
| CGN22263 | 8.3 | 7.0 |
| CGN22932 | 9.0 | 8.4 |
| Susceptible | 3.3 | 2.5 |
| control | | |

As these wild donors have an identical SNP haplotype for the four QTLs (QTL1.1, QTL1.2, QTL2.1 and QTL3.1), except that SNP_09 and SNP_15 of PI605996 is different, it is assumed that these donors contain the same (or variant) QTLs, conferring ToLCNDV-ES resistance.

### Example 4

In WO2021019069 (and the priority document WO2021019272) cucumber plants that are tolerant to Tomato Leaf Curl New Delhi Virus are described, which comprise a first QTL, QTL1, on chromosome 1 and a second QTL, QTL2 on chromosome 2, both derived from a tolerant donor CUC29. Twelve SNP markers are said to be linked to QTL1 (Table 3 therein) and 15 to QTL2 (Table 4 therein), with the nucleotide indicative of ‘Tolerance’ being shown under the heading T-allele in Tables 3 and 4. The position of the SNPs is given with respect to the Chinese Long V2 genome. This position corresponds to the Chinese Long V3 genome as indicated in the Table below, to be able to compare the position on the chromosomes.

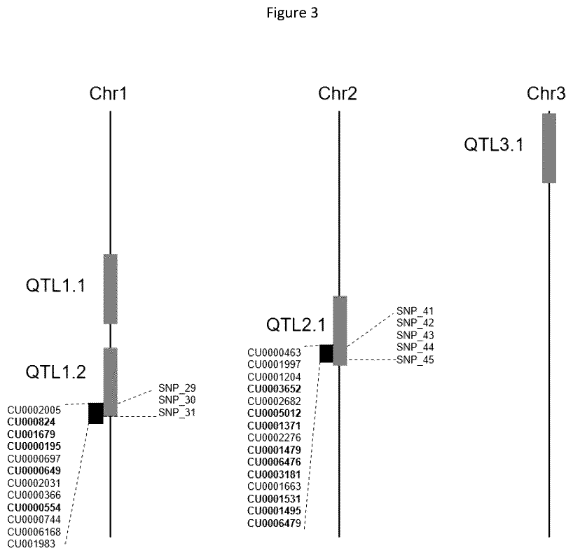

The SNP markers of WO2021019069 have been analyzed in the recurrent parent elite cucumber line and in the ToLCNDV-ES resistant donor used in the Examples above. SNP markers that are different in the CUC29 donor and in the resistant donor of the instant invention are highlighted in bold in Table 7 below and in FIG. 3.

TABLE 7

| | | | SNP haplotype | Resistant donor | |
|---|---|---|---|---|---|
| | | Position on | of donor | used to introgress | Recurrent |
| | | Chinese | CUC29 | QTL1.1, QTL1.2, | parent |
| WO2021019069 | | Long V3 | described in | QTL2.1 and | elite line |
| marker | Chromosome | genome | WO2021019069 | QTL3.1 | C13_4231-7 |
| CU0002005 | Chr_01 | 24370641 | A | R | A |
| CU0000824 | Chr_{—}01 | 24376744 | G | T | T |
| CU001679 | Chr_{—}01 | 24418455 | A | C | C |
| CU0000195 | Chr_{—}01 | 24425353 | G | C | C |
| CU0000697 | Chr_01 | 24551586 | A | R | G |
| CU0000649 | Chr_{—}01 | 24559663 | G | C | T |
| CU0002031 | Chr_01 | 24636150 | A | A | G |
| CU0000366 | Chr_01 | 25541454 | A | na | na |
| CU0000554 | Chr_{—}01 | 25707202 | A | T | G |
| CU0000744 | Chr_01 | 25715858 | A | A | G |
| CU0006168 | Chr_01 | 26222543 | G | G | G |
| CU001983 | Chr_01 | 26927420 | G | G | G |
| CU0000463 | Chr_02 | 17392473 | A | A | G |
| CU0001997 | Chr_02 | 17489172 | A | A | G |
| CU0001204 | Chr_02 | 17567541 | A | A | A |
| CU0003652 | Chr_{—}02 | 17619208 | A | C | T |
| CU0002682 | Chr_02 | 17627083 | A | na | na |
| CU0005012 | Chr_{—}02 | 17862600 | A | G | G |
| CU0001371 | Chr_{—}02 | 17869284 | A | G | G |
| CU0002276 | Chr_02 | 18054980 | A | A | G |
| CU0001479 | Chr_{—}02 | 18447612 | C | G | T |
| CU0006476 | Chr_{—}02 | 18462207 | A | T | C |
| CU0003181 | Chr_{—}02 | 18547980 | G | C | G |
| CU0001663 | Chr_02 | 18551116 | A | A | G |
| CU0001531 | Chr_{—}02 | 18597194 | A | G | G |
| CU0001495 | Chr_{—}02 | 18748949 | C | G | G |
| CU0006479 | Chr_{—}02 | 18837247 | G | A | G |

(nucleotide R is ‘A or G’)

As can be seen, the resistant donor used to introgress QTL1.1, QTL1.2, QTL2.1 and QTL3.1 (to generate NCIMB 43745) has a different SNP nucleotide/SNP haplotype from that of CUC29 for at least 5 SNPs on chromosome 1 and at least 9 SNPs on chromosome 2. Also, the other donors described herein (e.g. CGN22263, P1605996, CGN22932) each have at least 4 SNP nucleotides which are different from those of CUC29 for chromosome 1 and at least 6 SNP nucleotides which are different from those of CUC29 for chromosome 2 (data not shown). Therefore, all QTL donors described herein are different donors than CUC29.

As can be seen in Table 7 and as illustrated in FIG. 3, the chromosome 1 markers described in WO2021019069 lie in a region that overlaps partly with QTL 1.2 and the chromosome 2 markers described in WO2021019069 lie in a region that overlaps partly with QTL2.1:

TABLE 8

| WO2021019069 Chromosome 1 QTL | QTL 1.2 herein |
|---|---|
| From nucleotide 24370641 to | From nucleotide 22942981 to |
| 26927420 on chromosome 1 | 25543032 on chromosome 1 |
| WO2021019069 Chromosome 2 QTL | QTL2.1 herein |
| From nucleotide 17392473 to | From nucleotide 15218569 to |
| 18837247 on chromosome 2 | nucleotide 19535432 on |
| | chromosome 2 |

However, it can also be seen that the SNP haplotype of the markers of the ToLCNDV tolerant donor CUC29 described in WO2021019069 is completely different from the SNP haplotype of the ToLCNDV-ES donor used herein. QTL1 of WO2021019069 differs in 5 of the 12 SNP markers and QTL2 of WO2021019069 differs in 9 of the 15 SNP markers, see Table 7 above. The donor CUC29 described in WO2021019069, therefore, contains a different nucleotide sequence and different QTLs than the QTLs described herein.

Once seeds of NCIMB43427 (containing QTL1 and QTL2 of CUC29) are available for testing, the SNP markers provided herein, and the ToLCNDV phenotype will be analyzed and compared. Especially, the SNP nucleotides for markers SNP_29, SNP_30 and/or SNP_31 will be analyzed in those seeds, as these are in the same region as QTL1 of WO2021019069, and the SNP nucleotides for markers for SNP_41, SNP_42, SNP_43, SNP_44 and/or SNP_45 will be analyzed in those seeds, as these are in the same region as QTL2 of WO2021019069, see FIG. 3. It is expected that the SNP haplotype of NCIMB43427 for SNP_29, SNP_30 and SNP_31 is not C-A-G (Cytosine-Adenine-Guanine). It is also expected that the SNP haplotype of NCIMB43427 for SNP_41, SNP_42, SNP_43, SNP_44 and SNP_45 is not T-A-T-T-G (Thymine-Adenine-Thymine-Thymine-Guanine).

Thus, in one aspect the plants and plant parts provided herein do not contain the QTL 1 and/or QTL2 of CUC29 or of NCIMB43427.

### Example 5

### Materials and Methods

| | Average disease | Average disease |
|---|---|---|
| Genotype of materials used | score 25 dpi | score 32 dpi |
| Deposit NCIMB43744 (susceptible | 2.2 | 2.4 |
| BC1F4 line without QTLs) | | |
| Deposit NCIMB43745 (F3 line | 9.0 | 9.0 |
| with all four QTLs introgressed | | |
| from wild donor) | | |
| Wild donor of QTLs | 9.0 | 9.0 |

Eight replicates per genotype were grown in the field. Leaf sampling moments were at 25 dpi and 32 dpi. Leaf sampled was the first expanded leaf from the top of the plant.

A qPCR (quantitative PCR, TaqMan) was performed to quantify the viral load and calculate the fold change with 2-(ΔΔ Ct).

Statistical analysis was performed on ΔΔ Ct values to determine the significant difference of viral load between the genotypes.

Analysis was performed using deposit seeds susceptible BC1F4 line without any QTLs as reference line.

### Results:

Plants of the wild donor and deposit NCIMB43745 with all QTLs had uniformly low viral loads, with 2-(AA Ct) values 2.7×104 and 5.3×103 fold decrease of viral load measured compared to the susceptible plants on the first and second evaluation, respectively.

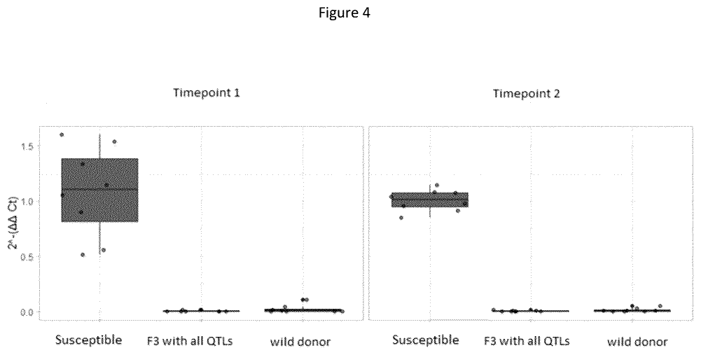

FIG. 4 shows the results in a boxplot, wherein the scale shows the fold changes. Timepoint 1 is 25 dpi and timepoint 2 is 32 dpi.

### Example 6

To determine the effect of individual QTLs and combinations of QTLs a range of lines was developed and evaluated in a filed trial. The lines were homozygous for the QTLs.

The same trial as in Example 5 was used, with eight replicates per genotype. The ToLCNDV-ES assay was done as in Example 1 and ToLCNDV disease scoring was done as in Example 1, at three timepoints with the first one being at 25 dpi. SE=Standard Error; p values are only included for the first evaluation relative to the susceptible control genotype lacking all QTLs.

The average ToLCNDV disease score of each line is presented below.

| | Average | Average | Average | | | | p.value_with |
|---|---|---|---|---|---|---|---|
| | disease | disease | disease | | | | respect of no |
| Genotype of | score at | score at | score at | SE_first | SE_second | SE_third | QTL/_first |
| the line | timepoint 1 | timepoint 2 | timepoint 3 | evaluation | evaluation | evaluation | evaluation |
| no QTLs | 2.21875 | 2.1875 | 2.4375 | 0.28087563 | 0.2612319 | 0.24545131 | 0.99727214 |
| no QTLs | 2.5 | 2.1875 | 2.40625 | 0.28087563 | 0.2612319 | 0.24545131 | |
| recurrent | 2.53125 | 2.4375 | 2.46875 | 0.28087563 | 0.2612319 | 0.24545131 | 1 |
| parent, no | | | | | | | |
| QTLs | | | | | | | |

One QTL

| | | | | | | | |
|---|---|---|---|---|---|---|---|
| QTL3.1 | 3.44598778 | 3.63519073 | 3.54519655 | 0.2832296 | 0.26349841 | 0.24695847 | 0.26822235 |
| QTL3.1 | 3.57798464 | 3.46347842 | 3.71207562 | 0.28563966 | 0.26911944 | 0.24849181 | 0.14530372 |
| QTL1.1 | 3.88914926 | 3.81845562 | 4.5625 | 0.2832296 | 0.26349841 | 0.24545131 | 0.01429821 |
| QTL2.1 | 4.5625 | 4.25 | 4.32170245 | 0.28087563 | 0.2612319 | 0.24695851 | 0.00010074 |

Two QTLs

| | | | | | | | |
|---|---|---|---|---|---|---|---|
| QTL1.1 + | 4.59375 | 4.78125 | 5 | 0.28087563 | 0.2612319 | 0.24545131 | 0.00011207 |
| QTL3.1 | | | | | | | |
| QTL1.1 + | 5.09375 | 4.48854165 | 4.97497026 | 0.28087563 | 0.26582085 | 0.24849179 | 0.00012332 |
| QTL3.1 | | | | | | | |
| QTL1.1 + | 5.25 | 5.40625 | 5.10853475 | 0.28087563 | 0.2612319 | 0.25005196 | 0.00013451 |
| QTL3.1 | | | | | | | |
| QTL1.1 + | 6.1875 | 6.15625 | 6.96875 | 0.28087563 | 0.2612319 | 0.24545131 | 0.00014563 |
| QTL2.1 | | | | | | | |
| QTL2.1 + | 6.4375 | 6.5625 | 5.22157144 | 0.28087563 | 0.2612319 | 0.24849178 | 0.00026925 |
| QTL3.1 | | | | | | | |
| QTL1.1 + | 6.43878309 | 6.11045919 | 6.50145167 | 0.2832296 | 0.26349844 | 0.2469585 | 0.00025791 |
| QTL2.1 | | | | | | | |
| QTL1.1 + | 6.53125 | 6.71875 | 6.875 | 0.28087563 | 0.2612319 | 0.24545131 | 0.00028057 |
| QTL1.2 | | | | | | | |
| QTL1.1 + | 6.65625 | 6.25 | 6.75 | 0.28087563 | 0.2612319 | 0.24545131 | 0.00015671 |
| QTL2.1 | | | | | | | |
| QTL1.2 + | 6.6875 | 6.75 | 6.52310873 | 0.28087563 | 0.2612319 | 0.24695851 | 0.00029186 |
| QTL2.1 | | | | | | | |
| QTL2.1 + | 7.0480117 | 7.16418787 | 5.52310873 | 0.28322963 | 0.26349844 | 0.24695851 | 0.00030312 |
| QTL3.1 | | | | | | | |
| QTL2.1 + | 7.40625 | 7.21875 | 5.19738114 | 0.28087563 | 0.2612319 | 0.2469585 | 0.00036124 |
| QTL3.1 | | | | | | | |
| QTL2.1 + | 7.96875 | 7.59375 | 6.6875 | 0.28087563 | 0.2612319 | 0.24545131 | 0.00035451 |
| QTL3.1 | | | | | | | |

Three QTLs

| | | | | | | | |
|---|---|---|---|---|---|---|---|
| QTL1.1 + | 8.09375 | 7.71875 | 7.5 | 0.28087563 | 0.2612319 | 0.24545131 | 0.00020822 |
| QTL2.1 + | | | | | | | |
| QTL3.1 | | | | | | | |
| QTL1.1 + | 8.3125 | 8.03125 | 7.75 | 0.28087563 | 0.2612319 | 0.24545131 | 0.00020243 |
| QTL2.1 + | | | | | | | |
| QTL3.1 | | | | | | | |
| QTL1.1 + | 8.34375 | 8.15625 | 7.65625 | 0.28087563 | 0.2612319 | 0.24545131 | 0.00020628 |
| QTL2.1 + | | | | | | | |
| QTL3.1 | | | | | | | |
| QTL1.1 + | 8.34375 | 7.875 | 8.0625 | 0.28087563 | 0.2612319 | 0.24545131 | 0.00036465 |
| QTL2.1 + | | | | | | | |
| QTL3.1 | | | | | | | |
| QTL1.1 + | 8.4375 | 8.28125 | 7.78125 | 0.28087563 | 0.2612319 | 0.24545131 | 0.0003512 |
| QTL2.1 + | | | | | | | |
| QTL3.1 | | | | | | | |
| QTL1.1 + | 8.5625 | 8.34375 | 8.0625 | 0.28087563 | 0.2612319 | 0.24545131 | 0.00020435 |
| QTL2.1 + | | | | | | | |
| QTL3.1 | | | | | | | |
| QTL1.1 + | 8.59375 | 8.46875 | 7.90625 | 0.28087563 | 0.2612319 | 0.24545131 | 0.00035786 |
| QTL2.1 + | | | | | | | |
| QTL3.1 | | | | | | | |
| QTL1.1 + | 8.59375 | 8.5625 | 8.125 | 0.28087563 | 0.2612319 | 0.24545131 | 0.00021019 |
| QTL2.1 + | | | | | | | |
| QTL3.1 | | | | | | | |
| QTL 1.1+ | 8.71875 | 8.59375 | 8.375 | 0.28087563 | 0.2612319 | 0.24545131 | 0.00021218 |
| QTL1.2 + | | | | | | | |
| QTL2.1 | | | | | | | |

Four QTLs

| | | | | | | | |
|---|---|---|---|---|---|---|---|
| QTL1.1 + | 8.9375 | 9 | 8.96875 | 0.28087563 | 0.2612319 | 0.24545131 | 0.00034791 |
| QTL1.2 + | | | | | | | |
| QTL2.1 + | | | | | | | |
| QTL3.1 | | | | | | | |
| QTL1.1 + | 8.9375 | 8.78125 | 8.70358421 | 0.28087563 | 0.2612319 | 0.24849178 | 0.00020054 |
| QTL1.2 + | | | | | | | |
| QTL2.1 + | | | | | | | |
| QTL3.1 | | | | | | | |
| wild donor | 9 | 8.96875 | 8.96875 | 0.28087563 | 0.2612319 | 0.24545131 | 0.0003681 |
| of QTLs | | | | | | | |

As can be seen, four QTLs results in the highest resistance level, but also a combination of three QTLs results in a resistance level with a score of above 8.0. Individual QTLs give a slight increase in resistance and combinations of two QTLs give a higher increase than single QTLs. The resistance level if maintained over the three evaluations, except for the combination of QTL2.1 and QTL3.1, for which a decline was seen at the third evaluation time point.

Preferred combinations are, therefore, plants comprising at least three QTLs selected from QTL1.1, QTL1.2, QTL2.1 and QTL3.1. Another preferred combination is QTL 1.1 with at least one other QTL, preferably with at least two other QTLs selected from QTL1.2, QTL2.1 and QTL3.1. Further, also QTL3.1 with at least one other QTL, preferably at least two other QTLs selected from QTL1.1, QTL1.2 and QTL2.1 is a specific embodiment.

1. A cultivated Cucumis sativus var. sativus plant comprising at least two introgression fragments on chromosome 1, 2 and/or 3 from a wild cucumber donor wherein one of said fragments comprises QTL1.1, or said plant comprises at least three introgression fragments on chromosome 1, 2 and/or 3, wherein each of said introgression fragments comprises a Quantitative Trait Locus (QTL) selected from the QTLs designated QTL1.1, QTL1.2, QTL2.1 and QTL3.1, wherein QTL1.1 is located on chromosome 1 between the Single Nucleotide Polymorphism marker SNP_01 at nucleotide 51 of SEQ ID NO: 1 and SNP_16 at nucleotide 51 of SEQ ID NO: 16, QTL1.2 is located on chromosome 1 between SNP_17 at nucleotide 51 of SEQ ID NO: 17 and SNP_31 at nucleotide 51 of SEQ ID NO: 31, QTL2.1 is located on chromosome 2 between SNP_32 at nucleotide 51 of SEQ ID NO: 32 and SNP_47 at nucleotide 51 of SEQ ID NO: 47, and QTL3.1 is located on chromosome 3 between SNP_48 at nucleotide 51 of SEQ ID NO: 48 and SNP_62 at nucleotide 51 of SEQ ID NO: 62, which QTLs confer an increase in Tomato Leaf Curl New Delhi Virus strain ToLCNDV-ES and
wherein said introgression fragment on chromosome 1 comprising QTL1.1 comprises a SNP haplotype or SNP genotype of at least 5, preferably-at-last markers selected from the group consisting of:
a) the TX or TT genotype for the Single Nucleotide Polymorphism marker SNP_01 at nucleotide 51 of SEQ ID NO: 1 or at the equivalent position in a sequence comprising at least 95% sequence identity to SEQ ID NO: 1;
b) the TX or TT genotype for the Single Nucleotide Polymorphism marker SNP_02 at nucleotide 51 of SEQ ID NO: 2 or at the equivalent position in a sequence comprising at least 95% sequence identity to SEQ ID NO: 2;
c) the TX or TT genotype for the Single Nucleotide Polymorphism marker SNP_03 at nucleotide 51 of SEQ ID NO: 3 or at the equivalent position in a sequence comprising at least 95% sequence identity to SEQ ID NO: 3;
d) the TX or TT genotype for the Single Nucleotide Polymorphism marker SNP_04 at nucleotide 51 of SEQ ID NO: 4 or at the equivalent position in a sequence comprising at least 95% sequence identity to SEQ ID NO: 4;
e) the AX or AA genotype for the Single Nucleotide Polymorphism marker SNP_05 at nucleotide 51 of SEQ ID NO: 5 or at the equivalent position in a sequence comprising at least 95% sequence identity to SEQ ID NO: 5;
f) the CX or CC genotype for the Single Nucleotide Polymorphism marker SNP_06 at nucleotide 51 of SEQ ID NO: 6 or at the equivalent position in a sequence comprising at least 95% sequence identity to SEQ ID NO: 6;
g) the TX or TT genotype for the Single Nucleotide Polymorphism marker SNP_07 at nucleotide 51 of SEQ ID NO: 7 or at the equivalent position in a sequence comprising at least 95% sequence identity to SEQ ID NO: 7;
h) the AX or AA genotype for the Single Nucleotide Polymorphism marker SNP_08 at nucleotide 51 of SEQ ID NO: 8 or at the equivalent position in a sequence comprising at least 95% sequence identity to SEQ ID NO: 8;
i) the GX or GG genotype for the Single Nucleotide Polymorphism marker SNP_09 at nucleotide 51 of SEQ ID NO: 9 or at the equivalent position in a sequence comprising at least 95% sequence identity to SEQ ID NO: 9; or the AX or AA genotype for the Single Nucleotide Polymorphism marker SNP_09 at nucleotide 51 of SEQ ID NO: 9 or at the equivalent position in a sequence comprising at least 95% sequence identity to SEQ ID NO: 9;
j) the CX or CC genotype for the Single Nucleotide Polymorphism marker SNP_10 at nucleotide 51 of SEQ ID NO: 10 or at the equivalent position in a sequence comprising at least 95% sequence identity to SEQ ID NO: 10;
k) the CX or CC genotype for the Single Nucleotide Polymorphism marker SNP_11 at nucleotide 51 of SEQ ID NO: 11 or at the equivalent position in a sequence comprising at least 95% sequence identity to SEQ ID NO: 11;
l) the CX or CC genotype for the Single Nucleotide Polymorphism marker SNP_12 at nucleotide 51 of SEQ ID NO: 12 or at the equivalent position in a sequence comprising at least 95% sequence identity to SEQ ID NO: 12;
m) the TX or TT genotype for the Single Nucleotide Polymorphism marker SNP_13 at nucleotide 51 of SEQ ID NO: 13 or at the equivalent position in a sequence comprising at least 95% sequence identity to SEQ ID NO: 13;
n) the AX or AA genotype for the Single Nucleotide Polymorphism marker SNP_14 at nucleotide 51 of SEQ ID NO: 14 or at the equivalent position in a sequence comprising at least 95% sequence identity to SEQ ID NO: 14;
o) the CX or CC genotype for the Single Nucleotide Polymorphism marker SNP_15 at nucleotide 51 of SEQ ID NO: 15 or at the equivalent position in a sequence comprising at least 95% sequence identity to SEQ ID NO: 15; or the TX or TT genotype for the Single Nucleotide Polymorphism marker SNP_15 at nucleotide 51 of SEQ ID NO: 15 or at the equivalent position in a sequence comprising at least 95% sequence identity to SEQ ID NO: 15; and
p) the GX or GG genotype for the Single Nucleotide Polymorphism marker SNP_16 at nucleotide 51 of SEQ ID NO: 16 or at the equivalent position in a sequence comprising at least 95% sequence identity to SEQ ID NO: 16;
wherein said introgression fragment on chromosome 1 comprising QTL1.2 comprises a SNP haplotype or SNP genotype of at least 5, markers selected from the group consisting of:
a) the CX or CC genotype for the Single Nucleotide Polymorphism marker SNP_17 at nucleotide 51 of SEQ ID NO: 17 or at the equivalent position in a sequence comprising at least 95% sequence identity to SEQ ID NO: 17;
b) the GX or GG genotype for the Single Nucleotide Polymorphism marker SNP_18 at nucleotide 51 of SEQ ID NO: 18 or at the equivalent position in a sequence comprising at least 95% sequence identity to SEQ ID NO: 18;
c) the GX or GG genotype for the Single Nucleotide Polymorphism marker SNP_19 at nucleotide 51 of SEQ ID NO: 19 or at the equivalent position in a sequence comprising at least 95% sequence identity to SEQ ID NO: 19;
d) the TX or TT genotype for the Single Nucleotide Polymorphism marker SNP_20 at nucleotide 51 of SEQ ID NO: 20 or at the equivalent position in a sequence comprising at least 95% sequence identity to SEQ ID NO: 20;
e) the GX or GG genotype for the Single Nucleotide Polymorphism marker SNP_21 at nucleotide 51 of SEQ ID NO: 21 or at the equivalent position in a sequence comprising at least 95% sequence identity to SEQ ID NO: 21;
f) the GX or GG genotype for the Single Nucleotide Polymorphism marker SNP_22 at nucleotide 51 of SEQ ID NO: 22 or at the equivalent position in a sequence comprising at least 95% sequence identity to SEQ ID NO: 22;
g) the TX or TT genotype for the Single Nucleotide Polymorphism marker SNP_23 at nucleotide 51 of SEQ ID NO: 23 or at the equivalent position in a sequence comprising at least 95% sequence identity to SEQ ID NO: 23;
h) the CX or CC genotype for the Single Nucleotide Polymorphism marker SNP_24 at nucleotide 51 of SEQ ID NO: 24 or at the equivalent position in a sequence comprising at least 95% sequence identity to SEQ ID NO: 24;
i) the AX or AA genotype for the Single Nucleotide Polymorphism marker SNP_25 at nucleotide 51 of SEQ ID NO: 25 or at the equivalent position in a sequence comprising at least 95% sequence identity to SEQ ID NO: 25;
j) the CX or CC genotype for the Single Nucleotide Polymorphism marker SNP_26 at nucleotide 51 of SEQ ID NO: 26 or at the equivalent position in a sequence comprising at least 95% sequence identity to SEQ ID NO: 26;
k) the CX or CC genotype for the Single Nucleotide Polymorphism marker SNP_27 at nucleotide 51 of SEQ ID NO: 27 or at the equivalent position in a sequence comprising at least 95% sequence identity to SEQ ID NO: 27;
l) the CX or CC genotype for the Single Nucleotide Polymorphism marker SNP_28 at nucleotide 51 of SEQ ID NO: 28 or at the equivalent position in a sequence comprising at least 95% sequence identity to SEQ ID NO: 28;
m) the CX or CC genotype for the Single Nucleotide Polymorphism marker SNP_29 at nucleotide 51 of SEQ ID NO: 29 or at the equivalent position in a sequence comprising at least 95% sequence identity to SEQ ID NO: 29;
n) the AX or AA genotype for the Single Nucleotide Polymorphism marker SNP_30 at nucleotide 51 of SEQ ID NO: 30 or at the equivalent position in a sequence comprising at least 95% sequence identity to SEQ ID NO: 30; and
o) the GX or GG genotype for the Single Nucleotide Polymorphism marker SNP_31 at nucleotide 51 of SEQ ID NO: 31 or at the equivalent position in a sequence comprising at least 95% sequence identity to SEQ ID NO: 31;
wherein said introgression fragment on chromosome 2 comprising QTL2.1 comprises a SNP haplotype or SNP genotype of at least 5 markers selected from the group consisting of:
a) the TX or TT genotype for the Single Nucleotide Polymorphism marker SNP_32 at nucleotide 51 of SEQ ID NO: 32 or at the equivalent position in a sequence comprising at least 95% sequence identity to SEQ ID NO: 32;
b) the TX or TT genotype for the Single Nucleotide Polymorphism marker SNP_33 at nucleotide 51 of SEQ ID NO: 33 or at the equivalent position in a sequence comprising at least 95% sequence identity to SEQ ID NO: 33;
c) the CX or CC genotype for the Single Nucleotide Polymorphism marker SNP_34 at nucleotide 51 of SEQ ID NO: 34 or at the equivalent position in a sequence comprising at least 95% sequence identity to SEQ ID NO: 34;
d) the AX or AA genotype for the Single Nucleotide Polymorphism marker SNP_35 at nucleotide 51 of SEQ ID NO: 35 or at the equivalent position in a sequence comprising at least 95% sequence identity to SEQ ID NO: 35;
e) the TX or TT genotype for the Single Nucleotide Polymorphism marker SNP_36 at nucleotide 51 of SEQ ID NO: 36 or at the equivalent position in a sequence comprising at least 95% sequence identity to SEQ ID NO: 36;
f) the CX or CC genotype for the Single Nucleotide Polymorphism marker SNP_37 at nucleotide 51 of SEQ ID NO: 37 or at the equivalent position in a sequence comprising at least 95% sequence identity to SEQ ID NO: 37;
g) the GX or GG genotype for the Single Nucleotide Polymorphism marker SNP_38 at nucleotide 51 of SEQ ID NO: 38 or at the equivalent position in a sequence comprising at least 95% sequence identity to SEQ ID NO: 38;
h) the CX or CC genotype for the Single Nucleotide Polymorphism marker SNP_39 at nucleotide 51 of SEQ ID NO: 39 or at the equivalent position in a sequence comprising at least 95% sequence identity to SEQ ID NO: 39;
i) the GX or GG genotype for the Single Nucleotide Polymorphism marker SNP_40 at nucleotide 51 of SEQ ID NO: 40 or at the equivalent position in a sequence comprising at least 95% sequence identity to SEQ ID NO: 40;
j) the TX or TT genotype for the Single Nucleotide Polymorphism marker SNP_41 at nucleotide 51 of SEQ ID NO: 41 or at the equivalent position in a sequence comprising at least 95% sequence identity to SEQ ID NO: 41;
k) the AX or AA genotype for the Single Nucleotide Polymorphism marker SNP_42 at nucleotide 51 of SEQ ID NO: 42 or at the equivalent position in a sequence comprising at least 95% sequence identity to SEQ ID NO: 42;
l) the TX or TT genotype for the Single Nucleotide Polymorphism marker SNP_43 at nucleotide 51 of SEQ ID NO: 43 or at the equivalent position in a sequence comprising at least 95% sequence identity to SEQ ID NO: 43;
m) the TX or TT genotype for the Single Nucleotide Polymorphism marker SNP_44 at nucleotide 51 of SEQ ID NO: 44 or at the equivalent position in a sequence comprising at least 95% sequence identity to SEQ ID NO: 44;
n) the GX or GG genotype for the Single Nucleotide Polymorphism marker SNP_45 at nucleotide 51 of SEQ ID NO: 45 or at the equivalent position in a sequence comprising at least 95% sequence identity to SEQ ID NO: 45;
o) the AX or AA genotype for the Single Nucleotide Polymorphism marker SNP_46 at nucleotide 51 of SEQ ID NO: 46 or at the equivalent position in a sequence comprising at least 95% sequence identity to SEQ ID NO: 46; and
p) the GX or GG genotype for the Single Nucleotide Polymorphism marker SNP_47 at nucleotide 51 of SEQ ID NO: 47 or at the equivalent position in a sequence comprising at least 95% sequence identity to SEQ ID NO: 47;
wherein said introgression fragment on chromosome 3 comprising QTL3.1 comprises a SNP haplotype or SNP genotype of at least 5 markers selected from the group consisting of:
a) the AX or AA genotype for the Single Nucleotide Polymorphism marker SNP_48 at nucleotide 51 of SEQ ID NO: 48 or at the equivalent position in a sequence comprising at least 95% sequence identity to SEQ ID NO: 48;
b) the CX or CC genotype for the Single Nucleotide Polymorphism marker SNP_49 at nucleotide 51 of SEQ ID NO: 49 or at the equivalent position in a sequence comprising at least 95% sequence identity to SEQ ID NO: 49;
c) the CX or CC genotype for the Single Nucleotide Polymorphism marker SNP_50 at nucleotide 51 of SEQ ID NO: 50 or at the equivalent position in a sequence comprising at least 95% sequence identity to SEQ ID NO: 50;
d) the CX or CC genotype for the Single Nucleotide Polymorphism marker SNP_51 at nucleotide 51 of SEQ ID NO: 51 or at the equivalent position in a sequence comprising at least 95% sequence identity to SEQ ID NO: 51;
e) the TX or TT genotype for the Single Nucleotide Polymorphism marker SNP_52 at nucleotide 51 of SEQ ID NO: 52 or at the equivalent position in a sequence comprising at least 95% sequence identity to SEQ ID NO: 52;
f) the CX or CC genotype for the Single Nucleotide Polymorphism marker SNP_53 at nucleotide 51 of SEQ ID NO: 53 or at the equivalent position in a sequence comprising at least 95% sequence identity to SEQ ID NO: 53;
g) the CX or CC genotype for the Single Nucleotide Polymorphism marker SNP_54 at nucleotide 51 of SEQ ID NO: 54 or at the equivalent position in a sequence comprising at least 95% sequence identity to SEQ ID NO: 54;
h) the CX or CC genotype for the Single Nucleotide Polymorphism marker SNP_55 at nucleotide 51 of SEQ ID NO: 55 or at the equivalent position in a sequence comprising at least 95% sequence identity to SEQ ID NO: 55;
i) the GX or GG genotype for the Single Nucleotide Polymorphism marker SNP_56 at nucleotide 51 of SEQ ID NO: 56 or at the equivalent position in a sequence comprising at least 95% sequence identity to SEQ ID NO: 56;
j) the CX or CC genotype for the Single Nucleotide Polymorphism marker SNP_57 at nucleotide 51 of SEQ ID NO: 57 or at the equivalent position in a sequence comprising at least 95% sequence identity to SEQ ID NO: 57;
k) the TX or TT genotype for the Single Nucleotide Polymorphism marker SNP_58 at nucleotide 51 of SEQ ID NO: 58 or at the equivalent position in a sequence comprising at least 95% sequence identity to SEQ ID NO: 58;
l) the TX or TT genotype for the Single Nucleotide Polymorphism marker SNP_59 at nucleotide 51 of SEQ ID NO: 59 or at the equivalent position in a sequence comprising at least 95% sequence identity to SEQ ID NO: 59;
m) the GX or GG genotype for the Single Nucleotide Polymorphism marker SNP_60 at nucleotide 51 of SEQ ID NO: 60 or at the equivalent position in a sequence comprising at least 95% sequence identity to SEQ ID NO: 60;
n) the AX or AA genotype for the Single Nucleotide Polymorphism marker SNP_61 at nucleotide 51 of SEQ ID NO: 61 or at the equivalent position in a sequence comprising at least 95% sequence identity to SEQ ID NO: 61; and
o) the AX or AA genotype for the Single Nucleotide Polymorphism marker SNP_62 at nucleotide 51 of SEQ ID NO: 62 or at the equivalent position in a sequence comprising at least 95% sequence identity to SEQ ID NO: 62.

2. The plant according to claim 1, wherein the plant comprises at least the introgression fragment comprising QTL1.1 and QTL1.2.

3. The plant according to claim 1, wherein the plant comprises at least the introgression fragment comprising QTL1.1 or QTL1.2 and QTL2.1.

4. The plant according to claim 1, wherein the introgression fragments are in homozygous form.

5. The plant according to claim 1, wherein the introgression fragment comprising the QTL is obtainable from NCIMB43745, P1605996, CGN22263, CGN22932 or P1197087.

6. The plant according to claim 1, wherein the QTL is the QTL present in seeds deposited under accession number NCIMB 43745.

7. The plant according to claim 1, wherein said introgression fragment is obtainable by crossing a plant grown from seeds deposited under accession number NCIMB 43745 with another cucumber plant.

8. Seeds from which a plant according to claim 1 can be grown.

9. A cucumber fruit harvested from a plant according to claim 1.

10. A plant cell, tissue or plant part of a plant according to claim 1.

11. A method for identifying a wild cucumber comprising a ToLCNDV-ES resistance QTL on chromosome 1, 2 and/or 3, said method comprising:
a) providing a wild or primitive cucumber accession or several accessions;
b) screening said accession(s) using a molecular marker assay which detects at least 1, 2, 3, 4, 5, 6, 7, 8, 9, 10, 11, 12, 13, 14, 15 or more of the SNP markers linked to at least one QTL selected from QTL1.1, QTL1.2, QTL2.1 and/or QTL3.1; and
c) identifying and/or selecting an accession from b) comprising the SNP haplotype or SNP genotype of at least 5, 6, 7, 8, 9, 10, 11, 12, 13, 14, 15 or 16 of the SNP markers linked to a QTL, selected from:
for QTL1.1:
a) the TX or TT genotype for the Single Nucleotide Polymorphism marker SNP_01 at nucleotide 51 of SEQ ID NO: 1 (or at nucleotide 51 of a sequence comprising at least 95% sequence identity to SEQ ID NO: 1);
b) the TX or TT genotype for the Single Nucleotide Polymorphism marker SNP_02 at nucleotide 51 of SEQ ID NO: 2 (or at nucleotide 51 of a sequence comprising at least 95% sequence identity to SEQ ID NO: 2);
c) the TX or TT genotype for the Single Nucleotide Polymorphism marker SNP_03 at nucleotide 51 of SEQ ID NO: 3 (or at nucleotide 51 of a sequence comprising at least 95% sequence identity to SEQ ID NO: 3);
d) the TX or TT genotype for the Single Nucleotide Polymorphism marker SNP_04 at nucleotide 51 of SEQ ID NO: 4 (or at nucleotide 51 of a sequence comprising at least 95% sequence identity to SEQ ID NO: 4);
e) the AX or AA genotype for the Single Nucleotide Polymorphism marker SNP_05 at nucleotide 51 of SEQ ID NO: 5 (or at nucleotide 51 of a sequence comprising at least 95% sequence identity to SEQ ID NO: 5);
f) the CX or CC genotype for the Single Nucleotide Polymorphism marker SNP_06 at nucleotide 51 of SEQ ID NO: 6 (or at nucleotide 51 of a sequence comprising at least 95% sequence identity to SEQ ID NO: 6);
g) the TX or TT genotype for the Single Nucleotide Polymorphism marker SNP_07 at nucleotide 51 of SEQ ID NO: 7 (or at nucleotide 51 of a sequence comprising at least 95% sequence identity to SEQ ID NO: 7);
h) the AX or AA genotype for the Single Nucleotide Polymorphism marker SNP_08 at nucleotide 51 of SEQ ID NO: 8 (or at nucleotide 51 of a sequence comprising at least 95% sequence identity to SEQ ID NO: 8);
i) the GX or GG genotype for the Single Nucleotide Polymorphism marker SNP_09 at nucleotide 51 of SEQ ID NO: 9 (or at nucleotide 51 of a sequence comprising at least 95% sequence identity to SEQ ID NO: 9); or the AX or AA genotype for the Single Nucleotide Polymorphism marker SNP_09 at nucleotide 51 of SEQ ID NO: 9 (or at nucleotide 51 of a sequence comprising at least 95% sequence identity to SEQ ID NO: 9);
j) the CX or CC genotype for the Single Nucleotide Polymorphism marker SNP_10 at nucleotide 51 of SEQ ID NO: 10 (or at nucleotide 51 of a sequence comprising at least 95% sequence identity to SEQ ID NO: 10);
k) the CX or CC genotype for the Single Nucleotide Polymorphism marker SNP_11 at nucleotide 51 of SEQ ID NO: 11 (or at nucleotide 51 of a sequence comprising at least 95% sequence identity to SEQ ID NO: 11);
l) the CX or CC genotype for the Single Nucleotide Polymorphism marker SNP_12 at nucleotide 51 of SEQ ID NO: 12 (or at nucleotide 51 of a sequence comprising at least 95% sequence identity to SEQ ID NO: 12);
m) the TX or TT genotype for the Single Nucleotide Polymorphism marker SNP_13 at nucleotide 51 of SEQ ID NO: 13 (or at nucleotide 51 of a sequence comprising at least 95% sequence identity to SEQ ID NO: 13);
n) the AX or AA genotype for the Single Nucleotide Polymorphism marker SNP_14 at nucleotide 51 of SEQ ID NO: 14 (or at nucleotide 51 of a sequence comprising at least 95% sequence identity to SEQ ID NO: 14);
o) the CX or CC genotype for the Single Nucleotide Polymorphism marker SNP_15 at nucleotide 51 of SEQ ID NO: 15 (or at nucleotide 51 of a sequence comprising at least 95% sequence identity to SEQ ID NO: 15); or the TX or TT genotype for the Single Nucleotide Polymorphism marker SNP_15 at nucleotide 51 of SEQ ID NO: 15 (or at nucleotide 51 of a sequence comprising at least 95% sequence identity to SEQ ID NO: 15);
p) the GX or GG genotype for the Single Nucleotide Polymorphism marker SNP_16 at nucleotide 51 of SEQ ID NO: 16 (or at nucleotide 51 of a sequence comprising at least 95% sequence identity to SEQ ID NO: 16).
for QTL1.2:
a) the CX or CC genotype for the Single Nucleotide Polymorphism marker SNP_17 at nucleotide 51 of SEQ ID NO: 17 (or at nucleotide 51 of a sequence comprising at least 95% sequence identity to SEQ ID NO: 17);
b) the GX or GG genotype for the Single Nucleotide Polymorphism marker SNP_18 at nucleotide 51 of SEQ ID NO: 18 (or at nucleotide 51 of a sequence comprising at least 95% sequence identity to SEQ ID NO: 18);
c) the GX or GG genotype for the Single Nucleotide Polymorphism marker SNP_19 at nucleotide 51 of SEQ ID NO: 19 (or at nucleotide 51 of a sequence comprising at least 95% sequence identity to SEQ ID NO: 19);
d) the TX or TT genotype for the Single Nucleotide Polymorphism marker SNP_20 at nucleotide 51 of SEQ ID NO: 20 (or at nucleotide 51 of a sequence comprising at least 95% sequence identity to SEQ ID NO: 20);
e) the GX or GG genotype for the Single Nucleotide Polymorphism marker SNP_21 at nucleotide 51 of SEQ ID NO: 21 (or at nucleotide 51 of a sequence comprising at least 95% sequence identity to SEQ ID NO: 21);
f) the GX or GG genotype for the Single Nucleotide Polymorphism marker SNP_22 at nucleotide 51 of SEQ ID NO: 22 (or at nucleotide 51 of a sequence comprising at least 95% sequence identity to SEQ ID NO: 22);
g) the TX or TT genotype for the Single Nucleotide Polymorphism marker SNP_23 at nucleotide 51 of SEQ ID NO: 23 (or at nucleotide 51 of a sequence comprising at least 95% sequence identity to SEQ ID NO: 23);
h) the CX or CC genotype for the Single Nucleotide Polymorphism marker SNP_24 at nucleotide 51 of SEQ ID NO: 24 (or at nucleotide 51 of a sequence comprising at least 95% sequence identity to SEQ ID NO: 24);
i) the AX or AA genotype for the Single Nucleotide Polymorphism marker SNP_25 at nucleotide 51 of SEQ ID NO: 25 (or at nucleotide 51 of a sequence comprising at least 95% sequence identity to SEQ ID NO: 25);
j) the CX or CC genotype for the Single Nucleotide Polymorphism marker SNP_26 at nucleotide 51 of SEQ ID NO: 26 (or at nucleotide 51 of a sequence comprising at least 95% sequence identity to SEQ ID NO: 26);
k) the CX or CC genotype for the Single Nucleotide Polymorphism marker SNP_27 at nucleotide 51 of SEQ ID NO: 27 (or at nucleotide 51 of a sequence comprising at least 95% sequence identity to SEQ ID NO: 27);
l) the CX or CC genotype for the Single Nucleotide Polymorphism marker SNP_28 at nucleotide 51 of SEQ ID NO: 28 (or at nucleotide 51 of a sequence comprising at least 95% sequence identity to SEQ ID NO: 28);
m) the CX or CC genotype for the Single Nucleotide Polymorphism marker SNP_29 at nucleotide 51 of SEQ ID NO: 29 (or at nucleotide 51 of a sequence comprising at least 95% sequence identity to SEQ ID NO: 29);
n) the AX or AA genotype for the Single Nucleotide Polymorphism marker SNP_30 at nucleotide 51 of SEQ ID NO: 30 (or at nucleotide 51 of a sequence comprising at least 95% sequence identity to SEQ ID NO: 30);
o) the GX or GG genotype for the Single Nucleotide Polymorphism marker SNP_31 at nucleotide 51 of SEQ ID NO: 31 (or at nucleotide 51 of a sequence comprising at least 95% sequence identity to SEQ ID NO: 31).
for QTL2.1:
a) the TX or TT genotype for the Single Nucleotide Polymorphism marker SNP_32 at nucleotide 51 of SEQ ID NO: 32 (or at nucleotide 51 of a sequence comprising at least 95% sequence identity to SEQ ID NO: 32);
b) the TX or TT genotype for the Single Nucleotide Polymorphism marker SNP_33 at nucleotide 51 of SEQ ID NO: 33 (or at nucleotide 51 of a sequence comprising at least 95% sequence identity to SEQ ID NO: 33);
c) the CX or CC genotype for the Single Nucleotide Polymorphism marker SNP_34 at nucleotide 51 of SEQ ID NO: 34 (or at nucleotide 51 of a sequence comprising at least 95% sequence identity to SEQ ID NO: 34);
d) the AX or AA genotype for the Single Nucleotide Polymorphism marker SNP_35 at nucleotide 51 of SEQ ID NO: 35 (or at nucleotide 51 of a sequence comprising at least 95% sequence identity to SEQ ID NO: 35);
e) the TX or TT genotype for the Single Nucleotide Polymorphism marker SNP_36 at nucleotide 51 of SEQ ID NO: 36 (or at nucleotide 51 of a sequence comprising at least 95% sequence identity to SEQ ID NO: 36);
f) the CX or CC genotype for the Single Nucleotide Polymorphism marker SNP_37 at nucleotide 51 of SEQ ID NO: 37 (or at nucleotide 51 of a sequence comprising at least 95% sequence identity to SEQ ID NO: 37);
g) the GX or GG genotype for the Single Nucleotide Polymorphism marker SNP_38 at nucleotide 51 of SEQ ID NO: 38 (or at nucleotide 51 of a sequence comprising at least 95% sequence identity to SEQ ID NO: 38);
h) the CX or CC genotype for the Single Nucleotide Polymorphism marker SNP_39 at nucleotide 51 of SEQ ID NO: 39 (or at nucleotide 51 of a sequence comprising at least 95% sequence identity to SEQ ID NO: 39);
i) the GX or GG genotype for the Single Nucleotide Polymorphism marker SNP_40 at nucleotide 51 of SEQ ID NO: 40 (or at nucleotide 51 of a sequence comprising at least 95% sequence identity to SEQ ID NO: 40);
j) the TX or TT genotype for the Single Nucleotide Polymorphism marker SNP_41 at nucleotide 51 of SEQ ID NO: 41 (or at nucleotide 51 of a sequence comprising at least 95% sequence identity to SEQ ID NO: 41);
k) the AX or AA genotype for the Single Nucleotide Polymorphism marker SNP_42 at nucleotide 51 of SEQ ID NO: 42 (or at nucleotide 51 of a sequence comprising at least 95% sequence identity to SEQ ID NO: 42);
l) the TX or TT genotype for the Single Nucleotide Polymorphism marker SNP_43 at nucleotide 51 of SEQ ID NO: 43 (or at nucleotide 51 of a sequence comprising at least 95% sequence identity to SEQ ID NO: 43);
m) the TX or TT genotype for the Single Nucleotide Polymorphism marker SNP_44 at nucleotide 51 of SEQ ID NO: 44 (or at nucleotide 51 of a sequence comprising at least 95% sequence identity to SEQ ID NO: 44);
n) the GX or GG genotype for the Single Nucleotide Polymorphism marker SNP_45 at nucleotide 51 of SEQ ID NO: 45 (or at nucleotide 51 of a sequence comprising at least 95% sequence identity to SEQ ID NO: 45);
o) the AX or AA genotype for the Single Nucleotide Polymorphism marker SNP_46 at nucleotide 51 of SEQ ID NO: 46 (or at nucleotide 51 of a sequence comprising at least 95% sequence identity to SEQ ID NO: 46);
p) the GX or GG genotype for the Single Nucleotide Polymorphism marker SNP_47 at nucleotide 51 of SEQ ID NO: 47 (or at nucleotide 51 of a sequence comprising at least 95% sequence identity to SEQ ID NO: 47).
for QTL3.1:
a) the AX or AA genotype for the Single Nucleotide Polymorphism marker SNP_48 at nucleotide 51 of SEQ ID NO: 48 (or at nucleotide 51 of a sequence comprising at least 95% sequence identity to SEQ ID NO: 48);
b) the CX or CC genotype for the Single Nucleotide Polymorphism marker SNP_49 at nucleotide 51 of SEQ ID NO: 49 (or at nucleotide 51 of a sequence comprising at least 95% sequence identity to SEQ ID NO: 49);
c) the CX or CC genotype for the Single Nucleotide Polymorphism marker SNP_50 at nucleotide 51 of SEQ ID NO: 50 (or at nucleotide 51 of a sequence comprising at least 95% sequence identity to SEQ ID NO: 50);
d) the CX or CC genotype for the Single Nucleotide Polymorphism marker SNP_51 at nucleotide 51 of SEQ ID NO: 51 (or at nucleotide 51 of a sequence comprising at least 95% sequence identity to SEQ ID NO: 51);
e) the TX or TT genotype for the Single Nucleotide Polymorphism marker SNP_52 at nucleotide 51 of SEQ ID NO: 52 (or at nucleotide 51 of a sequence comprising at least 95% sequence identity to SEQ ID NO: 52);
f) the CX or CC genotype for the Single Nucleotide Polymorphism marker SNP_53 at nucleotide 51 of SEQ ID NO: 53 (or at nucleotide 51 of a sequence comprising at least 95% sequence identity to SEQ ID NO: 53);
g) the CX or CC genotype for the Single Nucleotide Polymorphism marker SNP_54 at nucleotide 51 of SEQ ID NO: 54 (or at nucleotide 51 of a sequence comprising at least 95% sequence identity to SEQ ID NO: 54);
h) the CX or CC genotype for the Single Nucleotide Polymorphism marker SNP_55 at nucleotide 51 of SEQ ID NO: 55 (or at nucleotide 51 of a sequence comprising at least 95% sequence identity to SEQ ID NO: 55);
i) the GX or GG genotype for the Single Nucleotide Polymorphism marker SNP_56 at nucleotide 51 of SEQ ID NO: 56 (or at nucleotide 51 of a sequence comprising at least 95% sequence identity to SEQ ID NO: 56);
j) the CX or CC genotype for the Single Nucleotide Polymorphism marker SNP_57 at nucleotide 51 of SEQ ID NO: 57 (or at nucleotide 51 of a sequence comprising at least 95% sequence identity to SEQ ID NO: 57);
k) the TX or TT genotype for the Single Nucleotide Polymorphism marker SNP_58 at nucleotide 51 of SEQ ID NO: 58 (or at nucleotide 51 of a sequence comprising at least 95% sequence identity to SEQ ID NO: 58);
l) the TX or TT genotype for the Single Nucleotide Polymorphism marker SNP_59 at nucleotide 51 of SEQ ID NO: 59 (or at nucleotide 51 of a sequence comprising at least 95% sequence identity to SEQ ID NO: 59);
m) the GX or GG genotype for the Single Nucleotide Polymorphism marker SNP_60 at nucleotide 51 of SEQ ID NO: 60 (or at nucleotide 51 of a sequence comprising at least 95% sequence identity to SEQ ID NO: 60);
n) the AX or AA genotype for the Single Nucleotide Polymorphism marker SNP_61 at nucleotide 51 of SEQ ID NO: 61 (or at nucleotide 51 of a sequence comprising at least 95% sequence identity to SEQ ID NO: 61);
o) the AX or AA genotype for the Single Nucleotide Polymorphism marker SNP_62 at nucleotide 51 of SEQ ID NO: 62 (or at nucleotide 51 of a sequence comprising at least 95% sequence identity to SEQ ID NO: 62).


SEQUENCE LISTING (62 sequences)

SEQ ID NO: 1 - DNA, 101 bp, Cucumis sativus
aacacaaaaatgaagatatgaagttgatgaattccatctgctgaaataaatgaagctttaccctgtaaaacacaatcaatcatcatcaccacattattatg

SEQ ID NO: 2 - DNA, 101 bp, Cucumis sativus
tttaatcaaatccctagctagggattcatttacaaccaagtgacatgtaatgtaatgttctattttgatccatacatgtagcaaagggttagggtgattta

SEQ ID NO: 3 - DNA, 101 bp, Cucumis sativus
tgagatggtaaattaaaactaatataaaatcctaaatataagtaacaaaatggtattacaactttataaccataaaattatcataggaaccaaatttttag

SEQ ID NO: 4 - DNA, 101 bp, Cucumis sativus
aaacagcagctcaaaactcagcttcttcatcatcttctaattaaagactttgatttcttctttgtataaccaatcaaacccaatcttcttttggtttgtgg

SEQ ID NO: 5 - DNA, 101 bp, Cucumis sativus
actatttcttgcagtgtttagtataatttattggaaaattgcaatggacgactattttagcaataataattaatgatatagcaacattttaaaaaattgca

SEQ ID NO: 6 - DNA, 101 bp, Cucumis sativus
tattactatttttgctcaacttcatttcaaacaaactcaaacggatcttactaaaataagatgattctctctctcaaattcaactagttatcaagcaaaat

SEQ ID NO: 7 - DNA, 101 bp, Cucumis sativus
ttgtttatttagaagactttaacaaaaatcaatttatttagaagacctaatattgaatcatatatacaaactaattatattaagtatggaataatgaggcc

SEQ ID NO: 8 - DNA, 101 bp, Cucumis sativus
ataatcaaatctaaacaatcgtgtactaattcaaacaattaaatctaaataatcgtgtaccaagtctgatcgtttaccaaaaaaaatcttgaaaaacatct

SEQ ID NO: 9 - DNA, 101 bp, Cucumis sativus
acaaacaagaaacaacaactacaaattcgggtcctcaaatcaataatgctgagtattgaagaaaaaatacaacaatttgctccactcaagaaaaatattgc

SEQ ID NO: 10 - DNA, 101 bp, Cucumis sativus
aatattgtaagtaaaatggacattcagcactataattaagaaacaattaactaatcaaatttaataaatgcatagttgcatcctctttttaacctttttct

SEQ ID NO: 11 - DNA, 101 bp, Cucumis sativus
tatcaaatacaaagtaagttgtatccacaatgttaccaggataaggtacccagccttatccttatattatagaccctttaagctaatcttgaacatagatc

SEQ ID NO: 12 - DNA, 101 bp, Cucumis sativus
tctttcccctgtccaaacaggaaatgacatttttttagctatcaatacaaccaaagattaatgcactgtaccataatccttgagagtgatcctaatgaaat

SEQ ID NO: 13 - DNA, 101 bp, Cucumis sativus
atcagtaacgttgcctatggatcccaatctaaaactctcaaaagatgaagtaaaattcattgttgacgaggatatcacttgctacacaagattgattggaa

SEQ ID NO: 14 - DNA, 101 bp, Cucumis sativus
aggaacgtaagtgaaagatcaaaggacattttttttttcttttatccgataagcataccaaaacaaatctgagaggtacggtaagagtaggtagttcaaat

SEQ ID NO: 15 - DNA, 101 bp, Cucumis sativus
caactatttccctttatattgttctgttttaaaaaaaatatttccctttcctcaatttcctttcctttacatattttaaatgttagtctaaaataaaatga

SEQ ID NO: 16 - DNA, 101 bp, Cucumis sativus
tgaactaatttaaaattttttaaaaatatttaaaattatcagtttggtttgcacccctaatgtatggtaaattatatgaaagaaaaaataatttggttaag

SEQ ID NO: 17 - DNA, 101 bp, Cucumis sativus
cagtgaatagaaatagagaactgaccttgaatagagtcaaccatcgtgatcgagtcctctttccacaaaataaggataccacccgaagaaccataagcttc

SEQ ID NO: 18 - DNA, 101 bp, Cucumis sativus
tgaggaaccagcatctttcttgattttgtcaccatatggaggaaagatgaggcaaatttctgatttggaaactccatttccacatagaaaagggaatactt

SEQ ID NO: 19 - DNA, 101 bp, Cucumis sativus
aactatttaaattaataatttagttgtttttacttccaaactaagttataggttttattactaatattcatatttatattgagatcaatagatgataattt

SEQ ID NO: 20 - DNA, 101 bp, Cucumis sativus
acatatttaaatgttggttggagagaatctatttgttcaattcacttaaatacctaattaaaaattttcacatacaaacaatatttattttttacaaaaat

SEQ ID NO: 21 - DNA, 101 bp, Cucumis sativus
tggggaacactactcggggcctgccacattcatggaaatgttgagcttgcggaagtggcatcaaaacatctatttgatttagaccctttgaactctgggta

SEQ ID NO: 22 - DNA, 101 bp, Cucumis sativus
ggaatcgtatttctaaatggggttttgtgtatgtagagaactttgttttcgatatggggttatcttccaaaaagcaatccattggtttaggaagcattttc

SEQ ID NO: 23 - DNA, 101 bp, Cucumis sativus
ttaaatgaattagtcacccttaagctctcctaaacttgtcagcttctacttggtgatttacatgtatgagcttaatgtcctttctaattatgcttagctta

SEQ ID NO: 24 - DNA, 101 bp, Cucumis sativus
ttataaaacataacactttaagatatggtaattgaatagacacataaaaccaaaaaggtttaaccaaaatatgtattactttatctgccactaaattaagt

SEQ ID NO: 25 - DNA, 101 bp, Cucumis sativus
gctaataaactcgtataatttatcgttgatagaccaatttttacaacataatttattagtgatagactttatcattgatagaatttgacaaattttgctat

SEQ ID NO: 26 - DNA, 101 bp, Cucumis sativus
aaagggagttcatggttttcaaagtcacacataccaagtgaatcttggagcagttgtgtgagtttactgttcctataagggacgtgtgcattccttagagc

SEQ ID NO: 27 - DNA, 101 bp, Cucumis sativus
ttccttctctcacttgtaagatttcttttattattattacaaacaaaaatctaagtttttcttctttcttttttcccttcttcccaccatggtccattgct

SEQ ID NO: 28 - DNA, 101 bp, Cucumis sativus
aaaatgttacaattttaccattgacatctagattttatttcaatttggtccctagattacatgtcaatctcaaattttcacgaaatacttattaatgttaa

SEQ ID NO: 29 - DNA, 101 bp, Cucumis sativus
tatactaaaatatttaccgtatataattaaattttaaatgttatcataacctttgttatattttataaatattttgttaaatttgtgatttttgataattt

SEQ ID NO: 30 - DNA, 101 bp, Cucumis sativus
gggctctaagtttagttatgaagtttggattggaaatttgtgttaggacaatggaataagttagacaattgtgttatgacaatgcaatgagttagatattt

SEQ ID NO: 31 - DNA, 101 bp, Cucumis sativus
cgttggtttgaattatatagtctattttttaaaaaaatagttggatcatagaataaattaatattttggaattacatctaaaaagtaaaaatgaagagaat

SEQ ID NO: 32 - DNA, 101 bp, Cucumis sativus
cattggatttgtagccaaaacgtagtatctgaagttattggtatgccgcatatataaattcattagtttgcaccatgcacaagggggacaaatataatttt

SEQ ID NO: 33 - DNA, 101 bp, Cucumis sativus
gattcattctctctttttttatttttaaacatatttccctccagaaaatgtacaaggtgggagaaatttggatgtgacaatttccacaatctatcactcag

SEQ ID NO: 34 - DNA, 101 bp, Cucumis sativus
agatctagctcatgacattttgtaacttccaaaatttgaacgtcaaatcccaaagtaatgatatagttgtttgatgtgatttagtgtgatcctacatctgt

SEQ ID NO: 35 - DNA, 101 bp, Cucumis sativus
ggaaaccctacttttagctttggcttgaagtccttataaaaataaataaaaaaataaaataaaatagctttggcttgaaatccaaggttgggatgccagag

SEQ ID NO: 36 - DNA, 101 bp, Cucumis sativus
aatgcaaagagtataattcttagtcaagttgtgagagaaaaacatgtttttaaagggttaattttcataaatataataaatcggaaaatatttatgactcg

SEQ ID NO: 37 - DNA, 101 bp, Cucumis sativus
cattcagacatgattggaggcataatccatagttgacagagtggtatgttcagagttacatcatctggcatcatcaaggtagtggctaaattcgccaatgc

SEQ ID NO: 38 - DNA, 101 bp, Cucumis sativus
gattttaaatccataaatctattaaagaaaactgggggtaaattttttttgactccataatggtaacaaatagagtcaattaaaaaaattaaaatatttat

SEQ ID NO: 39 - DNA, 101 bp, Cucumis sativus
agaaatttagaaagaaggaaaagaaaaaacccttcctttctcattccatccatcgcttctgctatacaaattgaaaactatggcttttctcttccatagaa

SEQ ID NO: 40 - DNA, 101 bp, Cucumis sativus
atgaaaatgaaatggttcatcctctttacatcaaattcacaatcttgtaagactaaaattatggtggagggactacggtggtttttcttctccataatttt

SEQ ID NO: 41 - DNA, 101 bp, Cucumis sativus
tttatctacggtcttatatgttcaactcctcaaatggaaaaatgcaaggctttgttgttcttgtttttgttcatatatatcttcatggaagataaggtttt

SEQ ID NO: 42 - DNA, 101 bp, Cucumis sativus
tttcaagaacattcgaaaaagaaacttgctccacggtgcttatagataagaacaaaaatggaatgttaaaagaaacaataaaatattagttagtaattatt

SEQ ID NO: 43 - DNA, 101 bp, Cucumis sativus
atatttgttataatgtgtcatttccaataatttccttttttcatagtattttttttttctactaggatgagtcatagttttttctagttttcttacctaaa

SEQ ID NO: 44 - DNA, 101 bp, Cucumis sativus
aaccaaatagatttattggaaaaacaattaagatatattagtgcacctcctgatcttgttctcctgcttgttacttaacctatcagtgcaccaagactaat

SEQ ID NO: 45 - DNA, 101 bp, Cucumis sativus
actttcaaattttgtttcttttataatctatacaatgggaatgagttggtgcacccaattatcctaattagattaacataatctcatcacccttctcatcc

SEQ ID NO: 46 - DNA, 101 bp, Cucumis sativus
aaaattaaagaaaataatcgaaaggtccattttcggtgaacatttttactataagaactaaaacaaaccgagtaaaatgaaccaaaatcaagagtataaag

SEQ ID NO: 47 - DNA, 101 bp, Cucumis sativus
aaggggaaatggtgttacctgtatttcaatcaaattgcaaagaatgcaaagaatgcaagaatgggaggaccaacaaatgcagtgtttttgggggcaaaact

SEQ ID NO: 48 - DNA, 101 bp, Cucumis sativus
tttggcgctaccgtcgtacaccacgaacaagccccactcgacccctaggcatgggctatttttatgtgtcatacaatgtgtaatatatatgggttgccaca

SEQ ID NO: 49 - DNA, 101 bp, Cucumis sativus
ttaactcggtttggaatacagttttgggcttttagattcacaaaattccacgcccattacgccctttatggatgaaaaaattcgaattggaattaagtgac

SEQ ID NO: 50 - DNA, 101 bp, Cucumis sativus
ctgccaaagtgcggttgtagaaggaagataaatatttgaccctctcttgactgggaaatgaagttgtggaagaccataaagttgaaaagaagaaatggtgg

SEQ ID NO: 51 - DNA, 101 bp, Cucumis sativus
aactacaatcactttgattattttaatctgtattttttgagaaataatcccttgttcgtcttgttggacaatttcttgtatttaaaaaattattgaaaata

SEQ ID NO: 52 - DNA, 101 bp, Cucumis sativus
attgttattctgaagcctggtcctgataagatgtgcgtccatgaagagcattggaaaaattcctcagggaatttaggagaaagagttagtattgaagattt

SEQ ID NO: 53 - DNA, 101 bp, Cucumis sativus
cgccatgtgtccaaaaggtctctgcatcgccgggtcggggttatgtggatctggatttctgaggcgatgcggttcggatctaacatttgggaaccctaacg

SEQ ID NO: 54 - DNA, 101 bp, Cucumis sativus
atcttcattttttctttttcttttatgatttacatctatcaaacaattttccgtttagttcattcacaaaagaaaaagggtcttctgttcttggttttttt

SEQ ID NO: 55 - DNA, 101 bp, Cucumis sativus
cagagtaattttttcaattgtttgataagcaacactagtgataaaaaagaccacttactagttgtgaatttgatagaattgctaatcaaatttgtgaatat

SEQ ID NO: 56 - DNA, 101 bp, Cucumis sativus
accaaatttcattggaatacaaataaacatttggggaagatatattgaaggttgtagtgaaggtggacaaaaacaaaaagtttaatataaatttatttgga

SEQ ID NO: 57 - DNA, 101 bp, Cucumis sativus
ctcctaaattataataaagagggttttggaagacatgggagggagaggttcagtatttgcttcctcttacacccaaacagctttttcttggtccctgtttc

SEQ ID NO: 58 - DNA, 101 bp, Cucumis sativus
ctcttatgatttatcagtgatagatcaatatttccaacacgatctattagtgataaacttctatcattgttagattttgacagaatttgttatatttgtaa

SEQ ID NO: 59 - DNA, 101 bp, Cucumis sativus
gttcgataccatgttaaattaagttttcattgaatgaatgaaagtaaatttgataacatgaatcaatactcaaacaaaagagatatatttctagagaggta

SEQ ID NO: 60 - DNA, 101 bp, Cucumis sativus
aacttctaccaacttctatcagtgtcacttaagggtgagttagccgatcggcattaacaaagtggtcagacgaagataaggaaacgttcaacttattcaaa

SEQ ID NO: 61 - DNA, 101 bp, Cucumis sativus
tacactaacatcttcgtgaaatgaattctctttactaccaagctcaaggcacttgattttttcctgaaactcagcacccattctaacatgagaatccatgt

SEQ ID NO: 62 - DNA, 101 bp, Cucumis sativus
accaatttttaaaagtaatctaaaattgccaatagcttcaattgcaactgatttgccattgcccacatagatgaatctttcagcatcatttggcggtcgac